(19) United States
(12) Patent Application Publication
(10) Pub. No.: US 2023/0084927 A1
(43) Pub. Date: Mar. 16, 2023

(54) GLUCOSE UPTAKE INHIBITORS AND USES THEREOF

(71) Applicant: METABOMED LTD., Yavne (IL)

(72) Inventors: Philippe NAKACHE, Ness Ziona (IL); Omri EREZ, Rehovot (IL); Simone BOTTI, Rehovot (IL); Andreas GOUTOPOULOS, Boston, MA (US)

(73) Assignee: METABOMED LTD., Yavne (IL)

(21) Appl. No.: 17/771,820

(22) PCT Filed: Nov. 11, 2020
(86) PCT No.: PCT/IL2020/051171

§ 371 Date: Apr. 26, 2022

(30) Foreign Application Priority Data
Nov. 13, 2019 (IL) 270639

(51) Int. Cl. A61K 31/519; C07D 495/22; C07D 495/14; A61K 45/06; A61K 31/5377; A61P 35/00

(52) U.S. Cl. CPC A61K 31/519; C07D 495/22; C07D 495/14; A61K 45/06; A61K 31/5377; A61P 35/00

(57) ABSTRACT

The present invention relates to novel compounds that modulate cellular glucose uptake by affecting various targets, including, but not limited to those related to glycolysis and known transporters/co-transporters of the GLUT family. The compounds according to the invention are useful for treating cancer such as: neuroendocrine neoplasms, gastrointestinal stromal tumors (GIST), renal cell carcinoma, paraganglioma, pheochromocytoma, pituitary adenoma, colorectal cancer, lung cancer, gastric cancer, pancreatic cancer sarcoma, head and neck cancer, melanoma, ovarian cancer and other cancers that rely on high levels of glycolysis for survival and proliferation; as well as in treating of autoimmune diseases, inflammation, infectious diseases, and metabolic diseases.

### FIELD OF THE INVENTION

The present invention relates to novel compounds that modulate cellular glucose uptake by affecting various targets, including, but not limited to those related to glycolysis and known transporters/co-transporters of the GLUT family. The compounds according to the invention are useful for treating cancer such as: neuroendocrine neoplasms, gastrointestinal stromal tumors (GIST), renal cell carcinoma, paraganglioma, pheochromocytoma, pituitary adenoma, colorectal cancer, lung cancer, gastric cancer, pancreatic cancer sarcoma, head and neck cancer, melanoma, ovarian cancer and other cancers that rely on high levels of glycolysis for survival and proliferation; as well as in treating of autoimmune diseases, inflammation, infectious diseases, and metabolic diseases.

### BACKGROUND OF THE INVENTION

Glucose is a central nutrient for many organisms and cell types. Control of glucose signaling and consumption is tightly regulated. Accordingly, many disease states are associated with defects in this regulation and therefore may be susceptible to therapeutic intervention using glucose uptake inhibitors, or other ways to affect glucose metabolism. Glucose uptake inhibitors or other inhibitors of glycolysis may have utility in disease areas such as oncology, autoimmunity and inflammation, infection diseases/virology, and metabolic disease.

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). The rate of new cancer cases decreased by an average 0.6% per year among men between 2000 and 2009 and stayed the same for women. From 2000 through 2009, death rates from all cancers combined decreased on average 1.8% per year among men and 1.4% per year among women. This improvement in survival reflects progress in diagnosing at an earlier stage and improvements in treatment. Discovering highly effective anticancer agents with low toxicity is a primary goal of cancer research.

Inactivating mutations in the TCA cycle enzymes succinate dehydrogenase (SDH) and fumarate hydratase (FH) result in a “broken TCA cycle” phenotype characterized by the accumulation of oncometabolites succinate and fumarate, respectively. Germline and sporadic homozygous loss of these enzymes' function and tumor suppressive activity is associated with susceptibility to various types of cancers. These tumors are generally refractory to conventional targeted therapeutic approaches and often affect young patients. Specifically, SDH mutations have been linked to different types of hereditary and sporadic forms of cancer, including hereditary paraganglioma and pheochromocytoma (PGC/PCC), renal carcinoma, gastrointestinal stromal tumor (GIST) and others.

Succinate dehydrogenase (SDH), also known as succinate ubiquinone oxydoreductase, is a mitochondrial enzyme complex composed of four subunits: SDHA, SDHB, SDHC, and SDHD, that functions both in the mitochondrial tricarboxylic acid (TCA) cycle, the Krebs cycle, and in the electron transport chain enzyme complex, mitochondrial complex II. SDH is responsible for the oxidation of succinate to fumarate. Over the past 18 years, oncogenic mutations in three TCA-cycle-related enzymes, succinate dehydrogenase (SDH), Fumarate hydratase (FH) and Isocitrate dehydrogenase (IDH) have been identified. Inactivating mutations in any of the SDH subunits, or the SDH complex assembly factor (SDHAF2), are associated with susceptibility to develop neuroendocrine neoplasms and gastrointestinal stromal tumors as well as renal cell carcinoma. Loss of function of the SDH complex characterizes a rare group of human tumors including some gastrointestinal stromal tumors (SDH-deficient “KIT wild-type” GIST), paragangliomas, renal carcinomas and pituitary adenomas. These tumors are generally refractory to conventional targeted therapeutic approaches.

Inactivating germline missense mutations of the genes that encode the SDH subunits are also found to cause familial pheochromocytoma and familial paraganglioma. These SDH gene alterations, lead to the loss of enzyme activity and expression, and observed in several tumors such as renal cell carcinoma, gastrointestinal stromal tumors, colorectal cancer, gastric cancer, and ovarian cancer. Thus, the SDH subunit genes are considered to be tumor suppressor genes.

Pheochromocytoma (PCC or PHEO) and paraganglioma (PGL) are neuroendocrine tumors arising in the adrenal medulla and in paraganglia of the autonomous nervous system, respectively. Most present as benign, yet show high morbidity and mortality due to excessive catecholamine production, leading to hypertension, arrhythmia, and stroke. Up to 25% are malignant, as defined by distant metastases to non-chromaffin tissues. Since the original discovery of SDHD mutations in hereditary PGL in 2000, the genes encoding all four proteins that constitute the succinate dehydrogenase (SDH) complex as well as the required assembly factor (SDH assembly factor 2: SDHAF2) have been shown as tumor-suppressor genes in familial and apparently sporadic PCC and PGL. Interestingly, SDHB mutation carriers were specifically shown to be predisposed to malignant and particularly aggressive forms of the disease. The effects of all these mutations are to abolish SDH activity, which results in high steady-state intracellular concentrations of Succinate. Patients with metastatic PCCs/PGLs have limited treatment options and poor prognosis, often with less than 50% surviving at 5 years. Despite a low incidence (0.8 per 100,000 for PCCs), over one-third of PCCs/PGLs are associated with inherited cancer susceptibility syndromes, which is the highest rate among all tumor types.

Sporadic SDH-deficient phenotype is also appearing in other type of cancers such as metastatic Leiomyosarcoma with SDHA deficiency, bladder paragangliomas (Mason et al., Identification of succinate dehydrogenase-deficient bladder paragangliomas. Am J Surg Pathol. 2013) and pancreatic neuroendocrine tumors (Niemeijer et al. Succinate dehydrogenase (SDH)-deficient pancreatic neuroendocrine tumor expands the SDH-related tumor spectrum. J. Clin. Endocrinol. Metab. 2015). In addition, many tumors rely on aerobic glycolysis as their energy source rather than the much more efficient oxidative phosphorylation pathway. This phenomenon that called the Warburg effect make these tumors dependent on the supply of glucose by the cellular glucose transporters (GluTs).

Loss of SDH causes succinate accumulation in cells, which activates hypoxia-inducible factors at normal oxygen level and inhibits α-ketoglutarate-dependent histone and DNA demethylases, thereby establishing a pseudohypoxic and hypermethylator phenotype in tumors.

These finding have demonstrated once again that mutations in metabolic genes such SDHx are leading to rewiring metabolic pathways to support the growth of the cancer cells. These mutations may lead to dependencies of the cancer cells on other metabolic pathways/proteins that are serving as synthetic lethal pair with the cancerous mutation and can be exploit for targeting specifically the tumor cells.

Glucose uptake inhibitors and other compounds that are targeting SDH-deficient cells and glycolytic tumor types may also be useful as anti-inflammatory agents. The current assumption is that activated macrophages produce Itaconate that in turn inhibits SDH and lead to succinate accumulation (“Itaconate Links Inhibition of Succinate Dehydrogenase with Macrophage Metabolic Remodeling and Regulation of Inflammation”. Lampropoulou et al., 2016, Cell Metabolism 24, 1-9). Hence, it is possible that compounds that are able to target SDH-deficient cells will eliminate also activated macrophages and could potentially be used as specific anti-inflammatory treatment.

### SUMMARY OF THE INVENTION

In various embodiments, this invention relates to a compound represented by the structure of any one of formulas I-IX, or its pharmaceutically acceptable salt, optical isomer, tautomer, hydrate, N-oxide, pharmaceutical product, prodrug, isotopic variant (e.g. deuterated analogs), PROTAC or any combination thereof. In other embodiments, this invention relates to a compound represented by the structure of compounds 100-335 as presented herein below in Table 1, or its pharmaceutically acceptable salt, optical isomer, tautomer, hydrate, N-oxide, pharmaceutical product, prodrug, isotopic variant (e.g. deuterated analogs), PROTAC or any combination thereof. In various embodiments, the compound destroys SDH-deficient cells. In various embodiments, the compound is a glucose uptake inhibitor. In various embodiments, the compound is selective to cells with broken TCA cycle. In various embodiments, the broken TCA cycle is genetic or chemically induced. In various embodiments, the compound inhibits a GluT receptor. In various embodiments, the compound is selective to Class I GluT receptors. In various embodiments, the compound is selective to GluT1, GluT2, GluT3, GluT4, or any combination thereof. In various embodiments, the compound inhibits all classes of GluT receptors. In various embodiments, the compound inhibits Class I, Class II, Class III GluT receptor, or any combination thereof; each represents a separate embodiment according to this invention.

In various embodiments, this invention relates to a method of treating, suppressing, reducing the severity, reducing the risk of developing or inhibiting cancer comprising administering a compound according to this invention to a subject suffering from cancer under conditions effective to treat, suppress, reduce the severity, reduce the risk of developing, or inhibit said cancer. In other embodiments, the cancer is selected from the list of: renal cell carcinoma, leiomyosarcoma, gastrointestinal stromal cancer, paraganglioma (e.g., bladder paraganglioma), pituitary adenoma, pheochromocytoma, colorectal cancer, gastric cancer, ovarian cancer, or in general, cancers characterized by high glycolytic rate. In other embodiments, the cancer is familial, sporadic, early cancer, advanced cancer, invasive cancer, metastatic cancer, drug resistant cancer or any combination thereof.

In various embodiments, this invention relates to a method of suppressing, reducing or inhibiting tumor growth in a subject, comprising administering a compound according to this invention, to a subject suffering from tumor growth under conditions effective to suppress, reduce or inhibit said tumor growth in said subject. In other embodiments, the tumor is selected from the list of: gastrointestinal stromal tumor, pheochromocytoma, pituitary adenoma, pheochromocytoma, leiomyoma (e.g., uterine fibroids), pancreatic neuroendocrine tumor and paraganglioma. In other embodiments, the tumor is benign, invasive, malignant, cancerous, carcinoma, familial, sporadic, or any combination thereof. In other embodiments, the tumor cells have a broken TCA cycle. In other embodiments, the tumor growth is stimulated by SDH-deficient cells, by SDH deactivating mutation or combination thereof. In other embodiments, the tumor growth is stimulated by a broken TCA cycle.

In various embodiments, this invention relates to a method of treating, suppressing, reducing the severity, reducing the risk of developing or inhibiting SDH-associated neoplasms comprising administering the compound according to this invention to a subject suffering from SDH-associated neoplasms under conditions effective to treat, suppress, reduce the severity, reduce the risk of developing, or inhibit the SDH-associated neoplasms in said subject. In other embodiments, the neoplasms are neuroendocrine neoplasms.

In various embodiments, this invention relates to a method of treating, suppressing, reducing the severity, reducing the risk of developing or inhibiting gastrointestinal stromal tumors (GIST) comprising administering a compound according to this invention to a subject suffering from gastrointestinal stromal tumors under conditions effective to treat, suppress, reduce the severity, reduce the risk of developing, or inhibit the gastrointestinal stromal tumors.

In various embodiments, this invention relates to a method of treating, suppressing, reducing the severity, reducing the risk of developing or inhibiting renal cell carcinoma comprising administering a compound according to this invention to a subject suffering from renal cell carcinoma under conditions effective to treat, suppress, reduce the severity, reduce the risk of developing, or inhibit the renal cell carcinoma.

In various embodiments, this invention relates to a method of treating, suppressing, reducing the severity, reducing the risk of developing or inhibiting paraganglioma comprising administering a compound according to this invention to a subject suffering from paraganglioma under conditions effective to treat, suppress, reduce the severity, reduce the risk of developing, or inhibit the paraganglioma. In other embodiments, the paraganglioma is familial. In other embodiments, the paraganglioma is sporadic. In other embodiments, the paraganglioma is benign. In other embodiments, the paraganglioma is malignant. In other embodiments, the paraganglioma is bladder paraganglioma.

In various embodiments, this invention relates to a method of treating, suppressing, reducing the severity, reducing the risk of developing or inhibiting pheochromocytoma (PHEO) comprising administering a compound according to this invention to a subject suffering from pheochromocytoma under conditions effective to treat, suppress, reduce the severity, reduce the risk of developing, or inhibit the pheochromocytoma. In other embodiments, the pheochromocytoma is familial pheochromocytoma. In other embodiments, the pheochromocytoma is sporadic pheochromocytoma. In other embodiments, the pheochromocytoma is benign. In other embodiments, the pheochromocytoma is malignant.

In various embodiments, this invention relates to a method of treating, suppressing, reducing the severity, reducing the risk of developing or inhibiting pituitary adenoma comprising administering a compound according to this invention to a subject suffering from pituitary adenoma under conditions effective to treat, suppress, reduce the severity, reduce the risk of developing, or inhibit the pituitary adenoma. In other embodiments, the pituitary adenoma is benign. In other embodiments, the pituitary adenoma is malignant.

In various embodiments, this invention relates to a method of treating, suppressing, reducing the severity, reducing the risk of developing or inhibiting pituitary adenoma comprising administering a compound according to this invention to a subject suffering from pituitary adenoma under conditions effective to treat, suppress, reduce the severity, reduce the risk of developing, or inhibit the pituitary adenoma. In other embodiments, the pituitary adenoma is benign. In other embodiments, the pituitary adenoma is malignant.

In various embodiments, this invention relates to a method of treating, suppressing, reducing the severity, reducing the risk of developing or inhibiting colorectal cancer comprising administering a compound according to this invention to a subject suffering from colorectal cancer under conditions effective to treat, suppress, reduce the severity, reduce the risk of developing, or inhibit the colorectal cancer.

In various embodiments, this invention relates to a method of treating, suppressing, reducing the severity, reducing the risk of developing or inhibiting gastric cancer comprising administering a compound according to this invention to a subject suffering from gastric cancer under conditions effective to treat, suppress, reduce the severity, reduce the risk of developing, or inhibit the gastric cancer.

In various embodiments, this invention relates to a method of treating, suppressing, reducing the severity, reducing the risk of developing or inhibiting ovarian cancer comprising administering a compound according to this invention to a subject suffering from ovarian cancer under conditions effective to treat, suppress, reduce the severity, reduce the risk of developing, or inhibit the ovarian cancer.

In various embodiments, this invention relates to a method of treating, suppressing, reducing the severity, reducing the risk of developing or inhibiting Leiomyosarcoma comprising administering a compound according to this invention to a subject suffering from Leiomyosarcoma under conditions effective to treat, suppress, reduce the severity, reduce the risk of developing, or inhibit the Leiomyosarcoma.

In other embodiments, the subject has been previously treated with chemotherapy, immunotherapy, radiotherapy, biological therapy, surgical intervention, or any combination thereof. In other embodiments, the compound is administered in combination with an anti-cancer therapy. In other embodiments, the anti-cancer therapy is chemotherapy, immunotherapy, radiotherapy, biological therapy, surgical intervention, or any combination thereof. In other embodiments, the subject has advanced cancer, metastatic cancer, drug resistant cancer or any combination thereof. In other embodiments, the compound destroys SDH-deficient cells.

In various embodiments, this invention relates to a method of treating, suppressing, reducing the severity, reducing the risk of developing or inhibiting inflammation comprising administering a compound according to this invention to a subject suffering from inflammation under conditions effective to treat, suppress, reduce the severity, reduce the risk of developing, or inhibit the inflammation.

In various embodiments, this invention relates to a method of treating, suppressing, reducing the severity, reducing the risk of developing or inhibiting an autoimmune disease or disorder comprising administering a compound according to this invention to a subject suffering from an autoimmune disease or disorder under conditions effective to treat, suppress, reduce the severity, reduce the risk of developing, or inhibit the autoimmune disease or disorder. In some embodiments, the autoimmune disease or disorder is Guillain-Barré syndrome (GBS). In some embodiments, the autoimmune disease or disorder is Systemic lupus erythematosus (SLE). In some embodiments, the autoimmune disease or disorder is Rheumatoid arthritis (RA).

In various embodiments, this invention relates to a method of treating, suppressing, reducing the severity, reducing the risk of developing or inhibiting a parasitic or viral infection comprising administering a compound according to this invention to a subject suffering from a parasitic or viral infection under conditions effective to treat, suppress, reduce the severity, reduce the risk of developing, or inhibit the a parasitic or viral infection. In various embodiments, the parasitic infection is caused by malaria parasite. In various embodiments, the viral infection is caused by HIV or by human cytomegalovirus.

In various embodiments, this invention relates to a method of treating, suppressing, reducing the severity, reducing the risk of developing or inhibiting diabetes mellitus comprising administering a compound according to this invention to a subject suffering from diabetes mellitus under conditions effective to treat, suppress, reduce the severity, reduce the risk of developing, or inhibit the diabetes mellitus.

In various embodiments, this invention relates to a method of treating, suppressing, reducing the severity, reducing the risk of developing or inhibiting diabetic retinopathy comprising administering a compound according to this invention to a subject suffering from diabetic retinopathy under conditions effective to treat, suppress, reduce the severity, reduce the risk of developing, or inhibit the diabetic retinopathy.

In various embodiments, this invention relates to a method of treating, suppressing, reducing the severity, reducing the risk of developing or inhibiting diabetic nephropathy comprising administering a compound according to this invention to a subject suffering from diabetic nephropathy under conditions effective to treat, suppress, reduce the severity, reduce the risk of developing, or inhibit the diabetic nephropathy.

In various embodiments, this invention relates to a method of treating, suppressing, reducing the severity, reducing the risk of developing or inhibiting a metabolic disease comprising administering a compound according to this invention to a subject suffering from a metabolic disease under conditions effective to treat, suppress, reduce the severity, reduce the risk of developing, or inhibit the metabolic disease.

In various embodiments, this invention relates to a method of inhibiting SDH-deficient tumor growth, comprising providing a compound according to this invention, and contacting an SDH-deficient tumor with the compound, under conditions effective to inhibit the growth of said tumor.

In various embodiments, this invention relates to a pharmaceutical composition comprising a compound according to this invention and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is general synthetic schemes for compounds of the invention. FIG. 1A depicts a general synthesis for compounds with no middle scaffold substitutions. FIG. 1B and FIG. 1C depict a general synthesis for compounds with middle scaffold substitutions.

FIG. 2 depicts the activity of compound 145 with and without the Oxidative Phosphorylation (OxPhos) inhibitor Rotenone in SDH-deficient/proficient cells. A synergistic effect of compound 145 with OxPhos inhibitor Rotenone is demonstrated in SDH-proficient cells. FIG. 2A depicts synergistic effect for compound 145 with Rotenone in SDH-proficient cells. FIG. 2B depicts no effect for Rotenone in SDH-deficient cells.

FIG. 3 depicts the differential activity of compound 111 in 3 different SDH-proficient/deficient isogenic pairs. FIG. 3A: Murine SDH-WT/KO HRAS transformed, SDH-WT are SDH proficient cells; SDH-KO-HRAS-C17 are SDH deficient cells; FIG. 3B: UOK269, Human Tumor-derived SDHB-deficient RCC, UOK-269 WT are SDH proficient cells; UOK-269 are SDH deficient cells; and FIG. 3C: ACHN, RCC cells, SDHB-KO (CRISPR), ACHN+/+ are SDH proficient cells; ACHN−/− are SDH deficient cells. The results shown in the figures serve as a validation that the compound is active against different SDH-deficient cells.

FIG. 4 depicts the differential toxicity in SDH-WT/SDH-KO cells of compound 139, compound 111, compound 144 and compound 145.

FIG. 5 depicts experiments performed to deconvolute the mechanism of action of compounds described herein. Compound 145 effect on proliferation in a cancer cell line panel. Wide range of activities is demonstrated for the cancer cells. The arrow in the right side points on peripheral blood mononuclear cells (PBMC) as normal cell control. Strongest effect is shown in the cancer cell lines on the left side.

FIG. 6A depicts the inhibition of lactate secretion from SDH-KO cells by compound 145. SDH-KO cells were treated with different concentrations of compound 145 for 30 min together with (U-13C)-Glucose. Lactate labeling from (U-13C)-Glucose (Lactate m+3) was determined by LC-MS analysis and was presented as percent of the total lactate in the medium. The results show that compound 145 inhibits lactate secretion to the medium suggesting inhibition of glycolysis upstream of lactate secretion. FIG. 6B depicts that compound 145 inhibits 2-Deoxiglucose (2 DG) phosphorylation but not Hexokinase 1 and 2 activity.

FIG. 7 depicts the increased Glucose uptake's IC₅₀ and GluT1/3/4 mRNA expression level in SDH-KO resistant cells. SDH-KO cells were treated with increasing concentrations of Compound 145 for about 2 months. SDH-KO cells—regular SDH knockout cells; SDH-KO Res145: SDH-KO cells that are resistant to 20 nM of Compound 145. FIG. 7A: Seven-fold higher IC₅₀ is demonstrated in the resistant cells compare to the regular SDH-KO cells. FIG. 7B: Higher expression level of GluTs in the resistant cells. A quantity analysis of GluT1/3/4 mRNA expression level show 4-8 fold elevation in the resistant cells. Glut1 expression level is significantly higher, in both the normal and resistant cells, than of Glut3/4.

FIG. 8 depicts that compound 111 elevates plasma glucose level in mice. Oral administration in mice of compound 111 at 2 mg/Kg lead to an increase in level of glucose in the plasma as is expected for compound that inhibits glucose uptake by the cells.

FIG. 9 depicts the glucose levels as measured in mice after different dosages of compound 111 at different diets. Ketogenic diet doubled the Maximum Tolerated Dose (MTD) and delayed elevation in plasma glucose levels after compound 111 treatment.

FIG. 10 depicts the tumor volume vs. time in mice on ketogenic diet, treated for two weeks post-injection, but prior to tumor formation. Pre-treatment with compound 111 delays tumor onset and growth.

FIG. 11 depicts a differential toxicity assay of several compounds according to this invention in SDH-WT and SDH-KO cells: FIG. 11A: Compound 332; FIG. 11B: Compound 331; and FIG. 11C: Compound 330.

FIG. 12 depicts the differential activity (EC₅₀) of Compound 145 and the selective GluT1i BAY-876 in 3 distinct SDH-deficient/proficient isogenic pairs: Murine SDHB-WT/KO, HRAS transformed isogenic pair (FIG. 12A); UOK269, Human Tumor-derived SDHB-deficient RCC in their WT counterpart with re-expression of SDHB (FIG. 12B); and ACHN, RCC cells, and their SDHB-KO (SDHB CRISPRed out) isogenic cells (FIG. 12C). The results shown in the figures serve as a validation that the compound is active against different SDH-deficient cells, which rely heavily on glycolysis for proliferation and survival.

FIG. 13 depicts the Glucose uptake assay with Compound 145, in DLD1 cells with different concentrations of glucose. As the glucose concentration in the assay is rising, the inhibition decreases, which suggests that Compound 145 is a Glucose-competitive Glucose uptake inhibitor

FIG. 14 depicts Compound 332 PK profile. FIG. 14A depicts the Mean Plasma Concentration of Compound 332 after IP7, IV bolus1 and PO2 Dosing. FIG. 14B depicts the Mean Brain Concentration of Compound 332 after IP Dosing at 2.50 mg/kg and FIG. 14C depicts the Mean Brain Concentration of Compound 332 after IV bolus Dosing at 2.50 mg/kg.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In various embodiments, this invention is directed to a compound represented by the structure of formula (I):

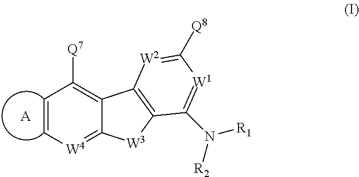

wherein

A is absent or is a substituted or unsubstituted C₃-C₁₀ cycloalkene (e.g., cyclohexene, cyclopentene) or a substituted or unsubstituted C₃-C₁₀ heterocyclic ring (e.g., dihydropyran, tetrahydropyran, tetrahydropyridine), (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ and Q⁸ are each independently H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O-cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 3, 4, and 5);

k is an integer number between 1 and 20 (e.g., 7, 11, and 15);

R₁ and R₂ are each independently H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁);

or R₁ and R₂ are joined to form a 5 or 6 membered substituted or unsubstituted, heterocyclic ring (e.g., pyrrolidine, morpoline, piperazine, piperidine, 4-(3-fluoro-4-methoxyphenyl)-1-piperidine, 2-(piperazin-1-yl)ethanol)

wherein substitutions include: F, Cl, Br, I, substituted or unsubstituted C₁-C₅ linear or branched alkyl, substituted or unsubstituted C₃-C₈ carbocyclic or heterocyclic ring (e.g., furan, tetrahydrofuran, morpholin), aryl, benzyl, OH, alkoxy, N(R)₂, CF₃, CN or NO₂;

R₈ is [CH₂]ₚ
wherein p is between 1 and 10 (e.g., 2);

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylethanesulfonamide, 4-N-methyl-N-phenylethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₁₀ (e.g., C(O)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH₂—O(C═O)—CH₃, CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

W¹, W² and W⁴ are each independently CH, C(R) or N;

W³ is S, SO, SO₂, O, N—OH, CH₂, C(R)₂ or N—OMe;

or its pharmaceutically acceptable salt, stereoisomer, tautomer, hydrate, N-oxide, isotopic variant, pharmaceutical product or any combination thereof.

In some embodiments, A is a substituted or unsubstituted cyclohexene ring. In some embodiments, A is cyclohexene ring. In some embodiments, A is 4-ethoxycyclohex-1-ene. In some embodiments, A is a substituted or unsubstituted cyclopentene ring. In some embodiments, A is cyclopentene ring. In some embodiments, A is a substituted or unsubstituted dihydropyran ring. In some embodiments, A is 2,2-dimethyl-3,6-dihydro-2H-pyran. In some embodiments, A is a substituted or unsubstituted tetrahydropyridine ring. In some embodiments, A is 1-methyl-1,2,3,6-tetrahydropyridine. In some embodiments, A is absent. In some embodiments, A is substituted with one or more selected from: F, Cl, Br, I, C₁-C₅ linear or branched alkyl, OH, alkoxy (e.g., ethoxy), O(C═O)—R₁₀, COOH, N(R)₂, CF₃, CN or NO₂; each substitution represents a separate embodiment according to this invention.

In some embodiments, R₁ is H. In other embodiments, R₁ is C₁-C₅ linear or branched alkyl. In other embodiments, R₁ is methyl. In other embodiments, R₁ is C(O)—R₁₀. In other embodiments, R₁ is C(O)—CH₃.

In some embodiments, R₂ is R₁₂, and R₁₂ is C₁-C₅ linear or branched alkyl. In other embodiments, R₁ is R₈-R₁₂, R₈ is (CH₂)ₚ wherein p is 1, 2 or 3; each is a separate embodiment according to the invention. In some embodiments, R₁₂ is substituted or unsubstituted single or fused aryl. In some embodiments, the aryl is 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, or benzene-1,2-diamine; each is a separate embodiment according to this invention. In some embodiments, R₁₂ is substituted or unsubstituted single or fused C₃-C₁₂ heterocyclic ring. In some embodiments, the substituted or unsubstituted single or fused C₃-C₁₂ heterocyclic ring is 1H-benzo[d]imidazol-2(3H)-one. In some embodiments, R₁₂ is a substituted or unsubstituted single or fused heteroaryl. In some embodiments, the heteroaryl is benzimidazolyl or benzooxazolyl. In some embodiments, the heteroaryl is 1-methyl-benzimidazolyl, 2-methyl-1H-benzo[d]imidazole, 2-methoxy-1H-benzo[d]imidazole; each is a separate embodiment according to this invention. In some embodiments, the aryl, C₃-C₁₂ heterocyclic ring or heteroaryl of R₁₂ is substituted. In some embodiments, the substitution is one or more selected from: F, Cl, Br, I, substituted or unsubstituted C₁-C₅ linear or branched alkyl (e.g. methyl, isopropyl, CH₂—OH, CH(OH)—CH₃), R₁₀ (e.g., C(O)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH₂—O(C═O)—CH₃, CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂, each represents a separate embodiment according to this invention.

In some embodiments, R₁ and R₂ are joined to form a 5 or 6 membered substituted or unsubstituted, heterocyclic ring. In some embodiments, R₁ and R₂ are joined to form a morpoline. In some embodiments, R₁ and R₂ are joined to form a piperazine. In some embodiments, R₁ and R₂ are joined to form a piperidine. In some embodiments, R₁ and R₂ are joined to form a 4-(3-fluoro-4-methoxyphenyl)-1-piperidine.

In some embodiments, W³ is S. In some embodiments, W⁵ is SO. In some embodiments, W⁵ is SO₂.

In various embodiments, this invention is directed to a compound represented by the structure of formula (II)

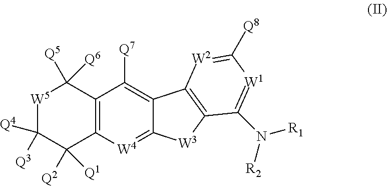

wherein

Q¹, Q², Q³, Q⁴, Q⁵, Q⁶, and Q⁸ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring, substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

R₁ and R₂ are each independently H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁),

or R₁ and R₂ are joined to form a 5 or 6 membered substituted or unsubstituted, heterocyclic ring (e.g., pyrrolidine, morpoline, piperazine, piperidine, 4-(3-fluoro-4-methoxyphenyl)-1-piperidine, 2-(piperazin-1-yl)ethanol)

wherein substitutions include: F, Cl, Br, I, substituted or unsubstituted C₁-C₅ linear or branched alkyl, substituted or unsubstituted C₃-C₈ carbocyclic or heterocyclic ring (e.g., furan, tetrahydrofuran, morpholin), aryl, benzyl, OH, alkoxy, N(R)₂, CF₃, CN or NO₂;

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

W¹, W² and W⁴ are each independently CH, C(R) or N;

W³ is S, SO, SO₂, O, N—OH, CH₂, C(R)₂ or N—OMe;

W⁵ is a bond, S, O, NH, N(R) (e.g., N—CH₃), N(R)₂, CH₂, CH(R), C(R)₂, N—OH, or N—OR;

or its pharmaceutically acceptable salt, stereoisomer, tautomer, hydrate, N-oxide, isotopic variant, pharmaceutical product or any combination thereof.

In some embodiments, W³ is S. In some embodiments, W⁵ is SO. In some embodiments, W⁵ is SO₂.

In various embodiments, this invention is directed to a compound represented by the structure of formula (III)

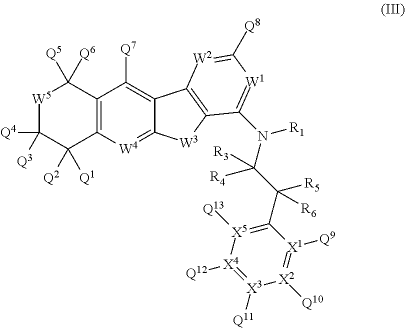

wherein

Q¹, Q², Q³, Q⁴, Q⁵, Q⁶, and Q⁸ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl (wherein substitutions include: F, Cl, Br, I, C₁-C₅ linear or branched alkyl, OH, alkoxy, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₁ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(Rn);

R₃, R₄, R₅ and R₆ are each independently H, C₁-C₅ linear or branched alkyl (e.g., methyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁) —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁);

or R₃ and R₄ are joined to form a 5 or 6 membered substituted or unsubstituted, carbocyclic or heterocyclic ring (e.g., cyclopentyl);

or R₅ and R₆ are joined to form a 5 or 6 membered substituted or unsubstituted, carbocyclic or heterocyclic ring (e.g., cyclopentyl); R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

W¹, W² and W⁴ are each independently CH, C(R) or N;

W³ is S, SO, SO₂, O, N—OH, CH₂, C(R)₂ or N—OMe;

W⁵ is a bond, S, O, NH, N(R) (e.g., N—CH₃), N(R)₂, CH₂, CH(R), C(R)₂, N—OH, or N—OR;

X¹, X², X³, X⁴ and X⁵ are each independently C or N,
wherein
if X¹, X², X³, X⁴ and/or X⁵ is N then Q⁹, Q¹⁰, Q¹¹, Q¹² and/or Q¹³ is absent respectively;

or its pharmaceutically acceptable salt, stereoisomer, tautomer, hydrate, N-oxide, isotopic variant, pharmaceutical product or any combination thereof.

In some embodiments, R₃ is H. In other embodiments, R₃ is C₁-C₅ linear or branched alkyl. In other embodiment, R₃ is methyl.

In some embodiments, R₄ is H. In other embodiments, R₄ is C₁-C₅ linear or branched alkyl. In other embodiment, R₄ is methyl.

In some embodiments, R₅ is H. In other embodiments, R₅ is C₁-C₅ linear or branched alkyl. In other embodiment, R₅ is methyl.

In some embodiments, R₆ is H. In other embodiments, R₆ is C₁-C₅ linear or branched alkyl. In other embodiment, R₆ is methyl.

In various embodiments, this invention is directed to a compound represented by the structure of formula (IV)

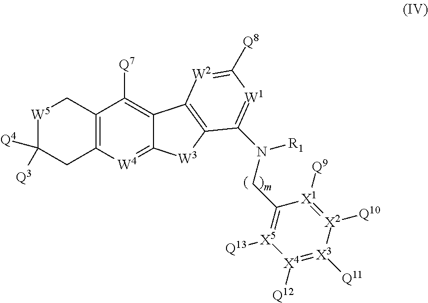

wherein

Q³, Q⁴, and Q⁸ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₁ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁),

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

W¹, W² and W⁴ are each independently CH, C(R) or N;

W³ is S, SO, SO₂, O, N—OH, CH₂, C(R)₂ or N—OMe;

W⁵ is a bond, S, O, NH, N(R) (e.g., N—CH₃), N(R)₂, CH₂, CH(R), C(R)₂, N—OH, or N—OR;

m is an integer between 0 and 5 (e.g., 1, 2);

X¹, X², X³, X⁴ and X⁵ are each independently C or N,
wherein
if X¹, X², X³, X⁴ and/or X⁵ is N then Q⁹, Q¹⁰, Q¹¹, Q¹² and/or Q¹³ is absent respectively;

or its pharmaceutically acceptable salt, stereoisomer, tautomer, hydrate, N-oxide, isotopic variant, pharmaceutical product or any combination thereof.

In various embodiments, this invention is directed to a compound represented by the structure of formula (V)

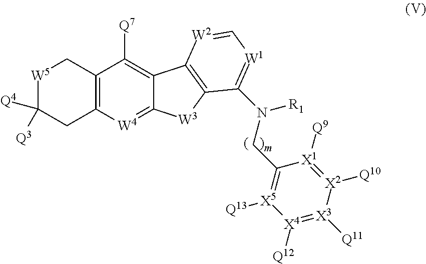

wherein

Q³ and Q⁴ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring, substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₁ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(Rn);

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); Rn is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

W¹, W² and W⁴ are each independently CH, C(R) or N;

W³ is S, SO, SO₂, O, N—OH, CH₂, C(R)₂ or N—OMe;

W⁵ is a bond, S, O, NH, N(R) (e.g., N—CH₃), N(R)₂, CH₂, CH(R), C(R)₂, N—OH, or N—OR;

m is an integer between 0 and 5;

X¹, X², X³, X⁴ and X⁵ are each independently C or N,
wherein
if X¹, X², X³, X⁴ and/or X⁵ is N then Q⁹, Q¹⁰, Q¹¹, Q¹² and/or Q¹³ is absent respectively;

or its pharmaceutically acceptable salt, stereoisomer, tautomer, hydrate, N-oxide, isotopic variant, pharmaceutical product or any combination thereof.

In various embodiments, this invention is directed to a compound represented by the structure of formula (VI)

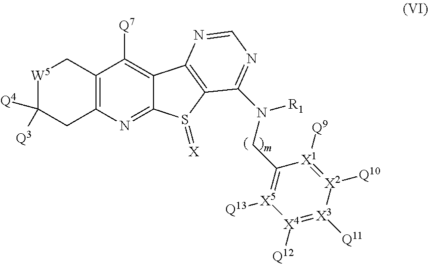

wherein

Q³ and Q⁴ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted C₃-C₈ heterocyclic ring, substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₁ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, (wherein substitutions include: F, Cl, Br, I, C₁-C₅ linear or branched alkyl, OH, alkoxy, N(R)₂, CF₃, CN or NO₂); F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholin, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁),

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), (wherein substitutions include: F, Cl, Br, I, substituted or unsubstituted C₁-C₅ linear or branched alkyl (e.g. methyl, isopropyl, CH₂—OH, CH(OH)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂); C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;

R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

S═X is S, S═O or SO₂;

W⁵ is a bond, S, O, NH, N(R) (e.g., N—CH₃), N(R)₂, CH₂, CH(R), C(R)₂, N—OH, or N—OR;

m is an integer between 0 and 5 (e.g., 1, 2);

X¹, X², X³, X⁴ and X⁵ are each independently C or N,
wherein
if X¹, X², X³, X⁴ and/or X⁵ is N then Q⁹, Q¹⁰, Q¹¹, Q¹² and/or Q¹³ is absent respectively;

or its pharmaceutically acceptable salt, stereoisomer, tautomer, hydrate, N-oxide, isotopic variant, pharmaceutical product or any combination thereof.

In various embodiments, this invention is directed to a compound represented by the structure of formula (VII):

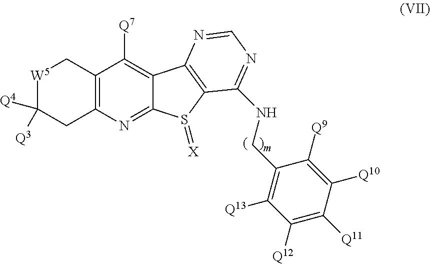

wherein

Q³ and Q⁴ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring, substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

S═X is S, S═O or SO₂;

W⁵ is a bond, S, O, NH, N(R) (e.g., N—CH₃), N(R)₂, CH₂, CH(R), C(R)₂, N—OH, or N—OR;

m is an integer between 0 and 5;

or its pharmaceutically acceptable salt, stereoisomer, tautomer, hydrate, N-oxide, isotopic variant, pharmaceutical product or any combination thereof.

In various embodiments, this invention is directed to a compound represented by the structure of formula (VIII):

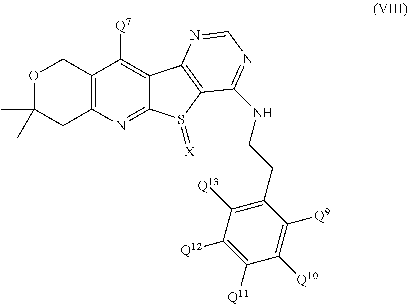

wherein

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O-CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

S═X is S, S═O or SO₂;

or its pharmaceutically acceptable salt, stereoisomer, tautomer, hydrate, N-oxide, isotopic variant, pharmaceutical product or any combination thereof.

In various embodiments, this invention is directed to a compound represented by the structure of formula (IX):

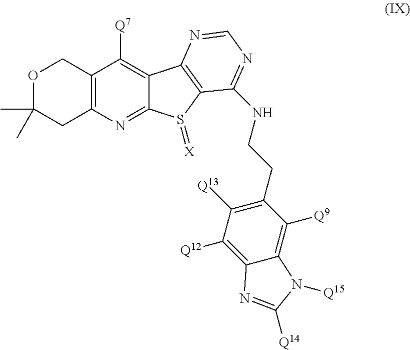

wherein

Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O— cyclopentyl, (CH₂)—O-cyclohexyl, (CH₂)—O-(1-methyl-piperidine), (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl) (wherein substitutions include: F, Cl, Br, I, C₁-C₅ linear or branched alkyl, OH, alkoxy (e.g., ethoxy), O(C═O)—R₁₀, COOH, COO—R₁₀, N(R)₂, CF₃, CN or NO₂);

n is an integer number between 1 and 20 (e.g., 1, 3, 4, and 5);

k is an integer number between 1 and 20 (e.g., 7, 11, and 15);

Q⁹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

Q¹⁴ is H, F, Cl, Br, I, OH, SH, R₈—OH (e.g., CH₂—OH), R₈—SH, —R₈—O—R₁₀, (e.g., —CH₂—O—(C═O)—CH₃, CH₂—O—CH₃), C₁-C₅ linear or branched alkylester (e.g., —CH₂—O—(C═O)—CH₃, CH(CH₃)—O(C═O)—CH₃), CF₃, CN, NO₂, —CH₂CN, —R₈CN, NH₂, NHR, N(R)₂, R₈—N(R₁₀)(R₁₁) (e.g., CH₂—N(CH₃)₂), —OC(O)CF₃, —OC(O)NH₂, —NHC(O)NH₂, —OCH₂Ph, NHC(O)—R₁₀ (e.g., NHC(O)CH₃), NHCO—N(R₁₀)(R₁₁) (e.g., NHC(O)N(CH₃)₂), N(R₁₀)C(O)R (e.g., N(CH₃)C(O)(CH₃)), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), substituted or unsubstituted C₁-C₅ linear or branched alkyl (e.g., methyl, ethyl, propyl, iso-propyl, t-Bu, iso-butyl, pentyl, CH(OH)CH₃), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀)

Q¹⁵ is H, C₁-C₅ linear or branched alkyl (e.g., methyl, ethyl, propyl, iso-propyl, t-Bu, iso-butyl, pentyl), substituted or unsubstituted C₃-C₈ heterocyclic ring (e.g., 1-methyl-pyrrolidine-2-one)

R₈ is [CH₂]ₚ
wherein p is between 1 and 10;

R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;

or R₁₀ and R₁₁ are joined to form a 5 or 6 membered substituted or unsubstituted, carbocyclic or heterocyclic ring (e.g., morpholine); R₁₃ is [(CH₂)₂O]ₙ—CH₃
wherein n is between 1 and 20 (e.g., 1, 3, 4, 5);

R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;

wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;

S═X is S, S═O or SO₂;

or its pharmaceutically acceptable salt, stereoisomer, tautomer, hydrate, N-oxide, isotopic variant, pharmaceutical product or any combination thereof.

In some embodiments, Q¹⁴ is H. In some embodiments, Q¹⁴ is OH. In some embodiments, Q¹⁴ is R₈—OH. In some embodiments, Q¹⁴ is CH₂—OH. In some embodiments, Q¹⁴ is —R₈—O—R₁₀. In some embodiments, Q¹⁴ is C₁-C₅ linear or branched alkylester. In some embodiments, Q¹⁴ is —CH₂—O—(C═O)—CH₃. In some embodiments, Q¹⁴ is CH(CH₃)—O(C═O)—CH₃. In some embodiments, Q¹⁴ is NO₂. In some embodiments, Q¹⁴ is NH₂. In some embodiments, Q¹⁴ is C(O)—R₁₀. In some embodiments, Q¹⁴ is C(O)—CH₃. In some embodiments, Q¹⁴ is substituted or unsubstituted C₁-C₅ linear or branched alkyl. In some embodiments, Q¹⁴ is methyl. In some embodiments, Q¹⁴ is iso-propyl. In some embodiments, Q¹⁴ is CH(OH)CH₃. In some embodiments, Q¹⁴ is C₁-C₅ linear or branched or C₃-C₈ cyclic alkoxy. In some embodiments, Q¹⁴ is methoxy. In some embodiments, Q¹⁴ is substituted or unsubstituted aryl. In some embodiments, Q¹⁴ is phenyl. In some embodiments, Q¹⁴ is substituted with at least one selected from: F, Cl, Br, I, C₁-C₅ linear or branched alkyl, OH, alkoxy, N(R)₂, CF₃, CN and NO₂; each represents a separate embodiment according to this invention.

In some embodiments, Q¹⁵ is H. In some embodiments, Q¹⁵ is C₁-C₅ linear or branched alkyl. In some embodiments, Q¹⁵ is methyl.

In various embodiments, this invention is directed to the compounds presented in Table 1, pharmaceutical compositions and/or method of use thereof; each is a separate embodiment according to this invention:

TABLE 1

| Compound | |
|---|---|
| No. | Structure |
| 100 | 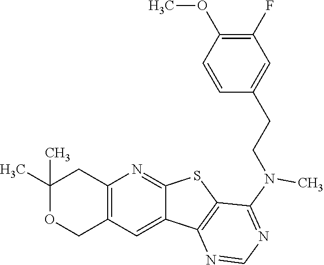 |
| 101 | 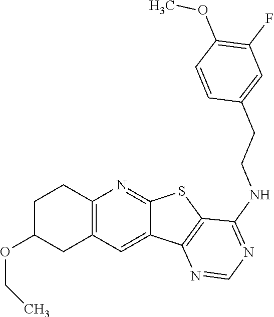 [SMILES: CCOC1CCc2nc3sc4c(NCCc5ccc(OC)c(F)c5)ncnc4c3cc2C1] |
| 102 | 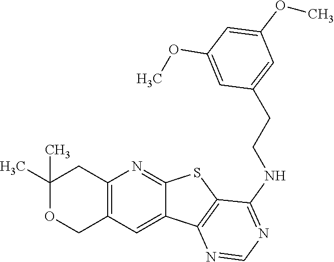 |
| 103 | 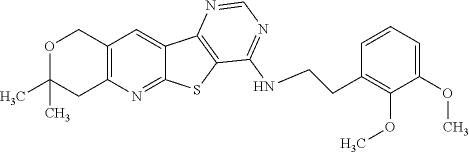 |
| 104 | 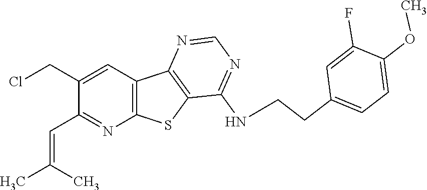 [SMILES: COc1ccc(CCNc2ncnc3c2sc2nc(C=C(C)C)c(CCl)cc23)cc1F] |
| 105 | 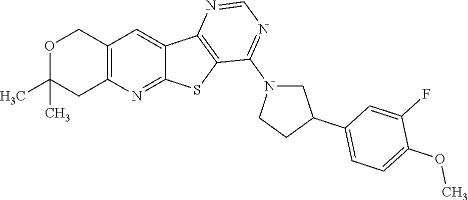 |
| 106 | 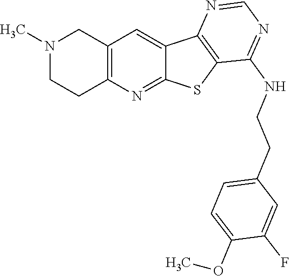 [SMILES: COc1ccc(CCNc2ncnc3c2sc2nc4c(cc23)CN(C)CC4)cc1F] |
| 107 | 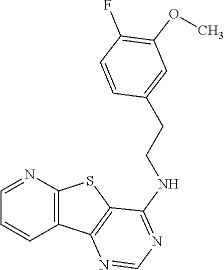 [SMILES: COc1cc(CCNc2ncnc3c2sc2ncccc23)ccc1F] |
| 108 | 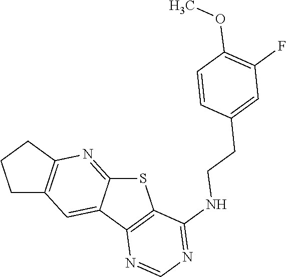 [SMILES: COc1ccc(CCNc2ncnc3c2sc2nc4c(cc23)CCC4)cc1F] |
| 109 | 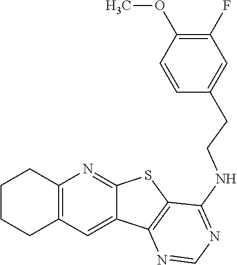 [SMILES: COc1ccc(CCNc2ncnc3c2sc2nc4c(cc23)CCCC4)cc1F] |
| 110 | 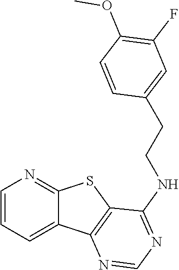 [SMILES: COc1ccc(CCNc2ncnc3c2sc2ncccc23)cc1F] |
| 111 | 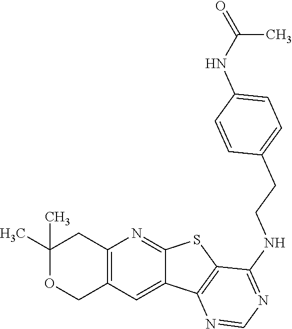 [SMILES: CC(=O)Nc1ccc(CCNc2ncnc3c2sc2nc4c(cc23)COC(C)(C)C4)cc1] |
| 112 | 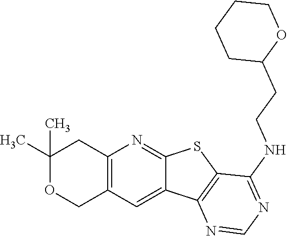 [SMILES: CC1(C)Cc2nc3sc4c(NCCC5CCCCO5)ncnc4c3cc2CO1] |
| 113 | 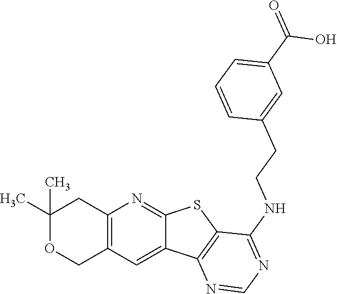 [SMILES: CC1(C)Cc2nc3sc4c(NCCc5cccc(C(=O)O)c5)ncnc4c3cc2CO1] |
| 114 | 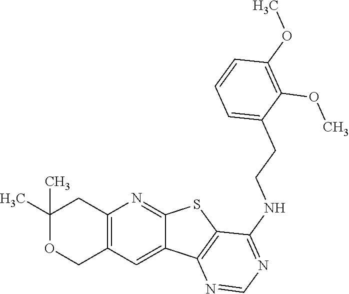 |
| 115 | 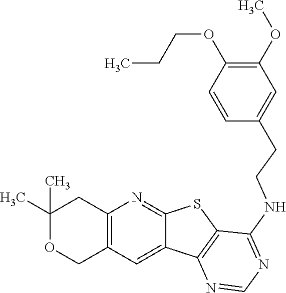 [SMILES: CCCOc1ccc(CCNc2ncnc3c2sc2nc4c(cc23)COC(C)(C)C4)cc1OC] |
| 116 | 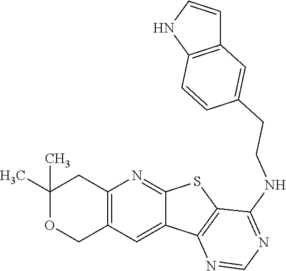 [SMILES: CC1(C)Cc2nc3sc4c(NCCc5ccc6[nH]ccc6c5)ncnc4c3cc2CO1] |
| 117 | 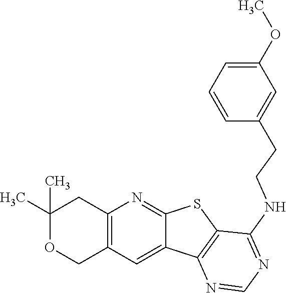 [SMILES: COc1cccc(CCNc2ncnc3c2sc2nc4c(cc23)COC(C)(C)C4)c1] |
| 120 | 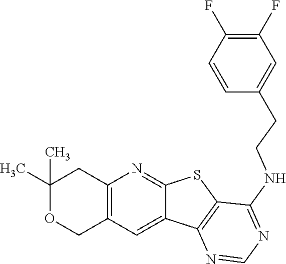 [SMILES: CC1(C)Cc2nc3sc4c(NCCc5ccc(F)c(F)c5)ncnc4c3cc2CO1] |
| 121 | 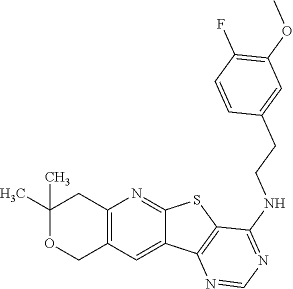 [SMILES: COc1cc(CCNc2ncnc3c2sc2nc4c(cc23)COC(C)(C)C4)ccc1F] |
| 122 | 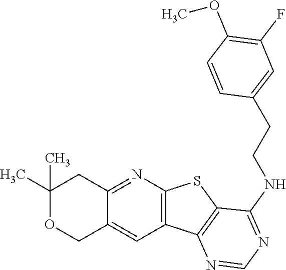 [SMILES: COc1ccc(CCNc2ncnc3c2sc2nc4c(cc23)COC(C)(C)C4)cc1F] |
| 123 | 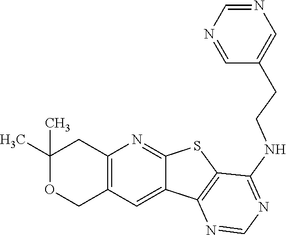 [SMILES: CC1(C)Cc2nc3sc4c(NCCc5cncnc5)ncnc4c3cc2CO1] |
| 124 | 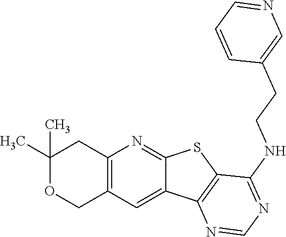 [SMILES: CC1(C)Cc2nc3sc4c(NCCc5cccnc5)ncnc4c3cc2CO1] |
| 125 | 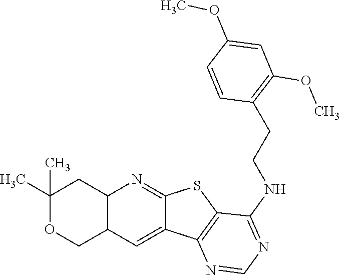 |
| 126 | 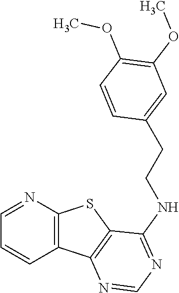 [SMILES: COc1ccc(CCNc2ncnc3c2sc2ncccc23)cc1OC] |
| 127 | 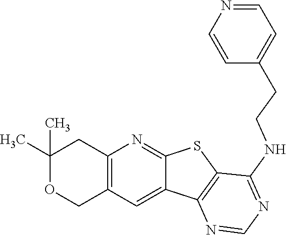 [SMILES: CC1(C)Cc2nc3sc4c(NCCc5ccncc5)ncnc4c3cc2CO1] |
| 128 | 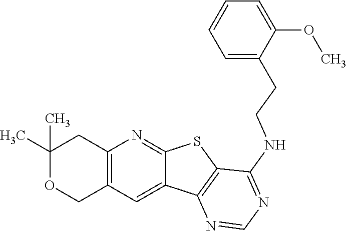 |
| 129 | 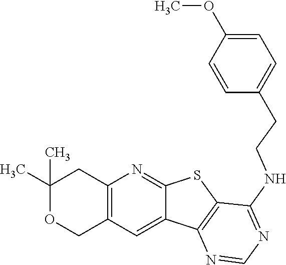 [SMILES: COc1ccc(CCNc2ncnc3c2sc2nc4c(cc23)COC(C)(C)C4)cc1] |
| 130 | 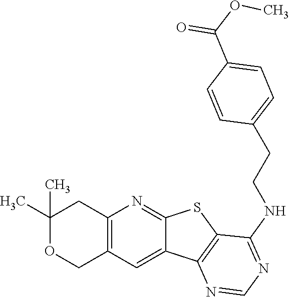 [SMILES: COC(=O)c1ccc(CCNc2ncnc3c2sc2nc4c(cc23)COC(C)(C)C4)cc1] |
| 131 | 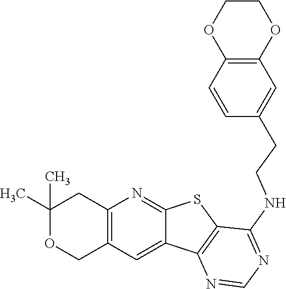 [SMILES: CC1(C)Cc2nc3sc4c(NCCc5ccc6c(c5)OCCO6)ncnc4c3cc2CO1] |
| 132 | 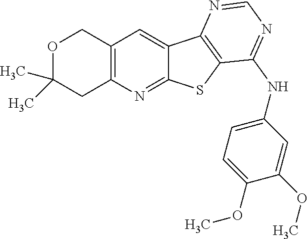 [SMILES: COc1ccc(Nc2ncnc3c2sc2nc4c(cc23)COC(C)(C)C4)cc1OC] |
| 133 | 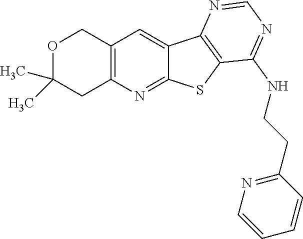 [SMILES: CC1(C)Cc2nc3sc4c(NCCc5ccccn5)ncnc4c3cc2CO1] |
| 134 | 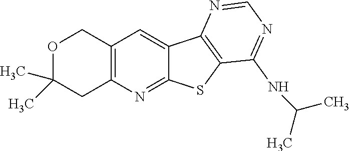 [SMILES: CC(C)Nc1ncnc2c1sc1nc3c(cc12)COC(C)(C)C3] |
| 135 | 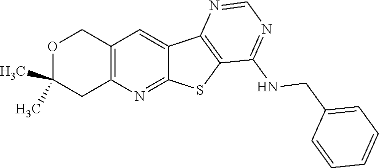 |
| 136 | 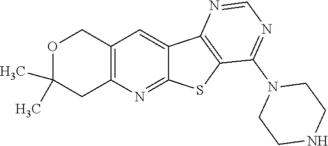 [SMILES: CC1(C)Cc2nc3sc4c(N5CCNCC5)ncnc4c3cc2CO1] |
| 137 | 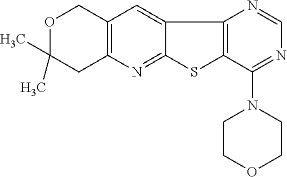 [SMILES: CC1(C)Cc2nc3sc4c(N5CCOCC5)ncnc4c3cc2CO1] |
| 138 | 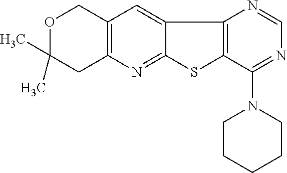 [SMILES: CC1(C)Cc2nc3sc4c(N5CCCCC5)ncnc4c3cc2CO1] |
| 139 | 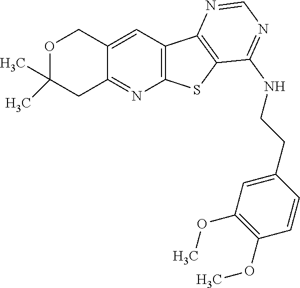 [SMILES: COc1ccc(CCNc2ncnc3c2sc2nc4c(cc23)COC(C)(C)C4)cc1OC] |
| 140 | 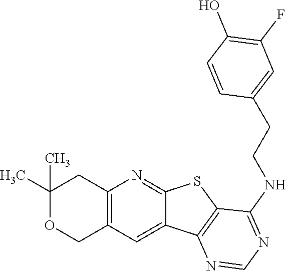 [SMILES: CC1(C)Cc2nc3sc4c(NCCc5ccc(O)c(F)c5)ncnc4c3cc2CO1] |
| 141 | 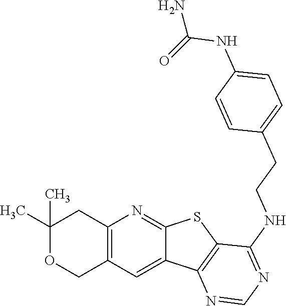 [SMILES: CC1(C)Cc2nc3sc4c(NCCc5ccc(NC(N)=O)cc5)ncnc4c3cc2CO1] |
| 142 | 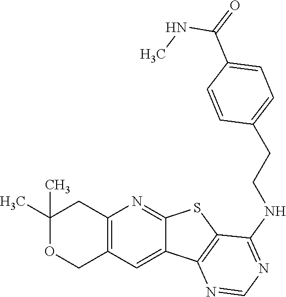 [SMILES: CNC(=O)c1ccc(CCNc2ncnc3c2sc2nc4c(cc23)COC(C)(C)C4)cc1] |
| 143 | 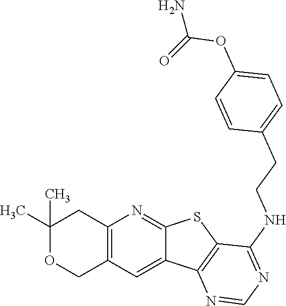 [SMILES: CC1(C)Cc2nc3sc4c(NCCc5ccc(OC(N)=O)cc5)ncnc4c3cc2CO1] |
| 144 | 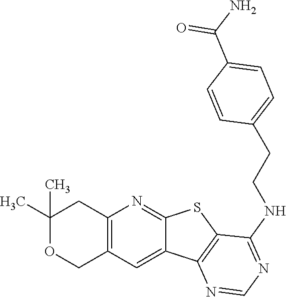 [SMILES: CC1(C)Cc2nc3sc4c(NCCc5ccc(C(N)=O)cc5)ncnc4c3cc2CO1] |
| 145 | 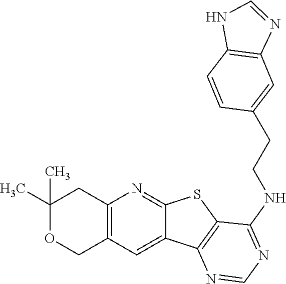 [SMILES: CC1(C)Cc2nc3sc4c(NCCc5ccc6[nH]cnc6c5)ncnc4c3cc2CO1] |
| 146 | 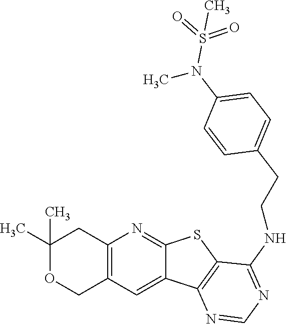 [SMILES: CN(c1ccc(CCNc2ncnc3c2sc2nc4c(cc23)COC(C)(C)C4)cc1)S(C)(=O)=O] |
| 147 | 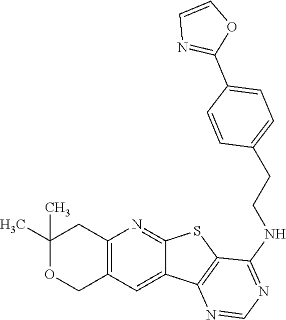 [SMILES: CC1(C)Cc2nc3sc4c(NCCc5ccc(-c6ncco6)cc5)ncnc4c3cc2CO1] |
| 148 | 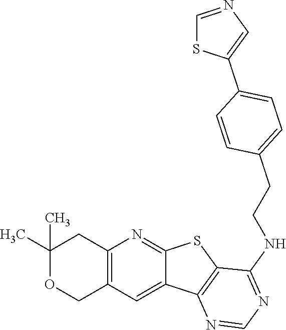 [SMILES: CC1(C)Cc2nc3sc4c(NCCc5ccc(-c6cncs6)cc5)ncnc4c3cc2CO1] |
| 149 | 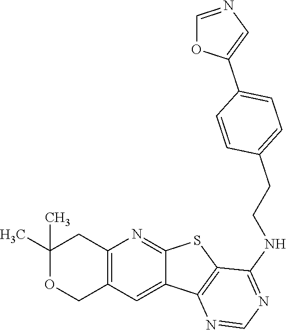 [SMILES: CC1(C)Cc2nc3sc4c(NCCc5ccc(-c6cnco6)cc5)ncnc4c3cc2CO1] |
| 150 | 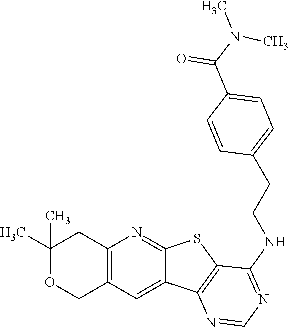 [SMILES: CN(C)C(=O)c1ccc(CCNc2ncnc3c2sc2nc4c(cc23)COC(C)(C)C4)cc1] |
| 151 | 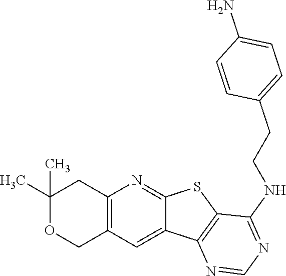 [SMILES: CC1(C)Cc2nc3sc4c(NCCc5ccc(N)cc5)ncnc4c3cc2CO1] |
| 152 | 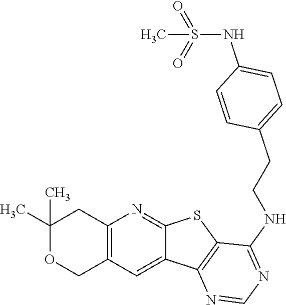 [SMILES: CC1(C)Cc2nc3sc4c(NCCc5ccc(NS(C)(=O)=O)cc5)ncnc4c3cc2CO1] |
| 153 | 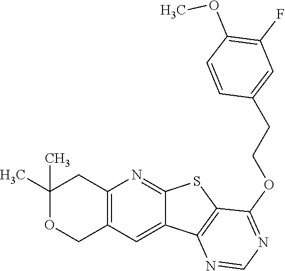 [SMILES: COc1ccc(CCOc2ncnc3c2sc2nc4c(cc23)COC(C)(C)C4)cc1F] |
| 154 | 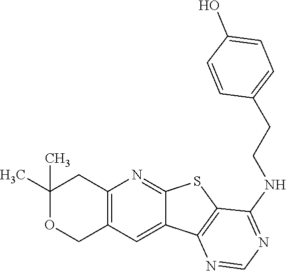 [SMILES: CC1(C)Cc2nc3sc4c(NCCc5ccc(O)cc5)ncnc4c3cc2CO1] |
| 156 | 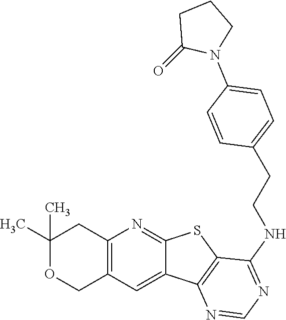 [SMILES: CC1(C)Cc2nc3sc4c(NCCc5ccc(N6CCCC6=O)cc5)ncnc4c3cc2CO1] |
| 157 | 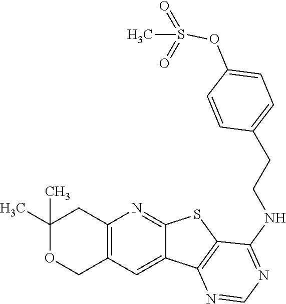 [SMILES: CC1(C)Cc2nc3sc4c(NCCc5ccc(OS(C)(=O)=O)cc5)ncnc4c3cc2CO1] |
| 158 | 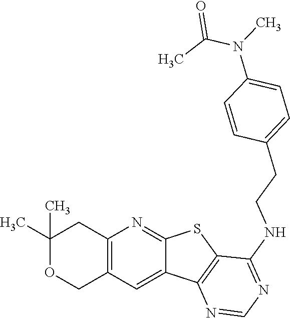 [SMILES: CC(=O)N(C)c1ccc(CCNc2ncnc3c2sc2nc4c(cc23)COC(C)(C)C4)cc1] |
| 159 | 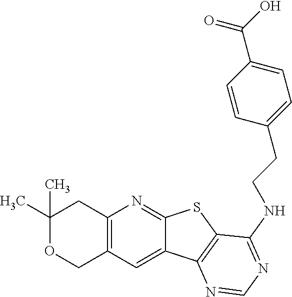 [SMILES: CC1(C)Cc2nc3sc4c(NCCc5ccc(C(=O)O)cc5)ncnc4c3cc2CO1] |
| 160 | 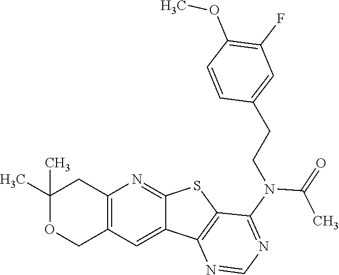 [SMILES: COc1ccc(CCN(C(C)=O)c2ncnc3c2sc2nc4c(cc23)COC(C)(C)C4)cc1F] |
| 161 | 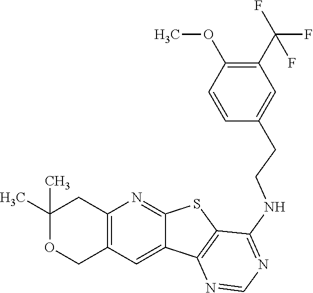 [SMILES: COc1ccc(CCNc2ncnc3c2sc2nc4c(cc23)COC(C)(C)C4)cc1C(F)(F)F] |
| 162 | 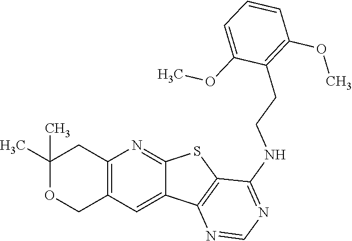 |
| 163 | 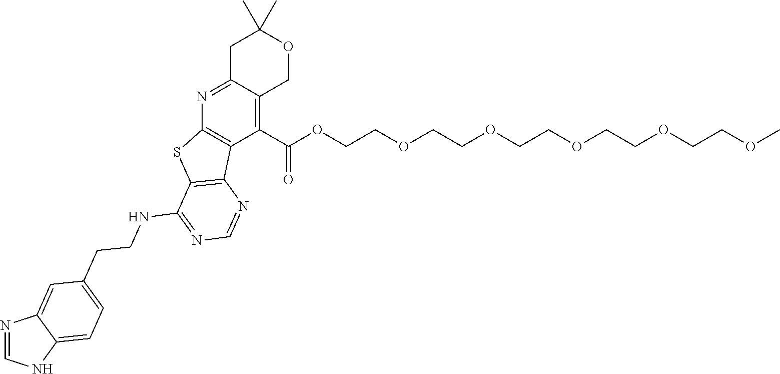 [SMILES: COCCOCCOCCOCCOCCOC(=O)c1c2c(nc3sc4c(NCCc5ccc6[nH]cnc6c5)ncnc4c13)CC(C)(C)OC2] |
| 164 | 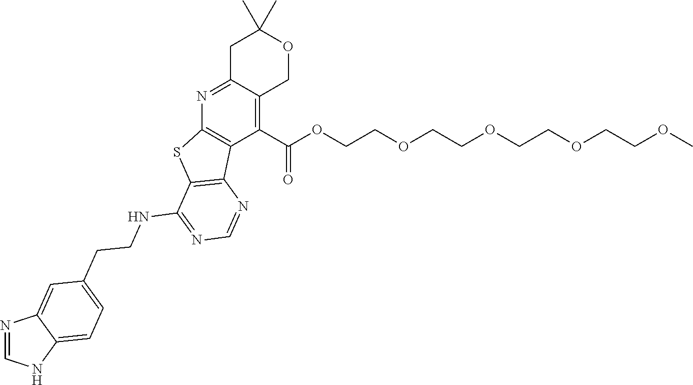 [SMILES: COCCOCCOCCOCCOC(=O)c1c2c(nc3sc4c(NCCc5ccc6[nH]cnc6c5)ncnc4c13)CC(C)(C)OC2] |
| 165 | 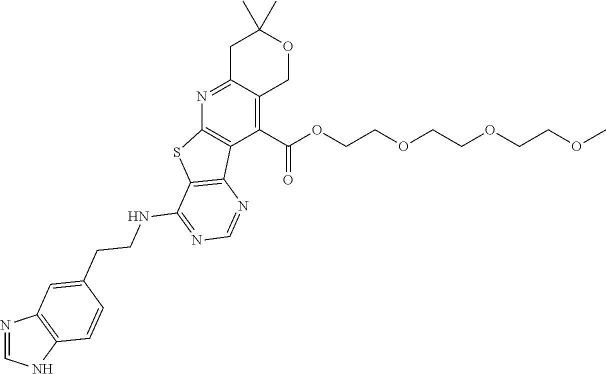 [SMILES: COCCOCCOCCOC(=O)c1c2c(nc3sc4c(NCCc5ccc6[nH]cnc6c5)ncnc4c13)CC(C)(C)OC2] |
| 166 | 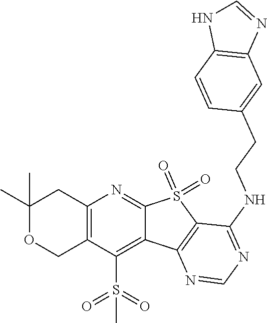 [SMILES: CC1(C)Cc2nc3c(c(S(C)(=O)=O)c2CO1)-c1ncnc(NCCc2ccc4[nH]cnc4c2)c1S3(=O)=O] |
| 167 | 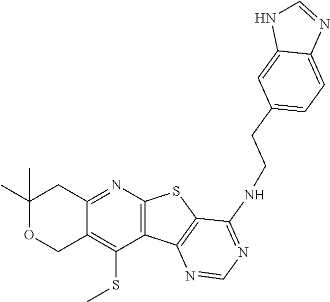 [SMILES: CSc1c2c(nc3sc4c(NCCc5ccc6nc[nH]c6c5)ncnc4c13)CC(C)(C)OC2] |
| 168 | 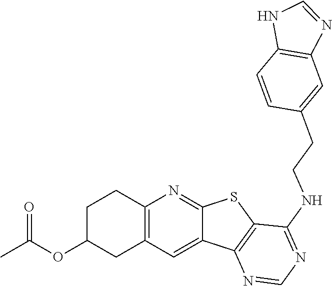 [SMILES: CC(=O)OC1CCc2nc3sc4c(NCCc5ccc6[nH]cnc6c5)ncnc4c3cc2C1] |
| 169 | 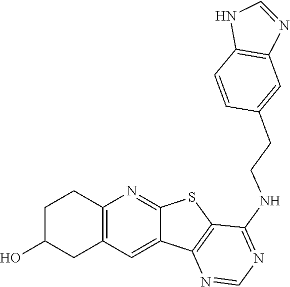 [SMILES: OC1CCc2nc3sc4c(NCCc5ccc6[nH]cnc6c5)ncnc4c3cc2C1] |
| 170 | 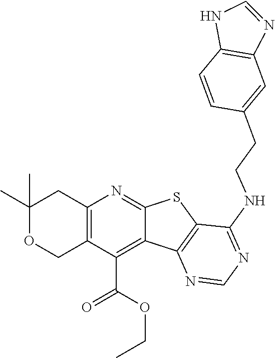 [SMILES: CCOC(=O)c1c2c(nc3sc4c(NCCc5ccc6[nH]cnc6c5)ncnc4c13)CC(C)(C)OC2] |
| 171 | 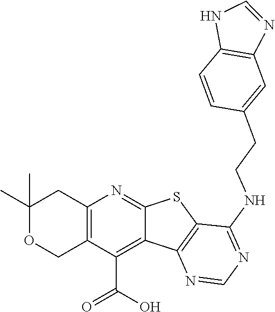 [SMILES: CC1(C)Cc2nc3sc4c(NCCc5ccc6[nH]cnc6c5)ncnc4c3c(C(=O)O)c2CO1] |
| 172 | 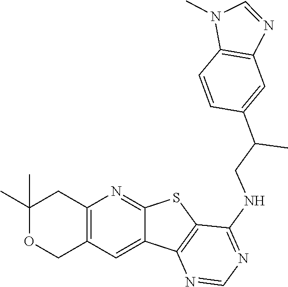 [SMILES: CC(CNc1ncnc2c1sc1nc3c(cc12)COC(C)(C)C3)c1ccc2c(c1)ncn2C] |
| 173 | 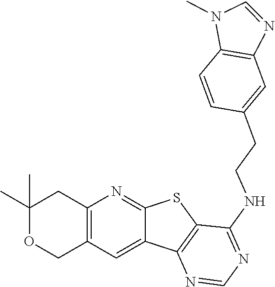 [SMILES: Cn1cnc2cc(CCNc3ncnc4c3sc3nc5c(cc34)COC(C)(C)C5)ccc21] |
| 174 | 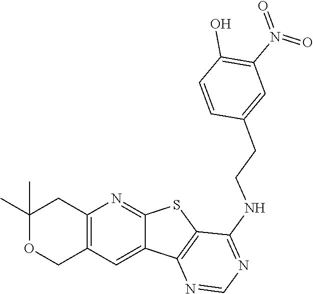 [SMILES: CC1(C)Cc2nc3sc4c(NCCc5ccc(O)c([N+](=O)[O-])c5)ncnc4c3cc2CO1] |
| 175 | 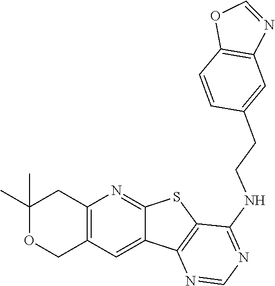 [SMILES: CC1(C)Cc2nc3sc4c(NCCc5ccc6ocnc6c5)ncnc4c3cc2CO1] |
| 176 | 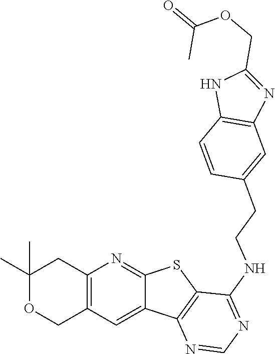 [SMILES: CC(=O)OCc1nc2cc(CCNc3ncnc4c3sc3nc5c(cc34)COC(C)(C)C5)ccc2[nH]1] |
| 177 | 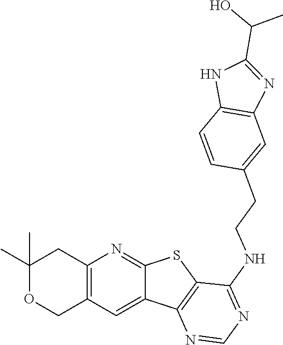 [SMILES: CC(O)c1nc2cc(CCNc3ncnc4c3sc3nc5c(cc34)COC(C)(C)C5)ccc2[nH]1] |
| 178 | 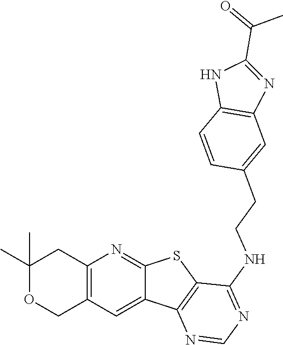 [SMILES: CC(=O)c1nc2cc(CCNc3ncnc4c3sc3nc5c(cc34)COC(C)(C)C5)ccc2[nH]1] |
| 179 | 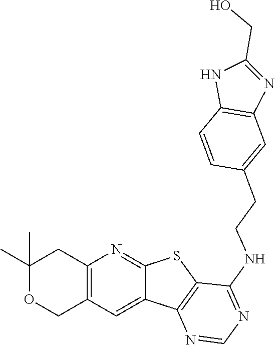 [SMILES: CC1(C)Cc2nc3sc4c(NCCc5ccc6[nH]c(CO)nc6c5)ncnc4c3cc2CO1] |
| 180 | 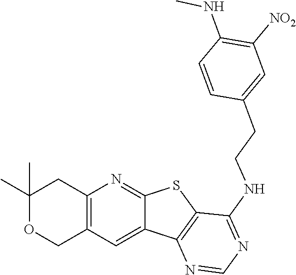 [SMILES: CNc1ccc(CCNc2ncnc3c2sc2nc4c(cc23)COC(C)(C)C4)cc1[N+](=O)[O-]] |
| 181 | 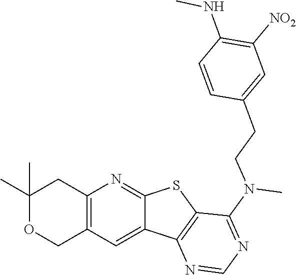 |
| 182 | 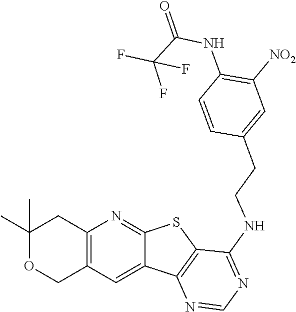 [SMILES: CC1(C)Cc2nc3sc4c(NCCc5ccc(NC(=O)C(F)(F)F)c([N+](=O)[O-])c5)ncnc4c3cc2CO1] |
| 183 | 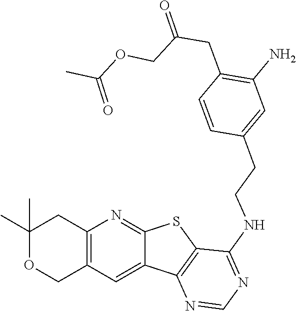 [SMILES: CC(=O)OCC(=O)Cc1ccc(CCNc2ncnc3c2sc2nc4c(cc23)COC(C)(C)C4)cc1N] |
| 184 | 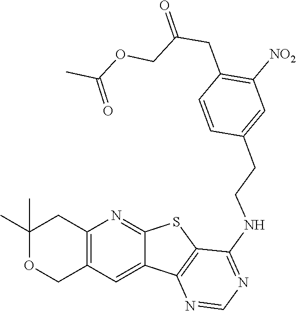 [SMILES: CC(=O)OCC(=O)Cc1ccc(CCNc2ncnc3c2sc2nc4c(cc23)COC(C)(C)C4)cc1[N+](=O)[O-]] |
| 185 | 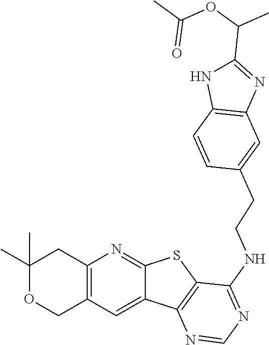 [SMILES: CC(=O)OC(C)c1nc2cc(CCNc3ncnc4c3sc3nc5c(cc34)COC(C)(C)C5)ccc2[nH]1] |
| 186 | 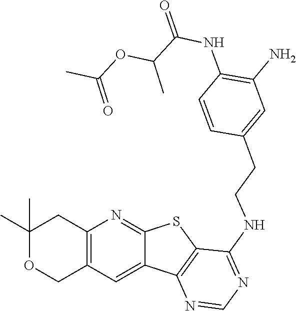 [SMILES: CC(=O)OC(C)C(=O)Nc1ccc(CCNc2ncnc3c2sc2nc4c(cc23)COC(C)(C)C4)cc1N] |
| 187 | 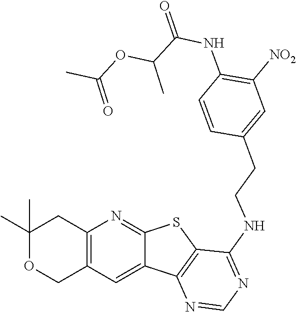 [SMILES: CC(=O)OC(C)C(=O)Nc1ccc(CCNc2ncnc3c2sc2nc4c(cc23)COC(C)(C)C4)cc1[N+](=O)[O-]] |
| 188 | 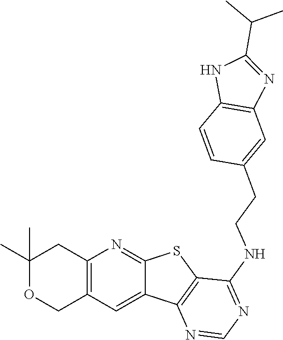 [SMILES: CC(C)c1nc2cc(CCNc3ncnc4c3sc3nc5c(cc34)COC(C)(C)C5)ccc2[nH]1] |
| 189 | 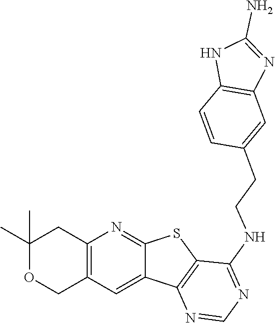 [SMILES: CC1(C)Cc2nc3sc4c(NCCc5ccc6[nH]c(N)nc6c5)ncnc4c3cc2CO1] |
| 190 | 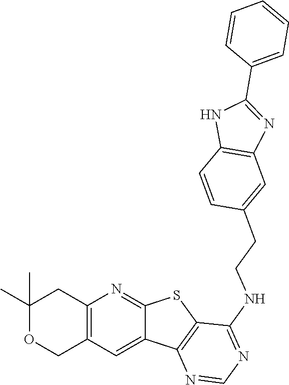 [SMILES: CC1(C)Cc2nc3sc4c(NCCc5ccc6[nH]c(-c7ccccc7)nc6c5)ncnc4c3cc2CO1] |
| 191 | 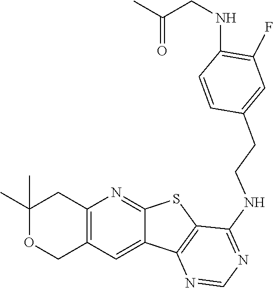 [SMILES: CC(=O)CNc1ccc(CCNc2ncnc3c2sc2nc4c(cc23)COC(C)(C)C4)cc1F] |
| 192 | 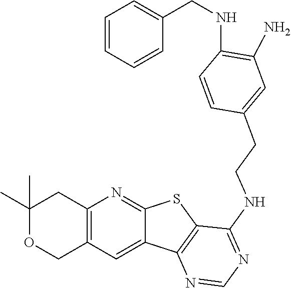 [SMILES: CC1(C)Cc2nc3sc4c(NCCc5ccc(NCc6ccccc6)c(N)c5)ncnc4c3cc2CO1] |
| 193 | 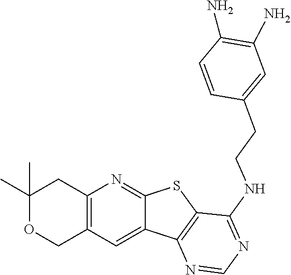 [SMILES: CC1(C)Cc2nc3sc4c(NCCc5ccc(N)c(N)c5)ncnc4c3cc2CO1] |
| 194 | 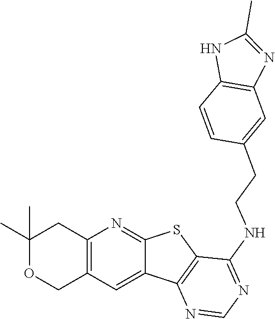 [SMILES: Cc1nc2cc(CCNc3ncnc4c3sc3nc5c(cc34)COC(C)(C)C5)ccc2[nH]1] |
| 195 | 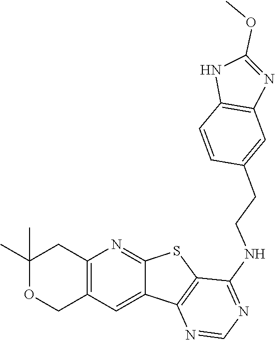 [SMILES: COc1nc2cc(CCNc3ncnc4c3sc3nc5c(cc34)COC(C)(C)C5)ccc2[nH]1] |
| 196 | 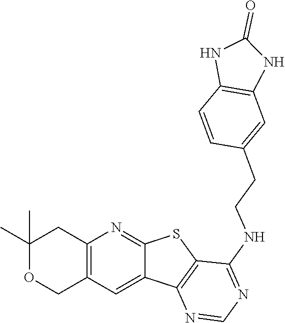 [SMILES: CC1(C)Cc2nc3sc4c(NCCc5ccc6[nH]c(=O)[nH]c6c5)ncnc4c3cc2CO1] |
| 197 | 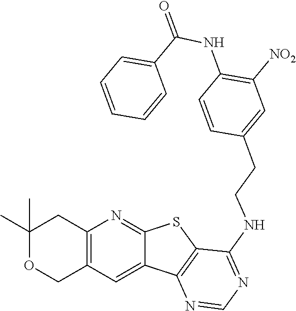 [SMILES: CC1(C)Cc2nc3sc4c(NCCc5ccc(NC(=O)c6ccccc6)c([N+](=O)[O-])c5)ncnc4c3cc2CO1] |
| 198 | 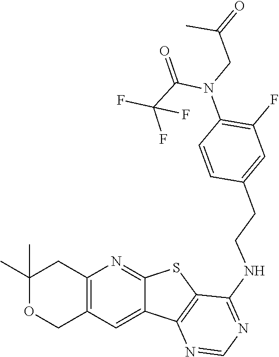 [SMILES: CC(=O)CN(C(=O)C(F)(F)F)c1ccc(CCNc2ncnc3c2sc2nc4c(cc23)COC(C)(C)C4)cc1F] |
| 199 | 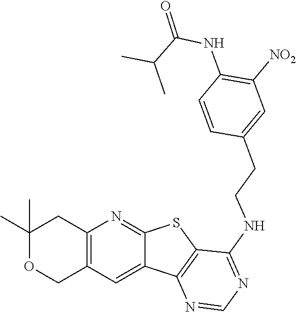 [SMILES: CC(C)C(=O)Nc1ccc(CCNc2ncnc3c2sc2nc4c(cc23)COC(C)(C)C4)cc1[N+](=O)[O-]] |
| 200 | 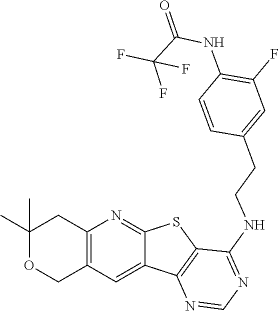 [SMILES: CC1(C)Cc2nc3sc4c(NCCc5ccc(NC(=O)C(F)(F)F)c(F)c5)ncnc4c3cc2CO1] |
| 201 | 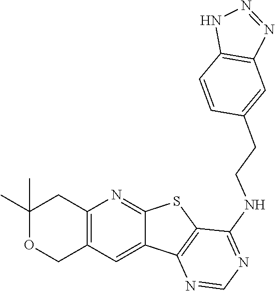 [SMILES: CC1(C)Cc2nc3sc4c(NCCc5ccc6[nH]nnc6c5)ncnc4c3cc2CO1] |
| 202 | 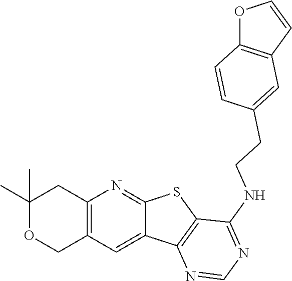 [SMILES: CC1(C)Cc2nc3sc4c(NCCc5ccc6occc6c5)ncnc4c3cc2CO1] |
| 203 | 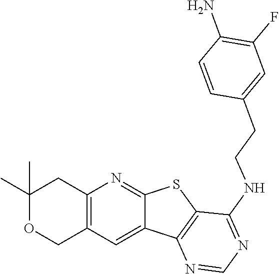 [SMILES: CC1(C)Cc2nc3sc4c(NCCc5ccc(N)c(F)c5)ncnc4c3cc2CO1] |
| 204 | 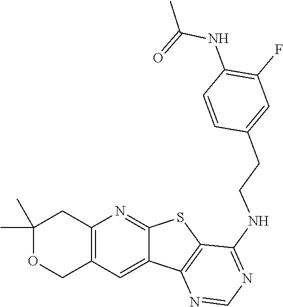 [SMILES: CC(=O)Nc1ccc(CCNc2ncnc3c2sc2nc4c(cc23)COC(C)(C)C4)cc1F] |
| 205 | 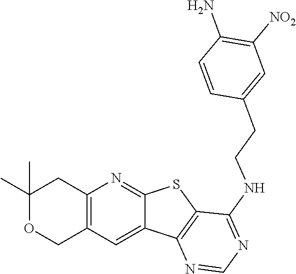 [SMILES: CC1(C)Cc2nc3sc4c(NCCc5ccc(N)c([N+](=O)[O-])c5)ncnc4c3cc2CO1] |
| 206 | 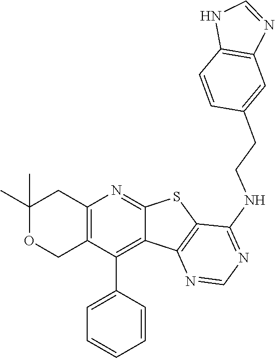 [SMILES: CC1(C)Cc2nc3sc4c(NCCc5ccc6[nH]cnc6c5)ncnc4c3c(-c3ccccc3)c2CO1] |
| 207 | 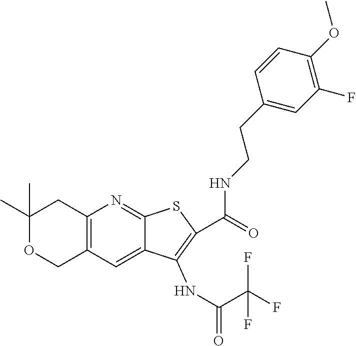 [SMILES: COc1ccc(CCNC(=O)c2sc3nc4c(cc3c2NC(=O)C(F)(F)F)COC(C)(C)C4)cc1F] |
| 208 | 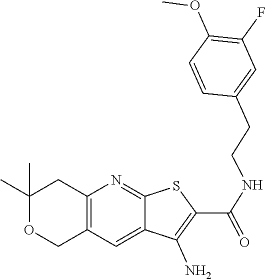 [SMILES: COc1ccc(CCNC(=O)c2sc3nc4c(cc3c2N)COC(C)(C)C4)cc1F] |
| 209 | 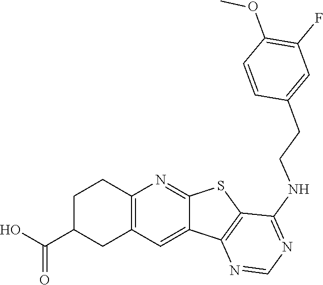 [SMILES: COc1ccc(CCNc2ncnc3c2sc2nc4c(cc23)CC(C(=O)O)CC4)cc1F] |
| 210 | 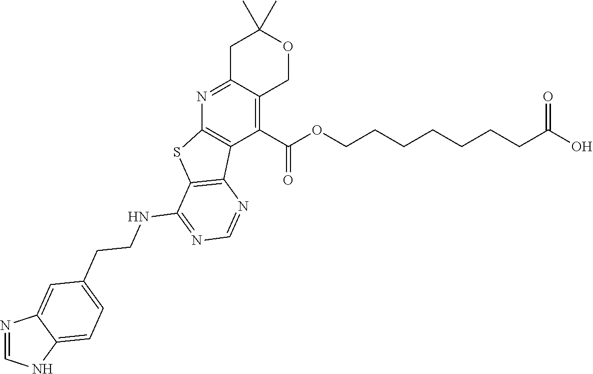 [SMILES: CC1(C)Cc2nc3sc4c(NCCc5ccc6[nH]cnc6c5)ncnc4c3c(C(=O)OCCCCCCCC(=O)O)c2CO1] |
| 211 | 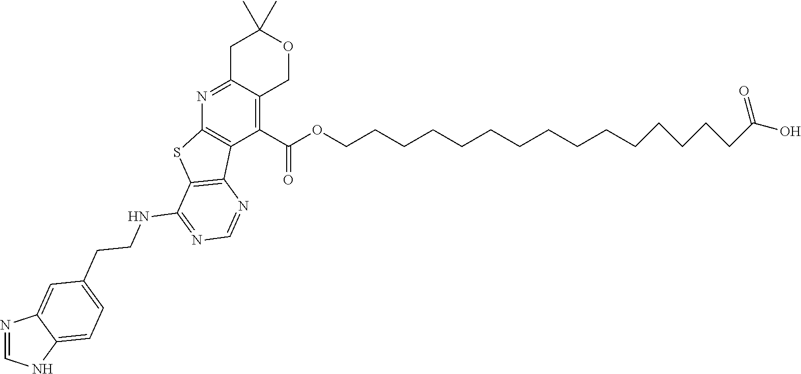 [SMILES: CC1(C)Cc2nc3sc4c(NCCc5ccc6[nH]cnc6c5)ncnc4c3c(C(=O)OCCCCCCCCCCCCCCCC(=O)O)c2CO1] |
| 212 | 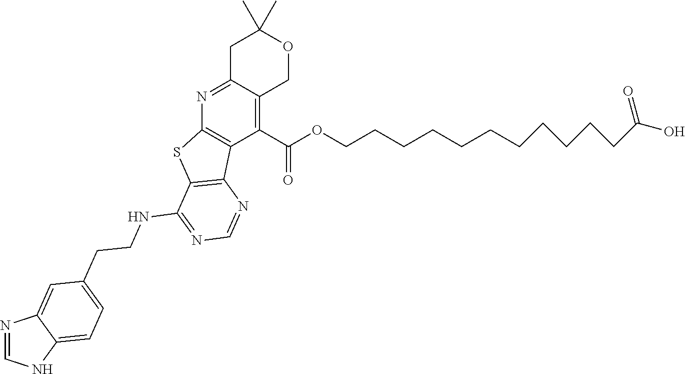 [SMILES: CC1(C)Cc2nc3sc4c(NCCc5ccc6[nH]cnc6c5)ncnc4c3c(C(=O)OCCCCCCCCCCCC(=O)O)c2CO1] |
| 213 | 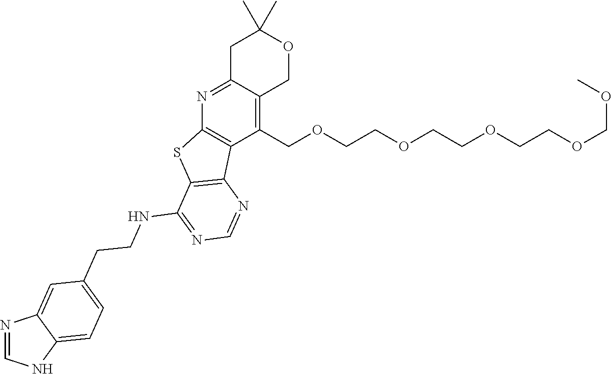 [SMILES: COCOCCOCCOCCOCc1c2c(nc3sc4c(NCCc5ccc6[nH]cnc6c5)ncnc4c13)CC(C)(C)OC2] |
| 214 | 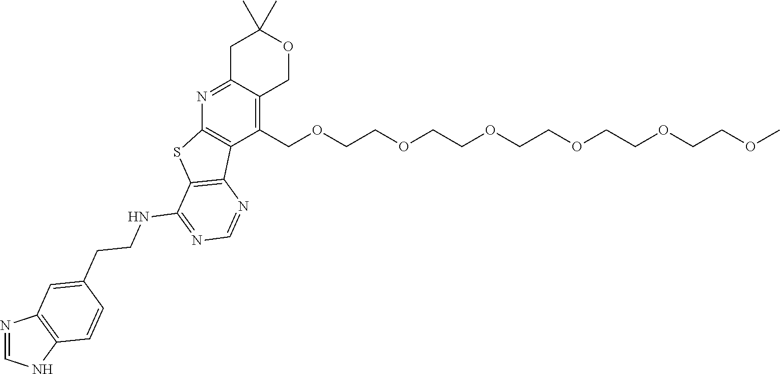 [SMILES: COCCOCCOCCOCCOCCOCc1c2c(nc3sc4c(NCCc5ccc6[nH]cnc6c5)ncnc4c13)CC(C)(C)OC2] |
| 215 | 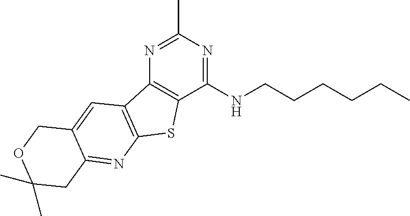 [SMILES: CCCCCCNc1nc(C)nc2c1sc1nc3c(cc12)COC(C)(C)C3] |
| 216 | 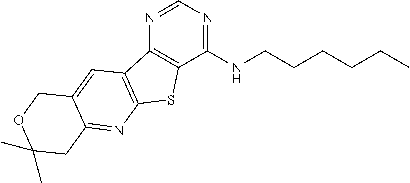 [SMILES: CCCCCCNc1ncnc2c1sc1nc3c(cc12)COC(C)(C)C3] |
| 217 | 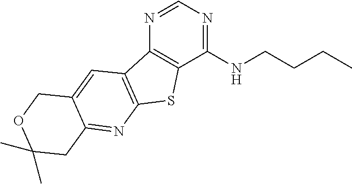 [SMILES: CCCCNc1ncnc2c1sc1nc3c(cc12)COC(C)(C)C3] |
| 218 | 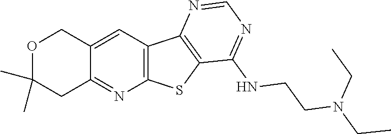 [SMILES: CCN(CC)CCNc1ncnc2c1sc1nc3c(cc12)COC(C)(C)C3] |
| 219 | 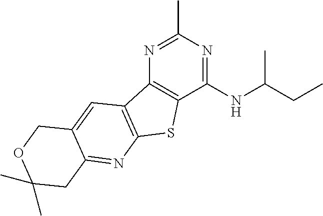 [SMILES: CCC(C)Nc1nc(C)nc2c1sc1nc3c(cc12)COC(C)(C)C3] |
| 220 | 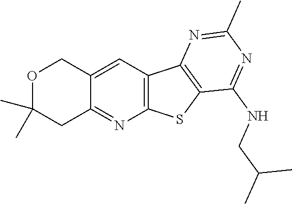 [SMILES: Cc1nc(NCC(C)C)c2sc3nc4c(cc3c2n1)COC(C)(C)C4] |
| 221 | 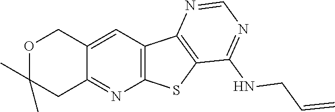 [SMILES: C=CCNc1ncnc2c1sc1nc3c(cc12)COC(C)(C)C3] |
| 222 | 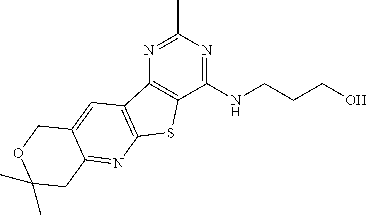 [SMILES: Cc1nc(NCCCO)c2sc3nc4c(cc3c2n1)COC(C)(C)C4] |
| 223 | 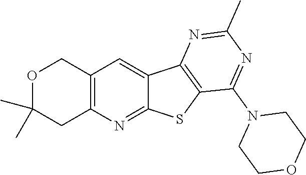 [SMILES: Cc1nc(N2CCOCC2)c2sc3nc4c(cc3c2n1)COC(C)(C)C4] |
| 224 | 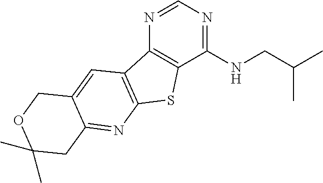 [SMILES: CC(C)CNc1ncnc2c1sc1nc3c(cc12)COC(C)(C)C3] |
| 225 | 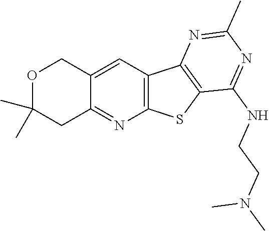 |
| 226 | 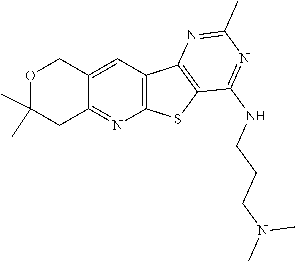 |
| 227 | 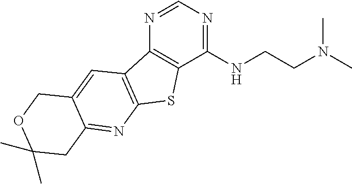 |
| 228 | 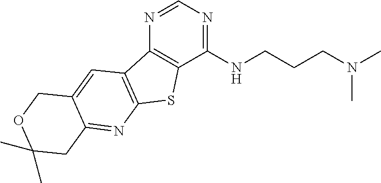 |
| 229 | 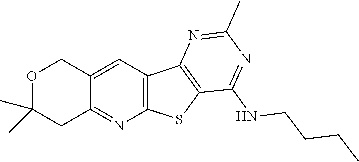 [SMILES: CCCCNc1nc(C)nc2c1sc1nc3c(cc12)COC(C)(C)C3] |
| 230 | 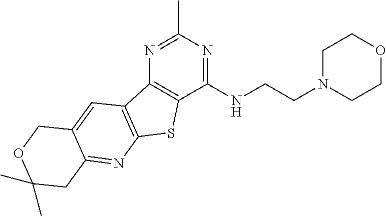 [SMILES: Cc1nc(NCCN2CCOCC2)c2sc3nc4c(cc3c2n1)COC(C)(C)C4] |
| 231 | 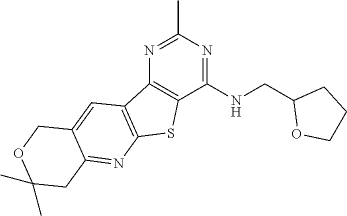 [SMILES: Cc1nc(NCC2CCCO2)c2sc3nc4c(cc3c2n1)COC(C)(C)C4] |
| 232 | 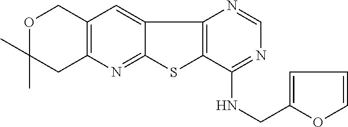 [SMILES: CC1(C)Cc2nc3sc4c(NCc5ccco5)ncnc4c3cc2CO1] |
| 233 | 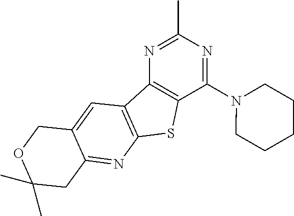 [SMILES: Cc1nc(N2CCCCC2)c2sc3nc4c(cc3c2n1)COC(C)(C)C4] |
| 234 | 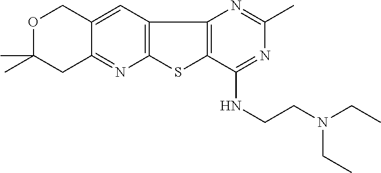 [SMILES: CCN(CC)CCNc1nc(C)nc2c1sc1nc3c(cc12)COC(C)(C)C3] |
| 235 | 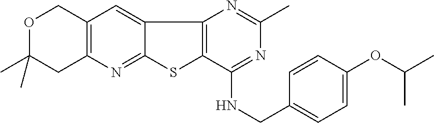 [SMILES: Cc1nc(NCc2ccc(OC(C)C)cc2)c2sc3nc4c(cc3c2n1)COC(C)(C)C4] |
| 236 | 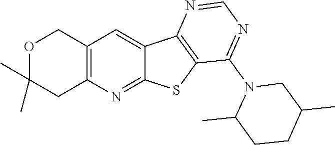 |
| 237 | 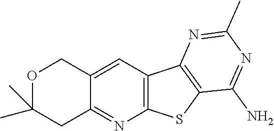 [SMILES: Cc1nc(N)c2sc3nc4c(cc3c2n1)COC(C)(C)C4] |
| 238 | 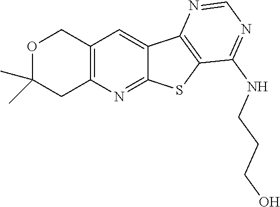 [SMILES: CC1(C)Cc2nc3sc4c(NCCCO)ncnc4c3cc2CO1] |
| 239 | 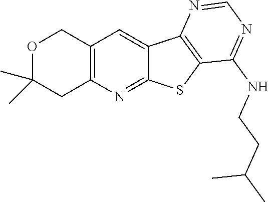 [SMILES: CC(C)CCNc1ncnc2c1sc1nc3c(cc12)COC(C)(C)C3] |
| 240 | 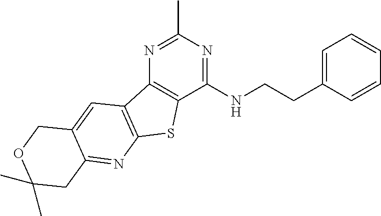 [SMILES: Cc1nc(NCCc2ccccc2)c2sc3nc4c(cc3c2n1)COC(C)(C)C4] |
| 241 | 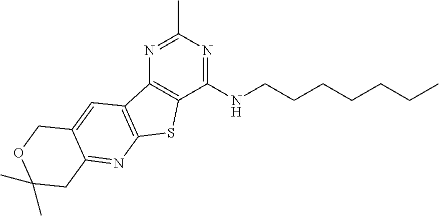 [SMILES: CCCCCCCNc1nc(C)nc2c1sc1nc3c(cc12)COC(C)(C)C3] |
| 242 | 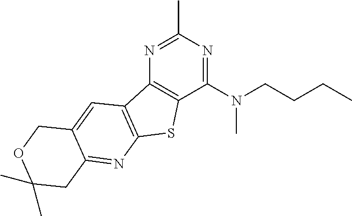 [SMILES: CCCCN(C)c1nc(C)nc2c1sc1nc3c(cc12)COC(C)(C)C3] |
| 243 | 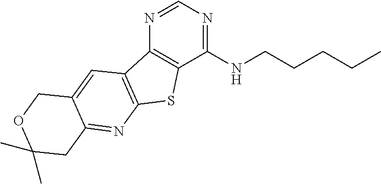 [SMILES: CCCCCNc1ncnc2c1sc1nc3c(cc12)COC(C)(C)C3] |
| 244 | 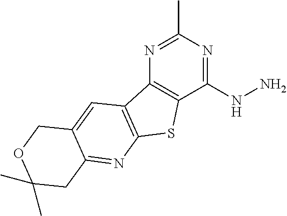 [SMILES: Cc1nc(NN)c2sc3nc4c(cc3c2n1)COC(C)(C)C4] |
| 245 | 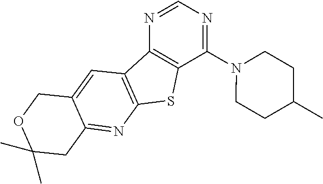 |
| 246 | 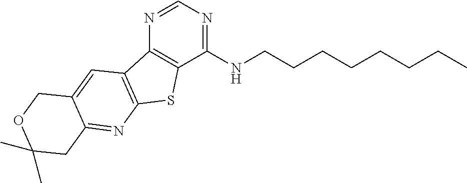 [SMILES: CCCCCCCCNc1ncnc2c1sc1nc3c(cc12)COC(C)(C)C3] |
| 247 | 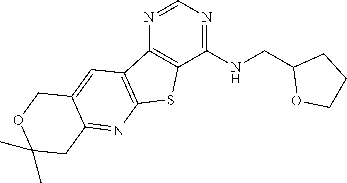 [SMILES: CC1(C)Cc2nc3sc4c(NCC5CCCO5)ncnc4c3cc2CO1] |
| 248 | 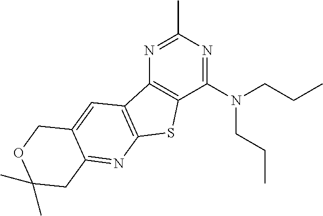 [SMILES: CCCN(CCC)c1nc(C)nc2c1sc1nc3c(cc12)COC(C)(C)C3] |
| 249 | 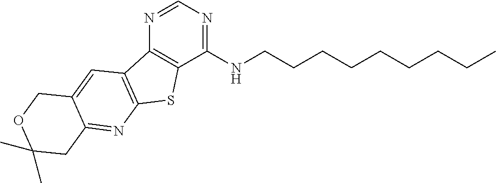 [SMILES: CCCCCCCCCNc1ncnc2c1sc1nc3c(cc12)COC(C)(C)C3] |
| 250 | 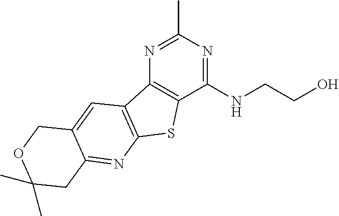 [SMILES: Cc1nc(NCCO)c2sc3nc4c(cc3c2n1)COC(C)(C)C4] |
| 251 | 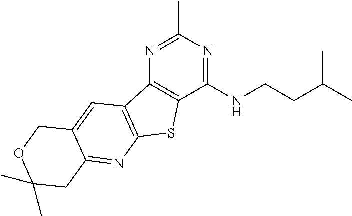 [SMILES: Cc1nc(NCCC(C)C)c2sc3nc4c(cc3c2n1)COC(C)(C)C4] |
| 252 | 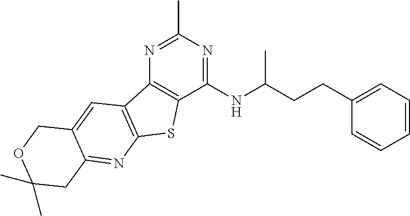 [SMILES: Cc1nc(NC(C)CCc2ccccc2)c2sc3nc4c(cc3c2n1)COC(C)(C)C4] |
| 253 | 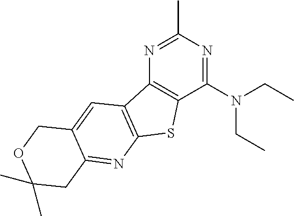 [SMILES: CCN(CC)c1nc(C)nc2c1sc1nc3c(cc12)COC(C)(C)C3] |
| 254 | 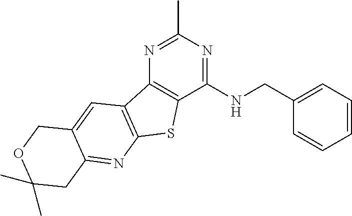 [SMILES: Cc1nc(NCc2ccccc2)c2sc3nc4c(cc3c2n1)COC(C)(C)C4] |
| 255 | 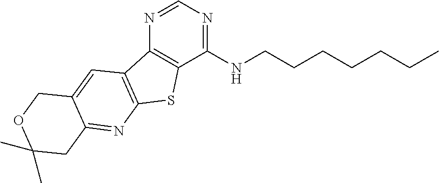 [SMILES: CCCCCCCNc1ncnc2c1sc1nc3c(cc12)COC(C)(C)C3] |
| 256 | 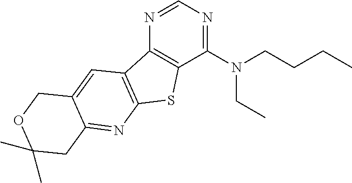 [SMILES: CCCCN(CC)c1ncnc2c1sc1nc3c(cc12)COC(C)(C)C3] |
| 257 | 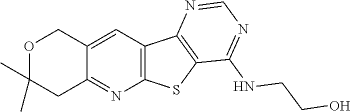 [SMILES: CC1(C)Cc2nc3sc4c(NCCO)ncnc4c3cc2CO1] |
| 258 | 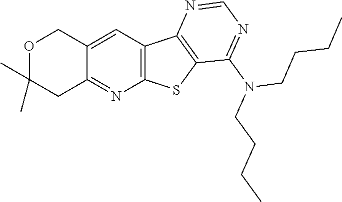 [SMILES: CCCCN(CCCC)c1ncnc2c1sc1nc3c(cc12)COC(C)(C)C3] |
| 259 | 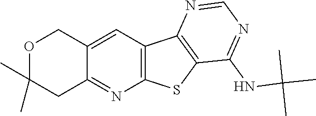 [SMILES: CC(C)(C)Nc1ncnc2c1sc1nc3c(cc12)COC(C)(C)C3] |
| 260 | 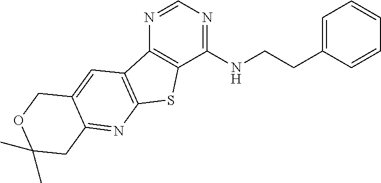 [SMILES: CC1(C)Cc2nc3sc4c(NCCc5ccccc5)ncnc4c3cc2CO1] |
| 261 | 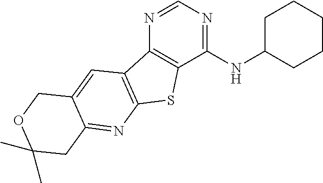 [SMILES: CC1(C)Cc2nc3sc4c(NC5CCCCC5)ncnc4c3cc2CO1] |
| 262 | 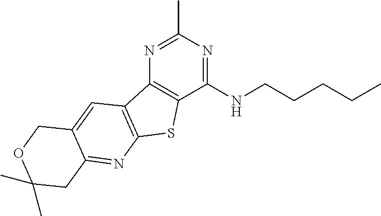 [SMILES: CCCCCNc1nc(C)nc2c1sc1nc3c(cc12)COC(C)(C)C3] |
| 263 | 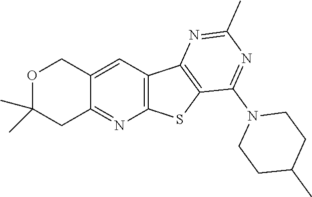 |
| 264 | 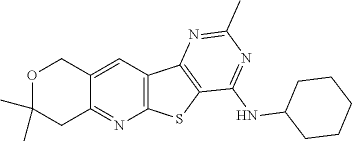 [SMILES: Cc1nc(NC2CCCCC2)c2sc3nc4c(cc3c2n1)COC(C)(C)C4] |
| 265 | 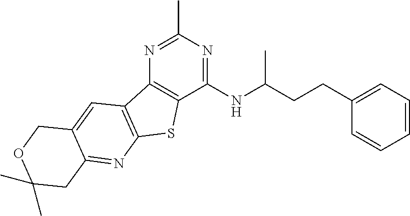 [SMILES: Cc1nc(NC(C)CCc2ccccc2)c2sc3nc4c(cc3c2n1)COC(C)(C)C4] |
| 266 | 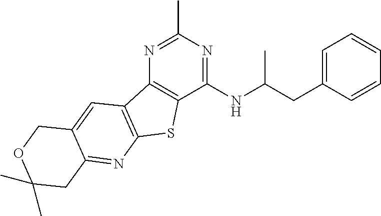 [SMILES: Cc1nc(NC(C)Cc2ccccc2)c2sc3nc4c(cc3c2n1)COC(C)(C)C4] |
| 267 | 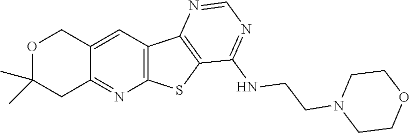 [SMILES: CC1(C)Cc2nc3sc4c(NCCN5CCOCC5)ncnc4c3cc2CO1] |
| 268 | 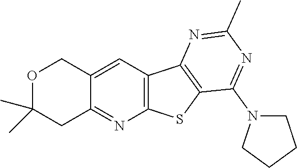 [SMILES: Cc1nc(N2CCCC2)c2sc3nc4c(cc3c2n1)COC(C)(C)C4] |
| 269 | 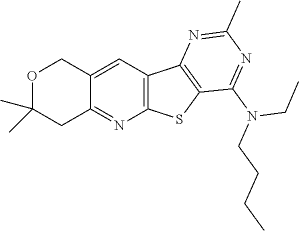 [SMILES: CCCCN(CC)c1nc(C)nc2c1sc1nc3c(cc12)COC(C)(C)C3] |
| 270 | 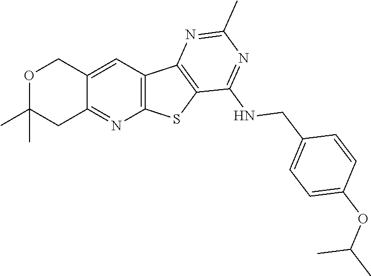 [SMILES: Cc1nc(NCc2ccc(OC(C)C)cc2)c2sc3nc4c(cc3c2n1)COC(C)(C)C4] |
| 271 | 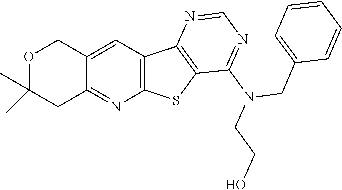 [SMILES: CC1(C)Cc2nc3sc4c(N(CCO)Cc5ccccc5)ncnc4c3cc2CO1] |
| 272 | 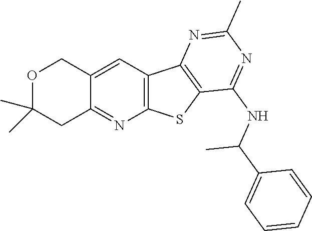 [SMILES: Cc1nc(NC(C)c2ccccc2)c2sc3nc4c(cc3c2n1)COC(C)(C)C4] |
| 273 | 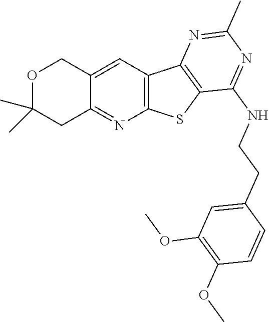 [SMILES: COc1ccc(CCNc2nc(C)nc3c2sc2nc4c(cc23)COC(C)(C)C4)cc1OC] |
| 274 | 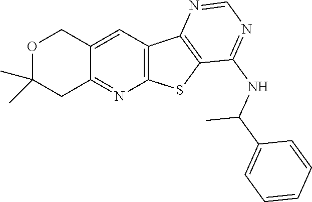 [SMILES: CC(Nc1ncnc2c1sc1nc3c(cc12)COC(C)(C)C3)c1ccccc1] |
| 275 | 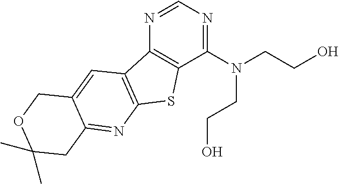 [SMILES: CC1(C)Cc2nc3sc4c(N(CCO)CCO)ncnc4c3cc2CO1] |
| 276 | 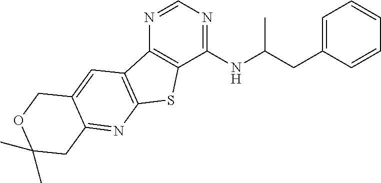 [SMILES: CC(Cc1ccccc1)Nc1ncnc2c1sc1nc3c(cc12)COC(C)(C)C3] |
| 277 | 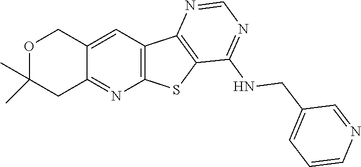 [SMILES: CC1(C)Cc2nc3sc4c(NCc5cccnc5)ncnc4c3cc2CO1] |
| 278 | 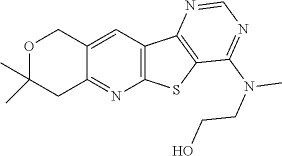 |
| 279 | 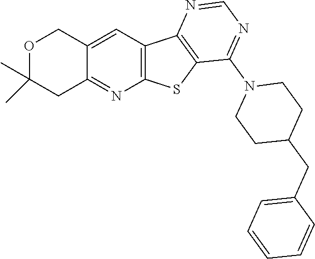 [SMILES: CC1(C)Cc2nc3sc4c(N5CCC(Cc6ccccc6)CC5)ncnc4c3cc2CO1] |
| 280 | 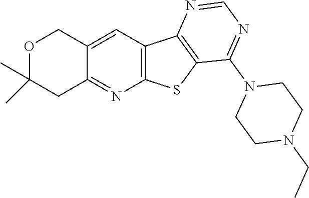 [SMILES: CCN1CCN(c2ncnc3c2sc2nc4c(cc23)COC(C)(C)C4)CC1] |
| 281 | 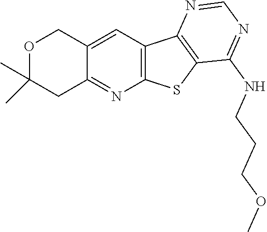 [SMILES: COCCCNc1ncnc2c1sc1nc3c(cc12)COC(C)(C)C3] |
| 282 | 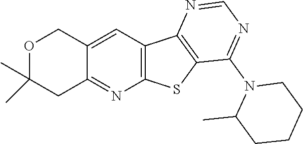 [SMILES: CC1CCCCN1c1ncnc2c1sc1nc3c(cc12)COC(C)(C)C3] |
| 283 | 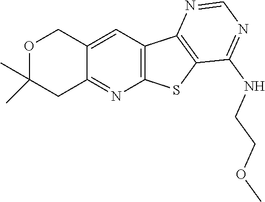 |
| 284 | 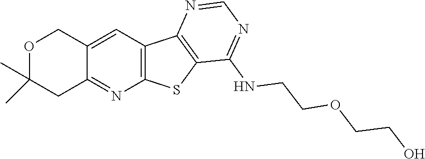 [SMILES: CC1(C)Cc2nc3sc4c(NCCOCCO)ncnc4c3cc2CO1] |
| 285 | 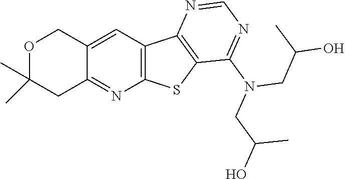 [SMILES: CC(O)CN(CC(C)O)c1ncnc2c1sc1nc3c(cc12)COC(C)(C)C3] |
| 286 | 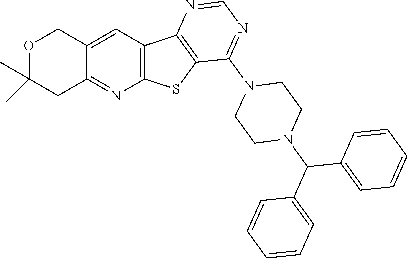 [SMILES: CC1(C)Cc2nc3sc4c(N5CCN(C(c6ccccc6)c6ccccc6)CC5)ncnc4c3cc2CO1] |
| 287 | 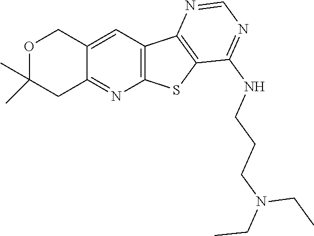 [SMILES: CCN(CC)CCCNc1ncnc2c1sc1nc3c(cc12)COC(C)(C)C3] |
| 288 | 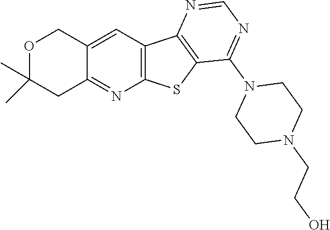 [SMILES: CC1(C)Cc2nc3sc4c(N5CCN(CCO)CC5)ncnc4c3cc2CO1] |
| 289 | 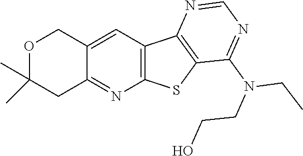 [SMILES: CCN(CCO)c1ncnc2c1sc1nc3c(cc12)COC(C)(C)C3] |
| 290 | 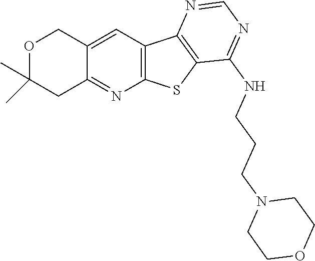 [SMILES: CC1(C)Cc2nc3sc4c(NCCCN5CCOCC5)ncnc4c3cc2CO1] |
| 291 | 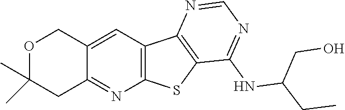 [SMILES: CCC(CO)Nc1ncnc2c1sc1nc3c(cc12)COC(C)(C)C3] |
| 292 | 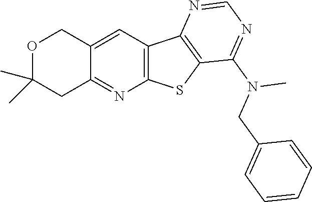 [SMILES: CN(Cc1ccccc1)c1ncnc2c1sc1nc3c(cc12)COC(C)(C)C3] |
| 293 | 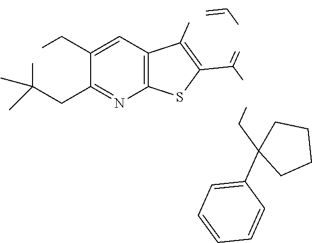 [SMILES: CC1(C)CCc2cc3c(nc2C1)sc1c(CCC2(c4ccccc4)CCCC2)cccc13] |
| 294 | 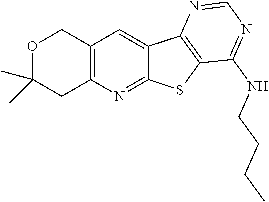 [SMILES: CCCCNc1ncnc2c1sc1nc3c(cc12)COC(C)(C)C3] |
| 295 | 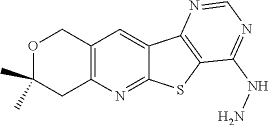 |
| 296 | 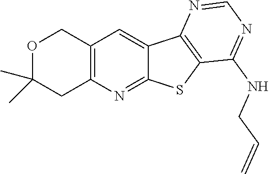 [SMILES: C=CCNc1ncnc2c1sc1nc3c(cc12)COC(C)(C)C3] |
| 297 | 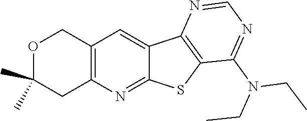 |
| 298 | 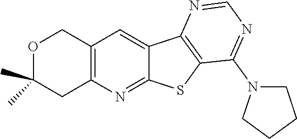 |
| 299 | 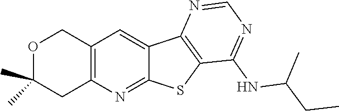 |
| 300 | 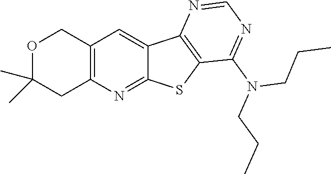 [SMILES: CCCN(CCC)c1ncnc2c1sc1nc3c(cc12)COC(C)(C)C3] |
| 301 | 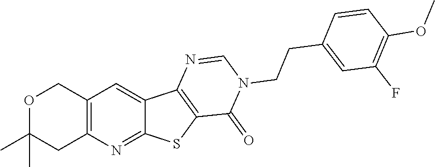 |
| 302 | 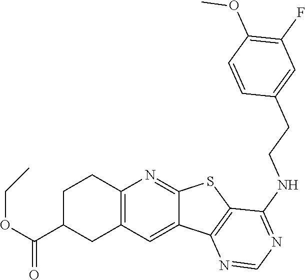 [SMILES: CCOC(=O)C1CCc2nc3sc4c(NCCc5ccc(OC)c(F)c5)ncnc4c3cc2C1] |
| 303 | 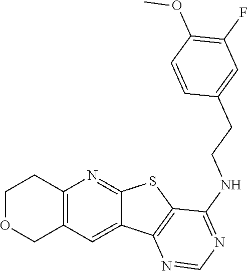 [SMILES: COc1ccc(CCNc2ncnc3c2sc2nc4c(cc23)COCC4)cc1F] |
| 304 | 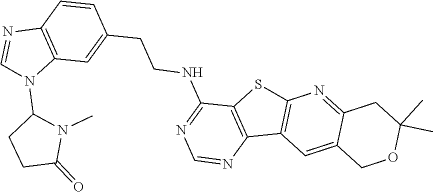 [SMILES: CN1C(=O)CCC1n1cnc2ccc(CCNc3ncnc4c3sc3nc5c(cc34)COC(C)(C)C5)cc21] |
| 305 | 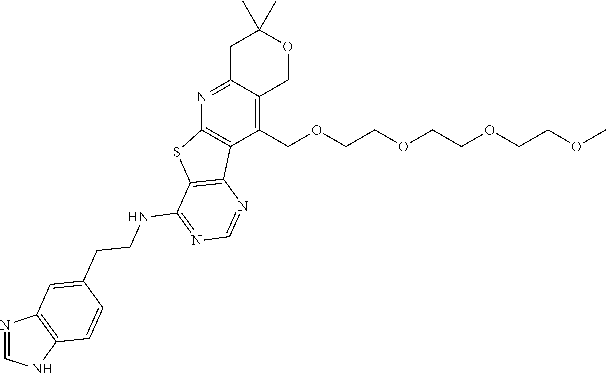 [SMILES: COCCOCCOCCOCc1c2c(nc3sc4c(NCCc5ccc6[nH]cnc6c5)ncnc4c13)CC(C)(C)OC2] |
| 306 | 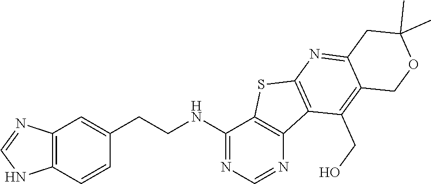 [SMILES: CC1(C)Cc2nc3sc4c(NCCc5ccc6[nH]cnc6c5)ncnc4c3c(CO)c2CO1] |
| 307 | 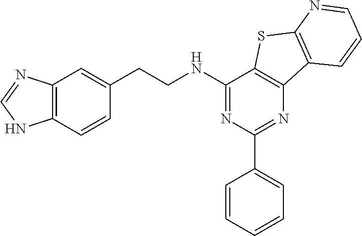 [SMILES: c1ccc(-c2nc(NCCc3ccc4[nH]cnc4c3)c3sc4ncccc4c3n2)cc1] |
| 308 | 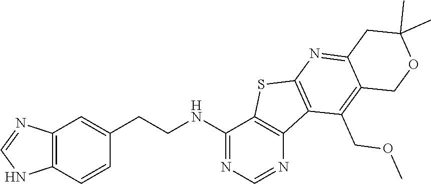 [SMILES: COCc1c2c(nc3sc4c(NCCc5ccc6[nH]cnc6c5)ncnc4c13)CC(C)(C)OC2] |
| 309 | 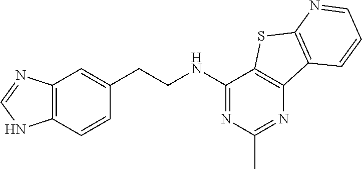 [SMILES: Cc1nc(NCCc2ccc3[nH]cnc3c2)c2sc3ncccc3c2n1] |
| 310 | 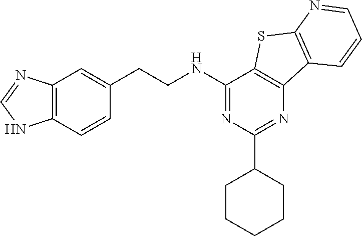 [SMILES: c1cnc2sc3c(NCCc4ccc5[nH]cnc5c4)nc(C4CCCCC4)nc3c2c1] |
| 311 | 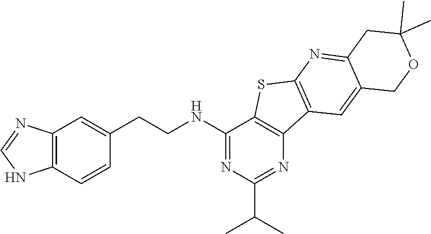 [SMILES: CC(C)c1nc(NCCc2ccc3[nH]cnc3c2)c2sc3nc4c(cc3c2n1)COC(C)(C)C4] |
| 312 | 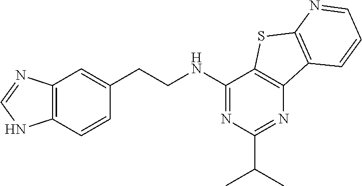 [SMILES: CC(C)c1nc(NCCc2ccc3[nH]cnc3c2)c2sc3ncccc3c2n1] |
| 313 | 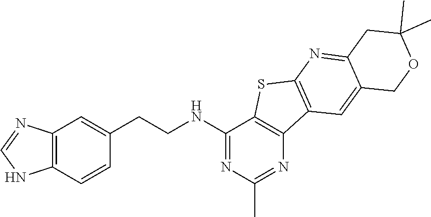 [SMILES: Cc1nc(NCCc2ccc3[nH]cnc3c2)c2sc3nc4c(cc3c2n1)COC(C)(C)C4] |
| 314 | 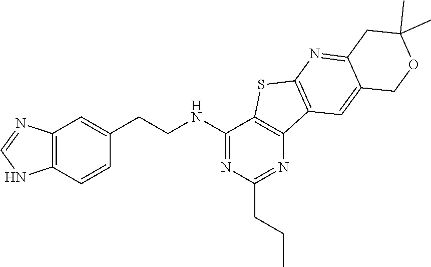 [SMILES: CCCc1nc(NCCc2ccc3[nH]cnc3c2)c2sc3nc4c(cc3c2n1)COC(C)(C)C4] |
| 315 | 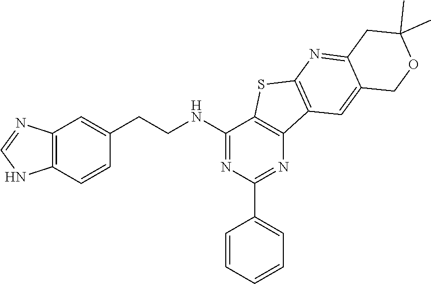 [SMILES: CC1(C)Cc2nc3sc4c(NCCc5ccc6[nH]cnc6c5)nc(-c5ccccc5)nc4c3cc2CO1] |
| 316 | 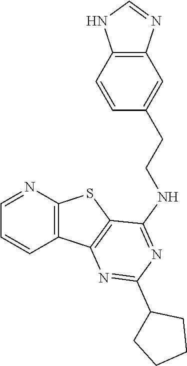 [SMILES: c1cnc2sc3c(NCCc4ccc5[nH]cnc5c4)nc(C4CCCC4)nc3c2c1] |
| 317 | 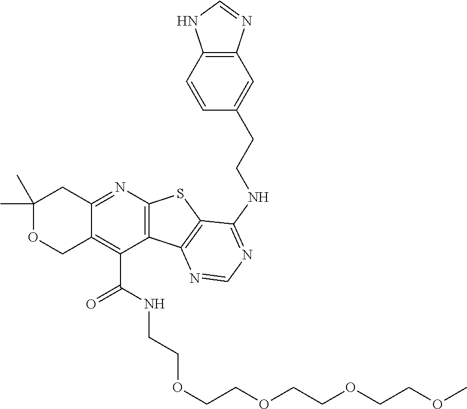 |
| 318 | 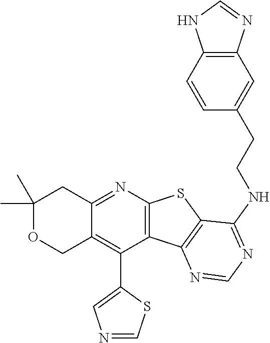 [SMILES: CC1(C)Cc2nc3sc4c(NCCc5ccc6[nH]cnc6c5)ncnc4c3c(-c3cncs3)c2CO1] |
| 319 | 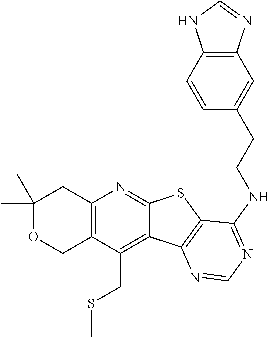 [SMILES: CSCc1c2c(nc3sc4c(NCCc5ccc6[nH]cnc6c5)ncnc4c13)CC(C)(C)OC2] |
| 320 | 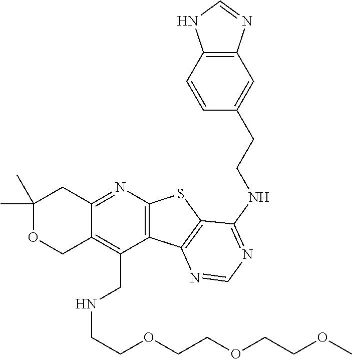 |
| 321 | 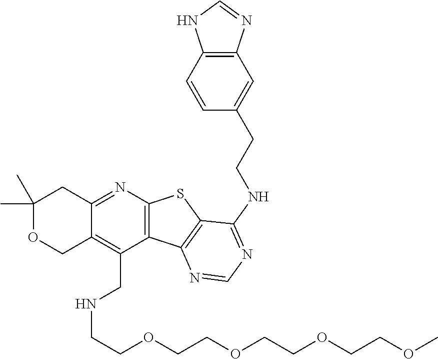 |
| 322 | 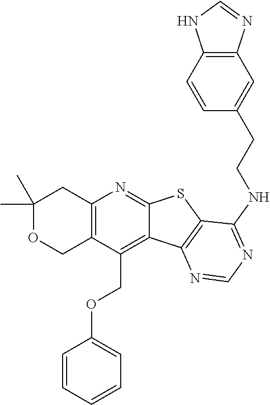 [SMILES: CC1(C)Cc2nc3sc4c(NCCc5ccc6[nH]cnc6c5)ncnc4c3c(COc3ccccc3)c2CO1] |
| 323 | 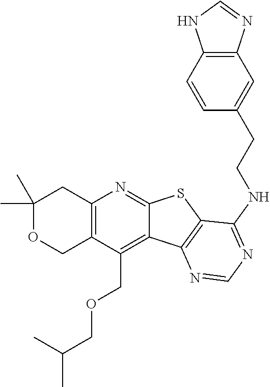 [SMILES: CC(C)COCc1c2c(nc3sc4c(NCCc5ccc6[nH]cnc6c5)ncnc4c13)CC(C)(C)OC2] |
| 324 | 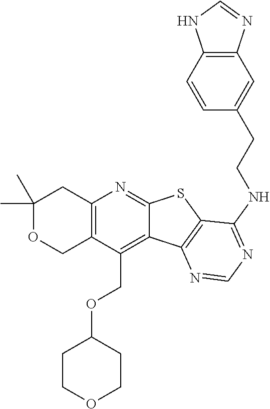 [SMILES: CC1(C)Cc2nc3sc4c(NCCc5ccc6[nH]cnc6c5)ncnc4c3c(COC3CCOCC3)c2CO1] |
| 325 | 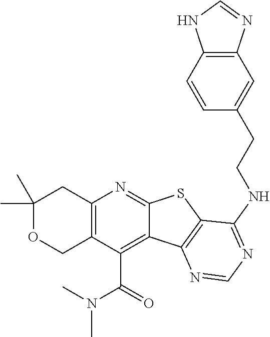 [SMILES: CN(C)C(=O)c1c2c(nc3sc4c(NCCc5ccc6[nH]cnc6c5)ncnc4c13)CC(C)(C)OC2] |
| 326 | 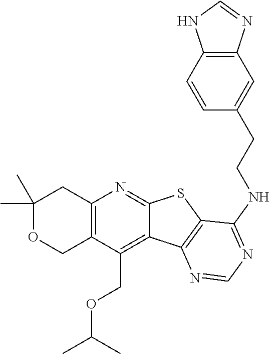 [SMILES: CC(C)OCc1c2c(nc3sc4c(NCCc5ccc6[nH]cnc6c5)ncnc4c13)CC(C)(C)OC2] |
| 327 | 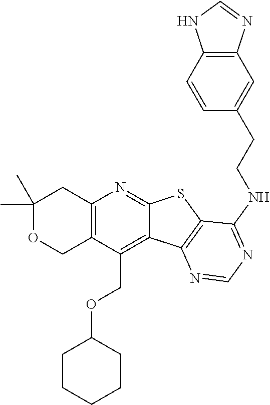 [SMILES: CC1(C)Cc2nc3sc4c(NCCc5ccc6[nH]cnc6c5)ncnc4c3c(COC3CCCCC3)c2CO1] |
| 328 | 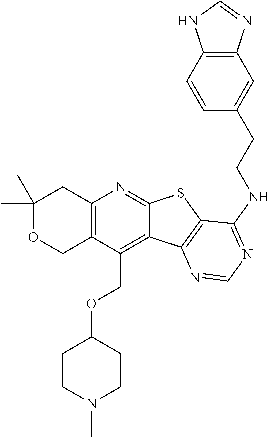 [SMILES: CN1CCC(OCc2c3c(nc4sc5c(NCCc6ccc7[nH]cnc7c6)ncnc5c24)CC(C)(C)OC3)CC1] |
| 329 | 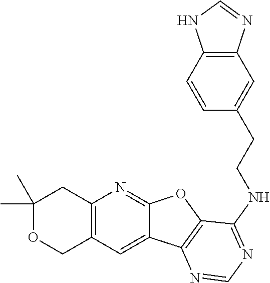 [SMILES: CC1(C)Cc2nc3oc4c(NCCc5ccc6[nH]cnc6c5)ncnc4c3cc2CO1] |
| 330 | 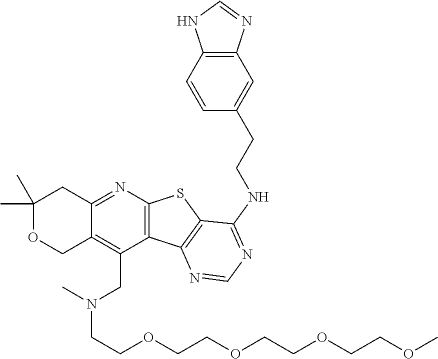 |
| 331 | 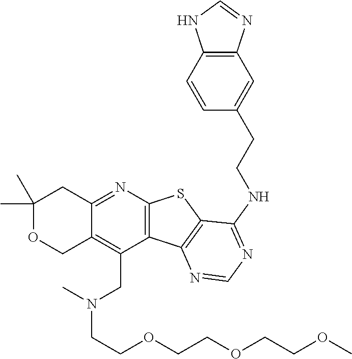 |
| 332 | 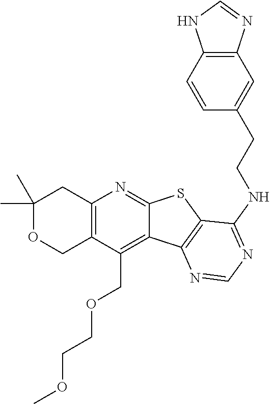 [SMILES: COCCOCc1c2c(nc3sc4c(NCCc5ccc6[nH]cnc6c5)ncnc4c13)CC(C)(C)OC2] |
| 333 | 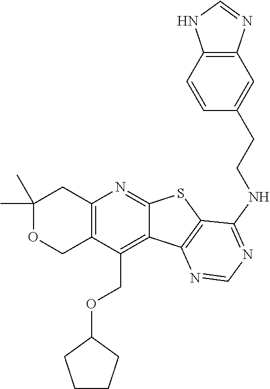 [SMILES: CC1(C)Cc2nc3sc4c(NCCc5ccc6[nH]cnc6c5)ncnc4c3c(COC3CCCC3)c2CO1] |
| 334 | 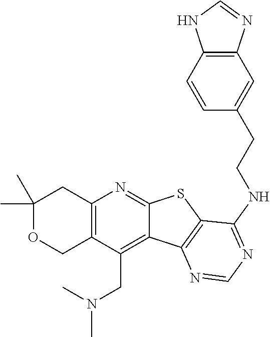 [SMILES: CN(C)Cc1c2c(nc3sc4c(NCCc5ccc6[nH]cnc6c5)ncnc4c13)CC(C)(C)OC2] |
| 335 | 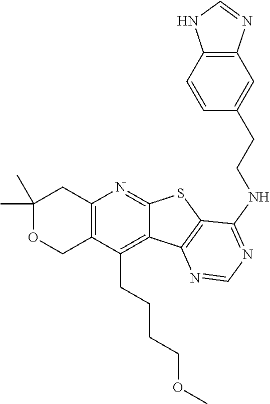 [SMILES: COCCCCc1c2c(nc3sc4c(NCCc5ccc6[nH]cnc6c5)ncnc4c13)CC(C)(C)OC2] |

It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, stereoisomer, tautomer, hydrate, N-oxide, pharmaceutical product or any combination thereof. In some embodiments, the compounds target SDH-deficient cells. In some embodiments, the compounds destroy SDH-deficient cells.

In various embodiments, the A ring of formula I is cyclohexene, cyclopentane, cycloheptene, cyclooctene, dihydropyran, tetrahydropyridine, which is optionally substituted with F, Cl, Br, I, C₁-C₅ linear or branched alkyl (e.g., methyl, 2,2-dimethyl), OH, C₁-C₅ linear or branched alkoxy (e.g. ethoxy), O(C═O)—R₁₀, COOH, N(R)₂, CF₃, CN or NO₂. In other embodiments, A is 2,2-dimethyl-3,6-dihydro-2H-pyran, 1-methyl-1,2,3,6-tetrahydropyridine, tetrahydropyran, piperidine, 1-(piperidin-1-yl)ethanone or morpholine. In other embodiments, A is absent.

In various embodiments, the A ring of formula I is absent. In other embodiments, the A ring of formula I is substituted or unsubstituted cyclohexene. In other embodiments, A is substituted or unsubstituted cyclopentane. In other embodiments, A is substituted or unsubstituted cycloheptene. In other embodiments, A is substituted or unsubstituted cyclooctene. In other embodiments, A is substituted or unsubstituted dihydropyran. In other embodiments, A is substituted or unsubstituted tetrahydropyridine. In other embodiments, A is optionally substituted with one or more of: F, Cl, Br, I, C₁-C₅ linear or branched alkyl (e.g., methyl, 2,2-dimethyl), OH, C₁-C₅ linear or branched alkoxy (e.g. ethoxy), N(R)₂, CF₃, CN or NO₂. In other embodiments, A is 2,2-dimethyl-3,6-dihydro-2H-pyran. In other embodiments, A is 1-methyl-1,2,3,6-tetrahydropyridine. In other embodiments, A is tetrahydropyran. In other embodiments, A is piperidine. In other embodiments, A is 1-(piperidin-1-yl)ethenone. In other embodiments, A is morpholine. In other embodiments, A is absent. In some embodiments, A ring is substituted with at least one substituent selected from: F, Cl, Br, I, C₁-C₅ linear or branched alkyl, OH, alkoxy (e.g., ethoxy), O(C═O)—R₁₀, COOH, COO—R₁₀, N(R)₂, CF₃, CN and NO₂; each is a separate embodiment according to this invention.

In various embodiments, R₁ of formula I-VI is H. In other embodiments, R₁ is C₁-C₅ linear or branched alkyl. In other embodiments, R₁ is a substituted C₁-C₅ linear or branched alkyl. In other embodiment, R₁ is methyl, ethyl, propyl, i-Pr, butyl, t-Bu, n-Bu, iso-Bu, pentyl, iso-pentyl, neo-pentyl, hexyl or heptyl; each one is a separate embodiment according to this invention. In other embodiments, R₁ is propanol. In other embodiments, R₁ is C₁-C₅ linear or branched alkenyl. In other embodiments, R₁ is ethylenyl, propylenyl, butylenyl, iso-butylenyl, pentylenyl; each one is a separate embodiment according to this invention. In other embodiments, R₁ is C₁-C₅ linear or branched haloalkyl. In other embodiments, R₁ is C₁-C₅ linear or branched or C₃-C₈ cyclic alkoxy. In other embodiments, R₁ is C₁-C₅ linear or branched thioalkoxy. In other embodiments, R₁ is C₁-C₅ linear or branched haloalkoxy. In other embodiments, R₁ is C₁-C₅ linear or branched alkoxyalkyl. In other embodiments, R₁ is substituted or unsubstituted C₃-C₈ cycloalkyl. In other embodiments, R₁ is cyclopropyl, cyclobutyl, cyclopentyl, cyclohexyl, cyclohexyl; each one is a separate embodiment according to this invention. In other embodiments, R₁ is substituted or unsubstituted C₃-C₈ heterocyclic ring. In other embodiments, R₁ is substituted or unsubstituted aryl (wherein substitutions include: F, Cl, Br, I, C₁-C₅ linear or branched alkyl, OH, alkoxy, N(R)₂, CF₃, CN or NO₂). In other embodiments, R₁ is F. In other embodiments, R₁ is Cl. In other embodiments, R₁ is Br. In other embodiments, R₁ is I. In other embodiments, R₁ is OH. In other embodiments, R₁ is SH. In other embodiments, R₁ is R₈-R₁₂. In other embodiments, R₁ is R₁₂. In other embodiments, R₁ is R₈—OH. In other embodiments, R₁ is R₈—SH. In other embodiments, R₁ is —R₈—O—R₁₀. In other embodiments, R₁ is (CH₂)₂—O—(CH₂)₂—OH. In other embodiments, R₁ is (CH₂)₂—O—(CH₃). In other embodiments, R₁ is (CH₂)₃—O—(CH₃). In other embodiments, R₁ is CF₃. In other embodiments, R₁ is CN. In other embodiments, R₁ is NO₂. In other embodiments, R₁ is —CH₂CN. In other embodiments, R₁ is —R₈CN. In other embodiments, R₁ is NH₂. In other embodiments, R₁ is NHR. In other embodiments, R₁ is N(R)₂. In other embodiments, R₁ is R₈—N(R₁₀)(R₁₁). In other embodiments, R₁ is (CH₂)₃-morpholin. In other embodiments, R₁ is (CH₂)₃—N(Et)₂. In other embodiments, R₁ is —OC(O)CF₃. In other embodiments, R₁ is —OCH₂Ph. In other embodiments, R₁ is —NHCO—R₁₀. In other embodiments, R₁ is NHCO—N(R₁₀)(R₁₁). In other embodiments, R₁ is COOH. In other embodiments, R₁ is —C(O)Ph. In other embodiments, R₁ is C(O)O—R₁₀. In other embodiments, R₁ is R₈—C(O)—R₁₀. In other embodiments, R₁ is C(O)H. In other embodiments, R₁ is C(O)—R₁₀. In other embodiments, R₁ is C(O)—CH₃. In other embodiments, R₁ is C₁-C₅ linear or branched C(O)-haloalkyl. In other embodiments, R₁ is —C(O)NH₂. In other embodiments, R₁ is C(O)NHR. In other embodiments, R₁ is C(O)N(R₁₀)(R₁₁). In other embodiments, R₁ is SO₂R. In other embodiments, R₁ is SO₂N(R₁₀)(R₁₁).

In various embodiments, R₂ of formula I and II is H. In other embodiments, R₂ is C₁-C₅ linear or branched alkyl. In other embodiments, R₂ is a substituted C₁-C₅ linear or branched alkyl. In other embodiment, R₂ is methyl, ethyl, propyl, i-Pr, butyl, t-Bu, n-Bu, iso-Bu, pentyl, iso-pentyl, neo-pentyl, hexyl or heptyl; each one is a separate embodiment according to this invention. In other embodiments, R₂ is propanol. In other embodiments, R₂ is C₁-C₅ linear or branched alkenyl. In other embodiments, R₂ is ethylenyl, propylenyl, butylenyl, iso-butylenyl, pentylenyl; each one is a separate embodiment according to this invention. In other embodiments, R₂ is C₁-C₅ linear or branched haloalkyl. In other embodiments, R₂ is C₁-C₅ linear or branched or C₃-C₈ cyclic alkoxy. In other embodiments, R₂ is C₁-C₅ linear or branched thioalkoxy. In other embodiments, R₂ is C₁-C₅ linear or branched haloalkoxy. In other embodiments, R₂ is C₁-C₅ linear or branched alkoxyalkyl. In other embodiments, R₂ is substituted or unsubstituted C₃-C₈ cycloalkyl. In other embodiments, R₂ is cyclopropyl, cyclobutyl, cyclopentyl, cyclohexyl, cyclohexyl; each one is a separate embodiment according to this invention. In other embodiments, R₂ is substituted or unsubstituted C₃-C₈ heterocyclic ring. In other embodiments, R₂ is substituted or unsubstituted aryl (wherein substitutions include: F, Cl, Br, I, C₁-C₅ linear or branched alkyl, OH, alkoxy, N(R)₂, CF₃, CN or NO₂). In other embodiments, R₂ is F. In other embodiments, R₂ is Cl. In other embodiments, R₂ is Br. In other embodiments, R₂ is I. In other embodiments, R₂ is OH. In other embodiments, R₂ is SH. In other embodiments, R₂ is R₈-R₁₂. In other embodiments, R₂ is R₁₂. In other embodiments, R₂ is R₈—OH. In other embodiments, R₂ is R₈—SH. In other embodiments, R₂ is —R₈—O—R₁₀. In other embodiments, R₂ is (CH₂)₂—O—(CH₂)₂—OH. In other embodiments, R₂ is (CH₂)₂—O—(CH₃). In other embodiments, R₂ is (CH₂)₃—O—(CH₃). In other embodiments, R₂ is CF₃. In other embodiments, R₂ is CN. In other embodiments, R₂ is NO₂. In other embodiments, R₂ is —CH₂CN. In other embodiments, R₂ is —R₈CN. In other embodiments, R₂ is NH₂. In other embodiments, R₂ is NHR. In other embodiments, R₂ is N(R)₂. In other embodiments, R₂ is R₈—N(R₁₀)(R₁₁). In other embodiments, R₂ is (CH₂)₃-morpholine. In other embodiments, R₂ is (CH₂)₃—N(Et)₂. In other embodiments, R₂ is —OC(O)CF₃. In other embodiments, R₂ is —OCH₂Ph. In other embodiments, R₂ is —NHCO—R₁₀. In other embodiments, R₂ is NHCO—N(R₁₀)(R₁₁). In other embodiments, R₂ is COOH. In other embodiments, R₂ is —C(O)Ph. In other embodiments, R₂ is C(O)O—R₁₀. In other embodiments, R₂ is R₈—C(O)—R₁₀. In other embodiments, R₂ is C(O)H. In other embodiments, R₂ is C(O)—R₁₀. In other embodiments, R₂ is C₁-C₅ linear or branched C(O)-haloalkyl. In other embodiments, R₂ is —C(O)NH₂. In other embodiments, R₂ is C(O)NHR. In other embodiments, R₂ is C(O)N(R₁₀)(R₁₁). In other embodiments, R₂ is SO₂R. In other embodiments, R₂ is SO₂N(R₁₀)(R₁₁).

In various embodiments, R₂ of formula I and II is represented by formula A:

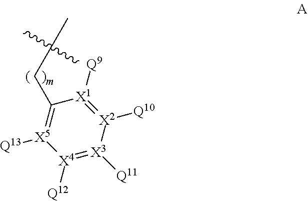

wherein

Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);

or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);

X¹, X², X³, X⁴ and X⁵ are each independently C or N,
wherein
   if X¹, X², X³, X⁴ and/or X⁵ is N then Q⁹, Q¹⁰, Q¹¹, Q¹² and/or Q¹³ is absent respectively.

In various embodiments, R₂ of formula I and II is represented by formula B:

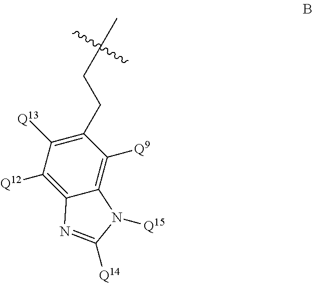

wherein

Q⁹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)-Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);

Q¹⁴ is H, F, Cl, Br, I, OH, SH, R₈—OH (e.g., CH₂—OH), R₈—SH, —R₈—O—R₁₀, (e.g., —CH₂—O—(C═O)—CH₃, CH₂—O—CH₃), C₁-C₅ linear or branched alkylester (e.g., —CH₂—O—(C═O)—CH₃, CH(CH₃)—O(C═O)—CH₃), CF₃, CN, NO₂, —CH₂CN, —R₈CN, NH₂, NHR, N(R)₂, R₈—N(R₁₀)(R₁₁) (e.g., CH₂—N(CH₃)₂), —OC(O)CF₃, —OC(O)NH₂, —NHC(O)NH₂, —OCH₂Ph, NHC(O)—R₁₀ (e.g., NHC(O)CH₃), NHCO—N(R₁₀)(R₁₁) (e.g., NHC(O)N(CH₃)₂), N(R₁₀)C(O)R (e.g., N(CH₃)C(O)(CH₃)), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), substituted or unsubstituted C₁-C₅ linear or branched alkyl (e.g., methyl, ethyl, propyl, iso-propyl, t-Bu, iso-butyl, pentyl, CH(OH)CH₃), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀); and

Q¹⁵ is H, C₁-C₅ linear or branched alkyl (e.g., methyl, ethyl, propyl, iso-propyl, t-Bu, iso-butyl, pentyl), substituted or unsubstituted C₃-C₈ heterocyclic ring (e.g., 1-methyl-pyrrolidine-2-one).

In various embodiments, R₁ and R₂ are joined to form a 5 or 6 membered substituted or unsubstituted, heterocyclic ring. In some embodiments, R₁ and R₂ are joined to form a 5 membered heterocyclic ring. In some embodiments, R₁ and R₂ are joined to form a pyrrolidine. In some embodiments, R₁ and R₂ are joined to form a 2-(piperazin-1-yl)ethanol. In some embodiments, R₁ and R₂ are joined to form a morpholine. In some embodiments, R₁ and R₂ are joined to form a piperazine. In some embodiments, R₁ and R₂ are joined to form a piperidine. In some embodiments, R₁ and R₂ are joined to form a 4-(3-fluoro-4-methoxyphenyl)-1-piperidine. In some embodiments, R₁ and R₂ are joined to form a 5 or 6 membered substituted heterocyclic ring. In some embodiments, the substitutions include at least one selected from: F, Cl, Br, I, substituted or unsubstituted C₁-C₅ linear or branched alkyl, substituted or unsubstituted C₃-C₈ carbocyclic or heterocyclic ring, furan, tetrahydrofuran, morpholine, aryl, benzyl, OH, alkoxy, N(R)₂, CF₃, CN and NO₂; each is a separate embodiment according to this invention.

In various embodiments, R₃, R₄, R₅ and R₆ of formula III are each independently H. In other embodiments, R₃, R₄, R₅ and R₆ are each independently C₁-C₅ linear or branched alkyl. In other embodiment, R₃, R₄, R₅ and R₆ are each independently methyl, ethyl, propyl, i-Pr, cyclopropyl, t-Bu, n-Bu, iso-Bu, pentyl, iso-pentyl, neo-pentyl, cyclopentyl, hexyl, cyclohexyl or heptyl; each one is a separate embodiment according to this invention. In other embodiments, R₃, R₄, R₅ and R₆ are each independently C₁-C₅ linear or branched haloalkyl. In other embodiments, R₃, R₄, R₅ and R₆ are each independently C₁-C₅ linear or branched or C₃-C₈ cyclic alkoxy. In other embodiments, R₃, R₄, R₅ and R₆ are each independently C₁-C₅ linear or branched thioalkoxy. In other embodiments, R₃, R₄, R₅ and R₆ are each independently C₁-C₅ linear or branched haloalkoxy. In other embodiments, R₃, R₄, R₅ and R₆ are each independently C₁-C₅ linear or branched alkoxyalkyl. In other embodiments, R₃, R₄, R₅ and R₆ are each independently substituted or unsubstituted C₃-C₈ cycloalkyl. In other embodiments, R₃, R₄, R₅ and R₆ are each independently substituted or unsubstituted C₃-C₈ heterocyclic ring. In other embodiments, R₃, R₄, R₅ and R₆ are each independently substituted or unsubstituted aryl (wherein substitutions include: F, Cl, Br, I, C₁-C₅ linear or branched alkyl, OH, alkoxy, N(R)₂, CF₃, CN or NO₂; each is a separate embodiment according to this invention. In other embodiments, R₃, R₄, R₅ and R₆ are each independently Cl. In other embodiments, R₃, R₄, R₅ and R₆ are each independently Br. In other embodiments, R₃, R₄, R₅ and R₆ are each independently I. In other embodiments, R₃, R₄, R₅ and R₆ are each independently OH. In other embodiments, R₃, R₄, R₅ and R₆ are each independently SH. In other embodiments, R₃, R₄, R₅ and R₆ are each independently R₈-R₁₂. In other embodiments, R₃, R₄, R₅ and R₆ are each independently R₁₂. In other embodiments, R₃, R₄, R₅ and R₆ are each independently R₈—OH. In other embodiments, R₃, R₄, R₅ and R₆ are each independently R₈—SH. In other embodiments, R₃, R₄, R₅ and R₆ are each independently —R₈—O—R₁₀. In other embodiments, R₃, R₄, R₅ and R₆ are each independently CF₃. In other embodiments, R₃, R₄, R₅ and R₆ are each independently CN. In other embodiments, R₃, R₄, R₅ and R₆ are each independently NO₂. In other embodiments, R₃, R₄, R₅ and R₆ are each independently —CH₂CN. In other embodiments, R₃, R₄, R₅ and R₆ are each independently —R₈CN. In other embodiments, R₃, R₄, R₅ and R₆ are each independently NH₂ In other embodiments, R₃, R₄, R₅ and R₆ are each independently NHR. In other embodiments, R₃, R₄, R₅ and R₆ are each independently N(R)₂. In other embodiments, R₃, R₄, R₅ and R₆ are each independently R₈—N(R₁₀)(R₁₁). In other embodiments, R₃, R₄, R₅ and R₆ are each independently —OC(O)CF₃. In other embodiments, R₃, R₄, R₅ and R₆ are each independently —OCH₂Ph. In other embodiments, R₃, R₄, R₅ and R₆ are each independently —NHCO—R₁₀. In other embodiments, R₃, R₄, R₅ and R₆ are each independently NHCO—N(R₁₀)(R₁₁). In other embodiments, R₃, R₄, R₅ and R₆ are each independently COOH. In other embodiments, R₃, R₄, R₅ and R₆ are each independently —C(O)Ph. In other embodiments, R₃, R₄, R₅ and R₆ are each independently C(O)O—R₁₀. In other embodiments, R₃, R₄, R₅ and R₆ are each independently R₈—C(O)—R₁₀. In other embodiments, R₃, R₄, R₅ and R₆ are each independently C(O)H. In other embodiments, R₃, R₄, R₅ and R₆ are each independently C(O)—R₁₀. In other embodiments, R₃, R₄, R₅ and R₆ are each independently C(O)—CH₃. In other embodiments, R₃, R₄, R₅ and R₆ are each independently C₁-C₅ linear or branched C(O)-haloalkyl. In other embodiments, R₃, R₄, R₅ and R₆ are each independently —C(O)NH₂. In other embodiments, R₃, R₄, R₅ and R₆ are each independently C(O)NHR. In other embodiments, R₃, R₄, R₅ and R₆ are each independently C(O)N(R₁₀)(R₁₁). In other embodiments, R₃, R₄, R₅ and R₆ are each independently SO₂R. In other embodiments, R₃, R₄, R₅ and R₆ are each independently SO₂N(R₁₀)(R₁₁).

In various embodiments, R₃ and R₄ are joined to form a 5 or 6 membered substituted or unsubstituted, carbocyclic or heterocyclic ring. In some embodiments, R₃ and R₄ are joined to form a cyclopentyl ring. In some embodiments, R₃ and R₄ are joined to form a cyclopropyl ring. In some embodiments, R₃ and R₄ are joined to form a cyclopentyl ring. R₃ and R₄ are joined to form a cyclohexyl ring.

In various embodiments, R₅ and R₆ are joined to form a 5 or 6 membered substituted or unsubstituted, carbocyclic or heterocyclic ring. In some embodiments, R₅ and R₆ are joined to form a cyclopentyl ring. In some embodiments, R₅ and R₆ are joined to form a cyclopropyl ring. In some embodiments, R₅ and R₆ are joined to form a cyclopentyl ring. R₅ and R₆ are joined to form a cyclohexyl ring.

In various embodiments, R₈ of formulas I to IX is [CH₂]ₚ wherein p is between 1 and 10. In other embodiment, p is between 1 and 5. In other embodiments, p is between 1 and 3. In other embodiment, p is 1, 2, 3, 4, 5, 6, 7, 8, 9, or 10; each is a separate embodiment according to this invention.

In various embodiments, R₁₀ of formulas I to IX is H. In other embodiments, R₁₀ is C₁-C₅ linear or branched alkyl. In other embodiments, R₁₀ is methyl. In other embodiments, R₁₀ is ethyl. In other embodiments, R₁₀ is propyl. In other embodiments, R₁₀ is i-Pr. In other embodiments, R₁₀ is butyl. In other embodiments, R₁₀ is t-Bu. In other embodiments, R₁₀ is isobutyl. In other embodiments, R₁₀ is pentyl. In other embodiments, R₁₀ is neo-pentyl. In other embodiments, R₁₀ is a substituted or unsubstituted C₃-C₈ cycloalkyl. In other embodiments, R₁₀ is cyclopentyl. In other embodiments, R₁₀ is cyclohexyl. In other embodiments, R₁₀ is a substituted or unsubstituted C₃-C₈ heterocyclic ring. In other embodiments, R₁₀ is 1-methyl piperidine. In other embodiments, R₁₀ is tetrahydro-2H-pirane. In other embodiments, R₁₀ is aryl. In other embodiments, R₁₀ is phenyl. In other embodiments, R₁₀ is R₁₃. In other embodiments, R₁₀ is [(CH₂)₂O]₃—CH₃. In other embodiments, R₁₀ is [(CH₂)₂O]₄—CH₃. In other embodiments, R₁₀ is C(O)R. In other embodiments, R₁₀ is C(O)CH₃. In other embodiments, R₁₀ is O(C═O)R. In other embodiments, R₁₀ is O(C═O)—CH₃. In other embodiments, R₁₀ is R₈—C(O)R. In other embodiments, R₁₀ is CH₂—C(O)CH₃. In other embodiments, R₁₀ is S(O)₂R.

In various embodiments, R₁₁ of formulas I to IX is H. In other embodiments, R₁₁ is C₁-C₅ linear or branched alkyl. In other embodiments, R₁₁ is methyl. In other embodiments, R₁₁ is ethyl. In other embodiments, R₁₁ is propyl. In other embodiments, R₁₁ is i-Pr. In other embodiments, R₁₁ is butyl. In other embodiments, R₁₁ is t-Bu. In other embodiments, R₁₁ is isobutyl. In other embodiments, R₁₁ is pentyl. In other embodiments, R₁₁ is neo-pentyl. In other embodiments, R₁₁ is a substituted or unsubstituted C₃-C₈ cycloalkyl. In other embodiments, R₁₁ is cyclopentyl. In other embodiments, R₁₁ is cyclohexyl. In other embodiments, R₁₁ is a substituted or unsubstituted C₃-C₈ heterocyclic ring. In other embodiments, R₁₁ is 1-methyl piperidine. In other embodiments, R₁₁ is tetrahydro-2H-pirane. In other embodiments, R₁₁ is aryl. In other embodiments, R₁₁ is phenyl. In other embodiments, R₁₁ is R₁₃. In other embodiments, R₁₁ is [(CH₂)₂O]₃—CH₃. In other embodiments, R₁₁ is [(CH₂)₂O]₄—CH₃. In other embodiments, R₁₁ is C(O)R. In other embodiments, R₁₁ is C(O)CH₃. In other embodiments, R₁₁ is O(C═O)R. In other embodiments, R₁₁ is O(C═O)—CH₃. In other embodiments, R₁₁ is R₈—C(O)R. In other embodiments, R₁₁ is CH₂—C(O)CH₃. In other embodiments, R₁₁ is S(O)₂R.

In some embodiments, R₁₀ and R₁₁ are joined to form a 5 or 6 membered substituted or unsubstituted, carbocyclic or heterocyclic ring. In some embodiments, R₁₀ and R₁₁ are joined to form a morpholine ring. In some embodiments, R₁₀ and R₁₁ are joined to form a 5 membered substituted or unsubstituted heterocyclic ring. In some embodiments, R₁₀ and R₁₁ are joined to form a 6 membered substituted or unsubstituted heterocyclic ring. In some embodiments, R₁₀ and R₁₁ are joined to form a 5 or 6 membered substituted or unsubstituted carbocyclic ring. In some embodiments, R₁₀ and R₁₁ are joined to form a 5 or 6 membered substituted carbocyclic ring.

In various embodiments, R₁₂ of formulas I to IX is H. In other embodiments, R₁₂ is C₁-C₅ linear or branched alkyl. In other embodiments, R₁₂ is CH(CH₃)₂. In other embodiments, R₁₂ is methyl. In other embodiments, R₁₂ is ethyl. In other embodiments, R₁₂ is propyl. In other embodiments, R₁₂ is i-Pr. In other embodiments, R₁₂ is cyclopropyl. In other embodiments, R₁₂ is butyl. In other embodiments, R₁₂ is t-Bu. In other embodiments, R₁₂ is isobutyl. In other embodiments, R₁₂ is pentyl. In other embodiments, R₁₂ is neo-pentyl. In other embodiments, R₁₂ is cyclopentyl. In other embodiments, R₁₂ is C₁-C₅ linear or branched haloalkyl. In other embodiments, R₁₂ is substituted or unsubstituted C₃-C₁₂ single or fused cycloalkyl. In other embodiments, R₁₂ is substituted or unsubstituted single or fused C₃-C₁₂ heterocyclic ring. In other embodiments, R₁₂ is 2-tetrahydropyranyl. In other embodiments, R₁₂ is 3-tetrahydropyranyl. In other embodiments, R₁₂ is 4-tetrahydropyranyl. In other embodiments, R₁₂ is 1H-benzo[d]imidazol-2(3H)-one. In other embodiments, R₁₂ is substituted or unsubstituted single or fused aryl. In other embodiments, R₁₂ is substituted or unsubstituted single or fused aryl; wherein substitutions include: F, Cl, Br, I, substituted or unsubstituted C₁-C₅ linear or branched alkyl (e.g. methyl, isopropyl, CH₂—OH, CH(OH)—CH₃), R₁₀ (e.g., C(O)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH₂—O(C═O)—CH₃, CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂. In other embodiments, R₁₂ is phenyl. In other embodiments, R₁₂ is 2-methoxyphenyl. In other embodiments, R₁₂ is 3-methoxyphenyl. In other embodiments, R₁₂ is 4-methoxyphenyl. In other embodiments, R₁₂ is 4-fluoro-3-methoxyphenyl. In other embodiments, R₁₂ is 3-fluoro-4-methoxyphenyl. In other embodiments, R₁₂ is 3,4-difluorophenyl. In other embodiments, R₁₂ is 3,5-dimethoxyphenyl. In other embodiments, R₁₂ is 2,3-dimethoxyphenyl. In other embodiments, R₁₂ is 2,4-dimethoxyphenyl. In other embodiments, R₁₂ is 3,4-dimethoxyphenyl. In other embodiments, R₁₂ is 4-acetamide-phenyl. In other embodiments, R₁₂ is 4-benzoicacid. In other embodiments, R₁₂ is 3-methoxy-4-propoxyphenyl. In other embodiments, R₁₂ is methyl-4-benzoate. In other embodiments, R₁₂ is 2,3-dihydrobenzo[b][1,4]dioxinyl. In other embodiments, R₁₂ is 3-fluoro-4-hydroxyphenyl. In other embodiments, R₁₂ is 4-phenylurea. In other embodiments, R₁₂ is 4-methylbenzamide. In other embodiments, R₁₂ is 4-phenylcarbamate. In other embodiments, R₁₂ is 4-benzamide. In other embodiments, R₁₂ is 4-N,N-dimethylbenzamide. In other embodiments, R₁₂ is aniline. In other embodiments, R₁₂ is N-phenylmethanesulfonamide. In other embodiments, R₁₂ is 4-N-methyl-N-phenylmethanesulfonamide. In other embodiments, R₁₂ is 4-phenol. In other embodiments, R₁₂ is methyl benzenesulfonate. In other embodiments, R₁₂ is N-methyl-N-phenylacetamide. In other embodiments, R₁₂ is 4-methoxy-3-(trifluoromethyl)benzene. In other embodiments, R₁₂ is 2-nitrophenolyl. In other embodiments, R₁₂ is N-methyl-2-nitroaniline. In other embodiments, R₁₂ is 2,2,2-trifluoro-N-(2-nitrophenyl)acetamide. In other embodiments, R₁₂ is 3-(2-aminophenyl)-2-oxopropyl acetate. In other embodiments, R₁₂ is 3-(2-nitrophenyl)-2-oxopropyl acetate. In other embodiments, R₁₂ is 1-((2-aminophenyl)amino)-1-oxopropan-2-yl acetate. In other embodiments, R₁₂ is 1-((2-fluorophenyl)amino)propan-2-one. In other embodiments, R₁₂ is N-(2-aminophenyl)benzamide. In other embodiments, R₁₂ is benzene-1,2-diamine. In other embodiments, R₁₂ is substituted or unsubstituted single or fused heteroaryl; wherein substitutions include: F, Cl, Br, I, substituted or unsubstituted C₁-C₅ linear or branched alkyl (e.g. methyl, isopropyl, CH₂—OH, CH(OH)—CH₃), R₁₀ (e.g., C(O)—CH₃), R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH₂—O(C═O)—CH₃, CH(CH₃)—O—(C═O)—CH₃), OH, alkoxy (e.g., methoxy), N(R)₂ (e.g., NH₂), NHR (e.g., NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂. In other embodiments, R₁₂ is benzimidazolyl. In other embodiments, R₁₂ is 1-methyl-benzimidazolyl. In other embodiments, R₁₂ is benzooxazolyl. In other embodiments, R₁₂ is 2-methyl-1H-benzo[d]imidazole. In other embodiments, R₁₂ is 2-methoxy-1H-benzo[d]imidazole. In other embodiments, R₁₂ is indolyl. In other embodiments, R₁₂ is 2-pyridinyl. In other embodiments, R₁₂ is 3-pyridinyl. In other embodiments, R₁₂ is 4-pyridinyl. In other embodiments, R₁₂ is pyrimidinyl. In other embodiments, R₁₂ is C₁-C₅ linear or branched or C₃-C₈ cyclic alkoxy. In other embodiments, R₁₂ is C₁-C₅ linear or branched thioalkoxy. In other embodiments, R₁₂ is C₁-C₅ linear or branched haloalkoxy. In other embodiments, R₁₂ is C₁-C₅ linear or branched alkoxyalkyl.

In various embodiments, R₁₃ of formulas I to IX is [(CH₂)₂O]ₙ—CH₃ wherein n is between 1 and 20 (e.g., 1, 3, 4, 5). In some embodiments, R₁₃ is [(CH₂)₂O]—CH₃. In some embodiments, R₁₃ is [(CH₂)₂O]₂—CH₃. In some embodiments, R₁₃ is [(CH₂)₂O]₃—CH₃. In some embodiments, R₁₃ is [(CH₂)₂O]₄—CH₃. In some embodiments, R₁₃ is [(CH₂)₂O]₅—CH₃. In some embodiments, R₁₃ is [(CH₂)₂O]₆—CH₃. In some embodiments, R₁₃ is [(CH₂)₂O]₇—CH₃. In some embodiments, R₁₃ is [(CH₂)₂O]₅—CH₃. In some embodiments, R₁₃ is [(CH₂)₂O]₉—CH₃.

In various embodiments, R of formulas I to IX is H. In other embodiments, R is C₁-C₅ linear or branched alkyl. In other embodiments, R is methyl. In other embodiments, R is ethyl. In other embodiments, R is propyl. In other embodiments, R is i-Pr. In other embodiments, R is cyclopropyl. In other embodiments, R is butyl. In other embodiments, R is t-Bu. In other embodiments, R is isobutyl. In other embodiments, R is pentyl. In other embodiments, R is neo-pentyl. In other embodiments, R is cyclopentyl. In other embodiments, R is C₁-C₅ linear or branched alkoxy. In other embodiments, R is methoxy. In other embodiments, R is ethoxy. In other embodiments, R is propoxy. In other embodiments, R is iso-propoxy. In other embodiments, R is C₁-C₅ linear or branched haloalkyl. In other embodiments, R is CF₃. In other embodiments, R is C₁-C₅ linear or branched alkyl ester. In other embodiments, R is CH₂—O—(CO)—CH₃. In other embodiments, R is (C═O)—CH(CH₃)—O—(C═O)—CH₃. In other embodiments, R is (CH₂—(C═O)—CH₃). In other embodiments, R is phenyl. In other embodiments, R is —(C═O)—CH₃. In other embodiments, R is —(C═O)—CF₃. In other embodiments, R is —(C═O)-Ph. In other embodiments, R is aryl. In other embodiments, R is heteroaryl. In other embodiments, two gem R substituents are joined to form a 5 or 6 membered heterocyclic ring.

In various embodiments, W¹ of formulas I-V is CH. In other embodiments, W¹ is C(R). In other embodiments, W¹ is N.

In various embodiments, W² of formulas I-V is CH. In other embodiments, W² is C(R). In other embodiments, W² is N.

In various embodiments, W⁴ of formulas I-V is CH. In other embodiments, W⁴ is C(R). In other embodiments, W⁴ is N.

In various embodiments, W³ of formulas I-V is S. In other embodiments, W³ is SO. In other embodiments, W³ is SO₂. In other embodiments, W³ is O. In other embodiments, W³ is N—OH. In other embodiments, W³ is CH₂. In other embodiments, W³ is C(R)₂. In other embodiments, W³ is N—OMe.

In various embodiments, W⁵ of formulas I-VII is a bond. In other embodiments, W⁵ is S. In other embodiments, W⁵ is O. In other embodiments, W⁵ is NH. In other embodiments, W⁵ is N(R). In other embodiments, W⁵ is N—CH₃. In other embodiments, W⁵ is N(R)₂. In other embodiments, W⁵ is CH₂. In other embodiments, W⁵ is CH(R). In other embodiments, W⁵ is C(R)₂. In other embodiments, W⁵ is N—OH. In other embodiments, W⁵ is N—OR.

In various embodiments, S═X of formulas VI-IX is S. In other embodiments, S═X is SO. In other embodiments, S═X is SO₂.

In various embodiments, Q¹ of formulas II and III is H. In other embodiments, Q¹ is F. In other embodiments, Q¹ is Cl. In other embodiments, Q¹ is Br. In other embodiments, Q¹ is I. In other embodiments, Q¹ is OH. In other embodiments, Q¹ is SH. In other embodiments, Q¹ is R₈—OH. In other embodiments, Q¹ is R₈—SH. In other embodiments, Q¹ is —R₈—O—R₁₀. In other embodiments, Q¹ is CF₃. In other embodiments, Q¹ is CN. In other embodiments, Q¹ is NO₂. In other embodiments, Q¹ is —CH₂CN. In other embodiments, Q¹ is —R₈CN. In other embodiments, Q¹ is NH₂. In other embodiments, Q¹ is NHR. In other embodiments, Q¹ is N(R)₂. In other embodiments, Q¹ is R₈—N(R₁₀)(R₁₁). In other embodiments, Q¹ is —OC(O)CF₃. In other embodiments, Q¹ is —OCH₂Ph. In other embodiments, Q¹ is NHC(O)—R₁₀. In other embodiments, Q¹ is NHCO—N(R₁₀)(R₁₁). In other embodiments, Q¹ is COOH. In other embodiments, Q¹ is —C(O)Ph. In other embodiments, Q¹ is C(O)O—R₁₀. In other embodiments, Q¹ is R₈—C(O)—R₁₀. In other embodiments, Q¹ is C(O)H. In other embodiments, Q¹ is C(O)—R₁₀. In other embodiments, Q¹ is C₁-C₅ linear or branched C(O)-haloalkyl. In other embodiments, Q¹ is —C(O)NH₂. In other embodiments, Q¹ is C(O)NHR. In other embodiments, Q¹ is C(O)N(R₁₀)(R₁₁). In other embodiments, Q¹ is SO₂R. In other embodiments, Q¹ is SO₂N(R₁₀)(R₁₁). In other embodiments, Q¹ is CH(CF₃)(NH—R₁₀). In other embodiments, Q¹ is C₁-C₅ linear or branched alkyl. In other embodiments, Q¹ is methyl. In other embodiments, Q¹ is ethyl. In other embodiments, Q¹ is propyl. In other embodiments, Q¹ is isopropyl. In other embodiments, Q¹ is cyclopropyl. In other embodiments, Q¹ is butyl. In other embodiments, Q¹ is t-Bu. In other embodiments, Q¹ is isobutyl. In other embodiments, Q¹ is pentyl. In other embodiments, Q¹ is neo-pentyl. In other embodiments, Q¹ is cyclopentyl. In other embodiments, Q¹ is C₁-C₅ linear or branched haloalkyl. In other embodiments, Q¹ is C₁-C₅ linear or branched or C₃-C₈ cyclic alkoxy. In other embodiments, Q¹ is C₁-C₅ linear or branched thioalkoxy. In other embodiments, Q¹ is C₁-C₅ linear or branched haloalkoxy. In other embodiments, Q¹ is C₁-C₅ linear or branched alkoxyalkyl. In other embodiments, Q¹ is substituted or unsubstituted C₃-C₈ cycloalkyl. In other embodiments, Q¹ is substituted or unsubstituted cyclopentyl. In other embodiments, Q¹ is substituted or unsubstituted cyclohexyl. In other embodiments, Q¹ is substituted or unsubstituted C₃-C₈ heterocyclic ring. In other embodiments, Q¹ is substituted or unsubstituted aryl. In other embodiments, Q¹ is phenyl. In some embodiments, Q¹ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q² of formulas II and III is H. In other embodiments, Q² is F. In other embodiments, Q² is Cl. In other embodiments, Q² is Br. In other embodiments, Q² is I. In other embodiments, Q² is OH. In other embodiments, Q² is SH. In other embodiments, Q² is R₈—OH. In other embodiments, Q² is R₈—SH. In other embodiments, Q² is —R₈—O—R₁₀. In other embodiments, Q² is CF₃. In other embodiments, Q² is CN. In other embodiments, Q² is NO₂. In other embodiments, Q² is —CH₂CN. In other embodiments, Q² is —R₈CN. In other embodiments, Q² is NH₂. In other embodiments, Q² is NHR. In other embodiments, Q² is N(R)₂. In other embodiments, Q² is R₈—N(R₁₀)(R₁₁). In other embodiments, Q² is —OC(O)CF₃. In other embodiments, Q² is —OCH₂Ph. In other embodiments, Q² is NHC(O)—R₁₀. In other embodiments, Q² is NHCO—N(R₁₀)(R₁₁). In other embodiments, Q² is COOH. In other embodiments, Q² is —C(O)Ph. In other embodiments, Q² is C(O)O—R₁₀. In other embodiments, Q² is R₈—C(O)—R₁₀. In other embodiments, Q² is C(O)H. In other embodiments, Q² is C(O)—R₁₀. In other embodiments, Q² is C₁-C₅ linear or branched C(O)-haloalkyl. In other embodiments, Q² is —C(O)NH₂. In other embodiments, Q² is C(O)NHR. In other embodiments, Q² is C(O)N(R₁₀)(R₁₁). In other embodiments, Q² is SO₂R. In other embodiments, Q² is SO₂N(R₁₀)(R₁₁). In other embodiments, Q² is CH(CF₃)(NH—R₁₀). In other embodiments, Q² is C₁-C₅ linear or branched alkyl. In other embodiments, Q² is methyl. In other embodiments, Q² is ethyl. In other embodiments, Q² is propyl. In other embodiments, Q² is isopropyl. In other embodiments, Q² is cyclopropyl. In other embodiments, Q² is butyl. In other embodiments, Q² is t-Bu. In other embodiments, Q² is isobutyl. In other embodiments, Q² is pentyl. In other embodiments, Q² is neo-pentyl. In other embodiments, Q² is cyclopentyl. In other embodiments, Q² is C₁-C₅ linear or branched haloalkyl. In other embodiments, Q² is C₁-C₅ linear or branched or C₃-C₈ cyclic alkoxy. In other embodiments, Q² is C₁-C₅ linear or branched thioalkoxy. In other embodiments, Q² is C₁-C₅ linear or branched haloalkoxy. In other embodiments, Q² is C₁-C₅ linear or branched alkoxyalkyl. In other embodiments, Q² is substituted or unsubstituted C₃-C₈ cycloalkyl. In other embodiments, Q² is substituted or unsubstituted cyclopentyl. In other embodiments, Q² is substituted or unsubstituted cyclohexyl In other embodiments, Q² is substituted or unsubstituted C₃-C₈ heterocyclic ring. In other embodiments, Q² is substituted or unsubstituted aryl. In other embodiments, Q² is phenyl. In some embodiments, Q² may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q³ of formulas II-VII is H. In other embodiments, Q³ is F. In other embodiments, Q³ is Cl. In other embodiments, Q³ is Br. In other embodiments, Q³ is I. In other embodiments, Q³ is OH. In other embodiments, Q³ is SH. In other embodiments, Q³ is R₈—OH. In other embodiments, Q³ is R₈—SH. In other embodiments, Q³ is —R₈—O—R₁₀. In other embodiments, Q³ is CF₃. In other embodiments, Q³ is CN. In other embodiments, Q³ is NO₂. In other embodiments, Q³ is —CH₂CN. In other embodiments, Q³ is —R₈CN. In other embodiments, Q³ is NH₂. In other embodiments, Q³ is NHR. In other embodiments, Q³ is N(R)₂. In other embodiments, Q³ is R₈—N(R₁₀)(R₁₁). In other embodiments, Q³ is —OC(O)CF₃. In other embodiments, Q³ is —OCH₂Ph. In other embodiments, Q³ is NHC(O)—R₁₀. In other embodiments, Q³ is NHCO—N(R₁₀)(R₁₁). In other embodiments, Q³ is COOH. In other embodiments, Q³ is —C(O)Ph. In other embodiments, Q³ is C(O)O—R₁₀. In other embodiments, Q³ is R₈—C(O)—R₁₀. In other embodiments, Q³ is C(O)H. In other embodiments, Q³ is C(O)—R₁₀. In other embodiments, Q³ is C₁-C₅ linear or branched C(O)-haloalkyl. In other embodiments, Q³ is —C(O)NH₂. In other embodiments, Q³ is C(O)NHR. In other embodiments, Q³ is C(O)N(R₁₀)(R₁₁). In other embodiments, Q³ is SO₂R. In other embodiments, Q³ is SO₂N(R₁₀)(R₁₁). In other embodiments, Q³ is CH(CF₃)(NH—R₁₀). In other embodiments, Q³ is C₁-C₅ linear or branched alkyl. In other embodiments, Q³ is methyl. In other embodiments, Q³ is ethyl. In other embodiments, Q³ is propyl. In other embodiments, Q¹ is isopropyl. In other embodiments, Q³ is cyclopropyl. In other embodiments, Q³ is butyl. In other embodiments, Q³ is t-Bu. In other embodiments, Q³ is isobutyl. In other embodiments, Q³ is pentyl. In other embodiments, Q³ is neo-pentyl. In other embodiments, Q³ is cyclopentyl. In other embodiments, Q³ is C₁-C₅ linear or branched haloalkyl. In other embodiments, Q³ is C₁-C₅ linear or branched or C₃-C₈ cyclic alkoxy. In other embodiments, Q³ is C₁-C₅ linear or branched thioalkoxy. In other embodiments, Q³ is C₁-C₅ linear or branched haloalkoxy. In other embodiments, Q³ is C₁-C₅ linear or branched alkoxyalkyl. In other embodiments, Q³ is substituted or unsubstituted C₃-C₈ cycloalkyl. In other embodiments, Q³ is substituted or unsubstituted cyclopentyl. In other embodiments, Q³ is substituted or unsubstituted cyclohexyl In other embodiments, Q³ is substituted or unsubstituted C₃-C₈ heterocyclic ring. In other embodiments, Q³ is substituted or unsubstituted aryl. In other embodiments, Q³ is phenyl. In some embodiments, Q³ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁴ of formulas II-VII is H. In other embodiments, Q⁴ is F. In other embodiments, Q⁴ is Cl. In other embodiments, Q⁴ is Br. In other embodiments, Q⁴ is I. In other embodiments, Q⁴ is OH. In other embodiments, Q⁴ is SH. In other embodiments, Q⁴ is R₈—OH. In other embodiments, Q⁴ is R₈—SH. In other embodiments, Q⁴ is —R₈—O—R₁₀. In other embodiments, Q⁴ is CF₃. In other embodiments, Q⁴ is CN. In other embodiments, Q⁴ is NO₂. In other embodiments, Q⁴ is —CH₂CN. In other embodiments, Q⁴ is —R₈CN. In other embodiments, Q⁴ is NH₂. In other embodiments, Q⁴ is NHR. In other embodiments, Q⁴ is N(R)₂. In other embodiments, Q⁴ is R₈—N(R₁₀)(R₁₁). In other embodiments, Q⁴ is —OC(O)CF₃. In other embodiments, Q⁴ is —OCH₂Ph. In other embodiments, Q⁴ is NHC(O)—R₁₀. In other embodiments, Q⁴ is NHCO—N(R₁₀)(R₁₁). In other embodiments, Q⁴ is COOH. In other embodiments, Q⁴ is —C(O)Ph. In other embodiments, Q⁴ is C(O)O—R₁₀. In other embodiments, Q⁴ is R₈—C(O)—R₁₀. In other embodiments, Q⁴ is C(O)H. In other embodiments, Q⁴ is C(O)—R₁₀. In other embodiments, Q⁴ is C₁-C₅ linear or branched C(O)-haloalkyl. In other embodiments, Q⁴ is —C(O)NH₂. In other embodiments, Q⁴ is C(O)NHR. In other embodiments, Q⁴ is C(O)N(R₁₀)(R₁₁). In other embodiments, Q⁴ is SO₂R. In other embodiments, Q⁴ is SO₂N(R₁₀)(R₁₁). In other embodiments, Q⁴ is CH(CF₃)(NH—R₁₀). In other embodiments, Q⁴ is C₁-C₅ linear or branched alkyl. In other embodiments, Q⁴ is methyl. In other embodiments, Q⁴ is ethyl. In other embodiments, Q⁴ is propyl. In other embodiments, Q⁴ is isopropyl. In other embodiments, Q⁴ is cyclopropyl. In other embodiments, Q⁴ is butyl. In other embodiments, Q⁴ is t-Bu. In other embodiments, Q⁴ is isobutyl. In other embodiments, Q⁴ is pentyl. In other embodiments, Q⁴ is neo-pentyl. In other embodiments, Q⁴ is cyclopentyl. In other embodiments, Q⁴ is C₁-C₅ linear or branched haloalkyl. In other embodiments, Q⁴ is C₁-C₅ linear or branched or C₃-C₈ cyclic alkoxy. In other embodiments, Q⁴ is C₁-C₅ linear or branched thioalkoxy. In other embodiments, Q⁴ is C₁-C₅ linear or branched haloalkoxy. In other embodiments, Q⁴ is C₁-C₅ linear or branched alkoxyalkyl. In other embodiments, Q⁴ is substituted or unsubstituted C₃-C₈ cycloalkyl. In other embodiments, Q⁴ is substituted or unsubstituted cyclopentyl. In other embodiments, Q⁴ is substituted or unsubstituted cyclohexyl. In other embodiments, Q⁴ is substituted or unsubstituted C₃-C₈ heterocyclic ring. In other embodiments, Q⁴ is substituted or unsubstituted aryl. In other embodiments, Q⁴ is phenyl. In some embodiments, Q⁴ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁵ of formulas II and III is H. In other embodiments, Q⁵ is F. In other embodiments, Q⁵ is Cl. In other embodiments, Q⁵ is Br. In other embodiments, Q⁵ is I. In other embodiments, Q⁵ is OH. In other embodiments, Q⁵ is SH. In other embodiments, Q⁵ is R₈—OH. In other embodiments, Q⁵ is R₈—SH. In other embodiments, Q⁵ is —R₈—O—R₁₀. In other embodiments, Q⁵ is CF₃. In other embodiments, Q⁵ is CN. In other embodiments, Q⁵ is NO₂. In other embodiments, Q⁵ is —CH₂CN. In other embodiments, Q⁵ is —R₈CN. In other embodiments, Q⁵ is NH₂. In other embodiments, Q⁵ is NHR. In other embodiments, Q⁵ is N(R)₂. In other embodiments, Q⁵ is R₈—N(R₁₀)(R₁₁). In other embodiments, Q⁵ is —OC(O)CF₃. In other embodiments, Q⁵ is —OCH₂Ph. In other embodiments, Q⁵ is NHC(O)—R₁₀. In other embodiments, Q⁵ is NHCO—N(R₁₀)(R₁₁). In other embodiments, Q⁵ is COOH. In other embodiments, Q⁵ is —C(O)Ph. In other embodiments, Q⁵ is C(O)O—R₁₀. In other embodiments, Q⁵ is R₈—C(O)—R₁₀. In other embodiments, Q⁵ is C(O)H. In other embodiments, Q⁵ is C(O)—R₁₀. In other embodiments, Q⁵ is C₁-C₅ linear or branched C(O)-haloalkyl. In other embodiments, Q⁵ is —C(O)NH₂. In other embodiments, Q⁵ is C(O)NHR. In other embodiments, Q⁵ is C(O)N(R₁₀)(R₁₁). In other embodiments, Q⁵ is SO₂R. In other embodiments, Q⁵ is SO₂N(R₁₀)(R₁₁). In other embodiments, Q⁵ is CH(CF₃)(NH—R₁₀). In other embodiments, Q⁵ is C₁-C₅ linear or branched alkyl. In other embodiments, Q⁵ is methyl. In other embodiments, Q⁵ is ethyl. In other embodiments, Q⁵ is propyl. In other embodiments, Q⁵ is i-Pr. In other embodiments, Q⁵ is cyclopropyl. In other embodiments, Q⁵ is butyl. In other embodiments, Q⁵ is t-Bu. In other embodiments, Q⁵ is isobutyl. In other embodiments, Q⁵ is pentyl. In other embodiments, Q⁵ is neo-pentyl. In other embodiments, Q⁵ is cyclopentyl. In other embodiments, Q⁵ is C₁-C₅ linear or branched haloalkyl. In other embodiments, Q⁵ is C₁-C₅ linear or branched or C₃-C₈ cyclic alkoxy. In other embodiments, Q⁵ is C₁-C₅ linear or branched thioalkoxy. In other embodiments, Q⁵ is C₁-C₅ linear or branched haloalkoxy. In other embodiments, Q⁵ is C₁-C₅ linear or branched alkoxyalkyl. In other embodiments, Q⁵ is substituted or unsubstituted C₃-C₈ cycloalkyl. In other embodiments, Q⁵ is substituted or unsubstituted cyclopentyl. In other embodiments, Q⁵ is substituted or unsubstituted cyclohexyl. In other embodiments, Q⁵ is substituted or unsubstituted C₃-C₈ heterocyclic ring. In other embodiments, Q⁵ is substituted or unsubstituted aryl. In other embodiments, Q⁵ is phenyl. In some embodiments, Q⁵ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁶ of formulas II and III is H. In other embodiments, Q⁶ is F. In other embodiments, Q⁶ is Cl. In other embodiments, Q⁶ is Br. In other embodiments, Q⁶ is I. In other embodiments, Q⁶ is OH. In other embodiments, Q⁶ is SH. In other embodiments, Q⁶ is R₈—OH. In other embodiments, Q⁶ is R₈—SH. In other embodiments, Q⁶ is —R₈—O—R₁₀. In other embodiments, Q⁶ is CF₃. In other embodiments, Q⁶ is CN. In other embodiments, Q⁶ is NO₂. In other embodiments, Q⁶ is —CH₂CN. In other embodiments, Q⁶ is —R₈CN. In other embodiments, Q⁶ is NH₂. In other embodiments, Q⁶ is NHR. In other embodiments, Q⁶ is N(R)₂. In other embodiments, Q⁶ is R₈—N(R₁₀)(R₁₁). In other embodiments, Q⁶ is —OC(O)CF₃. In other embodiments, Q⁶ is —OCH₂Ph. In other embodiments, Q⁶ is NHC(O)—R₁₀. In other embodiments, Q⁶ is NHCO—N(R₁₀)(R₁₁). In other embodiments, Q⁶ is COOH. In other embodiments, Q⁶ is —C(O)Ph. In other embodiments, Q⁶ is C(O)O—R₁₀. In other embodiments, Q⁶ is R₈—C(O)—R₁₀. In other embodiments, Q⁶ is C(O)H. In other embodiments, Q⁶ is C(O)—R₁₀. In other embodiments, Q⁶ is C₁-C₅ linear or branched C(O)-haloalkyl. In other embodiments, Q⁶ is —C(O)NH₂. In other embodiments, Q⁶ is C(O)NHR. In other embodiments, Q⁶ is C(O)N(R₁₀)(R₁₁). In other embodiments, Q⁶ is SO₂R. In other embodiments, Q⁶ is SO₂N(R₁₀)(R₁₁). In other embodiments, Q⁶ is CH(CF₃)(NH—R₁₀). In other embodiments, Q⁶ is C₁-C₅ linear or branched alkyl. In other embodiments, Q⁶ is methyl. In other embodiments, Q⁶ is ethyl. In other embodiments, Q⁶ is propyl. In other embodiments, Q⁶ is isopropyl. In other embodiments, Q⁶ is cyclopropyl. In other embodiments, Q⁶ is butyl. In other embodiments, Q⁶ is t-Bu. In other embodiments, Q⁶ is isobutyl. In other embodiments, Q⁶ is pentyl. In other embodiments, Q⁶ is neo-pentyl. In other embodiments, Q⁶ is cyclopentyl. In other embodiments, Q⁶ is C₁-C₅ linear or branched haloalkyl. In other embodiments, Q⁶ is C₁-C₅ linear or branched or C₃-C₈ cyclic alkoxy. In other embodiments, Q⁶ is C₁-C₅ linear or branched thioalkoxy. In other embodiments, Q⁶ is C₁-C₅ linear or branched haloalkoxy. In other embodiments, Q⁶ is C₁-C₅ linear or branched alkoxyalkyl. In other embodiments, Q⁶ is substituted or unsubstituted C₃-C₈ cycloalkyl. In other embodiments, Q⁶ is substituted or unsubstituted cyclopentyl. In other embodiments, Q⁶ is substituted or unsubstituted cyclohexyl. In other embodiments, Q⁶ is substituted or unsubstituted C₃-C₈ heterocyclic ring. In other embodiments, Q⁶ is substituted or unsubstituted aryl. In other embodiments, Q⁶ is phenyl. In some embodiments, Q⁶ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁷ of formulas I-IX is H. In other embodiments, Q⁷ is C(O)O—R₁₃ wherein n is an integer number between 1 and 20 (e.g., n=1, 3, 4, 5). In other embodiments, Q⁷ is R₈—O—R₁₃. In other embodiments, Q⁷ is CH₂—O—(CH₂)₂O—CH₃. In other embodiments, Q⁷ is CH₂—[O—(CH₂)₂O]₃—CH₃. In other embodiments, Q⁷ is CH₂—[O—(CH₂)₂O]₄—CH₃. In other embodiments, Q⁷ is CH₂—[O—(CH₂)₂O]₅—CH₃. In other embodiments, Q⁷ is C(O)O—(CH₂)ₖ—COOH wherein k is an integer number between 1 and 20 (e.g., k=7, 11, 15). In other embodiments, Q⁷ is F. In other embodiments, Q⁷ is Cl. In other embodiments, Q⁷ is Br. In other embodiments, Q⁷ is I. In other embodiments, Q⁷ is OH. In other embodiments, Q⁷ is SH. In other embodiments, Q⁷ is R₈—OH. In other embodiments, Q⁷ is (CH₂)—OH). In other embodiments, Q⁷ is R₈—SH. In other embodiments, Q⁷ is —R₈—O—R₁₀. In other embodiments, Q⁷ is (CH₂)—O—CH₃. In other embodiments, Q⁷ is (CH₂)₄O—CH₃. In other embodiments, Q⁷ is (CH₂)—O-cyclopentyl. In other embodiments, Q⁷ is (CH₂)—O— cyclohexyl. In other embodiments, Q⁷ is (CH₂)—O-(1-methyl-piperidine). In other embodiments, Q⁷ is (CH₂)—O-iPr). In other embodiments, Q⁷ is —R₈—S—R₁₀. In other embodiments, Q⁷ is CH₂—S—CH₃. In other embodiments, Q⁷ is CF₃. In other embodiments, Q⁷ is CN. In other embodiments, Q⁷ is NO₂. In other embodiments, Q⁷ is —CH₂CN. In other embodiments, Q⁷ is —R₈CN. In other embodiments, Q⁷ is NH₂. In other embodiments, Q⁷ is NHR. In other embodiments, Q⁷ is N(R)₂. In other embodiments, Q⁷ is R₈—N(R₁₀)(R₁₁). In other embodiments, Q⁷ is (CH₂)—N(CH₃)₂. In other embodiments, Q⁷ is (CH₂)—N(CH₃)([(CH₂)₂O]ₙ—CH₃); wherein n is 1, 3, 4; each is a separate embodiment according to this invention. In other embodiments, Q⁷ is —OC(O)CF₃. In other embodiments, Q⁷ is —OCH₂Ph. In other embodiments, Q⁷ is NHC(O)—R₁₀. In other embodiments, Q⁷ is NHCO—N(R₁₀)(R₁₁). In other embodiments, Q⁷ is COOH. In other embodiments, Q⁷ is —C(O)Ph. In other embodiments, Q⁷ is C(O)O—R₁₀. In other embodiments, Q⁷ is C(═O)O—C₂H₅. In other embodiments, Q⁷ is R₈—C(O)—R₁₀. In other embodiments, Q⁷ is C(O)H. In other embodiments, Q⁷ is C(O)—R₁₀. In other embodiments, Q⁷ is C₁-C₅ linear or branched C(O)-haloalkyl. In other embodiments, Q⁷ is —C(O)NH₂. In other embodiments, Q⁷ is C(O)NHR. In other embodiments, Q⁷ is C(O)N(R₁₀)(R₁₁). In other embodiments, Q⁷ is C(O)N(CH₃)₂. In other embodiments, Q⁷ is C(O)N(H)(R₁₃); wherein n is 4. In other embodiments, Q⁷ is SO₂R. In other embodiments, Q⁷ is SO₂—CH₃. In other embodiments, Q⁷ is SO₂N(R₁₀)(R₁₁). In other embodiments, Q⁷ is CH(CF₃)(NH—R₁₀). In other embodiments, Q⁷ is C₁-C₅ linear or branched alkyl. In other embodiments, Q⁷ is methyl. In other embodiments, Q⁷ is ethyl. In other embodiments, Q⁷ is propyl. In other embodiments, Q⁷ is isopropyl. In other embodiments, Q⁷ is cyclopropyl. In other embodiments, Q⁷ is butyl. In other embodiments, Q⁷ is t-Bu. In other embodiments, Q⁷ is isobutyl. In other embodiments, Q⁷ is pentyl. In other embodiments, Q⁷ is neo-pentyl. In other embodiments, Q⁷ is cyclopentyl. In other embodiments, Q⁷ is C₁-C₅ linear or branched haloalkyl. In other embodiments, Q⁷ is C₁-C₅ linear or branched or C₃-C₈ cyclic alkoxy. In other embodiments, Q⁷ is C₁-C₅ linear or branched thioalkyl. In other embodiments, Q⁷ is S—CH₃. In other embodiments, Q⁷ is C₁-C₅ linear or branched thioalkoxy. In other embodiments, Q⁷ is C₁-C₅ linear or branched haloalkoxy. In other embodiments, Q⁷ is C₁-C₅ linear or branched alkoxyalkyl. In other embodiments, Q⁷ is substituted or unsubstituted C₃-C₈ cycloalkyl. In other embodiments, Q⁷ is cyclopentyl. In other embodiments, Q⁷ is cyclohexyl. In other embodiments, Q⁷ is substituted or unsubstituted C₃-C₈ heterocyclic ring. In other embodiments, Q⁷ is thiazole. In other embodiments, Q⁷ is substituted or unsubstituted aryl. In other embodiments, Q⁷ is phenyl. In some embodiments, Q⁷ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁸ of formulas I-IV is H. In other embodiments, Q⁸ is F. In other embodiments, Q⁸ is Cl. In other embodiments, Q⁸ is Br. In other embodiments, Q⁸ is I. In other embodiments, Q⁸ is OH. In other embodiments, Q⁸ is SH. In other embodiments, Q⁸ is R₈—OH. In other embodiments, Q⁸ is R₈—SH. In other embodiments, Q⁸ is —R₈—O—R₁₀. In other embodiments, Q⁸ is CF₃. In other embodiments, Q⁸ is CN. In other embodiments, Q⁸ is NO₂. In other embodiments, Q⁸ is —CH₂CN. In other embodiments, Q⁸ is —R₈CN. In other embodiments, Q⁸ is NH₂. In other embodiments, Q⁸ is NHR. In other embodiments, Q⁸ is N(R)₂. In other embodiments, Q⁸ is R₈—N(R₁₀)(R₁₁). In other embodiments, Q⁸ is —OC(O)CF₃. In other embodiments, Q⁸ is —OCH₂Ph. In other embodiments, Q⁸ is NHC(O)—R₁₀. In other embodiments, Q⁸ is NHCO—N(R₁₀)(R₁₁). In other embodiments, Q⁸ is COOH. In other embodiments, Q⁸ is —C(O)Ph. In other embodiments, Q⁸ is C(O)O—R₁₀. In other embodiments, Q⁸ is R₈—C(O)—R₁₀. In other embodiments, Q⁸ is C(O)H. In other embodiments, Q⁸ is C(O)—R₁₀. In other embodiments, Q⁸ is C₁-C₅ linear or branched C(O)-haloalkyl. In other embodiments, Q⁸ is —C(O)NH₂. In other embodiments, Q⁸ is C(O)NHR. In other embodiments, Q⁸ is C(O)N(R₁₀)(R₁₁). In other embodiments, Q⁸ is SO₂R. In other embodiments, Q⁸ is SO₂N(R₁₀)(R₁₁). In other embodiments, Q⁸ is CH(CF₃)(NH—R₁₀). In other embodiments, Q⁸ is C₁-C₅ linear or branched alkyl. In other embodiments, Q⁸ is methyl. In other embodiments, Q⁸ is ethyl. In other embodiments, Q⁸ is propyl. In other embodiments, Q⁸ is isopropyl. In other embodiments, Q⁸ is butyl. In other embodiments, Q⁸ is t-Bu. In other embodiments, Q⁸ is isobutyl. In other embodiments, Q⁸ is pentyl. In other embodiments, Q⁸ is neo-pentyl. In other embodiments, Q⁸ is C₁-C₅ linear or branched haloalkyl. In other embodiments, Q⁸ is C₁-C₅ linear or branched or C₃-C₈ cyclic alkoxy. In other embodiments, Q⁸ is C₁-C₅ linear or branched thioalkoxy. In other embodiments, Q⁸ is C₁-C₅ linear or branched haloalkoxy. In other embodiments, Q⁸ is C₁-C₅ linear or branched alkoxyalkyl. In other embodiments, Q⁸ is substituted or unsubstituted C₃-C₈ cycloalkyl. In other embodiments, Q⁸ is cyclopropyl. In other embodiments, Q⁸ is cyclopentyl. In other embodiments, Q⁸ is cyclohexyl. In other embodiments, Q⁸ is substituted or unsubstituted C₃-C₈ heterocyclic ring. In other embodiments, Q⁸ is phenyl. In other embodiments, Q⁸ is substituted or unsubstituted aryl. In some embodiments, Q⁸ may be further substituted with at least one selected from: F, Cl, Br, I, C₁-C₅ linear or branched alkyl, OH, alkoxy, ethoxy, O(C═O)—R₁₀, COOH, COO—R₁₀, N(R)₂, CF₃, CN and NO₂; each is a separate embodiment according to this invention.

In various embodiments, Q⁹, Q¹² and Q¹³ of formulas III-IX and Q¹⁰ and Q¹¹ of formulas III-VIII are each independently H. In other embodiments, Q⁹, Q¹⁰, Q¹¹, Q¹² and Q¹³ are each independently F. In other embodiments, Q⁹, Q¹⁰, Q¹¹, Q¹² and Q¹³ are each independently Cl. In other embodiments, Q⁹, Q¹⁰, Q¹¹, Q¹² and Q¹³ are each independently Br. In other embodiments, Q⁹, Q¹⁰, Q¹¹, Q¹² and Q¹³ are each independently I. In other embodiments, Q⁹, Q¹⁰, Q¹¹, Q¹² and Q¹³ are each independently OH. In other embodiments, Q⁹, Q¹⁰, Q¹¹, Q¹² and Q¹³ are each independently SH. In other embodiments, Q⁹, Q¹⁰, Q¹¹, Q¹² and Q¹³ are each independently R₈—OH. In other embodiments, Q⁹, Q¹⁰, Q¹¹, Q¹² and Q¹³ are each independently CH₂—OH. In other embodiments, Q⁹, Q¹⁰, Q¹¹, Q¹² and Q¹³ are each independently R₈—SH. In other embodiments, Q⁹, Q¹⁰, Q, Q¹² and Q¹³ are each independently —R₈—O—R₁₀. In other embodiments, Q⁹, Q¹⁰, Q¹¹, Q¹² and Q¹³ are each independently —CH₂—O—CH₃. In other embodiments, Q⁹, Q¹⁰, Q¹¹, Q¹² and Q¹³ are each independently CF₃. In other embodiments, Q⁹, Q¹⁰, Q¹¹, Q¹² and Q¹³ are each independently CN. In other embodiments, Q⁹, Q¹⁰, Q¹¹, Q¹² and Q¹³ are each independently NO₂. In other embodiments, Q⁹, Q¹⁰, Q¹¹, Q¹² and Q¹³ are each independently —CH₂CN. In other embodiments, Q⁹, Q¹⁰, Q¹¹, Q¹² and Q¹³ are each independently —R₈CN. In other embodiments, Q⁹, Q¹⁰, Q¹¹, Q¹² and Q¹³ are each independently NH₂. In other embodiments, Q⁹, Q¹⁰, Q¹¹, Q¹² and Q¹³ are each independently NHR. In other embodiments, Q⁹, Q¹⁰, Q¹¹, Q¹² and Q¹³ are each independently NH—R₈—R₁₀. In other embodiments, Q⁹, Q¹⁰, Q¹¹, Q¹² and Q¹³ are each independently NH—CH₂—C(O)—CH₃. In other embodiments, Q⁹, Q¹⁰, Q¹¹, Q¹² and Q³ are each independently N(R₁₀)(R₁₁). In other embodiments, Q⁹, Q¹⁰, Q¹¹, Q¹² and Q¹³ are each independently N[C(O)CF₃][CH₂C(O)CH₃]). In other embodiments, Q⁹, Q¹⁰, Q¹¹, Q¹² and Q¹³ are each independently R₈—C(O)—CH₂—O—R₁₀. In other embodiments, Q⁹, Q¹⁰, Q¹¹, Q¹² and Q¹³ are each independently CH₂—C(O)—CH₂—OC(O)—CH₃. In other embodiments, Q⁹, Q¹⁰, Q¹, Q¹² and Q¹³ are each independently N(R)₂. In other embodiments, Q⁹, Q¹⁰, Q¹¹, Q¹² and Q¹³ are each independently R₈—N(R₁₀)(R₁₁). In other embodiments, Q⁹, Q¹⁰, Q¹¹, Q¹² and Q¹³ are each independently CH₂—N(CH₃)₂. In other embodiments, Q⁹, Q¹⁰, Q¹¹, Q¹² and Q³ are each independently —OC(O)CF₃. In other embodiments, Q⁹, Q¹⁰, Q¹¹, Q¹² and Q¹³ are each independently —OC(O)NH₂. In other embodiments, Q⁹, Q¹⁰, Q¹¹, Q¹² and Q¹³ are each independently —NHC(O)NH₂. In other embodiments, Q⁹, Q¹⁰, Q¹¹, Q¹² and Q¹³ are each independently —OCH₂Ph. In other embodiments, Q⁹, Q¹⁰, Q¹¹, Q¹² and Q¹³ are each independently NHC(O)—R₁₀. In other embodiments, Q⁹, Q¹⁰, Q¹¹, Q¹² and Q¹³ are each independently NHC(O)CF₃. In other embodiments, Q⁹, Q¹⁰, Q¹¹, Q¹² and Q¹³ are each independently NHC(O)CH₃. In other embodiments, Q⁹, Q¹⁰, Q¹¹, Q¹² and Q¹³ are each independently NHCO—N(R₁₀)(R₁₁). In other embodiments, Q⁹, Q¹⁰, Q¹¹, Q¹² and Q¹³ are each independently NHC(O)N(CH₃)₂. In other embodiments, Q⁹, Q¹⁰, Q¹¹, Q¹² and Q¹³ are each independently N(R₁₀)C(O)R. In other embodiments, Q⁹, Q¹⁰, Q¹¹, Q¹² and Q¹³ are each independently N(CH₃)C(O)(CH₃). In other embodiments, Q⁹, Q¹⁰, Q¹¹, Q¹² and Q¹³ are each independently NHC(O)-Ph, NHC(O)-iPr. In other embodiments, Q⁹, Q¹⁰, Q¹¹, Q¹² and Q¹³ are each independently —NHC(O)—C(H)(CH₃)—O—R₁₀. In other embodiments, Q⁹, Q¹⁰, Q¹¹, Q¹² and Q¹³ are each independently —NHC(O)C(H)(CH₃)—OC(O)—CH₃. In other embodiments, Q⁹, Q¹⁰, Q¹¹, Q¹² and Q¹³ are each independently COOH. In other embodiments, Q⁹, Q¹⁰, Q¹¹, Q¹² and Q¹³ are each independently —C(O)Ph. In other embodiments, Q⁹, Q¹⁰, Q¹¹, Q¹² and Q¹³ are each independently C(O)O—R₁₀. In other embodiments, Q⁹, Q¹⁰, Q¹¹, Q¹² and Q¹³ are each independently C(O)O—CH₃. In other embodiments, Q⁹, Q¹⁰, Q¹¹, Q¹² and Q¹³ are each independently C(O)O—CH₂CH₃. In other embodiments, Q⁹, Q¹⁰, Q¹¹, Q¹² and Q¹³ are each independently R₈—C(O)—R₁₀. In other embodiments, Q⁹, Q¹⁰, Q¹¹, Q¹² and Q¹³ are each independently CH₂C(O)CH₃. In other embodiments, Q⁹, Q¹⁰, Q¹¹, Q¹² and Q¹³ are each independently C(O)H. In other embodiments, Q⁹, Q¹⁰, Q¹¹, Q¹² and Q¹³ are each independently C(O)—R₁₀. In other embodiments, Q⁹, Q¹⁰, Q¹¹, Q¹² and Q¹³ are each independently C(O)—CH₃. In other embodiments, Q⁹, Q¹⁰, Q¹¹, Q¹² and Q¹³ are each independently C(O)—CH₂CH₃. In other embodiments, Q⁹, Q¹⁰, Q¹¹, Q¹² and Q³ are each independently C(O)—CH₂CH₂CH₃. In other embodiments, Q⁹, Q¹⁰, Q¹¹, Q¹² and Q¹³ are each independently C₁-C₅ linear or branched C(O)-haloalkyl. In other embodiments, Q⁹, Q¹⁰, Q¹¹, Q¹² and Q¹³ are each independently C(O)—CF₃. In other embodiments, Q⁹, Q¹⁰, Q¹¹, Q¹² and Q¹³ are each independently —C(O)NH₂. In other embodiments, Q⁹, Q¹⁰, Q¹¹, Q¹² and Q¹³ are each independently C(O)NHR. In other embodiments, Q⁹, Q¹⁰, Q¹¹, Q¹² and Q¹³ are each independently C(O)NH—CH₃). In other embodiments, Q⁹, Q¹⁰, Q¹¹, Q¹² and Q¹³ are each independently OSO₂R. In other embodiments, Q⁹, Q¹⁰, Q¹¹, Q¹² and Q¹³ are each independently OSO₂(CH₃). In other embodiments, Q⁹, Q¹⁰, Q¹¹, Q¹² and Q¹³ are each independently NHSO₂R. In other embodiments, Q⁹, Q¹⁰, Q¹¹, Q¹² and Q¹³ are each independently NHSO₂(CH₃). In other embodiments, Q⁹, Q¹⁰, Q¹¹, Q¹² and Q¹³ are each independently N(R₁₀)SO₂R. In other embodiments, Q⁹, Q¹⁰, Q¹¹, Q¹² and Q¹³ are each independently N(CH₃)SO₂(CH₃). In other embodiments, Q⁹, Q¹⁰, Q¹¹, Q¹² and Q¹³ are each independently C(O)N(R₁₀)(R₁₁). In other embodiments, Q⁹, Q¹⁰, Q¹¹, Q¹² and Q¹³ are each independently C(O)N(CH₃)₂. In other embodiments, Q⁹, Q¹⁰, Q¹¹, Q¹² and Q¹³ are each independently C(O)NH(CH₃). In other embodiments, Q⁹, Q¹⁰, Q¹¹, Q¹² and Q¹³ are each independently SO₂R. In other embodiments, Q⁹, Q¹⁰, Q¹¹, Q¹² and Q¹³ are each independently SO₂N(R₁₀)(R₁₁). In other embodiments, Q⁹, Q¹⁰, Q¹¹, Q¹² and Q¹³ are each independently SO₂N(CH₃)₂. In other embodiments, Q⁹, Q¹⁰, Q¹¹, Q¹² and Q¹³ are each independently C₁-C₅ linear or branched alkyl. In other embodiments, Q⁹, Q¹⁰, Q¹¹, Q¹² and Q¹³ are each independently methyl. In other embodiments, Q⁹, Q¹⁰, Q¹¹, Q¹² and Q¹³ are each independently ethyl. In other embodiments, Q⁹, Q¹⁰, Q¹¹, Q¹² and Q¹³ are each independently propyl. In other embodiments, Q⁹, Q¹⁰, Q¹, Q² and Q¹³ are each independently iso-propyl. In other embodiments, Q⁹, Q¹⁰, Q¹¹, Q¹² and Q¹³ are each independently t-Bu. In other embodiments, Q⁹, Q¹⁰, Q¹¹, Q¹² and Q¹³ are each independently iso-butyl. In other embodiments, Q⁹, Q¹⁰, Q¹¹, Q¹² and Q¹³ are each independently pentyl. In other embodiments, Q⁹, Q¹⁰, Q¹¹, Q¹² and Q¹³ are each independently C₁-C₅ linear or branched haloalkyl. In other embodiments, Q⁹, Q¹⁰, Q¹¹, Q¹² and Q¹³ are each independently CF₂CH₃. In other embodiments, Q⁹, Q¹⁰, Q¹¹, Q¹² and Q¹³ are each independently CH₂CF₃. In other embodiments, Q⁹, Q¹⁰, Q¹¹, Q¹² and Q¹³ are each independently C₁-C₅ linear or branched or C₃-C₈ cyclic alkoxy. In other embodiments, Q⁹, Q¹⁰, Q¹¹, Q¹² and Q¹³ are each independently methoxy. In other embodiments, Q⁹, Q¹⁰, Q¹¹, Q¹² and Q¹³ are each independently ethoxy. In other embodiments, Q⁹, Q¹⁰, Q¹¹, Q¹² and Q¹³ are each independently propoxy. In other embodiments, Q⁹, Q¹⁰, Q¹¹, Q¹² and Q¹³ are each independently isopropoxy. In other embodiments, Q⁹, Q¹⁰, Q¹¹, Q¹² and Q¹³ are each independently O—CH₂-cyclopropyl. In other embodiments, Q⁹, Q¹⁰, Q¹¹, Q¹² and Q¹³ are each independently C₁-C₅ linear or branched thioalkoxy. In other embodiments, Q⁹, Q¹⁰, Q¹¹, Q¹² and Q¹³ are each independently C₁-C₅ linear or branched haloalkoxy. In other embodiments, Q⁹, Q¹⁰, Q¹¹, Q¹² and Q¹³ are each independently C₁-C₅ linear or branched alkoxyalkyl. In other embodiments, Q⁹, Q¹⁰, Q¹¹, Q¹² and Q¹³ are each independently substituted or unsubstituted C₃-C₈ cycloalkyl. In other embodiments, Q⁹, Q¹⁰, Q¹¹, Q¹² and Q¹³ are each independently cyclopropyl. In other embodiments, Q⁹, Q¹⁰, Q¹¹, Q¹² and Q¹³ are each independently cyclopentyl. In other embodiments, Q⁹, Q¹⁰, Q¹¹, Q¹² and Q¹³ are each independently substituted or unsubstituted C₃-C₈ heterocyclic ring. In other embodiments, Q⁹, Q¹⁰, Q¹¹, Q¹² and Q¹³ are each independently thiophene. In other embodiments, Q⁹, Q¹⁰, Q¹¹, Q¹² and Q¹³ are each independently oxazole. In other embodiments, Q⁹, Q¹⁰, Q¹¹, Q¹² and Q¹³ are each independently thiazole. In other embodiments, Q⁹, Q¹⁰, Q¹¹, Q¹² and Q¹³ are each independently imidazole. In other embodiments, Q⁹, Q¹⁰, Q¹¹, Q¹² and Q¹³ are each independently furane. In other embodiments, Q⁹, Q¹⁰, Q¹¹, Q¹² and Q¹³ are each independently triazole. In other embodiments, Q⁹, Q¹⁰, Q¹¹, Q¹² and Q¹³ are each independently pyridine. In other embodiments, Q⁹, Q¹⁰, Q¹¹, Q¹² and Q¹³ are each independently 2-pyridine. In other embodiments, Q⁹, Q¹⁰, Q¹¹, Q¹² and Q¹³ are each independently 3-pyridine. In other embodiments, Q⁹, Q¹⁰, Q¹¹, Q¹² and Q¹³ are each independently 4-pyridine. In other embodiments, Q⁹, Q¹⁰, Q¹¹, Q¹² and Q¹³ are each independently pyrimidine. In other embodiments, Q⁹, Q¹⁰, Q¹¹, Q¹² and Q¹³ are each independently pyrazine. In other embodiments, Q⁹, Q¹⁰, Q¹¹, Q¹² and Q¹³ are each independently pyrrolidone. In other embodiments, Q⁹, Q¹⁰, Q¹¹, Q¹² and Q¹³ are each independently substituted or unsubstituted aryl. In other embodiments, Q⁹, Q¹⁰, Q¹¹, Q¹² and Q¹³ are each independently phenyl; wherein substitutions include: F, Cl, Br, I, C₁-C₅ linear or branched alkyl, OH, alkoxy, N(R)₂, CF₃, CN or NO₂. In other embodiments, Q⁹, Q¹⁰, Q¹¹, Q¹² and Q¹³ are each independently CH(CF₃)(NH—R₁₀). In some embodiments, Q⁹, Q¹⁰, Q¹¹, Q¹² and Q¹³ are each independently substituted with at least one selected from: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN and NO₂; each is a separate embodiment according to this invention.

In various embodiments, Q⁹ and Q¹⁰ of formulas III-VIII are joined to form a 5 or 6 membered substituted or unsubstituted, aliphatic or aromatic, carbocyclic or heterocyclic ring. In other embodiments, Q⁹ and Q¹⁰ are joined to form an unsubstituted aromatic heterocyclic ring. In other embodiments, Q⁹ and Q¹⁰ are joined to form an imidazole ring. In other embodiments, Q⁹ and Q¹⁰ are joined to form 1,4-dioxane. In other embodiments, Q⁹ and Q¹⁰ are joined to form 1,3-dioxane. In other embodiments, Q⁹ and Q¹⁰ are joined to form pyrrole.

In various embodiments, Q¹⁰ and Q¹¹ of formulas III-VIII are joined to form a 5 or 6 membered substituted or unsubstituted, aliphatic or aromatic, carbocyclic or heterocyclic ring. In other embodiments, Q¹⁰ and Q¹¹ are joined to form 1,4-dioxane. In other embodiments, Q¹⁰ and Q¹¹ are joined to form 1,3-dioxane. In other embodiments, Q¹⁰ and Q¹¹ are joined to form pyrrole. In other embodiments, Q¹⁰ and Q¹¹ are joined to form imidazole. In other embodiments, Q¹⁰ and Q¹¹ are joined to form 1-methylimidazole. In other embodiments, Q¹⁰ and Q¹¹ are joined to form oxazole. In other embodiments, Q¹⁰ and Q¹¹ are joined to form triazole. In other embodiments, Q¹⁰ and Q¹¹ are joined to form furane. In other embodiments, Q¹⁰ and Q¹¹ are joined to form 1H-imidazol-2(3H)-one.

In various embodiments, m of formulas III-VII is an integer between 0 and 5. In other embodiments, m is an integer between 1 and 3. In other embodiments, m is 0. In other embodiments, m is 1. In other embodiments, m is 2. In other embodiments, m is 0, 1, 2, 3, 4, or 5; each is a separate embodiment according to this invention.

In various embodiments, n of formulas I-IX is an integer number between 1 and 20. In various embodiments, n is an integer number between 2 and 6. In various embodiments, n is an integer number between 1 and 6. In various embodiments, n is an integer number between 1 and 16. In various embodiments, n is an integer number between 1 and 10. In various embodiments, n is an integer number between 2 and 8. In various embodiments, n is an integer number between 3 and 5. In various embodiments, n is an integer number between 3 and 8. In various embodiments, n is an integer number between 3 and 12. In various embodiments, n is 1, 2, 3, 4, 5, 6, 7, 8, 9, 10, 11, 12, 13, 14, 15, 16, 17, 18, 19 or 20; each is a separate embodiment according to this invention.

In various embodiments, k of formulas I-IX is an integer number between 1 and 20. In various embodiments, k is an integer number between 6 and 16. In various embodiments, k is an integer number between 4 and 15. In various embodiments, k is an integer number between 2 and 16. In various embodiments, k is an integer number between 3 and 18. In various embodiments, k is an integer number between 5 and 15. In various embodiments, k is an integer number between 4 and 18. In various embodiments, k is an integer number between 3 and 20. In various embodiments, k is 1, 2, 3, 4, 5, 6, 7, 8, 9, 10, 11, 12, 13, 14, 15, 16, 17, 18, 19 or 20; each is a separate embodiment according to this invention.

In various embodiments, X¹ of formulas III-VI is C. In other embodiments, X¹ is N.

In various embodiments, X² of formulas III-VI is C. In other embodiments, X² is N.

In various embodiments, X³ of formulas III-VI is C. In other embodiments, X³ is N.

In various embodiments, X⁴ of formulas III-VI is C. In other embodiments, X⁴ is N.

In various embodiments, X⁵ of formulas III-VI is C. In other embodiments, X⁵ is N.

In various embodiments, if X¹, X², X³, X⁴ and/or X⁵ is N then Q⁹, Q¹⁰, Q¹¹, Q¹² and/or Q¹³ is absent respectively.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, etc.

As used herein, the term “alkenyl” can be any linear- or branched-chain alkenyl group containing up to about 30 carbons as defined hereinabove for the term “alkyl” and at least one carbon-carbon double bond. Accordingly, the term alkenyl as defined herein includes also alkadienes, alkatrienes, alkatetraenes, and so on. In some embodiments, the alkenyl group contains one carbon-carbon double bond. In some embodiments, the alkenyl group contains two, three, four, five, six, seven or eight carbon-carbon double bonds; each represents a separate embodiment according to this invention. Non limiting examples of alkenyl groups include: Ethenyl, Propenyl, Butenyl (i.e., 1-Butenyl, trans-2-Butenyl, cis-2-Butenyl, and Isobutylenyl), Pentene (i.e., 1-Pentenyl, cis-2-Pentenyl, and trans-2-Pentenyl), Hexene (e.g., 1-Hexenyl, (E)-2-Hexenyl, (Z)-2-Hexenyl, (E)-3-Hexenyl, (Z)-3-Hexenyl, 2-Methyl-1-Pentene, etc.), which may all be substituted as defined herein above for the term “alkyl”.

As used herein, the term “alkynyl” can be any linear- or branched-chain alkynyl group containing up to about 30 carbons as defined hereinabove for the term “alkyl” and at least one carbon-carbon triple bond. Accordingly, the term alkynyl as defined herein includes also alkadiynes, alkatriynes, alkatetraynes, and so on. In some embodiments, the alkynyl group contains one carbon-carbon triple bond. In some embodiments, the alkynyl group contains two, three, four, five, six, seven or eight carbon-carbon triple bonds; each represents a separate embodiment according to this invention. Non limiting examples of alkynyl groups include: acetylenyl, Propynyl, Butynyl (i.e., 1-Butynyl, 2-Butynyl, and Isobutylynyl), Pentyne (i.e., 1-Pentynyl, 2-Pentynyl), Hexyne (e.g., 1-Hexynyl, 2-Hexynyl, 3-Hexynyl, etc.), which may all be substituted as defined herein above for the term “alkyl”.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

An “alkylester” group refers, in some embodiments, to an alkyl group as defined above, which is substituted by an ester group, e.g. by O—C(O)—CH₃, O—C(O)—CH₂CH₃ etc. Nonlimiting examples of alkylester groups are CH₂—O—(CO)—CH₃, CH(CH₃)—O—(C═O)—CH₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHC(O)-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclobutyl, cyclobutenyl, cyclooctyl, cyclooctadienyl (COD), cyclooctane (COE) etc.

A “cycloalkenyl” refers, in various embodiments, to a closed ring of carbon atoms, which is unsaturated but has no aromatic character. The ring can be substituted or unsubstituted, single or fused. In some embodiments the cycloalkenyl is a 3-10 membered ring. In some embodiments the cycloalkenyl is a 3-12 membered ring. In some embodiments the cycloalkenyl is a 6 membered ring. In some embodiments the cycloalkenyl is a 5-7 membered ring. In some embodiments the cycloalkenyl is a 3-8 membered ring. In some embodiments, the cycloalkenyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In some embodiments, the cycloalkenyl ring may be fused to another saturated or unsaturated carbocyclic or heterocyclic 3-8 membered ring. In some embodiments, the cycloalkenyl ring has one unsaturated carbon-carbon bond. In some embodiments, the cycloalkenyl ring has two unsaturated carbon-carbon bonds. Non limiting examples of a cycloalkenyl group comprise cyclohexenyl, cyclopropenyl, cyclopentenyl, cyclobutenyl, cyclooctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen, selenium or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thiol, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHC(O)-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, 5-methyl-1,2,4-oxadiazole, oxazolidin-2-one, methyloxazolidin-2-one or indole; each is a separate embodiment according to this invention.

In various embodiments, this invention provides a compound of this invention or its isomer, metabolite, pharmaceutically acceptable salt, pharmaceutical product, tautomer, hydrate, N-oxide, polymorph, prodrug, isotopic variant (e.g. deuterated analogs), PROTAC or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a polymorph of the compound of this invention. In some embodiments, this invention provides a prodrug of the compound of this invention. In some embodiments, this invention provides an isotopic variant of the compound of this invention. In some embodiments, this invention provides a deuterated analog of the compound of this invention. In some embodiments, this invention provides a PROTAC of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, polymorph, prodrug, isotopic variant (e.g. deuterated analogs), PROTAC or crystal of the compound of this invention.

In various embodiments, the term “isomer” includes, but is not limited to, stereoisomers including optical isomers and analogs, structural isomers and analogs, conformational isomers and analogs, and the like. In some embodiments, the isomer is a stereoisomer. In another embodiment, the isomer is an optical isomer.

Certain compounds of the present invention may exist in particular geometric or stereoisomeric forms. The present invention contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. The compounds according to this invention may further exist as stereoisomers which may be also optically-active isomers (e.g., enantiomers such as (R) or (S)), as enantiomerically enriched mixtures, racemic mixtures, or as single diastereomers, diastereomeric mixtures, or any other stereoisomers, including but not limited to: (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) stereoisomers. Some compounds may also exhibit polymorphism. It is to be understood that the present invention encompasses any racemic, optically-active, polymorphic, or stereoisomeric form, or mixtures thereof, which form possesses properties useful in the treatment of the various conditions described herein.

It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

Compounds of the present invention may exist in the form of one or more of the possible tautomers and depending on the particular conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included.

### Tautomerization of the Imidazole Ring

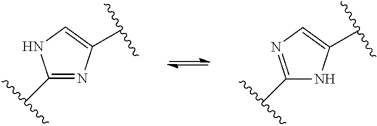 [SMILES: *c1c[nH]c(*)n1 ; *c1cnc(*)[nH]1]

### Tautomerization of the Pyrazolone Ring

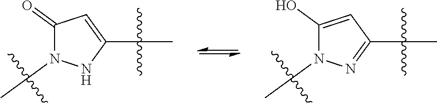 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically-acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an anti-cancer agent.

In various embodiments, the compounds of this invention are administered in combination with an anti-inflammatory agent.

In various embodiments, the compounds of this invention are administered in combination with an anti-viral agent.

In various embodiments, the compounds of this invention are administered in combination with an anti-parasitic agent.

In various embodiments, the compounds of this invention are administered in combination with an agent treating metabolic disorders.

In various embodiments, the compounds of this invention are administered in combination with an agent treating autoimmune disorders.

Yet another aspect of the present invention relates to a method of treating cancer that includes selecting a subject in need of treatment for cancer and administering to the subject a pharmaceutical composition comprising a compound according to the present invention and a pharmaceutically acceptable carrier under conditions effective to treat cancer.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing or stimulating a desired response in a subject, as will be understood by one skilled in the art. In some embodiments, the compositions may further comprise additional active ingredients, whose activity is useful for the particular application for which the compound of this invention is being administered.

In various embodiments, this invention is directed to a method of treating a patient suffering from a disease comprising administering to a patient in need of such treatment a therapeutically effective amount of a compound of the present invention. The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment, e.g. reasonable side effects applicable to any medical treatment.

Glucose represents a central nutrient for many organisms, and control of glucose signaling and consumption is tightly regulated. Accordingly, many disease states are associated with defects in this regulation and therefore may be susceptible to therapeutic intervention using glucose uptake inhibitors. Glucose uptake inhibitors have utility in disease areas such as oncology, autoimmunity and inflammation, infection diseases/virology, and metabolic disease.

One of the emerging hallmarks of cancer is reprogramming of cancer cell metabolism. In order to meet the energetic demands of cell growth and division, cancer cells adopt the process of “aerobic glycolysis.” While normal cells maintain a low rate of glycolysis, followed by full oxidization of pyruvate in the mitochondria, cancer cells rely on an increased rate of glycolysis followed by lactic acid fermentation (even in the presence of oxygen). Since mitochondrial oxidation phosphorylation generates more ATP than glycolysis alone, cancer cells rely heavily on increased rates of glucose consumption. One common way cancer cells achieve this goal is through the up-regulation of glucose transporters. In fact, many well-characterized oncogenes are thought to up-regulate both glycolytic enzymes and glucose transporters.

Furthermore, the increased rate of glucose consumption displayed by most tumors has already been employed in the field of diagnostics. Because of this cancer wide-phenomenon, one standard technique for imaging tumors is through PET imaging of a radio-labelled glucose analog (18 FDG) (Hanahan, D. and R. A. Weinberg, Hallmarks of cancer: the next generation. Cell, 2011. 144 (5): p. 646-74.) It is therefore predicted that inhibition of glucose uptake should affect cancer cells from a wide variety of tumor types while having little effect on normal cells.

Inactivating mutations in any of the SDH subunits, or the SDH complex assembly factor (SDHAF2), are associated with susceptibility to develop neuroendocrine neoplasms and gastrointestinal stromal tumors as well as renal cell carcinoma. Loss of function of the SDH complex characterizes a rare group of human tumors including some gastrointestinal stromal tumors (SDH-deficient “KIT wild-type” GIST), paragangliomas, renal carcinomas and pituitary adenomas. These tumors are generally refractory to conventional targeted therapeutic approaches.

The tumor/cancer may be familial or sporadic, depending on whether it is derived from germline or somatic mutations in the SDH complex, respectively.

Therefore, in various embodiments, this invention is directed to a method of treating, suppressing, reducing the severity, reducing the risk of developing or inhibiting cancer comprising administering a compound of this invention to a subject suffering from cancer under conditions effective to treat, suppress, reduce the severity, reduce the risk of developing, or inhibit the cancer. In some embodiments, the compound targets SDH-deficient cells. In some embodiments, the compound destroys SDH-deficient cells. In various embodiments, the compound is selective to cells with broken TCA cycle. In various embodiments, the broken TCA cycle is genetic or chemically induced. In some embodiments, the cancer is sporadic. In some embodiments, the cancer is familial. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer.

In some embodiments, the cancer is selected from: Multiple myeloma, bladder cancer, Myelodysplasia, breast cancer, cervix cancer, endometrium cancer, esophagus cancer, head and neck cancer (squamous cell carcinoma), kidney cancer (renal cell carcinoma), liver cancer (hepatocellular carcinoma), lung cancer (non-small cell; NSCLC), nasopharynx cancer, solid tumor cancer, stomach cancer, adrenocortical carcinoma, Glioblastoma multiforme, acute myeloid Leukemia, chronic lymphocytic Leukemia, Hodgkin's (classical) Lymphoma, diffuse large B-cell Lymphoma, primary central nervous system Lymphoma, malignant Melanoma, uveal Melanoma, Meningioma, breast cancer, anus cancer, anus (squamous cell) cancer, biliary cancer, bladder cancer, muscle invasive urothelial carcinoma, colorectal cancer, fallopian tube cancer, gastroesophageal junction cancer, larynx (squamous cell) cancer, lung cancer (small cell, SCLC), merkel cell cancer, mouth cancer, ovary cancer, pancreas cancer, penis cancer, peritoneum cancer, prostate cancer, rectum cancer, skin cancer (basal cell carcinoma, squamous cell carcinoma), small intestine cancer, testis cancer, thymus cancer, anaplastic thyroid cancer, Cholangiocarcinoma, Chordoma, Cutaneous T-cell lymphoma, Digestive-gastrointestinal cancer, Familial pheochromocytoma-paraganglioma, Glioma, HTLV-1-associated adult T-cell leukemia-lymphoma, Hematologic-blood cancer, uterine Leiomyosarcoma, acute lymphocytic Leukemia, chronic myeloid Leukemia, T-cell Lymphoma, follicular Lymphoma, primary mediastinal large B-cell Lymphoma, testicular diffuse large B-cell Lymphoma, Melanoma, malignant Mesothelioma, pleural Mesothelioma, Mycosis fungoides, Neuroendocrine cancer, Oral epithelial dysplasia, Sarcoma, Uterine cancer, myeloma Smoldering, Soft tissue sarcoma, nasal natural killer (NK) cell T-cell lymphoma and peripheral T-cell lymphoma; each represents a separate embodiment according to this invention.

In some embodiments, the cancer, is selected from the list of: gastrointestinal stromal tumors, renal cell carcinoma, bladder paragangliomas, pituitary adenoma, pheochromocytoma (familial and sporadic), paraganglioma (familiar and sporadic), colorectal cancer, gastric cancer, leiomyosarcoma (including metastatic) and/or ovarian cancer; each is a separate embodiment according to this invention. In some embodiments, the cancer is selected from the list of: melanoma, non-small cell lung cancer, kidney cancer, bladder cancer, head and neck cancers, Hodgkin lymphoma, Merkel cell skin cancer (Merkel cell carcinoma), esophagus cancer; gastroesophageal junction cancer; liver cancer, (hepatocellular carcinoma); lung cancer, (small cell) (SCLC); stomach cancer; upper urinary tract cancer, (urothelial carcinoma); multiforme Glioblastoma; Multiple myeloma; anus cancer, (squamous cell); cervix cancer; endometrium cancer; nasopharynx cancer; ovary cancer; metastatic pancreas cancer; solid tumor cancer; adrenocortical Carcinoma; HTLV-1-associated adult T-cell leukemia-lymphoma; uterine Leiomyosarcoma; acute myeloid Leukemia; chronic lymphocytic Leukemia; diffuse large B-cell Lymphoma; follicular Lymphoma; uveal Melanoma; Meningioma; pleural Mesothelioma; Myelodysplasia; Soft tissue sarcoma; breast cancer; colon cancer; Cutaneous T-cell lymphoma; and peripheral T-cell lymphoma; each is a separate embodiment according to this invention. In some embodiments, the cancer is selected from the list of: glioblastoma, melanoma, lymphoma, breast cancer, ovarian cancer, glioma, digestive system cancer, central nervous system cancer, hepatocellular cancer, hematological cancer, colon cancer or any combination thereof; each is a separate embodiment according to this invention. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting tumor growth, comprising administering a compound of this invention to a subject suffering from a tumor growth under conditions effective to treat, suppress, reduce the severity, reduce the risk of developing, or inhibit the tumor growth. In other embodiments, the tumor growth is stimulated by a broken TCA cycle. In other embodiments, the tumor cells have a broken TCA cycle. In some embodiments, the compound targets SDH-deficient cells. In some embodiments, the compound destroys SDH-deficient cells. In some embodiments, the tumor is benign. In some embodiments, the tumor is malignant. In some embodiments, the tumor is cancerous. In some embodiments, the tumor results in cancer. In some embodiments, the tumor is familial. In some embodiments, the tumor is sporadic. In some embodiments, the tumor is gastrointestinal stromal tumor (GIST). In some embodiments, the tumor is paraganglioma. In some embodiments, the tumor is pituitary adenoma. In some embodiments, the tumor is pheochromocytoma. In some embodiments, the pheochromocytoma is familial. In some embodiments, the pheochromocytoma is sporadic. In some embodiments, the tumor is paraganglioma. In some embodiments, the paraganglioma is familial. In some embodiments, the paraganglioma is sporadic. In some embodiments, the tumor is leiomyoma. In some embodiments, the leiomyoma is benign. In some embodiments, the leiomyoma is uterine fibroids. In some embodiments, the tumor results in colorectal cancer. In some embodiments, the tumor results in gastric cancer. In some embodiments, the tumor results in ovarian cancer. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting SDH-associated neoplasms comprising administering a compound of this invention to a subject suffering from SDH-associated neoplasms under conditions effective to treat, suppress, reduce the severity, reduce the risk of developing, or inhibit the SDH-associated neoplasms. In some embodiments, the compound targets SDH-deficient cells. In some embodiments, the compound destroys SDH-deficient cells. In some embodiments, the SDH-associated neoplasms are neuroendocrine neoplasms. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting neuroendocrine neoplasms comprising administering a compound of this invention to a subject suffering from neuroendocrine neoplasms under conditions effective to treat, suppress, reduce the severity, reduce the risk of developing, or inhibit the neuroendocrine neoplasms. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting gastrointestinal stromal tumors (GIST) comprising administering a compound of this invention to a subject suffering from gastrointestinal stromal tumors under conditions effective to treat, suppress, reduce the severity, reduce the risk of developing, or inhibit the gastrointestinal stromal tumors. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting renal cell carcinoma comprising administering a compound of this invention to a subject suffering from renal cell carcinoma under conditions effective to treat, suppress, reduce the severity, reduce the risk of developing, or inhibit the renal cell carcinoma. In some embodiments, the renal cell carcinoma is early renal cell carcinoma. In some embodiments, the renal cell carcinoma is advanced renal cell carcinoma. In some embodiments, the renal cell carcinoma is invasive renal cell carcinoma. In some embodiments, the renal cell carcinoma is metastatic renal cell carcinoma. In some embodiments, the renal cell carcinoma is drug resistant renal cell carcinoma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting paraganglioma comprising administering a compound of this invention to a subject suffering from paraganglioma under conditions effective to treat, suppress, reduce the severity, reduce the risk of developing, or inhibit the paraganglioma. In some embodiments, the paraganglioma is benign. In some embodiments, the paraganglioma is malignant. In some embodiments, the paraganglioma is advanced paraganglioma. In some embodiments, the paraganglioma is invasive paraganglioma. In some embodiments, the paraganglioma is metastatic paraganglioma. In some embodiments, the paraganglioma is drug resistant paraganglioma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting pituitary adenoma comprising administering a compound of this invention to a subject suffering from pituitary adenoma under conditions effective to treat, suppress, reduce the severity, reduce the risk of developing, or inhibit the pituitary adenoma. In some embodiments, the pituitary adenoma is benign. In some embodiments, the pituitary adenoma is invasive. In some embodiments, the pituitary adenoma is malignant. In some embodiments, the pituitary adenoma is carcinoma. In some embodiments, the pituitary adenoma is metastatic pituitary adenoma. In some embodiments, the pituitary adenoma is drug resistant pituitary adenoma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Pheochromocytomas (PHEOs)/paragangliomas (PGLs) are rare neuroendocrine tumors that produce catecholamines (Lenders et al. 2005). PHEOs/PGLs arise from three distinct parts of the neural crest: the adrenal medulla (PHEOs) and the sympathetic and parasympathetic paraganglia (extradrenal PGLs) (Papaspyrou et al. 2012). Most present as benign, yet show high morbidity and mortality due to excessive catecholamine production, leading to hypertension, arrhythmia, and stroke. Up to 25% are malignant (Ayala-Ramirez et al., 2011), as defined by distant metastases to non-chromaffin tissues. Patients with metastatic PCCs/PGLs have limited treatment options and poor prognosis, often with less than 50% surviving at 5 years (Hescot et al., 2013). Despite a low incidence (0.8 per 100,000 for PCCs) (Beard et al., 1983), over one-third of PCCs/PGLs are associated with inherited cancer susceptibility syndromes, which is the highest rate among all tumor types (Dahia, 2014).

Inactivating missense mutations of the genes that encode the SDH subunits are found to cause familial pheochromocytoma and familial paraganglioma. These SDH gene alterations, which lead to the loss of enzyme activity and expression, are also observed frequently in several tumors such as renal cell carcinoma, gastrointestinal stromal tumors, colorectal cancer, gastric cancer, and ovarian cancer. Thus, SDH subunit genes are considered to be tumor suppressor genes.

Accordingly, in various embodiments, this invention is directed to a method of treating, suppressing, reducing the severity, reducing the risk of developing or inhibiting pheochromocytoma (PHEO) comprising administering a compound of this invention to a subject suffering from pheochromocytoma under conditions effective to treat, suppress, reduce the severity, reduce the risk of developing, or inhibit the pheochromocytoma. In some embodiments, the pheochromocytoma is familial pheochromocytoma. In some embodiments, the pheochromocytoma is sporadic pheochromocytoma. In some embodiments, the pheochroinocytoina is benign. In some embodiments, the pheochromocytoma is malignant. In some embodiments, the pheochromocytoma is early pheochromocytoma. In some embodiments, the pheochromocytoma is advanced pheochromocytoma. In some embodiments, the pheochromocytoma is invasive pheochromocytoma. In some embodiments, the pheochromocytoma is metastatic pheochromocytoma. In some embodiments, the pheochromocytoma is drug resistant pheochromocytoma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting paraganglioma (PGL) comprising administering a compound of this invention to a subject suffering from paraganglioma under conditions effective to treat, suppress, reduce the severity, reduce the risk of developing, or inhibit the paraganglioma. In some embodiments, the paraganglioma is familial paraganglioma. In some embodiments, the paraganglioma is sporadic paraganglioma. In some embodiments, the paraganglioma is bladder paragangliomas. In some embodiments, the paraganglioma is benign. In some embodiments, the paraganglioma is malignant. In some embodiments, the paraganglioma is early paraganglioma. In some embodiments, the paraganglioma is advanced paraganglioma. In some embodiments, the paraganglioma is invasive paraganglioma. In some embodiments, the paraganglioma is metastatic paraganglioma. In some embodiments, the paraganglioma is drug resistant paraganglioma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting Leiomyosarcoma comprising administering a compound of this invention to a subject suffering from Leiomyosarcoma under conditions effective to treat, suppress, reduce the severity, reduce the risk of developing, or inhibit the Leiomyosarcoma. In some embodiments, the Leiomyosarcoma is early Leiomyosarcoma. In some embodiments, the Leiomyosarcoma is advanced Leiomyosarcoma. In some embodiments, the Leiomyosarcoma is invasive Leiomyosarcoma. In some embodiments, the Leiomyosarcoma is metastatic Leiomyosarcoma. In some embodiments, the Leiomyosarcoma is drug resistant Leiomyosarcoma. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound is selective towards specific glucose transporters, selected from: GluT1, GluT2, GluT3, GluT4 and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colorectal cancer comprising administering a compound of this invention to a subject suffering from colorectal cancer under conditions effective to treat, suppress, reduce the severity, reduce the risk of developing, or inhibit the colorectal cancer. In some embodiments, the colorectal cancer is early colorectal cancer. In some embodiments, the colorectal cancer is advanced colorectal cancer. In some embodiments, the colorectal cancer is invasive colorectal cancer. In some embodiments, the colorectal cancer is metastatic colorectal cancer. In some embodiments, the colorectal cancer is drug resistant colorectal cancer. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting gastric cancer comprising administering a compound of this invention to a subject suffering from gastric cancer under conditions effective to treat, suppress, reduce the severity, reduce the risk of developing, or inhibit the gastric cancer. In some embodiments, the gastric cancer is early gastric cancer. In some embodiments, the gastric cancer is advanced gastric cancer. In some embodiments, the gastric cancer is invasive gastric cancer. In some embodiments, the gastric cancer is metastatic gastric cancer. In some embodiments, the gastric cancer is drug resistant gastric cancer. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting ovarian cancer comprising administering a compound of this invention to a subject suffering from ovarian cancer under conditions effective to treat, suppress, reduce the severity, reduce the risk of developing, or inhibit the ovarian cancer. In some embodiments, the ovarian cancer is early ovarian cancer. In some embodiments, the ovarian cancer is advanced ovarian cancer. In some embodiments, the ovarian cancer is invasive ovarian cancer. In some embodiments, the ovarian cancer is metastatic ovarian cancer. In some embodiments, the ovarian cancer is drug resistant ovarian cancer. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting pancreatic neuroendocrine tumors comprising administering a compound of this invention to a subject having pancreatic neuroendocrine tumors under conditions effective to treat, suppress, reduce the severity, reduce the risk of developing, or inhibit the pancreatic neuroendocrine tumors. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Like cancer cells, activated T effector cells switch to the process of aerobic glycolysis to meet their energetic demands (MacIver, N. J., R. D. Michalek, and J. C. Rathmell, Metabolic regulation of T lymphocytes. Annu Rev Immunol, 2013. 31: p. 259-83.) Since hyper-activation of helper T-cells (e.g. Th17, Th2, Th1) play a large role in autoimmune disorders and inflammation, decreasing the rate of glycolysis in these cells would be predicted to curb their secretion of inflammatory cytokines. In addition, as inhibition of glucose uptake activates AMPK, a master regulator of T regulatory cells (Michalek, R. D., et al., Cutting edge: distinct glycolytic and lipid oxidative metabolic programs are essential for effector and regulatory CD4+ T cell subsets. J Immunol, 2011. 186 (6): p. 3299-303)), the use of glucose uptake inhibitors would also be predicted to increase the T regulatory cell population (which suppress inflammation), thereby “rebalancing” the immune system. In addition to T cells, other cells of the immune system (including but not limited to macrophages, dendritic cells and B cells) rely heavily on glycolysis for their development, activation and effector functions. Thus, it is suggested that glucose uptake inhibitors will have utility as immune suppressants and may provide benefit in many autoimmune and inflammatory conditions.

Compounds that are targeting SDH-deficient cells may therefore be useful as anti-inflammatory agents. The current assumption is that activated macrophages produce Itaconate that then inhibits SDH and lead to succinate accumulation. Hence, it is possible that compounds of the invention that kill SDH-deficient cells will eliminate also activated macrophages and could serves as specific anti-inflammatory treatment.

In certain embodiments, compounds according to this invention are used to treat inflammation, including, but not limited to, asthma, idiopathic pulmonary fibrosis, liver fibrosis, renal fibrosis, LAM, nephrogenic systemic fibrosis, arthritis (especially rheumatoid arthritis and/or psoriatic arthritis and/or juvenile arthritis), sepsis and/or other autoimmune diseases such as but not limited to atherosclerosis, psoriasis, systemic lupus erythematosus, lupus nephritis, chronic graft vs. host disease, acute graft vs. host disease, multiple sclerosis, myasthenia gravis or symptoms of any of these, as well as one or more IBD, such as Crohn's disease, scleroderma, granulomatous colitis, ulcerative colitis, lymphocyte colitis, collagenous colitis and/or Coeliac disease, insulin-dependent diabetes mellitus, acquired immunodeficiency syndrome (“AIDS”), hemolytic anemias, rheumatic fever, Guillain-Barre syndrome and CIDP, thyroiditis, Graves' disease, glomerulonephritis, autoimmune hepatitis, cardiovascular inflammation, renal inflammation, and arteriosclerosis. In addition, the compounds according to this invention disclosed herein may also be used as immunosuppressants for preventing rejection of transplanted organs.

Accordingly, in various embodiments, this invention is directed to a method of treating, suppressing, reducing the severity, reducing the risk of developing or inhibiting inflammation comprising administering a compound of this invention to a subject suffering from inflammation under conditions effective to treat, suppress, reduce the severity, reduce the risk of developing, or inhibit the inflammation. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting autoimmune disease or disorder comprising administering a compound of this invention to a subject suffering from an autoimmune disease or disorder under conditions effective to treat, suppress, reduce the severity, reduce the risk of developing, or inhibit the autoimmune disease or disorder. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting inflammation or autoimmune disease comprising administering a compound of this invention to a subject suffering from inflammation or an autoimmune disease under conditions effective to treat, suppress, reduce the severity, reduce the risk of developing, or inhibit the inflammation or the autoimmune disease. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Many infectious agents (including viruses, parasites, etc.) rely heavily on glucose consumption for growth and expansion and therefore the use of the glucose uptake inhibitors disclosed herein will be useful for inhibiting infectious diseases. In particular, the malaria parasites of the genus Plasmodia rely exclusively on glucose for energy production. These parasites infect red blood cells and induce a vast increase in glucose uptake modulated through the host cell GLUT1 transporters. Subsequent to transport of glucose into the red blood cells, parasites must also transport glucose across its own cellular membrane through additional, parasite-encoded hexose transporters such as Plasmodium falciparum: hexose transporter 1 (PfHT1). Interfering with glucose uptake at either step with the compounds disclosed herein is predicted to have anti-malarial activity. In addition to malaria, many other infectious agents also hijack the mammalian cellular machinery to support their own growth, often targeting host glucose metabolism. For example, the activity of the glucose transporter GLUT1 is critically important for the infection and replication of HIV-1 in cultured T cells (Loisel-Meyer, S., et al., Glut1-mediated glucose transport regulates HIV infection. Proc Natl Acad Sci USA, 2012. 109 (7): p. 2549-54). The induction of glucose uptake has also been noted in other viruses, such as the substantial increase in GLUT4 levels in human cytomegalovirus-infected fibroblasts (HCMV) (Yu, Y., T. G. Maguire, and J. C. Alwine, Human cytomegalovirus activates glucose transporter 4 expression to increase glucose uptake during infection. J Virol, 2011. 85 (4): p. 1573-80). Given that infectious disease causing agents often rely on glucose metabolism, the use of the glucose uptake inhibitors (that target host and/or parasite cellular components) disclosed herein against these diseases is promising.

Therefore, in various embodiments, this invention is directed to a method of treating, suppressing, reducing the severity, reducing the risk of developing or inhibiting a parasitic or viral infection comprising administering a compound of this invention to a subject suffering from a parasitic or viral infection under conditions effective to treat, suppress, reduce the severity, reduce the risk of developing, or inhibit the a parasitic or viral infection. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the parasitic infection is caused by malaria parasite. In some embodiments, the viral infection is caused by HIV. In some embodiments, the viral infection is caused by human cytomegalovirus (HCMV). In some embodiments, the viral infection is caused by acute malaria and African trypanosomiasis. In some embodiments, the viral infection is caused by tuberculosis. In some embodiments, the viral infection is caused by herpes virus. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In certain embodiments, a glucose uptake inhibitor of the invention is used to treat metabolic disease. In various embodiments, this invention is directed to a method of treating, suppressing, reducing the severity, reducing the risk of developing or inhibiting a metabolic disease comprising administering a compound of this invention to a subject suffering from a metabolic disease under conditions effective to treat, suppress, reduce the severity, reduce the risk of developing, or inhibit the metabolic disease. Metabolic disease includes but not limited to: diabetes (type 1 and type 2), insulin resistance, metabolic syndrome, hyperinsulinemia, nonalcoholic steatohepatitis (NASH), dyslipidemia, and hypercholesterolemia, obesity, hypertension, retinal degeneration, retinal detachment, cardiovascular diseases including vascular disease, atherosclerosis, coronary heart disease, cerebrovascular disease, heart failure and peripheral vascular disease in a subject. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Hyperglycemia resulting from diabetes mellitus can lead to various long-term consequences. Because certain cells rely solely on passive glucose transporters (where glucose flows down a concentration gradient), such cells consume damaging levels of glucose under these conditions. Inhibitors of glucose uptake (particular those that inhibit GLUT1) could protect such cells from damage. For example, the compounds disclosed herein may have utility in both diabetic retinopathy (Lu, L., et al., Suppression of GLUT1; a new strategy to prevent diabetic complications. J Cell Physiol, 2013. 228 (2): p. 251-7) and nephropathy (Marques, T., et al., Association of single nucleotide polymorphisms in the gene encoding GLUT1 and diabetic nephropathy in Brazilian patients with type 1 diabetes mellitus. Clin Chim Acta, 2015).

Therefore, in various embodiments, this invention is directed to a method of treating, suppressing, reducing the severity, reducing the risk of developing or inhibiting Hyperglycemia comprising administering a compound of this invention to a subject suffering from a hyperglycemia under conditions effective to treat, suppress, reduce the severity, reduce the risk of developing, or inhibit the hyperglycemia. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting diabetes mellitus comprising administering a compound of this invention to a subject suffering from diabetes mellitus under conditions effective to treat, suppress, reduce the severity, reduce the risk of developing, or inhibit the diabetes mellitus. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting diabetic retinopathy comprising administering a compound of this invention to a subject suffering from a diabetic retinopathy under conditions effective to treat, suppress, reduce the severity, reduce the risk of developing, or inhibit the diabetic retinopathy. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting diabetic nephropathy comprising administering a compound of this invention to a subject suffering from a diabetic nephropathy under conditions effective to treat, suppress, reduce the severity, reduce the risk of developing, or inhibit the diabetic nephropathy. In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention is directed to a method of suppressing, reducing or inhibiting tumor growth in a subject, comprising administering a compound according to this invention, to a subject suffering from a proliferative disorder (e.g., cancer) under conditions effective to suppress, reduce or inhibit said tumor growth in said subject. In some embodiments, the compound destroys SDH-deficient cells. In some embodiments, the compound is any one of the compounds listed in Table 1; each compound represents a separate embodiment according to this invention. In some embodiments, the tumor is gastrointestinal stromal tumor. In some embodiments, the tumor is pheochromocytoma. In some embodiments, the tumor is pituitary adenoma. In some embodiments, the tumor is leiomyoma. In some embodiments, the leiomyoma is uterine fibroids. In some embodiments, the tumor is pancreatic neuroendocrine tumor. In some embodiments, the tumor is paraganglioma. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Mutations in metabolic genes such SDHx are leading to rewiring metabolic pathways to support the growth of the cancer cells. These mutations may lead to dependencies of the cancer cells on new pathways/proteins that are serving as synthetic lethal pair with the cancerous mutation and can be exploit for targeting specifically the tumor cells. SDHB mutation carriers were specifically shown to be predisposed to malignant and particularly aggressive forms of the disease. The effects of all these mutations are to abolish SDH activity thereby creating cell-deficient cells, which results in high steady-state intracellular concentrations of Succinate.

Therefore, in various embodiments, this invention provides methods for destroying an SDH-deficient cell, comprising contacting a compound according to this invention, with an SDH-deficient cell, under conditions effective to destroy said cell. In some embodiments, the method is carried out in vitro. In some embodiments, the method is carried out in vivo. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention provides methods for inhibiting an SDH-deficient tumor growth in a subject, comprising administering a compound according to this invention, to a subject under conditions effective to inhibit the growth of said SDH-deficient tumor in said subject. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Ketogenic diet is a high fat, moderate protein, and low carbohydrate diet that causes the body to produce ketones for fuel in the absence of glucose. These ketones can act as an alternative fuel source that feed the TCA cycle, most importantly in the brain, when glucose is not available. This diet can support normal function of the various cells but can't be used by tumor cells with TCA cycle deficiency including but not limited to the SDH-deficient tumors. The ability of most of the body's cells, but not the tumor cells, to use ketones as an energy source can be used to increase the therapeutic window of glucose transporters inhibitors, and to reduce their toxicity.

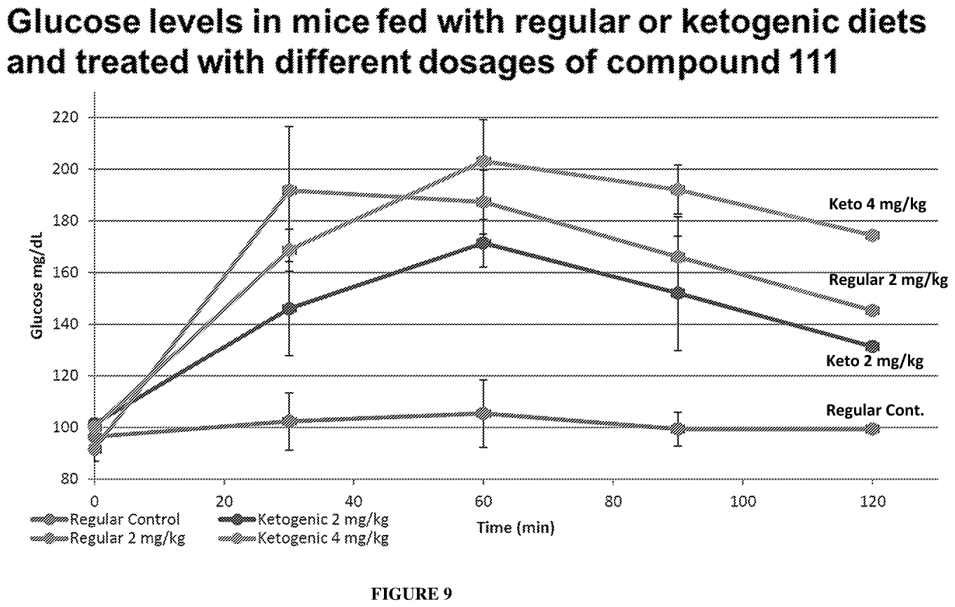

FIG. 9 depicts the glucose levels as measured in mice after different dosages of compound 111 at different diets. According to the figure, ketogenic diet doubled the Maximum Tolerated Dose (MTD) and delayed elevation in plasma glucose levels after compound 111 treatment.

Accordingly, in various embodiments, this invention provides methods for treating, suppressing, reducing the severity, reducing the risk of developing or inhibiting cancer in a subject receiving ketogenic diet, comprising administering a compound of this invention to a subject suffering from cancer and receiving ketogenic diet, at a dose that is larger than the Maximum Tolerated Dose (MTD) of said subject when the subject does not receive a ketogenic diet. In some embodiments, the compound dose is at least 50% higher than the MTD of the subject when he does not receive a ketogenic diet. In some embodiments, the compound dose is at least 80% higher than the MTD of the subject when he does not receive a ketogenic diet. In some embodiments, the compound dose is at least double the MTD of the subject when he does not receive a ketogenic diet. In some embodiments, the compound targets SDH-deficient cells. In some embodiments, the compound destroys SDH-deficient cells. In some embodiments, the ketogenic diet increases the use of fat instead of glucose as primary source of energy in normal cells (i.e., not cancerous). In some embodiments, the use of ketogenic diet in conjugation with the administration of the compound to the subject, increases the therapeutic window of the compound. In some embodiments, the compound is any one of the compounds listed in Table 1; each compound represents a separate embodiment according to this invention. In some embodiments, the compound is a Glucose uptake (GluT) inhibitor.

In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

In various embodiments, this invention provides methods for treating, suppressing, reducing the severity, reducing the risk, or inhibiting metastatic cancer comprising the step of administering to said subject a compound of this invention and/or an isomer, metabolite, pharmaceutically acceptable salt, pharmaceutical product, tautomer, hydrate, N-oxide, polymorph, or crystal of said compound, or any combination thereof. In some embodiments, the compound targets SDH-deficient cells. In some embodiments, the compound destroys SDH-deficient cells. In some embodiments, the cancer is a result of gastrointestinal stromal tumors. In some embodiments, the cancer is renal cell carcinoma. In some embodiments, the cancer is pheochromocytoma. In some embodiments, the cancer is paraganglioma. In some embodiments, the cancer is pituitary adenoma. In some embodiments, the cancer is familial pheochromocytoma. In some embodiments, the cancer is familial paraganglioma. In some embodiments, the cancer is colorectal cancer. In some embodiments, the cancer is gastric cancer. In some embodiments, the cancer is ovarian cancer. In some embodiments, the cancer is leiomyosarcoma.

In various embodiments, this invention provides methods for increasing the survival of a subject suffering from metastatic cancer comprising the step of administering to said subject a compound of this invention and/or an isomer, metabolite, pharmaceutically acceptable salt, pharmaceutical product, tautomer, hydrate, N-oxide, polymorph, prodrug, isotopic variant (e.g. deuterated analog), PROTAC or crystal of said compound, or any combination thereof. In some embodiments, the compound targets SDH-deficient cells. In some embodiments, the cancer is gastrointestinal stromal tumors. In some embodiments, the cancer is renal cell carcinoma. In some embodiments, the cancer is paragangliomas. In some embodiments, the cancer is pituitary adenomas. In some embodiments, the cancer is familial pheochromocytoma. In some embodiments, the cancer is familial paraganglioma. In some embodiments, the cancer is colorectal cancer. In some embodiments, the cancer is gastric cancer. In some embodiments, the cancer is ovarian cancer. In some embodiments, the cancer is leiomyosarcoma.

In various embodiments, this invention provides methods for treating, suppressing, reducing the severity, reducing the risk, or inhibiting advanced cancer comprising the step of administering to said subject a compound of this invention and/or an isomer, metabolite, pharmaceutically acceptable salt, pharmaceutical product, tautomer, hydrate, N-oxide, polymorph, prodrug, isotopic variant (e.g. deuterated analog), PROTAC or crystal of said compound, or any combination thereof. In some embodiments, the compound targets SDH-deficient cells. In some embodiments, the cancer is gastrointestinal stromal tumors. In some embodiments, the cancer is renal cell carcinoma. In some embodiments, the cancer is paragangliomas. In some embodiments, the cancer is pituitary adenomas. In some embodiments, the cancer is familial pheochromocytoma. In some embodiments, the cancer is familial paraganglioma. In some embodiments, the cancer is colorectal cancer. In some embodiments, the cancer is gastric cancer. In some embodiments, the cancer is ovarian cancer. In some embodiments, the cancer is leiomyosarcoma.

In various embodiments, this invention provides methods for increasing the survival of a subject suffering from advanced cancer comprising the step of administering to said subject a compound of this invention and/or an isomer, metabolite, pharmaceutically acceptable salt, pharmaceutical product, tautomer, hydrate, N-oxide, polymorph, prodrug, isotopic variant (e.g. deuterated analog), PROTAC or crystal of said compound, or any combination thereof. In some embodiments, the compound targets SDH-deficient cells. In some embodiments, the cancer is gastrointestinal stromal tumors. In some embodiments, the cancer is renal cell carcinoma. In some embodiments, the cancer is paragangliomas. In some embodiments, the cancer is pituitary adenomas. In some embodiments, the cancer is familial pheochromocytoma. In some embodiments, the cancer is familial paraganglioma. In some embodiments, the cancer is colorectal cancer. In some embodiments, the cancer is gastric cancer. In some embodiments, the cancer is ovarian cancer. In some embodiments, the cancer is leiomyosarcoma.

The compounds of the present invention are useful in the treatment, reducing the severity, reducing the risk, or inhibition of cancer, metastatic cancer, advanced cancer, drug resistant cancer, and various forms of cancer. In a preferred embodiment the cancer is gastrointestinal stromal tumors, renal cell carcinoma, pituitary adenoma, pheochromocytoma, paraganglioma, leiomyosarcoma, colorectal cancer, gastric cancer, and/or ovarian cancer; each represents a separate embodiment according to this invention. Based upon their believed mode of action, it is believed that other forms of cancer will likewise be treatable or preventable upon administration of the compounds or compositions of the present invention to a patient. Preferred compounds of the present invention are selectively disruptive to SDH-deficient cells, causing ablation of SDH-deficient cells but preferably not normal cells. Significantly, harm to normal cells is minimized because the SDH-deficient cells are susceptible to disruption at much lower concentrations of the compounds of the present invention.

In various embodiments, other types of cancers that may be treatable with the compounds according to this invention include: adrenocortical carcinoma, anal cancer, bladder cancer, brain tumor, brain stem tumor, breast cancer, glioma, cerebellar astrocytoma, cerebral astrocytoma, ependymoma, medulloblastoma, supratentorial primitive neuroectodermal, pineal tumors, hypothalamic glioma, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, hepatocellular cancer, hematological cancer or any combination thereof. In some embodiments the cancer is invasive. In some embodiments the cancer is metastatic cancer. In some embodiments the cancer is advanced cancer. In some embodiments the cancer is drug resistant cancer.

In various embodiments “metastatic cancer” refers to a cancer that spread (metastasized) from its original site to another area of the body. Virtually all cancers have the potential to spread. Whether metastases develop depends on the complex interaction of many tumor cell factors, including the type of cancer, the degree of maturity (differentiation) of the tumor cells, the location and how long the cancer has been present, as well as other incompletely understood factors. Metastases spread in three ways—by local extension from the tumor to the surrounding tissues, through the bloodstream to distant sites or through the lymphatic system to neighboring or distant lymph nodes. Each kind of cancer may have a typical route of spread. The tumor is called by the primary site (ex. breast cancer that has spread to the brain is called metastatic breast cancer to the brain).

In various embodiments “drug-resistant cancer” refers to cancer cells that acquire resistance to chemotherapy. Cancer cells can acquire resistance to chemotherapy by a range of mechanisms, including the mutation or overexpression of the drug target, inactivation of the drug, or elimination of the drug from the cell. Tumors that recur after an initial response to chemotherapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the chemotherapy mutate (change) and become resistant to the drug. Once they multiply, there may be more resistant cells than cells that are sensitive to the chemotherapy; b) Gene amplification. A cancer cell may produce hundreds of copies of a particular gene. This gene triggers an overproduction of protein that renders the anticancer drug ineffective; c) cancer cells may pump the drug out of the cell as fast as it is going in using a molecule called p-glycoprotein; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism; g) Cells and tumors with activating RAS mutations are relatively resistant to most anti-cancer agents. Thus, the resistance to anticancer agents used in chemotherapy is the main cause of treatment failure in malignant disorders, provoking tumors to become resistant. Drug resistance is the major cause of cancer chemotherapy failure.

In various embodiments “resistant cancer” refers to drug-resistant cancer as described herein above. In some embodiments “resistant cancer” refers to cancer cells that acquire resistance to any treatment such as chemotherapy, radiotherapy or biological therapy.

In various embodiments, this invention is directed to treating, suppressing, reducing the severity, reducing the risk, or inhibiting cancer in a subject, wherein the subject has been previously treated with chemotherapy, radiotherapy or biological therapy.

In various embodiments “Chemotherapy” refers to chemical treatment for cancer such as drugs that kill cancer cells directly. Such drugs are referred as “anti-cancer” drugs or “antineoplastics.” Today's therapy uses more than 100 drugs to treat cancer. To cure a specific cancer. Chemotherapy is used to control tumor growth when cure is not possible; to shrink tumors before surgery or radiation therapy; to relieve symptoms (such as pain); and to destroy microscopic cancer cells that may be present after the known tumor is removed by surgery (called adjuvant therapy). Adjuvant therapy is given to prevent a possible cancer reoccurrence.

In various embodiments, “Radiotherapy” (also referred herein as “Radiation therapy”) refers to high energy x-rays and similar rays (such as electrons) to treat disease. Many people with cancer will have radiotherapy as part of their treatment. This can be given either as external radiotherapy from outside the body using x-rays or from within the body as internal radiotherapy. Radiotherapy works by destroying the cancer cells in the treated area. Although normal cells can also be damaged by the radiotherapy, they can usually repair themselves. Radiotherapy treatment can cure some cancers and can also reduce the chance of a cancer coming back after surgery. It may be used to reduce cancer symptoms.

In various embodiments “Biological therapy” refers to substances that occur naturally in the body to destroy cancer cells. There are several types of treatment including: monoclonal antibodies, cancer growth inhibitors, vaccines and gene therapy. Biological therapy is also known as immunotherapy.

When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

Accordingly, and in various embodiments, the compound according to this invention, is administered in combination with an anti-cancer therapy. Examples of such therapies include but are not limited to: chemotherapy, immunotherapy, radiotherapy, biological therapy, surgical intervention, and combinations thereof. In some embodiments, the compound according to this invention is administered in combination with radiotherapy.

Compounds according to this invention may be co-administered with other antineoplastic (anti-cancer) agents, including chemotherapeutic agents and radiation. Anti-neoplastic agents can be grouped into a variety of classes including, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, steroids and anti-angiogenesis agents. Examples of alkylating agents include, but are not limited to, cisplatin, cyclophosphamide, melphalan, and dacarbazine. Examples of antimetabolites include, but are not limited to, doxorubicin, daunorubicin, and paclitaxel, gemcitabine. Non-limiting examples of topoisomerase inhibitors are irinotecan (CPT-11), aminocamptothecin, camptothecin, DX-8951f, and topotecan (topoisomerase I) and etoposide (VP-16) and teniposide (VM-26) (topoisomerase II). When the anti-neoplastic agent is radiation, the source of the radiation can be either external (e.g., external beam radiation therapy—EBRT) or internal (i.e., brachytherapy—BT) to the patient being treated. The dose of anti-neoplastic agent administered depends on numerous factors, including, for example, the type of agent, the type and severity tumor being treated and the route of administration of the agent. It should be emphasized, however, that the present invention is not limited to any particular dose.

In various embodiments, the compound is administered in combination with an antineoplastic agent (or anti-cancer agent) by administering the compounds as herein described, alone or in combination with other agents.

Compounds according to this invention may be co-administered with a corticosteroid hormone, anti-inflammatory drugs and cytokine targeting agents to achieve a better therapeutic activity on relieving/reducing the symptoms associated with autoimmune conditions. Examples of corticosteroid hormone include, but are not limited to, prednisolone, prednisone, hydrocortisone, methylprednisolone, and dexamethasone, cortisol, cortisone, triamcinolone, betamethasone, etc.

In various embodiments, the compound is administered in combination with an anti-inflammatory agent by administering the compounds as herein described, alone or in combination with other agents.

In various embodiments, the compound is administered in combination with an agents used for treatment of metabolic disease by administering the compounds as herein described, alone or in combination with other agents. Non-limiting examples for these are: biguanides (e.g., metformin), which reduce hepatic glucose output and increase uptake of glucose by the periphery, SGLT inhibitors, insulin secretagogues (e.g., sulfonylureas and meglitinides) which trigger or enhance insulin release by pancreatic β-cells, and PPARy, PPARa, and PPARa/γ modulators (e.g., thiazolidinediones such as pioglitazone and rosiglitazone). Further examples may include statins, lipid lowering drugs such as MTP inhibitors and LDLR upregulators, antihypertensive agents such as angiotensin antagonists, e.g., losartan, irbesartan, olmesartan, candesartan, and telmisartan, calcium channel antagonists, e.g. lacidipine, ACE inhibitors, e.g., enalapril, and β-adrenergic blockers (β-blockers), e.g., atenolol, labetalol, and nebivolol.

In various embodiments, the compound is administered in combination with an anti-parasitic agent by administering the compounds as herein described, alone or in combination with other agents.

In various embodiments, the compound is administered in combination with an anti-viral agent by administering the compounds as herein described, alone or in combination with other agents.

In various embodiments, the composition for cancer treatment of the present invention can be used together with existing chemotherapy drugs or be made as a mixture with them. Such a chemotherapy drug includes, for example, alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, they can be used together with hypoleukocytosis (neutrophil) medicines that are cancer treatment adjuvant, thrombopenia medicines, antiemetic drugs, and cancer pain medicines for patient's QOL recovery or be made as a mixture with them.

In various embodiments, this invention is directed to a method of destroying a cancerous cell comprising: providing a compound of this invention and contacting the cancerous cell with the compound under conditions effective to destroy the contacted cancerous cell. According to various embodiments of destroying the cancerous cells, the cells to be destroyed can be located either in vivo or ex vivo (i.e., in culture).

In some embodiments, the cancer is selected from the group consisting of gastrointestinal stromal tumors, renal cell carcinoma, paragangliomas, pituitary adenoma, familial pheochromocytoma, familial paraganglioma, colorectal cancer, gastric cancer, ovarian cancer and combinations thereof. In some embodiments, the cancer is selected from the group consisting of: melanoma, non-small cell lung cancer, kidney cancer, bladder cancer, head and neck cancers, Hodgkin lymphoma, glioblastoma, Merkel cell skin cancer (Merkel cell carcinoma), esophagus cancer; gastroesophageal junction cancer; liver cancer, (hepatocellular carcinoma); lung cancer, (small cell) (SCLC); stomach cancer; upper urinary tract cancer, (urothelial carcinoma); multiforme Glioblastoma; Multiple myeloma; anus cancer, (squamous cell); cervix cancer; endometrium cancer; nasopharynx cancer; ovary cancer; metastatic pancreas cancer; solid tumor cancer; adrenocortical Carcinoma; HTLV-1-associated adult T-cell leukemia-lymphoma; uterine Leiomyosarcoma; acute myeloid Leukemia; chronic lymphocytic Leukemia; diffuse large B-cell Lymphoma; follicular Lymphoma; uveal Melanoma; Meningioma; pleural Mesothelioma; Myelodysplasia; Soft tissue sarcoma; breast cancer; colon cancer; pancreatic cancer, Cutaneous T-cell lymphoma; peripheral T-cell lymphoma or any combination thereof.

A still further aspect of the present invention relates to a method of treating or preventing a cancerous condition that includes: providing a compound of the present invention and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to another embodiment, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

Naive lymphocytes resemble many of the somatic cells in the body and progress through metabolic pathways in a textbook fashion, relying on glycolysis and subsequent TCA cycling to produce a maximum amount of ATP. However, upon activation, inflammatory cells, including activated macrophages, Th1 cells, and Th17 cells, generally undergo metabolic reprogramming and rely mainly on glycolysis to exert functions. In the innate immune system, once activated by the pro-inflammatory stimuli, macrophages and dendritic cells (DC) undergo a metabolic switch away from oxidative phosphorylation (OXPHOS) towards glycolysis (Krawczyk et al. “Toll-like receptor-induced changes in glycolytic metabolism regulate dendritic cell activation”. Blood. 2010; Palsson-McDermott et al. “Pyruvate kinase M2 regulates Hif-1alpha activity and IL-1 beta induction and is a critical determinant of the Warbug effect in LPS-activated macrophages.” Cell Metab. 2015), even in the presence of abundant oxygen, similar to the Warburg effect observed in cancer cells.

In various embodiments, this invention is directed to a method of treating, suppressing, reducing the severity, reducing the risk of developing or inhibiting an autoimmune disease or disorder in a subject, comprising administering a compound of this invention to a subject suffering from an autoimmune disease or disorder under conditions effective to treat, suppress, reduce the severity, reduce the risk of developing, or inhibit the an autoimmune disease or disorder. In some embodiments, the autoimmune disorder is Guillain-Barré syndrome (GBS). In some embodiments, the autoimmune disorder is Systemic lupus erythematosus (SLE). In some embodiments, the autoimmune disorder is Rheumatoid arthritis (RA). In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Guillain-Barré syndrome (GBS) is a rapid-onset muscle weakness caused by the immune system damaging the peripheral nervous system. The underlying mechanism involves an autoimmune disorder in which the body's immune system mistakenly attacks the peripheral nerves and damages their myelin insulation. It is an acute immune-mediated inflammatory demyelinating disease of the peripheral nervous system, characterized by inflammatory cell infiltration. Glycolysis inhibition with 2-DG (2-deoxyglucose) not only inhibited the initiation, but also prevented the progression of experimental autoimmune neuritis (EAN), a classic model of GBS, evidenced by the improved clinical scores, weight loss, inflammatory cell infiltration, and demyelination of sciatic nerves. 2-DG inhibited the differentiation of Th1, Th17, and Tfh cells but enhanced Treg cell development, accompanied with reduced autoantibody secretion (Ru-Tao et al, “Enhanced glycolysis contributes to the pathogenesis of experimental autoimmune neuritis”, J Neuroinflammation. 2018).

Therefore, in various embodiments, this invention is directed to a method of treating, suppressing, reducing the severity, reducing the risk of developing or inhibiting Guillain-Barré syndrome (GBS) in a subject, comprising administering a compound of this invention to a subject suffering from a Guillain-Barré syndrome (GBS) under conditions effective to treat, suppress, reduce the severity, reduce the risk of developing, or inhibit the Guillain-Barré syndrome (GBS). In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Systemic lupus erythematosus (SLE), also known simply as lupus, is an autoimmune disease in which the body's immune system mistakenly attacks healthy tissue in many parts of the body. Symptoms vary between people and may be mild to severe. This is another condition of pathogenic T cell activation that has been targeted glycolysis inhibition. Cells from the lupus mice had higher expression of many genes in the glycolytic pathway, but unexpectedly had an increase in Cpt1a, a key regulator of fatty acid oxidation (Yin et al., “Normalization of CD4+ T cell metabolism reverses lupus”. Sci. Transl. Med. 2015). To capitalize on the metabolic differences between normal and SLE cells, experiments were undertaken to treat the mice with 2-DG and metformin. When these drugs are used to treat the CD4+ T cells from lupus mice ex vivo, they are capable of preventing the production of IFN-γ. When given in vivo, 2 DG and metformin were able to decrease the ECAR and OCR to levels comparable with those of disease-free control animals. Furthermore, they do not affect naive cells or the immune system as a whole, because circulating total Ab levels are unchanged.

Accordingly, in various embodiments, this invention is directed to a method of treating, suppressing, reducing the severity, reducing the risk of developing or inhibiting Systemic lupus erythematosus (SLE) in a subject, comprising administering a compound of this invention to a subject suffering from a Systemic lupus erythematosus (SLE) under conditions effective to treat, suppress, reduce the severity, reduce the risk of developing, or inhibit the Systemic lupus erythematosus (SLE). In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

Rheumatoid arthritis (RA) is an autoimmune disease in which the body's immune system—which normally protects its health by attacking foreign substances like bacteria and viruses—mistakenly attacks the joints. It is characterized by synovial proliferation and leukocyte extravasation leading to joint destruction. The increased proliferation and rapid activation of cells in the inflamed joint require the switch of glucose metabolism to a highly metabolically glycolytic state, in order to maintain energy homoeostasis (Bustamante et al., “Fibroblast-like synoviocyte metabolism in the pathogenesis of rheumatoid arthritis” Arthritis Res Ther. 2017; Pucino et al., “Lactate at the crossroads of metabolism, inflammation, and autoimmunity”. Eur J Immunol. 2017). Accordingly, inhibition of glycolysis with 2 DG significantly reduced joint inflammation and the activation of both adaptive and innate immune cells, as well as the production of pathogenic autoantibodies (Abboud et al., “Inhibition of Glycolysis Reduces Disease Severity in an Autoimmune Model of Rheumatoid Arthritis.” Front Immunol. 2018).

Accordingly, in various embodiments, this invention is directed to a method of treating, suppressing, reducing the severity, reducing the risk of developing or inhibiting Rheumatoid arthritis (RA) in a subject, comprising administering a compound of this invention to a subject suffering from a Rheumatoid arthritis (RA) under conditions effective to treat, suppress, reduce the severity, reduce the risk of developing, or inhibit the Rheumatoid arthritis (RA). In some embodiments, the compound targets SDH-deficient cells. In some embodiments, the compound destroys SDH-deficient cells. In some embodiments, the compound is any one of the compounds listed in Table 1; each compound represents a separate embodiment according to this invention. In some embodiments, the compound is a Glucose uptake (GluT) inhibitor. In some embodiments, the compound inhibits all Glucose uptake transporters (GluT). In some embodiments, the compound is selective towards a specific glucose transporter. In some embodiments, the compound is selective towards specific glucose transporters, selected from: GluT1, GluT2, GluT3, GluT4, GluT5, GluT6, GluT7, GluT8, GluT9, GluT10, GluT11, GluT12, GluT13, GluT14, and combinations thereof. In some embodiments, the compound is selective towards specific glucose transporters, selected from: GluT1, GluT2, GluT3, GluT4, and combinations thereof. In some embodiments, the compound inhibits the activity of all Type I glucose transporters. In some embodiments, the compound inhibits the activity of GluT1, GluT2 and GluT3. In some embodiments, the compound is a competitive with glucose in the inhibition of glucose transporters. In some embodiments, the compound is compound 145. In other embodiments, the compound is compound 111. In other embodiments, the compound is compound 308. In other embodiments, the compound is compound 332. In some embodiments, the compound is any one of the compounds listed in Table 1; each compound represents a separate embodiment according to this invention.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The following examples are presented in order to more fully illustrate the preferred embodiments of the invention. They should in no way, however, be construed as limiting the broad scope of the invention.

### EXAMPLES

### Example 1

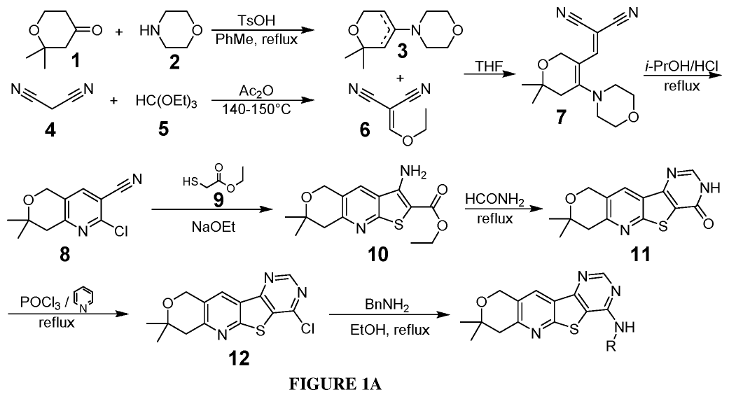

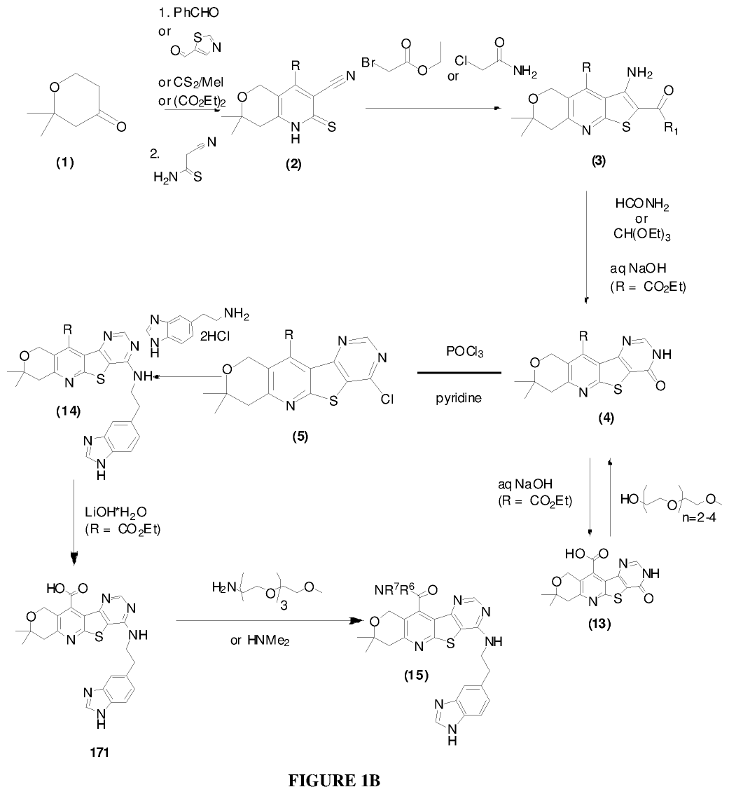

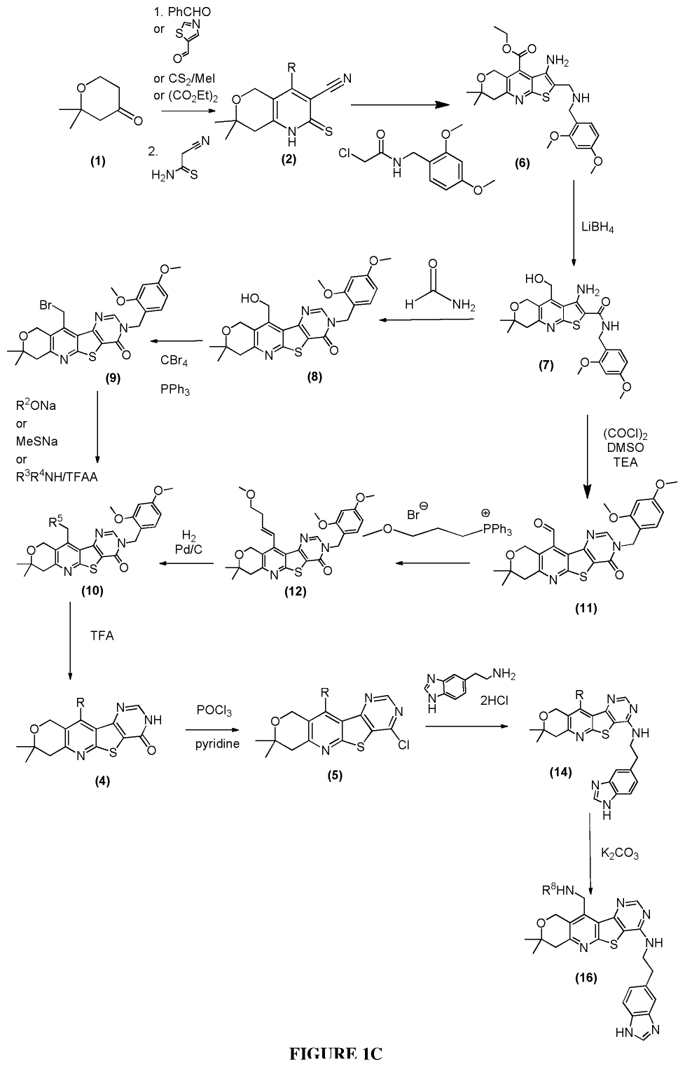

### Synthetic Details for Compounds of the Invention (FIG. 1)

### Experimental Procedure:

### General Synthetic Details for Compounds of the Invention (FIG. 1A):

### 4-(2,2-Dimethyl-3,6-dihydro-2H-pyran-4-yl)morpholine (3)

Into a 250-mL flask, equipped with a Dean-Stark trap and a reflux condenser, were placed 2,2-dimethyldihydro-2H-pyran-4(3H)-one (1) (13.47 g, 105 mmol, 1 equiv), morpholine (2) (15.8 ml, 184 mmol, 1.75 equiv), p-toluenesulfonic acid (100 mg) and 100 mL of toluene. The reaction mixture was refluxed for 4 h upon complete liberation of water. After toluene was evaporated, the residue was distilled in vacuo at 15-17 Torr to afford the target product (3) as a yellowish liquid (11.6 g, 56%, b.p. 135-140° C./15-17 Torr).

### 2-(Ethoxymethylene)malononitrile (6)

Into a 250-mL flask was placed malononitrile (4) (25 g, 378 mmol, 1 equiv), triethyl orthoformate (5) (94.3 ml, 567 mmol, 1.5 equiv) and acetic anhydride (71.5 ml, 756 mmol, 2 equiv). The resultant mixture was heated at 140° C. for 1 h, then at 150° C. for 1 h more. After Ac₂O was evaporated, the residue was distilled in vacuo at 10 Torr to afford the target product (6) as a yellow liquid (28.4 g, 62%, b.p. 140-150° C./10 Torr) which solidifies upon staying.

### 2-((6,6-Dimethyl-4-morpholino-5,6-dihydro-2H-pyran-3-yl)methylene)malononitrile (7)

A homogeneous mixture of 4-(2,2-dimethyl-3,6-dihydro-2H-pyran-4-yl)morpholine (3) (8.5 g, 43.1 mmol, 1 equiv) and 2-(ethoxymethylene)malononitrile (6) (5.26 g, 43.1 mmol, 1 equiv) in 30 mL of dry THF was left at room temperature for 8 hours. After THF was evaporated, the residue was crystallized from 30 mL of cold absolute MeOH to afford the target product (7) as a yellow solid (5.42 g, 46%).

### 2-Chloro-7,7-dimethyl-7,8-dihydro-5H-pyrano[4,3-b]pyridine-3-carbonitrile (8)

A suspension of 2-((6,6-dimethyl-4-morpholino-5,6-dihydro-2H-pyran-3-yl)methylene)malononitrile (7) (5.42 g, 19.8 mmol) in 60 mL i-PrOH was cooled to 0° C., followed by saturation with gaseous HCl during 1 h and subsequent reflux for 4 h. The solvent was removed in vacuo, the residue was treated with 60 mL of sat. aq. solution of NH₃. The precipitated crystals were filtered off, washed with water and dried. The resultant product was recrystallized from 20 mL of EtOH to afford the target compound (8) as a white solid (2.4 g, 54%).

### Ethyl 3-amino-7,7-dimethyl-7,8-dihydro-5H-pyrano[4,3-b]thieno[3,2-e]pyridine-2 carboxylate (10)

Sodium metal (195 mg, 8.47 mmol, 1.1 equiv) was dissolved in 40 mL of absolute EtOH. To this mixture were added 2-chloro-7,7-dimethyl-7,8-dihydro-5H-pyrano[4,3-b]pyridine-3-carbonitrile (8) (1.72 g, 7.7 mmol, 1 equiv) and 920 μL of ethyl 2-mercaptoacetate (9). The reaction mixture was refluxed for 8 h. After the solvent was evaporated, the residue was mixed with 50 mL of cold water, the precipitated solid was filtered off and recrystallized from 20 mL of EtOH to afford the target compound (10) as a white solid (1.9 g, 80%).

### 8,8-Dimethyl-7,8-dihydro-3H-pyrano[3″,4″:5′,6′]pyrido[3′,2′:4,5]thieno[3,2-d]pyrimidin-4(10H)-one (11)

Into a 50-mL flask were placed ethyl 3-amino-7,7-dimethyl-7,8-dihydro-5H-pyrano[4,3-b]thieno[3,2-e]pyridine-2-carboxylate (10) (1.88 g, 6.1 mmol) and 25 mL of formamide. The mixture was refluxed under Ar for 4 h. The reaction mixture was allowed to cool to rt followed by addition of 150 mL of H₂O. The resultant mixture was put into a fridge (˜5° C.) and the precipitate formed was filtered off and dried in vacuo to afford the target compound (11) as a dark-grey solid (1.26 g, 72%).

### 4-Chloro-8,8-dimethyl-8,10-dihydro-7H-pyrano[3″,4″:5′,6′]pyrido[3′,2′:4,5]thieno-[3,2-d]pyrimidine (12)

Into a 50-mL flask were placed 8,8-dimethyl-7,8-dihydro-3H-pyrano[3″,4″:5′,6′]pyrido-[3′,2′:4,5]thieno[3,2-d]pyrimidin-4(10H)-one (11) (1.26 g, 4.4 mmol), 15 mL of POCl₃ and 0.5 mL of pyridine. The mixture was refluxed for 5 h. The solvent was removed in vacuo, the residue was mixed with 150 mL of sat. aq. NaHCO₃ solution and the precipitate formed was filtered off and dried in vacuo to afford the product (12) as a light-grey solid (1.38 g, 97%).

### Compound 100-162

In a 10-mL flask were mixed 4-chloro-8,8-dimethyl-8,10-dihydro-7H-pyrano[3″,4″:5′,6′]-pyrido[3′,2′:4,5]thieno[3,2-d]pyrimidine (12) (70 mg, 0.23 mmol, 1 equiv) and any amine (54 mg, 0.51 mmol, 2.2 equiv) in 4 mL of EtOH. The reaction mixture was refluxed for 10 h. Then to the mixture was added 15 mL of H₂O, the precipitate was filtered off, dried and dissolved in 50 mL of hot EtOH. The insoluble solid was filtered off, the solvent from the solution was partially evaporated (˜5 mL was left in a flask). The precipitate was filtered off and the mother liquid was subjected to CC (eluent: DCM/MeOH=98/2) to afford the target product, Compound 100-162.

### Analytical Data (NMR)

100

¹H NMR (400 MHz, DMSO-d6) δ 8.61 (s, 1H), 8.39 (s, 1H), 7.20 (d, J=12.5 Hz, 1H), 7.12-7.06 (m, 2H), 4.94 (s, 2H), 3.97 (t, J=7.5 Hz, 2H), 3.79 (s, 3H), 3.37 (s, 3H), 2.98 (s, 2H), 2.92 (t, J=7.5 Hz, 2H), 1.28 (s, 6H). HRMS (ESI-TOF), m/z: found for [M+H⁺] (C₂₄H₂₆FN₄O₂S⁺) 453.1779; calculated 453.1755.

101

¹H NMR (400 MHz, DMSO-d6) δ 8.60 (s, 1H), 8.30 (s, 1H), 8.02 (t, J=5.2 Hz, 1H), 7.16-6.98 (m, 3H), 3.89-3.84 (m, 1H), 3.78 (s, 3H), 3.71 (q, J=6.7 Hz, 2H), 3.63-3.45 (m, 2H), 3.26-2.81 (m, 6H), 2.11-1.94 (m, 2H), 1.11 (t, J=7.0 Hz, 3H). HRMS (ESI-TOF), m/z: found for [M+H⁺] (C₂₄H₂₆FN₄O₂S⁺) 453.1777; calculated 453.1755.

102

¹H NMR (400 MHz, DMSO-d6) δ 8.62 (s, 1H), 8.35 (s, 1H), 8.07 (t, J=5.4 Hz, 1H), 6.43 (d, J=2.2 Hz, 2H), 6.33 (t, J=2.2 Hz, 1H), 4.93 (s, 2H), 3.79-3.69 (m, 2H), 3.69 (s, 6H), 2.97 (s, 2H), 2.89 (t, J=7.3 Hz, 2H), 1.28 (s, 6H). HRMS (ESI-TOF), m/z: found for [M+H⁺] (C₂₄H₂₇N₄O₃S⁺) 451.1819; calculated 451.1798.

103

¹H NMR (400 MHz, DMSO-d6) δ 8.63 (s, 1H), 8.35 (s, 1H), 8.12 (t, J=5.4 Hz, 1H), 6.99 (t, J=7.8 Hz, 1H), 6.91 (d, J=7.7 Hz, 1H), 6.82 (d, J=7.3 Hz, 1H), 4.93 (s, 2H), 3.79 (s, 3H), 3.75 (s, 3H), 3.71 (q, J=6.3 Hz, 2H), 2.98 (s, 2H), 2.95 (t, J=6.5 Hz, 2H), 1.28 (s, 6H). HRMS (ESI-TOF), m/z: found for [M+H⁺](C₂₄H₂₇N₄O₃S⁺) 451.1815; calculated 451.1798.

104

¹H NMR (400 MHz, DMSO-d6) δ 8.66 (s, 1H), 8.63 (s, 1H), 8.10 (t, J=5.4 Hz, 1H), 7.17-6.97 (m, 3H), 6.69 (s, 1H), 5.05 (s, 2H), 3.79 (s, 3H), 3.73 (q, J=6.6 Hz, 2H), 2.90 (t, J=7.2 Hz, 2H), 2.13 (s, 3H), 2.03 (s, 3H). HRMS (ESI-TOF), m/z: found for [M+H⁺] (C₂₃H₂₃ClFN₄OS⁺) 457.1271; calculated 457.1260.

105

¹H NMR (400 MHz, DMSO-d6) δ 8.57 (s, 1H), 8.37 (s, 1H), 7.30 (d, J=13.0 Hz, 1H), 7.18-7.12 (m, 2H), 4.93 (s, 2H), 4.44-4.04 (m, 2H), 3.83 (s, 3H), 3.97-3.45 (m, 3H), 2.97 (s, 2H), 2.42-2.34 (m, 1H), 2.13 (p, J=11.2, 10.6 Hz, 1H), 1.28 (s, 6H). HRMS (ESI-TOF), m/z: found for [M+H⁺] (C₂₅H₂₆FN₄O₂S⁺) 465.1770; calculated 465.1755.

107

¹H NMR (400 MHz, DMSO-d6) δ 8.82 (dd, J=4.6, 1.4 Hz, 1H), 8.64 (s, 1H), 8.62 (d, J=1.4 Hz, 1H), 8.12 (t, J=5.4 Hz, 1H), 7.64 (dd, J=7.9, 4.7 Hz, 1H), 7.24-6.98 (m, 2H), 6.81 (ddd, J=7.7, 4.1, 1.6 Hz, 1H), 3.79 (s, 3H), 3.78 (q, J=7.5 Hz, 2H), 2.94 (t, J=7.2 Hz, 2H). HRMS (ESI-TOF), m/z: found for [M+H⁺] (C₁₈H₁₆FN₄OS⁺) 355.1026; calculated 355.1023.

108

¹H NMR (400 MHz, DMSO-d6) δ 8.57 (s, 1H), 8.37 (s, 1H), 7.94 (t, J=5.3 Hz, 1H), 7.14-6.97 (m, 3H), 3.80 (s, 3H), 3.72 (q, J=6.8 Hz, 2H), 3.08 (dt, J=7.6, 4.1 Hz, 4H), 2.90 (t, J=7.3 Hz, 2H), 2.19 (p, J=7.5 Hz, 2H). HRMS (ESI-TOF), m/z: found for [M+H⁺] (C₂₁H₂₀FN₄OS⁺) 395.1338; calculated 395.1336.

109

¹H NMR (400 MHz, DMSO-d6) δ 8.59 (s, 1H), 8.27 (s, 1H), 7.97 (t, J=5.5 Hz, 1H), 7.21-6.88 (m, 3H), 3.79 (s, 3H), 3.71 (q, J=6.5 Hz, 2H), 3.01 (t, J=6.4 Hz, 2H), 2.95 (t, J=6.1 Hz, 2H), 2.89 (t, J=7.3 Hz, 2H), 1.89 (qd, J=6.6, 4.4, 2.6 Hz, 2H), 1.85-1.76 (m, 2H). HRMS (ESI-TOF), m/z: found for [M+H⁺](C₂₂H₂₂FN₄OS⁺) 409.1498; calculated 409.1493.

110

¹H NMR (400 MHz, DMSO-d6) δ 8.81 (dd, J=4.6, 1.4 Hz, 1H), 8.63 (s, 1H), 8.62 (d, J=1.5 Hz, 1H), 8.10 (t, J=5.3 Hz, 1H), 7.63 (dd, J=8.0, 4.6 Hz, 1H), 7.16-6.97 (m, 3H), 3.79 (s, 3H), 3.74 (q, J=6.8 Hz, 2H), 2.91 (t, J=7.3 Hz, 2H). HRMS (ESI-TOF), m/z: found for [M+H⁺] (C₁₈H₁₆FN₄OS⁺) 355.1036; calculated 355.1023.

111

¹H NMR (400 MHz, DMSO-d6) δ 9.83 (s, 1H), 8.59 (s, 1H), 8.33 (s, 1H), 8.01 (t, J=6.1 Hz, 1H), 7.48 (d, J=8.3 Hz, 2H), 7.17 (d, J=8.3 Hz, 2H), 4.92 (s, 2H), 3.71 (q, J=6.3 Hz, 2H), 2.97 (s, 2H), 2.90 (t, J=7.5 Hz, 2H), 2.01 (s, 3H), 1.29 (s, 6H). HRMS (ESI-TOF), m/z: found for [M+H⁺] (C₂₄H₂₆N₅O₂S⁺) 448.1817; calculated 448.1802.

112

¹H NMR (400 MHz, DMSO-d6) δ 8.57 (s, 1H), 8.33 (s, 1H), 7.89 (t, J=5.2 Hz, 1H), 4.92 (s, 2H), 3.88 (d, J=10.6 Hz, 1H), 3.57-3.50 (m, 2H), 2.97 (s, 2H), 1.88-1.37 (m, 8H), 1.28 (s, 6H), 1.28-1.17 (m, 2H). HRMS (ESI-TOF), m/z: found for [M+H⁺] (C₂₁H₂₇N₄O₂S⁺) 399.1864; calculated 399.1849.

113

¹H NMR (400 MHz, DMSO-d6) δ 12.88 (s, 1H), 8.62 (s, 1H), 8.35 (s, 1H), 8.07 (t, J=5.4 Hz, 1H), 7.86 (s, 1H), 7.79 (d, J=7.6 Hz, 1H), 7.52 (d, J=7.6 Hz, 1H), 7.42 (t, J=7.6 Hz, 1H), 4.93 (s, 2H), 3.78 (q, J=6.7 Hz, 2H), 3.03 (t, J=7.3 Hz, 2H), 2.98 (s, 2H), 1.28 (s, 6H). HRMS (ESI-TOF), m/z: found for [M+H⁺](C₂₃H₂₃N₄O₃S⁺) 435.1472; calculated 435.1485.

114

¹H NMR (400 MHz, DMSO-d6) δ 8.59 (s, 1H), 8.42 (t, J=5.8 Hz, 1H), 8.33 (s, 1H), 7.02 (s, 1H), 6.91-6.86 (m, 2H), 4.92 (s, 2H), 4.68 (d, J=5.8 Hz, 2H), 3.72 (d, J=7.6 Hz, 6H), 2.96 (s, 2H), 1.28 (s, 6H). HRMS (ESI-TOF), m/z: found for [M+H⁺] (C₂₃H₂₅N₄O₃S⁺) 437.1663; calculated 437.1642.

115

¹H NMR (400 MHz, DMSO-d6) δ 8.61 (s, 1H), 8.34 (s, 1H), 8.02 (t, J=5.4 Hz, 1H), 6.88-6.79 (m, 2H), 6.74 (dd, J=8.1, 1.5 Hz, 1H), 4.93 (s, 2H), 3.84 (t, J=6.6 Hz, 2H), 3.77-3.70 (m, 5H), 2.97 (s, 2H), 2.88 (t, J=7.3 Hz, 2H), 1.69 (h, J=7.1 Hz, 2H), 1.28 (s, 6H), 0.95 (t, J=7.4 Hz, 3H). HRMS (ESI-TOF), m/z: found for [M+H⁺] (C₂₆H₃₁N₄O₃S⁺) 479.2115; calculated 479.2111.

116

¹H NMR (400 MHz, DMSO-d6) δ 10.82 (s, 1H), 8.64 (s, 1H), 8.35 (s, 1H), 8.11 (t, J=5.5 Hz, 1H), 7.62 (d, J=7.8 Hz, 1H), 7.34 (d, J=8.1 Hz, 1H), 7.21 (d, J=2.0 Hz, 1H), 7.07 (t, J=7.4 Hz, 1H), 6.99 (t, J=7.2 Hz, 1H), 4.93 (s, 2H), 3.81 (t, J=6.4 Hz, 2H), 3.07 (t, J=7.8 Hz, 2H), 2.98 (s, 2H), 1.28 (s, 6H). HRMS (ESI-TOF), m/z: found for [M+H⁺] (C₂₄H₂₄N₅OS⁺) 430.1700; calculated 430.1696.

117

¹H NMR (400 MHz, DMSO-d6) δ 8.61 (s, 1H), 8.34 (s, 1H), 8.06 (t, J=5.4 Hz, 1H), 7.20 (t, J=8.1 Hz, 1H), 6.85-6.82 (m, 2H), 6.77 (dd, J=7.2, 1.5 Hz, 1H), 4.92 (s, 2H), 3.82-3.72 (m, 2H), 3.72 (s, 3H), 2.97 (s, 2H), 2.93 (t, J=7.4 Hz, 2H), 1.28 (s, 6H). HRMS (ESI-TOF), m/z: found for [M+H⁺] (C₂₃H₂₅N₄O₂S⁺) 421.1696; calculated 421.1693.

120

¹H NMR (400 MHz, DMSO-d6) δ 8.56 (s, 1H), 8.33 (s, 1H), 7.96 (t, J=5.5 Hz, 1H), 7.34-7.21 (m, 2H), 7.12-7.04 (m, 1H), 4.93 (s, 2H), 3.75 (q, J=6.7 Hz, 2H), 3.00-2.92 (m, 4H), 1.30 (s, 6H). HRMS (ESI-TOF), m/z: found for [M+H⁺] (C₂₂H₂₁F₂N₄OS⁺) 427.1404; calculated 427.1399.

121

¹H NMR (400 MHz, DMSO-d6) δ 8.56 (s, 1H), 8.33 (s, 1H), 7.96 (t, J=5.5 Hz, 1H), 7.09-6.98 (m, 2H), 6.83-6.75 (m, 1H), 4.93 (s, 2H), 3.81 (s, 3H), 3.76 (q, J=6.7 Hz, 2H), 2.99-2.90 (m, 4H), 1.30 (s, 6H). HRMS (ESI-TOF), m/z: found for [M+H⁺] (C₂₃H₂₄FN₄O₂S⁺) 439.1597, calculated 439.1599.

122

¹H NMR (400 MHz, DMSO-d6) δ 8.56 (s, 1H), 8.33 (s, 1H), 7.95 (t, J=5.4 Hz, 1H), 7.12-6.96 (m, 3H), 4.93 (s, 2H), 3.80 (s, 3H), 3.72 (q, J=6.5 Hz, 2H), 2.97 (s, 2H), 2.90 (t, J=7.4 Hz, 2H), 1.30 (s, 6H). HRMS (ESI-TOF), m/z: found for [M+H⁺] (C₂₃H₂₄FN₄O₂S⁺) 439.1587, calculated 439.1599.

123

¹H NMR (400 MHz, DMSO-d6) δ 9.01 (s, 1H), 8.69 (s, 2H), 8.58 (s, 1H), 8.35 (s, 1H), 8.06 (t, J=5.5 Hz, 1H), 4.93 (s, 2H), 3.82 (q, J=6.6 Hz, 2H), 3.03-2.95 (m, 4H), 1.28 (s, 6H). HRMS (ESI-TOF), m/z: found for [M+H⁺] (C₂₀H₂₁N₆OS⁺) 393.1498, calculated 393.1492.

124

¹H NMR (400 MHz, DMSO-d6) δ 8.57 (s, 1H), 8.47 (s, 1H), 8.40 (d, J=3.7 Hz, 2H), 8.34 (s, 1H), 8.01 (t, J=5.5 Hz, 1H), 7.67 (d, J=7.8 Hz, 1H), 7.33-7.24 (m, 1H), 4.93 (s, 2H), 3.79 (q, J=6.9 Hz, 2H), 3.04-2.93 (m, 4H), 1.31 (s, 6H). HRMS (ESI-TOF), m/z: found for [M+H⁺] (C₂₁H₂₂N₅OS⁺) 392.1542, calculated 392.1540.

125

¹H NMR (400 MHz, DMSO-d6) δ 8.55 (s, 1H), 8.32 (s, 1H), 7.88 (t, J=5.4 Hz, 1H), 7.04 (d, J=8.3 Hz, 1H), 6.50 (d, J=2.1 Hz, 1H), 6.41 (dd, J=8.2, 2.2 Hz, 1H), 4.93 (s, 2H), 3.77 (s, 3H), 3.74 (s, 3H), 3.66 (q, J=6.7 Hz, 2H), 2.97 (s, 2H), 2.86 (t, J=7.4 Hz, 2H), 1.30 (s, 6H). HRMS (ESI-TOF), m/z: found for [M+H⁺] (C₂₄H₂₇N₄O₃S⁺) 451.1796, calculated 451.1798.

126

¹H NMR (400 MHz, DMSO-d6) δ 8.83-8.77 (m, 1H), 8.61 (m, 2H), 8.06 (t, J=5.1 Hz, 1H), 7.62 (dd, J=7.9, 4.7 Hz, 1H), 6.88-6.79 (m, 2H), 6.77 (d, J=8.1 Hz, 1H), 3.73 (m, 8H), 2.90 (t, J=7.3 Hz, 2H). HRMS (ESI-TOF), m/z: found for (C₁₉H₁₉N₄O₂S⁺) [M+H⁺] 367.1215, calculated 367.1223.

127

¹H NMR (400 MHz, DMSO-d6) δ 8.61 (s, 1H), 8.46 (d, J=5.6 Hz, 2H), 8.35 (s, 1H), 8.06 (t, J=5.6 Hz, 1H), 7.29 (d, J=5.8 Hz, 2H), 4.93 (s, 2H), 3.80 (q, J=6.1 Hz, 2H), 2.98 (m, 4H), 1.29 (s, 6H). HRMS (ESI-TOF), m/z: found for 392.1549, calculated 392.1540.

128

¹H NMR (400 MHz, Chloroform-d) δ 8.69 (s, 1H), 8.28 (s, 1H), 7.27-7.17 (m, 2H), 6.94 (t, J=8.4 Hz, 2H), 5.72 (t, J=5.0 Hz, 1H), 4.98 (s, 2H), 3.97 (s, 3H), 3.86 (q, J=6.8, 4.6 Hz, 2H), 3.11-3.02 (m, 4H), 1.37 (s, 6H). HRMS (ESI-TOF), m/z: found for [M+H⁺] (C₂₃H₂₅N₄O₂S⁺) 421.1695, calculated 421.1693.

129

¹H NMR (400 MHz, Chloroform-d) δ 8.73 (s, 1H), 8.30 (s, 1H), 7.16 (d, J=8.6 Hz, 2H), 6.87 (d, J=8.6 Hz, 2H), 4.98 (m, 3H), 3.90 (q, J=6.8 Hz, 2H), 3.80 (s, 3H), 3.05 (s, 2H), 2.97 (t, J=7.0 Hz, 2H), 1.37 (s, 6H). HRMS (ESI-TOF), m/z: found for [M+H⁺] (C₂₃H₂₅N₄O₂S⁺) 421.1702, calculated 421.1693.

130

¹H NMR (400 MHz, DMSO-d6) δ 8.34 (s, 1H), 8.06 (s, 1H), 7.89 (d, J=7.6 Hz, 2H), 7.42 (d, J=7.6 Hz, 3H), 4.93 (s, 2H), 3.81 (m, 5H), 3.05 (t, J=6.5 Hz, 2H), 2.97 (s, 2H), 1.28 (s, 6H). HRMS (ESI-TOF), m/z: found for [M+H⁺] (C₂₄H₂₅N₄O₃S⁺) 449.1656, calculated 449.1642

131

¹H NMR (400 MHz, DMSO-d6) δ 8.61 (s, 1H), 8.35 (s, 1H), 6.80-6.70 (m, 3H), 4.93 (s, 2H), 4.19 (s, 4H), 3.68 (m, 2H), 2.98 (s, 3H), 2.84 (t, J=7.02 Hz, 2H), 1.29 (s, 6H). HRMS (ESI-TOF), m/z: found for [M+H⁺] (C₂₄H₂₅N₄O₃S⁺) 449.1636, calculated 449.1642.

132

¹H NMR (400 MHz, DMSO-d₆) δ 10.13 (s, 1H), 8.77 (s, 1H), 8.52 (d, J=5.4 Hz, 1H), 7.40-7.21 (m, 2H), 6.98 (d, J=8.5 Hz, 1H), 4.95 (s, 2H), 3.80 (d, J=6.4 Hz, 6H), 2.99 (s, 2H), 1.31 (s, 6H). HRMS (ESI-TOF), m/z: found for [M+H⁺] (C₂₂H₂₃N₄O₃S⁺) 423.1495; calculated 423.1485.

133

¹H NMR (400 MHz, DMSO-d₆) δ 8.56 (s, 1H), 8.50 (d, J=4.3 Hz, 1H), 8.32 (s, 1H), 7.99 (t, J=5.4 Hz, 1H), 7.67 (td, J=7.6, 1.8 Hz, 1H), 7.33-7.15 (m, 2H), 4.93 (s, 2H), 3.89 (q, J=8.3 Hz, 2H), 3.12 (t, J=7.4 Hz, 2H), 2.97 (s, 2H), 1.30 (s, 6H). HRMS (ESI-TOF), m/z: found for [M+H⁺] (C₂₁H₂₂N₅OS⁺) 392.1528; calculated 392.1540.

134

¹H NMR (400 MHz, DMSO-d₆) δ 8.58 (s, 1H), 8.34 (s, 1H), 7.74 (d, J=7.7 Hz, 1H), 4.93 (s, 2H), 4.57-4.41 (m, 1H), 2.98 (s, 2H), 1.30-1.23 (m, 12H). HRMS (ESI-TOF), m/z: found for [M+H⁺](C₁₇H₂₁N₄OS⁺) 329.1442; calculated 329.1431.

135

¹H NMR (400 MHz, DMSO-d₆) δ 8.54 (s, 1H), 8.45 (t, J=5.9 Hz, 1H), 8.34 (s, 1H), 7.44-7.16 (m, 5H), 4.93 (s, 2H), 4.77 (d, J=5.8 Hz, 2H), 2.97 (s, 2H), 1.30 (s, 6H). HRMS (ESI-TOF), m/z: found for [M+H⁺] (C₂₁H₂₁N₄OS⁺) 377.1447; calculated 377.1431.

136

¹H NMR (400 MHz, DMSO-d₆) δ 9.51 (s, 2H), 8.76 (s, 1H), 8.48 (s, 1H), 4.95 (s, 2H), 4.19 (t, J=5.2 Hz, 4H), 3.30 (s, 4H), 3.00 (s, 2H), 1.30 (s, 6H). HRMS (ESI-TOF), m/z: found for [M+H⁺−nHCl](C₁₈H₂₂N₅OS⁺) 356.1543; calculated 356.1540.

137

¹H NMR (400 MHz, DMSO-d₆) δ 8.65 (s, 1H), 8.39 (s, 1H), 4.94 (s, 2H), 3.94 (t, J=4.5 Hz, 4H), 3.79 (t, J=4.8 Hz, 4H), 2.98 (s, 2H), 1.30 (s, 6H). HRMS (ESI-TOF), m/z: found for [M+H⁺] (C₁₈H₂₁N₄O₂S⁺) 357.1393; calculated 357.1380.

138

¹H NMR (400 MHz, Chloroform-d) δ 8.66 (s, 1H), 8.30 (s, 1H), 4.98 (s, 2H), 3.97 (d, J=5.7 Hz, 4H), 3.05 (s, 2H), 1.81-1.71 (m, 6H), 1.37 (s, 6H). HRMS (ESI-TOF), m/z: found for [M+H⁺] (C₁₉H₂₃N₄OS⁺) 355.1597; calculated 355.1587.

139

¹H NMR (400 MHz, DMSO-d₆) δ 8.61 (s, 1H), 8.35 (s, 1H), 8.02 (t, J=5.2 Hz, 1H), 6.89-6.71 (m, 3H), 4.93 (s, 2H), 3.71 (d, J=3.1 Hz, 7H), 2.98 (s, 2H), 2.89 (t, J=7.3 Hz, 3H), 1.28 (s, 6H). HRMS (ESI-TOF), m/z: found for [M+H⁺] (C₂₄H₂₇N₄O₃S⁺) 451.1802; calculated 451.1798.

141

¹H NMR (400 MHz, DMSO-d6) δ 8.62 (s, 1H), 8.44 (s, 1H), 8.35 (s, 1H), 8.08 (t, J=5.3 Hz, 1H), 7.31 (d, J=8.4 Hz, 2H), 7.11 (d, J=8.4 Hz, 2H), 5.79 (s, 2H), 4.93 (s, 2H), 3.69 (q, J=6.2 Hz, 2H), 2.98 (s, 2H), 2.86 (t, J=7.5 Hz, 2H), 1.28 (s, 6H). HRMS (ESI-TOF), m/z: found for [M+H⁺] (C₂₃H₂₅N₆O₂S⁺) 449.1755; calculated 449.1754.

142

¹H NMR (400 MHz, DMSO-d6) δ 8.62 (s, 1H), 8.46-8.28 (m, 2H), 8.12 (t, J=5.5 Hz, 1H), 7.78 (d, J=8.2 Hz, 2H), 7.35 (d, J=8.1 Hz, 2H), 4.93 (s, 2H), 3.77 (q, 2H), 3.08-2.93 (m, 4H), 2.77 (d, J=4.5 Hz, 3H), 1.28 (s, 6H). HRMS (ESI-TOF), m/z: found for [M+H⁺] (C₂₄H₂₆N₅O₂S⁺) 448.1803; calculated 448.1802.

143

¹H NMR (400 MHz, DMSO-d6) δ 8.63 (s, 1H), 8.35 (s, 1H), 8.12 (t, J=5.4 Hz, 1H), 7.25 (d, J=8.4 Hz, 2H), 7.17 (s, 1H), 7.01 (d, J=8.4 Hz, 2H), 6.87 (s, 1H), 4.93 (s, 2H), 3.74 (q, J=6.5 Hz, 2H), 2.96 (t, J=10.9 Hz, 4H), 1.28 (s, 6H). HRMS (ESI-TOF), m/z: found for [M+H⁺] (C₂₃H₂₄N₅O₃S⁺) 450.1597; calculated 450.1594.

144

¹H NMR (400 MHz, DMSO-d6) δ 8.63 (s, 1H), 8.36 (s, 1H), 8.11 (s, 1H), 7.92 (s, 1H), 7.80 (d, J=8.2 Hz, 2H), 7.42-7.14 (m, 3H), 4.93 (s, 2H), 3.77 (q, J=6.4 Hz, 2H), 3.03-2.98 (m, 4H), 1.28 (s, 6H). HRMS (ESI-TOF), m/z: found for [M+H⁺] (C₂₃H₂₄N₅O₂S⁺) 434.1651; calculated 434.1645.

145

¹H NMR (400 MHz, DMSO-d6) δ 12.35 (s, 1H), 8.63 (s, 1H), 8.35 (s, 1H), 8.19-8.02 (m, 2H), 7.65-7.30 (m, 2H), 7.10 (d, J=8.0 Hz, 1H), 4.93 (s, 2H), 3.78 (q, J=6.4 Hz, 2H), 3.05 (t, J=7.2 Hz, 2H), 2.97 (s, 2H), 1.27 (s, 6H). HRMS (ESI-TOF), m/z: found for [M+H⁺] (C₂₃H₂₃N₆OS⁺) 431.1653; calculated 431.1649.

146

¹H NMR (400 MHz, DMSO-d6) δ 8.62 (s, 1H), 8.36 (s, 1H), 8.14 (t, J=5.4 Hz, 1H), 7.42-7.22 (m, 4H), 4.93 (s, 2H), 3.75 (q, J=6.2 Hz, 2H), 3.20 (s, 3H), 2.96 (d, J=11.4 Hz, 4H), 2.90 (s, 3H), 1.28 (s, 6H). HRMS (ESI-TOF), m/z: found for [M+H⁺] (C₂₄H₂₈N₅O₃S₂⁺) 498.1630; calculated 498.1628.

147

¹H NMR (400 MHz, DMSO-d6) δ 8.63 (s, 1H), 8.35 (s, 1H), 8.20 (s, 1H), 8.13 (t, J=5.1 Hz, 1H), 7.91 (d, J=7.7 Hz, 2H), 7.43 (d, J=7.8 Hz, 2H), 7.36 (s, 1H), 4.93 (s, 2H), 3.79 (q, J=6.5 Hz, 2H), 3.03 (t, J=7.2 Hz, 2H), 2.97 (s, 2H), 1.27 (s, 6H). HRMS (ESI-TOF), m/z: found for [M+H⁺] (C₂₅H₂₄N₅O₂S⁺) 458.1653; calculated 458.1645.

148

¹H NMR (400 MHz, DMSO-d6) δ 9.06 (s, 1H), 8.63 (s, 1H), 8.35 (s, 1H), 8.28 (s, 1H), 8.11 (t, J=5.4 Hz, 1H), 7.62 (d, J=8.1 Hz, 2H), 7.34 (d, J=8.2 Hz, 2H), 4.93 (s, 2H), 3.77 (q, J=6.7 Hz, 2H), 3.01-2.97 (m, 4H), 1.28 (s, 6H). HRMS (ESI-TOF), m/z: found for [M+H⁺] (C₂₅H₂₄N₅OS₂⁺) 474.1448; calculated 474.1417.

149

¹H NMR (400 MHz, DMSO-d6) δ 8.63 (s, 1H), 8.42 (s, 1H), 8.35 (s, 1H), 8.11 (t, J=5.2 Hz, 1H), 7.66 (d, J=8.9 Hz, 3H), 7.38 (d, J=8.0 Hz, 2H), 4.93 (s, 2H), 3.77 (q, J=6.4 Hz, 2H), 3.02-2.98 (m, J=8.3 Hz, 4H), 1.28 (s, 6H). HRMS (ESI-TOF), m/z: found for [M+H⁺] (C₂₅H₂₄N₅O₂S⁺) 458.1669; calculated 458.1645.

150

¹H NMR (400 MHz, DMSO-d6) δ 8.61 (s, 1H), 8.36 (s, 1H), 8.12 (t, J=5.5 Hz, 1H), 7.32 (s, 4H), 4.93 (s, 2H), 3.77 (q, J=7.2 Hz, 2H), 3.05-2.77 (m, 10H), 1.28 (s, 6H). HRMS (ESI-TOF), m/z: found for [M+H⁺] (C₂₅H₂₈N₅O₂S⁺) 462.1986; calculated 462.1958.

151

¹H NMR (400 MHz, DMSO-d6) δ 8.61 (s, 1H), 8.34 (s, 1H), 8.04 (t, J=5.6 Hz, 1H), 6.91 (d, J=7.9 Hz, 2H), 6.49 (d, J=7.9 Hz, 2H), 4.90 (d, J=19.7 Hz, 4H), 3.64 (q, J=6.5 Hz, 2H), 2.97 (s, 2H), 2.76 (t, J=7.7 Hz, 2H), 1.28 (s, 6H). HRMS (ESI-TOF), m/z: found for [M+H⁺] (C₂₂H₂₄N₅OS⁺) 406.1708; calculated 406.1696.

152

¹H NMR (400 MHz, DMSO-d6) δ 9.63 (s, 1H), 8.61 (s, 1H), 8.35 (s, 1H), 8.09 (t, J=5.3 Hz, 1H), 7.23 (d, J=8.2 Hz, 2H), 7.13 (d, J=8.3 Hz, 2H), 4.93 (s, 2H), 3.72 (q, J=7.0 Hz, 2H), 2.97 (s, 2H), 2.93-2.89 (m, 5H), 1.27 (s, 6H). HRMS (ESI-TOF), m/z: found for [M+H⁺] (C₂₃H₂₆N₅O₃S₂+) 484.1487; calculated 484.1472.

153

¹H NMR (400 MHz, DMSO-d6) δ 8.91 (s, 1H), 8.47 (s, 1H), 7.22 (d, J=12.6 Hz, 1H), 7.10 (d, J=4.0 Hz, 2H), 4.94 (s, 2H), 4.75 (t, J=6.6 Hz, 2H), 3.80 (s, 3H), 3.09 (t, J=6.6 Hz, 2H), 2.99 (s, 2H), 1.28 (s, 6H). HRMS (ESI-TOF), m/z: found for [M+H⁺] (C₂₃H₂₃FN₃O₃S⁺) 440.1641; calculated 440.1439.

154

¹H NMR (400 MHz, DMSO-d6) δ 9.21 (s, 1H), 8.61 (s, 1H), 8.35 (s, 1H), 8.07 (t, J=5.4 Hz, 1H), 7.05 (d, J=8.3 Hz, 2H), 6.68 (d, J=8.3 Hz, 2H), 4.93 (s, 2H), 3.67 (q, J=8.3 Hz, 2H), 2.97 (s, 2H), 2.83 (t, J=7.9 Hz, 2H), 1.28 (s, 6H). HRMS (ESI-TOF), m/z: found for [M+H⁺] (C₂₂H₂₃N₄O₂S⁺) 407.1553; calculated 407.1536.

156

¹H NMR (400 MHz, DMSO-d6) δ 8.62 (s, 1H), 8.35 (s, 1H), 8.08 (s, 1H), 7.57 (d, J=8.2 Hz, 2H), 7.26 (d, J=8.2 Hz, 2H), 4.93 (s, 2H), 3.81-3.70 (m, 4H), 3.02-2.88 (m, 4H), 2.05 (q, J=7.4 Hz, 2H), 1.28 (s, 6H). HRMS (ESI-TOF), m/z: found for [M+H⁺] (C₂₆H₂₈N₅O₂S⁺) 474.1979; calculated 474.1958.

157

¹H NMR (400 MHz, DMSO-d6) δ 8.61 (s, 1H), 8.35 (s, 1H), 8.12 (s, J=5.3 Hz, 1H), 7.38 (d, J=8.5 Hz, 2H), 7.27 (d, J=8.5 Hz, 2H), 4.93 (s, 2H), 3.76 (q, J=6.7 Hz, 2H), 3.35 (s, 3H), 3.01-2.97 (m, 4H), 1.28 (s, 6H). HRMS (ESI-TOF), m/z: found for [M+H⁺] (C₂₃H₂₅N₄O₄S₂⁺) 485.1324; calculated 485.1312.

158

¹H NMR (400 MHz, DMSO-d6) δ 8.60 (s, 1H), 8.35 (s, 1H), 8.11 (t, J=5.2 Hz, 1H), 7.33 (d, J=7.7 Hz, 2H), 7.23 (d, J=7.8 Hz, 2H), 4.93 (s, 2H), 3.77 (q, J=6.4 Hz, 2H), 3.10 (s, 3H), 2.97 (s, 4H), 1.70 (s, 3H), 1.28 (s, 6H). HRMS (ESI-TOF), m/z: found for [M+H⁺] (C₂₅H₂₈N₅O₂S⁺) 462.1988; calculated 462.1958.

159

¹H NMR (400 MHz, DMSO-d6) δ 12.78 (s, 1H), 8.58 (s, 1H), 8.33 (s, 1H), 8.05 (t, J=5.4 Hz, 1H), 7.87 (d, J=8.0 Hz, 2H), 7.38 (d, J=8.0 Hz, 2H), 4.92 (s, 2H), 3.77 (q, J=6.6 Hz, 2H), 3.03 (t, J=7.3 Hz, 2H), 2.96 (s, 2H), 1.29 (s, 6H). HRMS (ESI-TOF), m/z: found for [M+H⁺] (C₂₃H₂₃N₄O₃S⁺) 435.1487; calculated 435.1485.

160

¹H NMR (400 MHz, DMSO-d6) δ 9.21 (s, 1H), 8.53 (s, 1H), 6.95 (dd, J=12.6, 1.6 Hz, 1H), 6.85 (d, J=8.5 Hz, 1H), 6.77 (t, J=8.6 Hz, 1H), 4.97 (s, 2H), 4.28 (t, J=6.9 Hz, 2H), 3.57 (s, 3H), 3.02 (s, 2H), 2.81 (t, J=6.9 Hz, 2H), 2.21 (s, 3H), 1.29 (s, 6H). HRMS (ESI-TOF), m/z: found for [M+Na⁺] (C₂₅H₂₅FN₄NaO₃S⁺) 503.1552; calculated 503.1524.

161

¹H NMR (400 MHz, Chloroform-d) δ 8.74 (s, 1H), 8.30 (s, 1H), 7.46 (s, 1H), 7.36 (d, J=8.5 Hz, 1H), 6.95 (d, J=8.5 Hz, 1H), 5.04-4.99 (m, 3H), 3.98-3.85 (m, 5H), 3.10-2.96 (m, 4H), 1.37 (s, 6H). HRMS (ESI-TOF), m/z: found for [M+H⁺] (C₂₄H₂₄F₃N₄O₂S⁺) 489.1570; calculated 489.1567.

162

¹H NMR (400 MHz, DMSO-d6) δ 8.58 (s, 1H), 8.33 (s, 1H), 7.90 (t, J=6.0 Hz, 1H), 7.15 (t, J=8.2 Hz, 1H), 6.60 (d, J=8.3 Hz, 2H), 4.92 (s, 2H), 3.67 (s, 6H), 3.58 (q, J=7.2 Hz, 2H), 2.96 (s, 2H), 2.96 (t, J=7.3 Hz, 2H), 1.27 (s, 6H). HRMS (ESI-TOF), m/z: found for [M+H⁺] (C₂₄H₂₇N₄O₃S⁺) 451.1811; calculated 451.1798.

163

¹H NMR (400 MHz, DMSO-d6) δ 12.47 (br s, 1H), 8.58 (s, 1H), 8.23 (br s, 1H), 8.14 (s, 1H), 7.71-7.34 (m, 2H), 7.09 (d, J=8.1 Hz, 1H), 4.82 (s, 2H), 4.57 (s, 2H), 3.77 (s, 4H), 3.64-2.95 (m, 23H), 1.29 (s, 6H). LCMS (ESI), m/z: found for [M+H⁺] (C₃₅H₄₅N₆O₈S⁺) 709.21; calculated 709.30.

164

¹H NMR (400 MHz, DMSO-d6) δ 12.33 (s, 1H), 8.59 (s, 1H), 8.19 (t, J=5.4 Hz, 1H), 8.15 (s, 1H), 7.57-7.32 (m, 2H), 7.10 (d, J=8.2 Hz, 1H), 4.83 (s, 2H), 4.58 (dd, J=5.8, 3.4 Hz, 2H), 4.03-3.71 (m, 4H), 3.63-3.35 (m, 12H), 3.20 (s, 3H), 3.10-3.02 (m, 2H), 3.05 (s, 2H), 1.30 (s, 6H). LCMS (ESI), m/z: found for [M+H⁺] (C₃₃H₄₁N₆O₇S⁺) 665.13; calculated 665.28.

165

¹H NMR (400 MHz, DMSO-d6) δ 12.58 (br s, 1H), 8.58 (s, 1H), 8.24 (t, J=5.7 Hz, 1H), 8.13 (s, 1H), 7.50 (d, J=8.3 Hz, 1H), 7.44 (s, 1H), 7.09 (d, J=8.2 Hz, 1H), 4.82 (s, 2H), 4.57 (t, J=4.7 Hz, 2H), 3.93-3.68 (m, 4H), 3.58-3.26 (m, 8H), 3.18 (s, 3H), 3.06-3.02 (m, 2H), 3.04 (s, 2H), 1.29 (s, 6H). LCMS (ESI), m/z: found for [M+H⁺] (C₃₁H₃₇N₆O₆S⁺) 621.25; calculated 621.25.

166

¹H NMR (400 MHz, CDCl₃) δ 12.34 (br s, 1H), 8.83 (s, 1H), 8.61-8.56 (m, 1H), 8.15 (s, 1H), 7.59-7.31 (m, 2H), 7.09 (d, J=8.1 Hz, 1H), 5.28 (s, 2H), 3.90 (s, 3H), 3.77-3.86 (m, 2H), 3.13 (s, 2H), 3.06-2.98 (m, 2H), 1.31 (s, 6H). LCMS (ESI), m/z: found for [M+H⁺] (C₂₄H₂₅N₆O₅S₂⁺) 541.06; calculated 541.13.

167

¹H NMR (400 MHz, DMSO-d6) δ 12.5 (br s, 1H), 8.73 (s, 1H), 8.18 (s, 1H), 8.03 (t, J=5.5 Hz, 1H), 7.52 (d, J=8.2 Hz, 1H), 7.46 (s, 1H), 7.12 (dd, J=8.2, 1.4 Hz, 1H), 4.99 (s, 2H), 3.79 (q, J=6.3 Hz, 2H), 3.06 (q, J=7.8 Hz, 2H), 2.96 (s, 2H), 2.66 (s, 3H), 1.28 (s, 6H). LCMS (ESI), m/z: found for [M+H⁺](C₂₄H₂₅N₆OS₂⁺) 477.07; calculated 477.15.

168

¹H NMR (400 MHz, DMSO-d6) δ 8.61 (s, 1H), 8.48 (s, 1H), 8.35 (s, 1H), 8.07 (t, J=5.2 Hz, 1H), 7.58 (d, J=8.1 Hz, 1H), 7.52 (s, 1H), 7.21 (d, J=8.1 Hz, 1H), 5.22 (s, 1H), 3.79 (q, J=6.6 Hz, 3H), 3.14-3.05 (m, 5H), 2.16-2.06 (m, 2H), 2.00 (s, 3H). HRMS (ESI-TOF), m/z: found for [M+H⁺] (C₂₄H₂₃N₆O₂S⁺) 459.1591; calculated 459.1598.

169

¹H NMR (400 MHz, DMSO-d6) δ 12.4 (br s, 1H), 8.61 (s, 1H), 8.29 (s, 1H), 8.15 (s, 1H), 8.05 (t, J=5.2 Hz, 1H), 7.51 (d, J=8.1 Hz, 1H), 7.45 (s, 1H), 7.11 (d, J=8.1 Hz, 1H), 4.95 (d, J=3.5 Hz, 1H), 4.08 (s, 1H), 3.78 (q, J=6.6 Hz, 2H), 3.22-2.79 (m, 6H), 1.95 (m, 2H). LCMS (ESI), m/z: found for [M+H⁺](C₂₂H₂₁N₆OS⁺) 417.05; calculated 417.15.

170

¹H NMR (400 MHz, DMSO-d6) δ 12.33 (br s, 1H), 8.58 (s, 1H), 8.18 (t, J=5.5 Hz, 1H), 8.15 (s, 1H), 7.50 (d, J=8.2 Hz, 1H), 7.44 (s, 1H), 7.10 (d, J=8.1 Hz, 1H), 4.80 (s, 2H), 4.49 (q, J=7.1 Hz, 2H), 3.78 (q, J=6.3 Hz, 2H), 3.07-3.04 (m, 4H), 1.35 (t, J=7.1 Hz, 3H), 1.29 (s, 6H). LCMS (ESI), m/z: found for [M+H⁺](C₂₆H₂₇N₆O₃S⁺) 503.10; calculated 503.19.

171

¹H NMR (400 MHz, DMSO-d6) δ 8.61 (s, 1H), 8.25-8.12 (m, 2H), 7.51 (d, J=8.1 Hz, 1H), 7.45 (s, 1H), 7.11 (d, J=8.1 Hz, 1H), 4.83 (s, 2H), 3.79 (q, J=6.7 Hz, 2H), 3.12-2.89 (m, 4H), 1.29 (s, 6H). LCMS (ESI), m/z: found for [M+H⁺] (C₂₄H₂₃N₆O₃S⁺) 475.07; calculated 475.15.

172

¹H NMR (400 MHz, DMSO-d6) δ 8.61 (s, 1H), 8.33 (s, 1H), 8.12 (s, 1H), 8.04 (t, J=5.4 Hz, 1H), 7.55 (s, 1H), 7.47 (d, J=8.3 Hz, 1H), 7.20 (d, J=8.4 Hz, 1H), 4.92 (s, 2H), 3.80 (s, 3H), 3.78-3.60 (m, 2H), 3.49-3.36 (m, 1H), 2.96 (s, 2H), 1.32 (d, J=6.9 Hz, 3H), 1.27 (s, 6H). HRMS (ESI-TOF), m/z: found for [M+H⁺] (C₂₅H₂₇N₆OS⁺) 459.1998; calculated 459.1962.

173

¹H NMR (400 MHz, DMSO-d6) δ 8.63 (s, 1H), 8.35 (s, 1H), 8.14 (s, 1H), 8.09 (t, J=5.4 Hz, 1H), 7.53 (s, 1H), 7.48 (d, J=8.2 Hz, 1H), 7.19 (d, J=8.1 Hz, 1H), 4.93 (s, 2H), 3.81 (s, 3H), 3.80-3.74 (m, 2H), 3.07 (t, J=7.4 Hz, 2H), 2.97 (s, 2H), 1.27 (s, 6H). HRMS (ESI-TOF), m/z: found for [M+H⁺] (C₂₄H₂₅N₆OS⁺) 445.1854; calculated 445.1805.

174

¹H NMR (400 MHz, DMSO-d6) δ 10.77 (s, 1H), 8.60 (s, 1H), 8.35 (s, 1H), 8.06 (t, J=5.3 Hz, 1H), 7.78 (d, J=1.9 Hz, 1H), 7.44 (dd, J=8.5, 2.0 Hz, 1H), 7.04 (d, J=8.5 Hz, 1H), 4.93 (s, 2H), 3.74 (q, J=6.7 Hz, 2H), 2.97 (s, 2H), 2.93 (t, J=7.1 Hz, 2H), 1.27 (s, 6H). HRMS (ESI-TOF), m/z: found for [M+H⁺](C₂₂H₂₂N₅O₄S⁺) 452.1426; calculated 452.1387.

175

¹H NMR (400 MHz, DMSO-d6) δ 8.70 (s, 1H), 8.62 (s, 1H), 8.35 (s, 1H), 8.10 (t, J=5.4 Hz, 1H), 7.69 (s, 1H), 7.68 (d, J=8.4 Hz, 1H), 7.34 (dd, J=8.4, 1.6 Hz, 1H), 4.93 (s, 2H), 3.80 (q, J=6.7 Hz, 2H), 3.10 (t, J=7.3 Hz, 2H), 2.98 (s, 2H), 1.28 (s, 6H). HRMS (ESI-TOF), m/z: found for [M+H⁺] (C₂₃H₂₂N₅O₂S⁺) 432.1513; calculated 432.1489.

176

¹H NMR (400 MHz, DMSO-d6) δ 12.44 (br s, 1H), 8.63 (s, 1H), 8.35 (s, 1H), 8.09 (t, J=5.3 Hz, 1H), 7.59-7.28 (m, 2H), 7.11 (s, 1H), 5.23 (s, 2H), 4.93 (s, 2H), 3.77 (q, J=6.5 Hz, 2H), 3.05 (t, J=7.2 Hz, 2H), 2.97 (s, 2H), 2.11 (s, 3H), 1.27 (s, 6H). HRMS (ESI-TOF), m/z: found for [M+H⁺] (C₂₆H₂₇N₆O₃S⁺) 503.1874; calculated 503.1860.

177

¹H NMR (400 MHz, DMSO-d6) δ 12.13 (br s, 1H), 8.63 (s, 1H), 8.35 (s, 1H), 8.09 (t, J=5.4 Hz, 1H), 7.52-7.24 (m, 2H), 7.04 (d, J=8.2 Hz, 1H), 5.72 (d, J=4.8 Hz, 1H), 4.93 (s, 2H), 4.93-4.84 (m, 1H), 3.76 (q, J=6.7 Hz, 2H), 3.03 (t, J=7.5 Hz, 2H), 2.97 (s, 2H), 1.47 (d, J=6.5 Hz, 3H), 1.28 (s, 6H). HRMS (ESI-TOF), m/z: found for [M+H⁺] (C₂₅H₂₇N₆O₂S⁺) 475.1915; calculated 475.1911.

178

¹H NMR (400 MHz, DMSO-d6) δ 13.21 (d, J=12.5 Hz, 1H), 8.63 (s, 1H), 8.35 (s, 1H), 8.10 (t, J=5.2 Hz, 1H), 7.83-7.60 (m, 1H), 7.54-7.35 (m, 1H), 7.35-7.17 (m, 1H), 4.93 (s, 2H), 3.80 (q, J=6.7 Hz, 2H), 3.09 (t, J=7.2 Hz, 2H), 2.98 (s, 2H), 2.67 (s, 3H), 1.28 (s, 6H). HRMS (ESI-TOF), m/z: found for [M+H⁺](C₂₅H₂₅N₆O₂S⁺) 473.1774; calculated 473.1754.

179

¹H NMR (400 MHz, DMSO-d6) δ 12.23 (s, 1H), 8.63 (s, 1H), 8.35 (s, 1H), 8.10 (t, J=5.4 Hz, 1H), 7.52-7.23 (m, 2H), 7.05 (d, J=7.9 Hz, 1H), 5.67 (t, J=5.4 Hz, 1H), 4.93 (s, 2H), 4.65 (d, J=5.3 Hz, 2H), 3.77 (q, J=6.5 Hz, 2H), 3.03 (t, J=7.4 Hz, 2H), 2.98 (s, 2H), 1.28 (s, 6H). HRMS (ESI-TOF), m/z: found for [M+H⁺] (C₂₄H₂₅N₆O₂S⁺) 461.1803; calculated 461.1754.

180

¹H NMR (400 MHz, DMSO-d6) δ 8.61 (s, 1H), 8.35 (s, 1H), 8.10 (q, J=4.8 Hz, 1H), 8.05 (t, J=5.4 Hz, 1H), 7.94 (d, J=2.0 Hz, 1H), 7.49 (dd, J=8.8, 2.0 Hz, 1H), 6.94 (d, J=8.9 Hz, 1H), 4.93 (s, 2H), 3.73 (q, J=6.8 Hz, 2H), 3.16 (s, 2H), 2.98 (s, 2H), 2.95-2.85 (m, 3H), 1.28 (s, 6H). HRMS (ESI-TOF), m/z: found for [M+H⁺] (C₂₃H₂₅N₆O₃S⁺) 465.1677; calculated 465.1703.

181

¹H NMR (400 MHz, DMSO-d6) δ 8.58 (s, 1H), 8.35 (s, 1H), 8.09 (q, J=4.6 Hz, 1H), 7.98 (d, J=1.7 Hz, 1H), 7.52 (dd, J=8.8, 1.6 Hz, 1H), 6.92 (d, J=8.9 Hz, 1H), 4.93 (s, 2H), 4.01-3.89 (m, 2H), 3.39 (s, 3H), 2.97 (s, 2H), 2.95-2.88 (m, 5H), 1.27 (s, 6H). HRMS (ESI-TOF), m/z: found for [M+H⁺] (C₂₄H₂₇N₆O₃S⁺) 479.1848; calculated 479.1860.

182

¹H NMR (400 MHz, DMSO-d6) δ 11.67 (s, 1H), 8.61 (s, 1H), 8.35 (s, 1H), 8.10 (t, J=5.4 Hz, 1H), 7.99 (d, J=1.7 Hz, 1H), 7.68 (dd, J=8.3, 1.8 Hz, 1H), 7.52 (d, J=8.2 Hz, 1H), 4.93 (s, 2H), 3.82 (q, J=6.7 Hz, 2H), 3.09 (t, J=6.9 Hz, 2H), 2.97 (s, 2H), 1.28 (s, 6H). HRMS (ESI-TOF), m/z: found for [M+H⁺](C₂₄H₂₂F₃N₆O₄S⁺) 547.1342; calculated 547.1370.

183

¹H NMR (400 MHz, DMSO-d6) δ 9.23 (s, 1H), 8.63 (s, 1H), 8.36 (s, 1H), 8.12 (t, J=5.5 Hz, 1H), 7.02 (d, J=8.0 Hz, 1H), 6.64 (d, J=1.4 Hz, 1H), 6.46 (dd, J=8.0, 1.5 Hz, 1H), 4.93 (s, 2H), 4.90 (br s, 2H), 4.64 (s, 2H), 3.74-3.64 (m, 2H), 2.98 (s, 2H), 2.85-2.76 (m, 2H), 2.11 (s, 3H), 1.28 (s, 6H). HRMS (ESI-TOF), m/z: found for [M+H⁺] (C₂₆H₂₉N₆O₄S⁺) 521.1975; calculated 521.1966.

184

¹H NMR (400 MHz, DMSO-d6) δ 10.42 (s, 1H), 8.61 (s, 1H), 8.35 (s, 1H), 8.08 (t, J=5.4 Hz, 1H), 7.94 (d, J=1.7 Hz, 1H), 7.74 (d, J=8.4 Hz, 1H), 7.63 (dd, J=8.4, 1.7 Hz, 1H), 4.93 (s, 2H), 4.68 (s, 2H), 3.80 (q, J=6.7 Hz, 2H), 3.05 (t, J=7.0 Hz, 2H), 2.98 (s, 2H), 2.14 (s, 3H), 1.28 (s, 6H). HRMS (ESI-TOF), m/z: found for [M+H⁺] (C₂₆H₂₇N₆O₆S⁺) 551.1702; calculated 551.1707.

185

¹H NMR (400 MHz, DMSO-d6) δ 12.41 (br s, 1H), 8.63 (s, 1H), 8.35 (s, 1H), 8.09 (t, J=5.4 Hz, 1H), 7.53-7.31 (m, 2H), 7.10 (d, J=8.1 Hz, 1H), 5.95 (q, J=6.7 Hz, 1H), 4.93 (s, 2H), 3.77 (q, J=6.4 Hz, 2H), 3.05 (t, J=7.3 Hz, 2H), 2.98 (s, 2H), 2.09 (s, 3H), 1.61 (d, J=6.7 Hz, 3H), 1.28 (s, 6H). HRMS (ESI-TOF), m/z: found for [M+H⁺] (C₂₇H₂₉N₆O₃S⁺) 517.1985; calculated 517.2016.

186

¹H NMR (400 MHz, DMSO-d6) δ 9.28 (s, 1H), 8.63 (s, 1H), 8.35 (s, 1H), 8.10 (t, J=5.5 Hz, 1H), 7.00 (d, J=8.0 Hz, 1H), 6.64 (d, J=1.5 Hz, 1H), 6.47 (dd, J=8.0, 1.6 Hz, 1H), 5.04 (q, J=6.8 Hz, 1H), 4.93 (s, 2H), 4.77 (s, 2H), 3.69 (q, J=8.9 Hz, 2H), 2.98 (s, 2H), 2.81 (t, J=7.6 Hz, 2H), 2.08 (s, 3H), 1.42 (d, J=6.9 Hz, 3H), 1.28 (s, 6H). HRMS (ESI-TOF), m/z: found for [M+H⁺] (C₂₇H₃₁N₆O₄S⁺) 535.2084; calculated 535.2122.

187

¹H NMR (400 MHz, DMSO-d6) δ 10.42 (s, 1H), 8.61 (s, 1H), 8.34 (s, 1H), 8.07 (t, J=5.3 Hz, 1H), 7.92 (s, 1H), 7.70 (d, J=8.4 Hz, 1H), 7.61 (d, J=8.1 Hz, 1H), 5.09 (q, J=6.9 Hz, 1H), 4.92 (s, 2H), 3.79 (q, J=6.5 Hz, 2H), 3.04 (t, J=6.9 Hz, 2H), 2.97 (s, 2H), 2.11 (s, 3H), 1.42 (d, J=6.9 Hz, 3H), 1.27 (s, 6H). HRMS (ESI-TOF), m/z: found for [M+H⁺] (C₂₇H₂₉N₆O₆S⁺) 565.1861; calculated 565.1864.

188

¹H NMR (400 MHz, DMSO-d6) δ 12.64 (br s, 1H), 8.63 (s, 1H), 8.35 (s, 1H), 8.08 (t, J=4.8 Hz, 1H), 7.47-7.35 (m, 2H), 7.09 (d, J=8.0 Hz, 1H), 4.93 (s, 2H), 3.77 (q, J=6.8 Hz, 2H), 3.21-3.10 (m, 1H), 3.05 (t, J=7.3 Hz, 2H), 2.98 (s, 2H), 1.33 (d, J=6.6 Hz, 6H), 1.28 (s, 6H). HRMS (ESI-TOF), m/z: found for [M+H⁺] (C₂₆H₂₉N₆OS⁺) 473.2105; calculated 473.2118.

189

¹H NMR (400 MHz, DMSO-d6) δ 10.96 (br s, 1H), 8.62 (s, 1H), 8.35 (s, 1H), 8.07 (s, 1H), 7.09-7.00 (m, 2H), 6.82 (d, J=7.7 Hz, 1H), 6.44 (s, 2H), 4.93 (s, 2H), 3.72 (d, J=4.5 Hz, 2H), 3.02-2.90 (m, 4H), 1.28 (s, 6H). HRMS (ESI-TOF), m/z: found for [M+H⁺] (C₂₃H₂₃N₇OS⁺) 446.1772; calculated 446.1758.

190

¹H NMR (400 MHz, DMSO-d6) δ 12.81 (br s, 1H), 8.64 (s, 1H), 8.36 (s, 1H), 8.18-8.09 (m, 3H), 7.62-7.40 (m, 5H), 7.13 (d, J=7.9 Hz, 1H), 4.93 (s, 2H), 3.81 (d, J=5.2 Hz, 2H), 3.08 (t, J=7.1 Hz, 2H), 2.98 (s, 2H), 1.28 (s, 6H). HRMS (ESI-TOF), m/z: found for [M+H⁺] (C₂₉H₂₇N₆OS⁺) 507.1956; calculated 507.1962.

191

¹H NMR (400 MHz, DMSO-d6) δ 8.61 (s, 1H), 8.35 (s, 1H), 8.05 (s, 1H), 6.96 (d, J=12.6 Hz, 1H), 6.83 (d, J=7.7 Hz, 1H), 6.48 (t, J=8.7 Hz, 1H), 5.42 (s, 1H), 4.93 (s, 2H), 3.98 (d, J=4.9 Hz, 2H), 3.67 (s, 2H), 2.97 (s, 2H), 2.83 (t, J=6.9 Hz, 2H), 2.12 (s, 3H), 1.28 (s, 6H). HRMS (ESI-TOF), m/z: found for [M+H⁺](C₂₅H₂₇FN₅O₂S⁺) 480.1838; calculated 480.1864.

192

¹H NMR (400 MHz, DMSO-d6) δ 9.65 (s, 1H), 8.65 (s, 1H), 8.36 (s, 1H), 8.14 (s, 1H), 7.98 (d, J=7.0 Hz, 2H), 7.62-7.44 (m, 3H), 7.10 (d, J=7.9 Hz, 1H), 6.71 (s, 1H), 6.54 (d, J=6.9 Hz, 1H), 5.23-4.79 (m, 4H), 3.72 (s, 2H), 2.98 (s, 2H), 2.84 (t, J=7.0 Hz, 2H), 1.28 (s, 6H). HRMS (ESI-TOF), m/z: found for [M+H⁺] (C₂₉H₂₉N₆O₂S⁺) 525.2069; calculated 525.2067.

193

¹H NMR (400 MHz, DMSO-d6) δ 8.62 (s, 1H), 8.35 (s, 1H), 8.04 (s, 1H), 6.44 (d, J=7.0 Hz, 2H), 6.29 (d, J=7.1 Hz, 1H), 4.94 (s, 2H), 4.35 (d, J=46.4 Hz, 4H), 3.63 (s, 2H), 2.98 (s, 2H), 2.76-2.61 (m, 2H), 1.28 (s, 6H). HRMS (ESI-TOF), m/z: found for [M+H⁺] (C₂₂H₂₅N₆OS⁺) 421.1835; calculated 421.1805.

194

¹H NMR (400 MHz, DMSO-d6) δ 12.12 (s, 1H), 8.63 (s, 1H), 8.35 (s, 1H), 8.08 (s, 1H), 7.71-7.24 (m, 2H), 7.02 (d, J=7.7 Hz, 1H), 4.93 (s, 2H), 3.97-3.68 (m, 2H), 3.19-2.81 (m, 4H), 2.45 (s, 3H), 1.28 (s, 6H). HRMS (ESI-TOF), m/z: found for [M+H⁺] (C₂₄H₂₅N₆OS⁺) 445.1763; calculated 445.1805.

195

¹H NMR (400 MHz, DMSO-d6) δ 11.73 (d, J=4.1 Hz, 1H), 8.62 (s, 1H), 8.35 (s, 1H), 8.08 (s, 1H), 7.28 (d, J=7.8 Hz, 1H), 7.19-7.07 (m, 1H), 6.95 (d, J=5.6 Hz, 1H), 4.93 (s, 2H), 4.01 (s, 3H), 3.75-3.74 (m, 2H), 2.98 (s, 4H), 1.28 (s, 6H). HRMS (ESI-TOF), m/z: found for [M+H⁺] (C₂₄H₂₅N₆O₂S⁺) 461.1704; calculated 461.1754.

196

¹H NMR (400 MHz, DMSO-d6) δ 10.51 (d, J=7.4 Hz, 2H), 8.62 (s, 1H), 8.35 (s, 1H), 8.06 (s, 1H), 6.82 (s, 3H), 4.93 (s, 2H), 3.70 (s, 2H), 2.97 (s, 2H), 2.91 (s, 2H), 1.28 (s, 6H).

197

¹H NMR (400 MHz, DMSO-d6) δ 10.68 (s, 1H), 8.63 (s, 1H), 8.35 (s, 1H), 8.11 (s, 1H), 7.94 (d, J=6.2 Hz, 3H), 7.86-7.11 (m, 5H), 4.93 (s, 2H), 4.03-3.55 (m, 2H), 3.08 (t, J=5.9 Hz, 2H), 2.98 (s, 2H), 1.28 (s, 6H). HRMS (ESI-TOF), m/z: found for [M+H⁺] (C₂₉H₂₇N₆O₄S⁺) 555.1803; calculated 555.1809.

198

¹H NMR (400 MHz, DMSO-d6) δ 8.59 (s, 1H), 8.35 (s, 1H), 8.11-8.09 (m, 1H), 7.38-7.35 (m, 2H), 7.22-7.20 (m, 1H), 4.93-4.89 (m, 3H), 4.36-4.32 (m, 1H), 3.79-3.78 (m, 2H), 3.01-2.98 (m, 4H), 2.12 (s, 3H), 1.28 (s, 6H).

199

¹H NMR (400 MHz, DMSO-d6) δ 10.13 (s, 1H), 8.61 (s, 1H), 8.35 (s, 1H), 8.08 (s, 1H), 7.84 (s, 1H), 7.63-7.41 (m, 2H), 4.93 (s, 2H), 3.79-3.78 (m, 2H), 3.05-2.97 (m, 4H), 2.68-2.54 (m, 1H), 1.27 (s, 6H), 1.07 (d, J=6.8 Hz, 6H). HRMS (ESI-TOF), m/z: found for [M+H⁺] (C₂₆H₂₉N₆O₄S⁺) 521.2003; calculated 521.1966.

200

¹H NMR (400 MHz, DMSO-d6) δ 11.22 (s, 1H), 8.63 (s, 1H), 8.36 (s, 1H), 8.12 (s, 1H), 7.38 (t, J=8.1 Hz, 1H), 7.28 (d, J=11.4 Hz, 1H), 7.15 (d, J=8.0 Hz, 1H), 4.93 (s, 2H), 3.78 (d, J=5.4 Hz, 2H), 3.02-2.98 (m, 4H), 1.28 (s, 6H). HRMS (ESI-TOF), m/z: found for [M+H⁺] (C₂₄H₂₂F₄N₅O₂S⁺) 520.1419; calculated 520.1425.

201

¹H NMR (400 MHz, DMSO-d6) δ 15.61-15.55 (m, 1H), 8.62 (s, 1H), 8.35 (s, 1H), 8.10 (s, 1H), 7.97-7.90 (m, 1H), 7.70-7.59 (m, 1H), 7.44-7.29 (m, 1H), 4.93 (s, 2H), 3.83-3.82 (m, 2H), 3.14 (s, 2H), 2.97 (s, 2H), 1.27 (s, 6H).

202

¹H NMR (400 MHz, DMSO-d6) δ 8.63 (s, 1H), 8.36 (s, 1H), 8.11 (s, 1H), 7.95 (s, 1H), 7.57-7.48 (m, 2H), 7.21 (d, J=8.3 Hz, 1H), 6.91 (s, 1H), 4.93 (s, 2H), 3.77 (s, 2H), 3.05 (t, J=7.8 Hz, 2H), 2.98 (s, 2H), 1.28 (s, 6H). HRMS (ESI-TOF), m/z: found for [M+H⁺] (C₂₄H₂₃N₄O₂S⁺) 431.1535; calculated 431.1536.

203

¹H NMR (400 MHz, DMSO-d6) δ 8.61 (s, 1H), 8.35 (s, 1H), 8.05 (t, J=5.4 Hz, 1H), 6.89 (dd, J=12.5, 1.5 Hz, 1H), 6.76 (dd, J=8.1, 1.5 Hz, 1H), 6.67 (t, J=8.5 Hz, 1H), 5.03-4.87 (m, 4H), 3.67 (q, J=6.9 Hz, 2H), 2.98 (s, 2H), 2.79 (t, J=7.5 Hz, 2H), 1.28 (s, 6H). HRMS (ESI-TOF), m/z: found for [M+H⁺](C₂₂H₂₃FN₅OS⁺) 424.1608; calculated 424.1602.

204

¹H NMR (400 MHz, DMSO-d6) δ 9.67 (s, 1H), 8.62 (s, 1H), 8.34 (s, 1H), 8.10 (t, J=5.1 Hz, 1H), 7.74 (t, J=8.2 Hz, 1H), 7.15 (d, J=11.9 Hz, 1H), 7.03 (d, J=8.2 Hz, 1H), 4.92 (s, 2H), 3.74 (q, J=6.6 Hz, 2H), 3.05-2.83 (m, 4H), 2.05 (s, 3H), 1.27 (s, 6H). HRMS (ESI-TOF), m/z: found for [M+H⁺](C₂₄H₂₅FN₅O₂S⁺) 466.1708; calculated 466.1708.

205

¹H NMR (400 MHz, CDCl₃) δ 8.61 (s, 1H), 8.35 (s, 1H), 8.04 (t, J=5.4 Hz, 1H), 7.83 (d, J=1.6 Hz, 1H), 7.39-7.28 (m, 3H), 6.95 (d, J=8.7 Hz, 1H), 4.93 (s, 2H), 3.71 (q, J=6.2 Hz, 2H), 2.97 (s, 2H), 2.85 (t, J=7.2 Hz, 2H), 1.28 (s, 6H). HRMS (ESI-TOF), m/z: found for [M+H⁺] (C₂₂H₂₃N₆O₃S⁺) 451.1548; calculated 451.1547.

206

¹H NMR (400 MHz, DMSO-d6) δ 12.40 (br s, 1H), 8.23 (s, 1H), 8.17 (s, 1H), 7.95 (t, J=5.4 Hz, 1H), 7.55-7.39 (m, 5H), 7.37-7.24 (m, 2H), 7.08 (d, J=8.0 Hz, 1H), 4.41 (s, 2H), 3.74 (q, J=6.8 Hz, 2H), 3.03 (t, J=7.9 Hz, 4H), 1.27 (s, 6H). HRMS (ESI-TOF), m/z: found for [M+H⁺] (C₂₉H₂₇N₆OS⁺) 507.1964; calculated 507.1962.

207

¹H NMR (400 MHz, DMSO-d6) δ 11.57 (s, 1H), 8.59 (t, J=5.4 Hz, 1H), 7.87 (s, 1H), 7.15-6.94 (m, 3H), 4.87 (s, 2H), 3.80 (s, 3H), 3.43 (q, J=6.9 Hz, 2H), 2.95 (s, 2H), 2.77 (t, J=7.3 Hz, 2H), 1.26 (s, 6H). HRMS (ESI-TOF), m/z: found for [M+H⁺] (C₂₄H₂₄F₄N₃O₄S⁺) 526.1428; calculated 526.1418.

208

¹H NMR (400 MHz, DMSO-d6) δ 8.13 (s, 1H), 7.73 (t, J=5.5 Hz, 1H), 7.20-6.90 (m, 5H), 4.83 (s, 2H), 3.79 (s, 3H), 3.40 (d, J=8.3 Hz, 2H), 2.89 (s, 2H), 2.76 (t, J=7.3 Hz, 2H), 1.25 (s, 6H). HRMS (ESI-TOF), m/z: found for [M+H⁺] (C₂₂H₂₅FN₃O₃S⁺) 430.1606; calculated 430.1595

209

¹H NMR (400 MHz, DMSO-d6) δ 12.45 (s, 1H), 8.60 (s, 1H), 8.35 (s, 1H), 8.02 (t, J=5.3 Hz, 1H), 7.19-6.96 (m, 3H), 3.79 (s, 3H), 3.71 (q, J=6.3 Hz, 2H), 3.27-2.74 (m, 7H), 2.22 (d, J=9.9 Hz, 1H), 2.08-1.80 (m, 1H). HRMS (ESI-TOF), m/z: found for [M+H⁺] (C₂₃H₂₂FN₄O₃S⁺) 453.1392; calculated 453.1391.

213

¹H NMR (400 MHz, DMSO-d6) δ 13.43 (s, 1H), 8.65 (s, 1H), 8.52 (s, 1H), 8.07-8.05 (m, 1H), 7.58 (d, J=8.3 Hz, 1H), 7.52 (s, 1H), 7.21 (d, J=8.3 Hz, 1H), 5.59 (s, 2H), 5.03 (s, 2H), 3.80 (q, J=6.7 Hz, 2H), 3.64-3.57 (m, 2H), 3.56-3.52 (m, 2H), 3.49-3.45 (m, 9H), 3.39-3.36 (m, 1H), 3.19 (s, 3H), 3.11-3.05 (m, 4H), 2.98 (s, 2H), 1.28 (s, 6H). LCMS (ESI), m/z: found for [M+H⁺] (C₃₃H₄₃N₆O₆S⁺) 651.18; calculated 651.30.

301

¹H NMR (400 MHz, DMSO-d6) δ 8.41 (s, 1H), 8.32 (s, 1H), 7.15 (dd, J=12.5, 1.8 Hz, 1H), 7.05 (t, J=8.7 Hz, 1H), 6.94 (d, J=8.3 Hz, 1H), 4.92 (s, 2H), 4.28 (t, J=7.1 Hz, 2H), 3.78 (s, 3H), 3.10-2.90 (m, 4H), 1.27 (s, 6H). HRMS (ESI-TOF), m/z: found for [M+H⁺] (C₂₃H₂₃FN₃O₃S⁺) 440.1440; calculated 440.1439.

302

¹H NMR (400 MHz, DMSO-d6) δ 8.60 (s, 1H), 8.35 (s, 1H), 8.03 (t, J=5.4 Hz, 1H), 7.18-6.95 (m, 3H), 4.12 (qd, J=7.0, 2.1 Hz, 2H), 3.79 (s, 3H), 3.71 (q, J=6.7 Hz, 2H), 3.27-2.79 (m, 7H), 2.23 (d, J=9.5 Hz, 1H), 2.03-1.84 (m, 1H), 1.21 (t, J=7.1 Hz, 3H). HRMS (ESI-TOF), m/z: found for [M+H⁺](C₂₅H₂₆FN₄O₃S⁺) 481.1680; calculated 481.1704.

303

¹H NMR (400 MHz, DMSO-d6) δ 8.61 (s, 1H), 8.35 (s, 1H), 8.06 (t, J=5.4 Hz, 1H), 7.20-6.95 (m, 3H), 4.91 (s, 2H), 4.05 (t, J=5.8 Hz, 2H), 3.79 (s, 3H), 3.72 (q, J=6.8 Hz, 2H), 3.09 (t, J=5.8 Hz, 2H), 2.89 (t, J=7.3 Hz, 2H). HRMS (ESI-TOF), m/z: found for [M+H⁺] (C₂₁H₂₀FN₄O₂S⁺) 411.1285; calculated 411.1286.

304

### Data for a Mixture of Isomers:

¹H NMR (400 MHz, DMSO-d6) δ 8.61 (d, J=4.8 Hz, 2H), 8.34 (s, 3H), 8.30 (s, 1H), 8.08 (dt, J=10.1, 4.9 Hz, 2H), 7.62 (d, J=8.3 Hz, 1H), 7.60 (s, 1H), 7.38 (d, J=8.3 Hz, 1H), 7.32 (s, 1H), 7.20 (t, J=6.8 Hz, 2H), 6.21-6.11 (m, 2H), 4.92 (s, 4H), 3.84-3.74 (m, 4H), 3.07 (t, J=7.1 Hz, 4H), 2.97 (s, 4H), 2.81-2.55 (m, 4H), 2.47 (s, 3H), 2.45 (s, 3H), 2.48-2.35 (m, 2H), 2.22-2.05 (m, 2H), 1.28 (s, 12H). LCMS (ESI), m/z: found for [M+H⁺] (C₂₈H₃₀N₇O₂S⁺) 528.12; calculated 528.22.

305

¹H NMR (400 MHz, DMSO-d6) δ 12.33 (s, 1H), 8.66 (s, 1H), 8.15 (s, 1H), 8.04 (t, J=5.5 Hz, 1H), 7.58-7.37 (m, 2H), 7.10 (dd, J=8.2, 1.7 Hz, 1H), 5.59 (s, 2H), 5.03 (s, 2H), 3.85-3.73 (m, 2H), 3.60 (dd, J=5.8, 3.2 Hz, 2H), 3.53 (dd, J=5.8, 3.2 Hz, 2H), 3.46 (d, J=5.0 Hz, 6H), 3.38 (dd, J=5.8, 3.6 Hz, 2H), 3.20 (s, 3H), 3.07 (d, J=7.6 Hz, 2H), 2.98 (s, 2H), 1.28 (s, 6H). LCMS (ESI), m/z: found for [M+H⁺](C₃₁H₄₀N₆O₅S⁺) 607.16; calculated 607.27.

¹H NMR (400 MHz, DMSO-d₆) δ 13.03 (s, 1H), 8.42 (s, 1H), 5.47 (t, J=5.9 Hz, 1H), 5.29 (d, J=5.7 Hz, 2H), 5.06 (s, 2H), 2.97 (s, 2H), 1.28 (s, 6H). LCMS (ESI), m/z: found for [M+H⁺] (C₁₅H₁₆N₃O₃S⁺) 317.96; calculated 318.09.

307

¹H NMR (400 MHz, DMSO-d6) δ 12.77 (s, 1H), 8.84 (d, J=4.6 Hz, 1H), 8.76 (d, J=7.9 Hz, 1H), 8.56 (d, J=7.2 Hz, 2H), 8.29 (s, 1H), 8.25 (t, J=5.5 Hz, 1H), 7.67 (dd, J=8.0, 4.6 Hz, 1H), 7.59-7.49 (m, 5H), 7.22 (d, J=8.2 Hz, 1H), 3.93 (q, J=6.5 Hz, 2H), 3.18 (t, J=7.6 Hz, 2H). LCMS (ESI), m/z: found for [M+H⁺] (C₂₄H₁₉N₆S⁺) 423.06; calculated 423.14.

308

¹H NMR (400 MHz, DMSO-d6) δ 12.32 (br s, 1H), 8.67 (s, 1H), 8.14 (s, 1H), 8.05 (t, J=5.5 Hz, 1H), 7.51 (s, 1H), 7.45 (s, 1H), 7.11 (d, J=8.2 Hz, 1H), 5.51 (s, 2H), 5.00 (s, 2H), 3.79 (q, J=6.7 Hz, 2H), 3.33 (s, 3H), 3.06 (t, J=7.5 Hz, 2H), 2.99 (s, 2H), 1.28 (s, 6H). LCMS (ESI), m/z: found for [M+H⁺](C₂₅H₂₇N₆O₂S⁺) 475.11; calculated 475.19.

309

¹H NMR (400 MHz, DMSO-d6) δ 12.33 (br s, 1H), 8.79 (dd, J=4.7, 1.7 Hz, 1H), 8.60 (dd, J=7.9, 1.7 Hz, 1H), 8.14 (s, 1H), 8.01 (t, J=5.5 Hz, 1H), 7.60 (dd, J=8.0, 4.6 Hz, 1H), 7.51 (d, J=8.2 Hz, 1H), 7.47 (s, 1H), 7.12 (dd, J=8.2, 1.6 Hz, 1H), 3.82-3.73 (m, 2H), 3.06 (t, J=7.5 Hz, 2H), 2.57 (s, 3H). LCMS (ESI), m/z: found for [M+H⁺] (C₁₉H₁₇N₆S⁺) 361.01; calculated 361.12.

310

¹H NMR (400 MHz, DMSO-d6) δ 12.33 (br s, 1H), 8.78 (d, J=4.6 Hz, 1H), 8.60 (d, J=8.0 Hz, 1H), 8.14 (s, 1H), 8.03 (s, 1H), 7.59 (s, 1H), 7.55-7.40 (m, 2H), 7.12 (d, J=8.3 Hz, 1H), 3.79-3.74 (m, 2H), 3.10-3.06 (m, 2H), 2.82-2.70 (m, 1H), 2.04-1.92 (m, 2H), 1.89-1.77 (m, 2H), 1.77-1.61 (m, 3H), 1.52-1.18 (m, 4H). LCMS (ESI), m/z: found for [M+H⁺] (C₂₄H₂₅N₆S⁺) 429.09; calculated 429.19.

311

¹H NMR (400 MHz, DMSO-d6) δ 8.42 (br s, 1H), 8.34 (s, 1H), 7.99 (t, J=5.5 Hz, 1H), 7.56 (d, J=8.2 Hz, 1H), 7.50 (s, 1H), 7.19 (d, J=8.2 Hz, 1H), 4.93 (s, 2H), 3.82-3.73 (m, 2H), 3.13-3.02 (m, 4H), 2.97 (s, 2H), 1.33 (d, J=6.8 Hz, 6H), 1.28 (s, 6H). ^{LCMS} (ESI), m/z: found for [M+H⁺] (C₂₆H₂₉N₆OS⁺) 473.12; calculated 473.21.

312

¹H NMR (400 MHz, DMSO-d6) δ 12.33 (br s, 1H), 8.79 (dd, J=4.6, 1.8 Hz, 1H), 8.60 (dd, J=8.0, 1.8 Hz, 1H), 8.15 (s, 1H), 8.05 (t, J=5.5 Hz, 1H), 7.59 (dd, J=8.0, 4.6 Hz, 1H), 7.51 (d, J=8.2 Hz, 1H), 7.46 (s, 1H), 7.12 (dd, J=8.2, 1.6 Hz, 1H), 3.83-3.74 (m, 2H), 3.19-3.02 (m, 3H), 1.35 (d, J=6.9 Hz, 6H). LCMS (ESI), m/z: found for [M+H⁺] (C₂₁H₂₁N₆S⁺) 389.03; calculated 389.15.

313

¹H NMR (400 MHz, DMSO-d6) δ 8.33 (s, 1H), 8.27 (s, 1H), 7.95 (t, J=5.4 Hz, 1H), 7.53 (d, J=8.2 Hz, 1H), 7.48 (d, J=1.6 Hz, 1H), 7.15 (dd, J=8.2, 1.6^{Hz}, 1H), 4.91 (s, 2H), 3.81-3.72 (m, 2H), 3.06 (t, J=7.5 Hz, 2H), 2.96 (s, 2H), 2.55 (s, 3H), 1.27 (s, 6H). LCMS (ESI), m/z: found for [M+H⁺] (C₂₄H₂₅N₆OS⁺) 445.08; calculated 445.18.

314

¹H NMR (400 MHz, DMSO-d6) δ 12.37 (br s, 1H), 8.34 (s, 1H), 8.16 (s, 1H), 7.95 (t, J=5.5 Hz, 1H), 7.51 (d, J=8.2 Hz, 1H), 7.45 (s, 1H), 7.11 (dd, J=8.3, 1.6 Hz, 1H), 4.92 (s, 2H), 3.76 (dt, J=8.4, 6.0 Hz, 2H), 3.06 (dd, J=8.6, 6.4 Hz, 2H), 2.96 (s, 2H), 2.78 (t, J=7.4 Hz, 2H), 1.84 (h, J=7.4 Hz, 2H), 1.27 (s, 6H), 0.96 (t, J=7.4 Hz, 3H). LCMS (ESI), m/z: found for [M+H⁺] (C₂₆H₂₉N₆OS⁺) 473.13; calculated 473.21.

315

¹H NMR (400 MHz, DMSO-d6) δ 8.54 (dd, J=7.9, 1.8 Hz, 2H), 8.48 (s, 1H), 8.22 (s, 1H), 8.17 (t, J=5.4 Hz, 1H), 7.54 (dq, J=10.4, 4.3, 3.3 Hz, 5H), 7.19 (^{dd}, J=8.3, 1.6 Hz, 1H), 4.97 (s, 2H), 3.90 (dt, J=8.5, 5.9 Hz, 2H), 3.16 (t, J=7.5 Hz, 2H), 2.99 (s, 2H), 1.29 (s, 6H). LCMS (ESI), m/z: found for [M+H⁺](C₂₉H₂₇N₆OS⁺) 507.12; calculated 507.20.

316

¹H NMR (400 MHz, DMSO-d6) δ 8.78 (dd, J=4.6, 1.7 Hz, 1H), 8.59 (dd, J=8.0, 1.7 Hz, 1H), 8.16 (s, 1H), 8.04 (t, J=5.5 Hz, 1H), 7.59 (dd, J=8.0, 4.6 Hz, 1H), 7.51 (d, J=8.2 Hz, 1H), 7.45 (s, 1H), 7.11 (dd, J=8.3, 1.6 Hz, 1H), 3.81-3.72 (m, 2H), 3.31-3.21 (m, 1H), 3.07 (dd, J=8.7, 6.4 Hz, 2H), 2.06-1.91 (m, 4H), 1.89-1.77 (m, J=4.8, 4.4 Hz, 2H), 1.76-1.64 (m, 2H). LCMS (ESI), m/z: found for [M+H⁺](C₂₃H₂₃N₆S⁺) 415.08; calculated 415.17.

317

¹H NMR (400 MHz, DMSO-d6) δ 12.32 (d, J=10.8 Hz, 1H), 8.65 (t, J=5.7 Hz, 1H), 8.58 (s, 1H), 8.14 (s, 1H), 8.05 (t, J=5.5 Hz, 1H), 7.59-7.34 (m, 2H), 7.16-7.03 (m, 1H), 4.80 (s, 2H), 3.78 (q, J=6.8 Hz, 2H), 3.64 (t, J=5.9 Hz, 2H), 3.59-3.46 (m, 12H), 3.46-3.38 (m, 2H), 3.22 (s, 3H), 3.06 (t, J=7.4 Hz, 2H), 3.00 (s, 2H), 1.29 (s, 6H). LCMS (ESI), m/z: found for [M+H⁺] (C₃₃H₄₂N₇O₆S⁺) 664.18; calculated 664.29.

318

¹H NMR (400 MHz, DMSO-d6) δ 9.34 (s, 1H), 8.33 (s, 1H), 8.18 (s, 1H), 8.03 (t, J=5.5 Hz, 1H), 7.98 (s, 1H), 7.50 (d, J=8.2 Hz, 1H), 7.43 (s, 1H), 7.09 (dd, J=8.3, 1.6 Hz, 1H), 4.57 (s, 2H), 3.75 (q, J=6.8 Hz, 2H), 3.07-3.01 (m, 4H), 1.29 (s, 6H). LCMS (ESI), m/z: found for [M+H⁺] (C₂₆H₂₄N₇OS₂⁺) 514.07; calculated 514.65.

319

¹H NMR (400 MHz, DMSO-d6) δ 12.35 (br s, 1H), 8.66 (s, 1H), 8.15 (s, 1H), 8.05 (t, J=5.5 Hz, 1H), 7.51 (d, J=8.2 Hz, 1H), 7.45 (s, 1H), 7.11 (dd, J=8.2, 1.6 Hz, 1H), 5.03 (s, 2H), 4.75 (s, 2H), 3.83-3.74 (m, 2H), 3.06 (t, J=7.4 Hz, 2H), 2.97 (s, 2H), 1.95 (s, 3H), 1.27 (s, 6H). LCMS (ESI), m/z: found for [M+H⁺](C₂₅H₂₇N₆OS₂⁺) 491.12; calculated 491.17.

320

¹H NMR (400 MHz, DMSO-d6) δ 9.85 (br s, 1H), 8.70 (s, 1H), 8.46 (t, J=5.5 Hz, 1H), 8.17 (s, 1H), 7.51 (d, J=8.2 Hz, 1H), 7.46 (s, 1H), 7.11 (dd, J=8.3, 1.6 Hz, 1H), 5.12 (s, 2H), 4.58 (s, 2H), 3.87-3.78 (m, 2H), 3.76 (t, J=4.9 Hz, 2H), 3.59 (dd, J=5.8, 3.1 Hz, 2H), 3.52 (dd, J=5.8, 3.1 Hz, 2H), 3.48 (dd, J=5.9, 3.6 Hz, 2H), 3.42-3.37 (m, 5H), 3.16 (s, 3H), 3.07 (t, J=7.4 Hz, 2H), 3.01 (s, 2H), 1.29 (s, 6H). LCMS (ESI), m/z: found for [M+H⁺] (C₃₁H₄₀N₇O₄S⁺) 606.21; calculated 606.29.

321

¹H NMR (400 MHz, DMSO-d6) δ 12.35 (br s, 1H), 8.70 (s, 1H), 8.34 (br s, 1H), 8.15 (s, 1H), 7.61-7.39 (m, 2H), 7.10 (d, J=8.2 Hz, 1H), 5.09 (s, 2H), 4.55 (s, 2H), 3.87-3.77 (m, 2H), 3.66 (s, 2H), 3.58-3.41 (m, 12H), 3.37-3.34 (m, 2H), 3.19-3.15 (m, 4H), 3.07 (t, J=7.4 Hz, 2H), 3.01 (s, 2H), 1.29 (s, 6H). LCMS (ESI), m/z: found for [M+H⁺] (C₃₃H₄₄N₇O₅S⁺) 650.25; calculated 650.31.

322

¹H NMR (400 MHz, DMSO-d6) δ 8.64 (s, 1H), 8.16 (s, 1H), 8.11 (t, J=5.5 Hz, 1H), 7.51 (d, J=8.2 Hz, 1H), 7.46 (s, 1H), 7.32-7.23 (m, 2H), 7.11 (dd, J=8.2, 1.6 Hz, 1H), 7.07 (d, J=8.2 Hz, 2H), 6.94 (t, J=7.3 Hz, 1H), 6.23 (s, 2H), 5.02 (s, 2H), 3.⁸⁴-3.75 (m, 2H), 3.11-3.03 (m, 2H), 2.99 (s, 2H), 1.26 (s, 6H). LCMS (ESI), m/z: found for [M+H⁺] (C₃₀H₂₉N₆O₂S⁺) 537.18; calculated 537.21.

323

¹H NMR (400 MHz, DMSO-d6) δ 12.33 (br s, 1H), 8.65 (s, 1H), 8.15 (s, 1H), 8.04 (t, J=5.5 Hz, 1H), 7.51 (d, J=8.2 Hz, 1H), 7.45 (s, 1H), 7.¹⁰ (dd, J=8.2, 1.6 Hz, 1H), 5.57 (s, 2H), 5.02 (s, 2H), 3.83-3.74 (m, 2H), 3.24 (d, J=6.4 Hz, 2H), 3.06 (t, J=7.4 Hz, 2H), 2.99 (s, 2H), 1.80 (dt, J=13.2, 6.6 Hz, 1H), 1.28 (s, 6H), 0.82 (d, J=6.7 Hz, 6H). LCMS (ESI), m/z: found for [M+H⁺] (C₂₈H₃₃N₆O₂S⁺) 517.22; calculated 517.24.

324

¹H NMR (400 MHz, DMSO-d6) δ 8.64 (s, 1H), 8.17 (s, 1H), 8.05 (t, J=5.6 Hz, 1H), 7.51 (d, J=8.2 Hz, 1H), 7.45 (s, 1H), 7.11 (d, J=8.2 Hz, 1H), 5.61 (s, 2H), 5.04 (s, 2H), 3.80-3.72 (m, 4H), 3.67-3.58 (m, 2H), 3.06 (t, J=7.5 Hz, 2H), 2.98 (s, 2H), 2.04-1.95 (m, 1H), 1.93-1.86 (m, 2H), 1.48-1.41 (m, 2H), 1.28 (s, 6H). LCMS (ESI), m/z: found for [M+H⁺] (C₂₉H₃₃N₆O₃S⁺) 545.23; calculated 545.23.

325

¹H NMR (400 MHz, DMSO-d6) δ 8.56 (s, 1H), 8.28 (s, 1H), 8.11 (t, J=5.5 Hz, 1H), 7.53 (d, J=8.2 Hz, 1H), 7.47 (s, 1H), 7.14 (dd, J=8.3, 1.6 Hz, 1H), 4.72 (AB q, J_{AB}=16.0 Hz, 2H), 3.79 (q, J=8.0 Hz, 2H), 3.09 (s, 3H), 3.06 (q, J=4.3 Hz, 2H), 3.02 (s, 2H), 2.65 (s, 3H), 1.28 (d, J=9.8 Hz, 6H). LCMS (ESI), m/z: found for [M+H⁺] (C₂₆H₂₈N₇O₂S⁺) 502.19; calculated 502.20.

326

¹H NMR (400 MHz, DMSO-d6) δ¹²0.31 (br s, 1H), 8.66 (s, 1H), 8.14 (s, 1H), 8.05 (t, J=5.5 Hz, 1H), 7.62-7.35 (m, 2H), 7.10 (d, J=8.2 Hz, 1H), 5.57 (s, 2H), 5.03 (s, 2H), 3.84-3.74 (m, 2H), 3.67 (p, J=6.1 Hz, 1H), 3.06 (t, J=7.5 Hz, 2H), 2.99 (s, 2H), 1.28 (s, 6H), 1.13 (d, J=6.1 Hz, 6H). LCMS (ESI), m/z: found for [M+H⁺] (C₂₇H₃₁N₆O₂S⁺) 503.23; calculated 503.22.

327

¹H NMR (400 MHz, DMSO-d6) δ 12.35 (br s, 1H), 8.65 (s, 1H), 8.14 (s, 1H), 8.05 (t, J=5.6 Hz, 1H), 7.51 (d, J=8.2 Hz, 1H), 7.45 (s, 1H), 7.10 (dd, J=8.2, 1.6 Hz, 1H), 5.59 (s, 2H), 5.04 (s, 2H), 3.84-3.74 (m, 2H), 3.47-3.36 (m, 1H), 3.06 (t, J=7.5 Hz, 2H), 2.98 (s, 2H), 1.90-1.81 (m, 2H), 1.65-1.60 (m, 2H), 1.48-1.38 (m, 1H), 1.28 (s, 6H), 1.35-1.15 (m, 5H). LCMS (ESI), m/z: found for [M+H⁺] (C₃₀H₃₅N₆O₂S⁺) 543.24; calculated 543.25.

328

¹H NMR (400 MHz, DMSO-d6) δ 8.65 (s, 1H), 8.14 (s, 1H), 8.07 (br s, 1H), 7.51 (d, J=8.2 Hz, 1H), 7.46 (s, 1H), 7.09 (d, J=8.2 Hz, 1H), 5.59 (s, 2H), 5.04 (s, 2H), 3.79 (t, J=7.6 Hz, 2H), 3.47-3.39 (m, 2H), 3.06 (t, J=7.6 Hz, 2H), 2.99 (s, 2H), 2.09 (s, 3H), 2.02-1.95 (m, 2H), 1.90-1.78 (m, 2H), 1.56-1.43 (m, 2H), 1.28 (s, 7H). LCMS (ESI), m/z: found for [M+H⁺] (C₃₀H₃₆N₇O₂S⁺) 558.25; calculated 558.26.

329

¹H NMR (400 MHz, DMSO-d6) δ 12.49 (br s, 1H), 8.50 (s, 1H), 8.29 (br s, 1H), 8.27 (s, 1H), 8.20 (s, 1H), 7.51 (d, J=8.2 Hz, 1H), 7.47 (s, 1H), 7.13 (d, J=8.2 Hz, 1H), 4.90 (s, 2H), 3.79 (q, J=7.0 Hz, 2H), 3.06 (t, J=7.4 Hz, 2H), 2.93 (s, 2H), 1.27 (s, 6H). LCMS (ESI), m/z: found for [M+H⁺] (C₂₃H₂₃N₆O₂⁺) 415.18; calculated 415.19.

330

¹H NMR (400 MHz, DMSO-d6) δ 12.36 (br s, 1H), 8.67 (s, 1H), 8.15 (s, 1H), 8.01 (t, J=5.6 Hz, 1H), 7.52 (d, J=8.2 Hz, 1H), 7.46 (s, 1H), 7.11 (d, J=8.2 Hz, 1H), 5.12 (s, 2H), 4.62 (s, 2H), 3.79 (q, J=6.7 Hz, 2H), 3.54-3.43 (m, 12H), 3.41-3.34 (m, 2H), 3.20 (s, 3H), 3.06 (t, J=7.5 Hz, 2H), 2.98 (s, 2H), 2.59 (t, J=5.9 Hz, 2H), 2.19 (s, 3H), 1.28 (s, 6H). LCMS (ESI), m/z: found for [M+H⁺] (C₃₄H₄₆N₇O₅S⁺) 664.30; calculated 664.33.

331

¹H NMR (400 MHz, DMSO-d6) δ¹²0.33 (br s, 1H), 8.66 (s, 1H), 8.14 (s, 1H), 8.00 (t, J=5.6 Hz, 1H), 7.51 (d, J=8.2 Hz, 1H), 7.45 (s, 1H), 7.10 (d, J=8.2 Hz, 1H), 5.11 (s, 2H), 4.61 (s, 2H), 3.78 (q, J=6.8 Hz, 2H), 3.51-3.41 (m, 8H), 3.40-3.36 (m, 2H), 3.19 (s, 3H), 3.06 (t, J=7.4 Hz, 2H), 2.97 (s, 2H), 2.58 (t, J=5.9 Hz, 2H), 2.19 (s, 3H), 1.27 (s, 6H). LCMS (ESI), m/z: found for [M+H⁺] (C₃₂H₄₂N₇O₄S⁺) 620.28; calculated 620.30.

332

¹H NMR (400 MHz, DMSO-d6) δ 12.32 (s, 1H), 8.66 (s, 1H), 8.14 (s, 1H), 8.05 (t, J=5.5 Hz, 1H), 7.59-7.36 (m, 2H), 7.10 (d, J=8.2 Hz, 1H), 5.59 (s, 2H), 5.03 (s, 2H), 3.79 (q, J=6.8 Hz, 2H), 3.62-3.56 (m, 2H), 3.47-3.41 (m, 2H), 3.20 (s, 3H), 3.06 (t, J=7.3 Hz, 2H), 2.98 (s, 2H), 1.28 (s, 6H). LCMS (ESI), m/z: found for [M+H⁺] (C₂₇H₃₁N₆O₂S⁺) 519.23; calculated 519.22.

333

¹H NMR (400 MHz, DMSO-d6) δ 12.31 (d, J=14.6 Hz, 1H), 8.66 (s, 1H), 8.14 (br s, 1H), 8.04 (t, J=5.5 Hz, 1H), 7.59-7.51 (m, 1H), 7.46-7.35 (m, 1H), 7.10 (t, J=9.9 Hz, 1H), 5.53 (s, 2H), 5.01 (s, 2H), 4.08-3.98 (m, 1H), 3.79 (q, J=6.8 Hz, 2H), 3.06 (t, J=7.8 Hz, 2H), 2.98 (s, 2H), 1.66 (m, 4H), 1.64-1.51 (m, 2H), 1.47 (m, 2H), 1.28 (s, 6H). LCMS (ESI), m/z: found for [M+H⁺] (C₂₉H₃₃N₆O₂S⁺) 529.25; calculated 529.24.

334

¹H NMR (400 MHz, DMSO-d6) δ 12.34-12.30 (m, 1H), 8.66 (s, 1H), 8.14 (d, J=4.7 Hz, 1H), 7.99 (t, J=5.5 Hz, 1H), 7.74-7.51 (m, 1H), 7.49-7.30 (m, 1H), 7.11 (dd, J=13.7, 8.2 Hz, 1H), 5.07 (s, 2H), 4.47 (s, 2H), 3.78 (q, J=6.5 Hz, 2H), 3.06 (t, J=7.5 Hz, 2H), 2.97 (s, 2H), 2.19 (s, 6H), 1.27 (s, 6H). LCMS (ESI), m/z: found for [M+H⁺] (C₂₆H₃₀N₇OS⁺) 488.22; calculated 488.22.

335

¹H NMR (400 MHz, DMSO-d6) δ 12.36 (s, 1H), 8.65 (s, 1H), 8.14 (s, 1H), 7.98 (t, J=5.5 Hz, 1H), 7.50-7.49 (m, 2H), 7.10 (d, J=8.2 Hz, 1H), 4.93 (s, 2H), 3.78 (q, J=6.8 Hz, 2H), 3.42-3.31 (m, 4H), 3.20 (s, 3H), 3.06 (t, J=7.5 Hz, 2H), 2.95 (s, 2H), 1.63 (dt, J=15.1, 8.3 Hz, 4H), 1.27 (s, 6H). LCMS (ESI), m/z: found for [M+H⁺] (C₂₈H₃₃N₆O₂S⁺) 517.24; calculated 517.24.

### Synthetic Details for Compound 106:

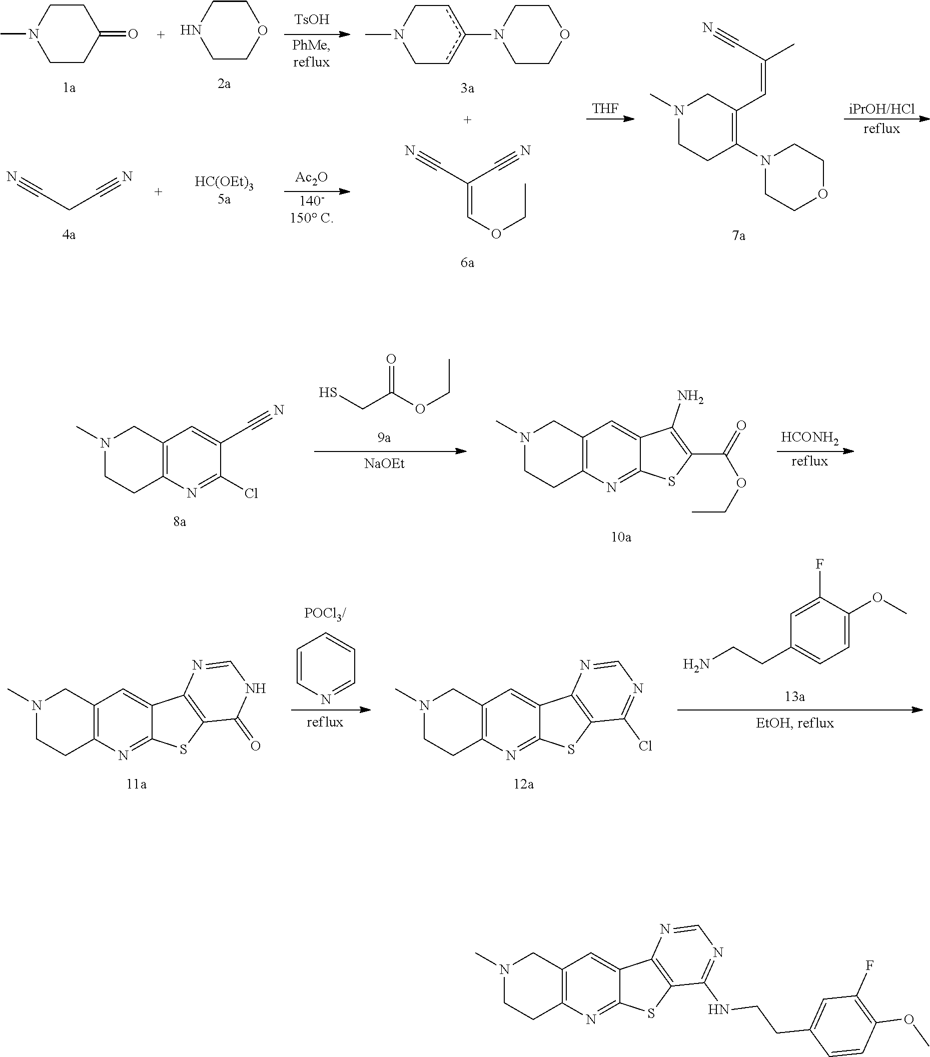

### 4-(1-Methylpiperidin-4-yl)morpholine (3a)

Into a 250-mL flask, equipped with a Dean-Stark trap and a reflux condenser, were placed 1-methylpiperidin-4-one (1a) (20.37 g, 180 mmol, 1 equiv), morpholine (2a) (24.8 ml, 288 mmol, 1.6 equiv), p-toluenesulfonic acid (150 mg) and 180 mL of toluene. The reaction mixture was refluxed for 16 h upon complete liberation of water. After toluene was evaporated, the residue was distilled in vacuo at 20 Torr to afford the target product (3a) as a yellowish liquid (22.88 g, 70%, b.p. 125-130° C./20 Torr).

### 2-(Ethoxymethylene)malononitrile (6a)

Into a 250-mL flask was placed malononitrile (4a) (25 g, 378 mmol, 1 equiv), triethyl orthoformate (5a) (94.3 ml, 567 mmol, 1.5 equiv) and acetic anhydride (71.5 ml, 756 mmol, 2 equiv). The resultant mixture was heated at 140° C. for 1 h, then at 150° C. for 1 h more. After Ac₂O was evaporated, the residue was distilled in vacuo at 10 Torr to afford the target product (6a) as a yellow liquid (28.4 g, 62%, b.p. 140-150° C./10 Torr) which solidifies upon staying.

### 2-((1-Methyl-4-morpholino-1,2,5,6-tetrahydropyridin-3-yl)methylene)malononitrile (7a)

A homogeneous mixture of 4-(1-methylpiperidin-4-yl)morpholine (3a) (22.87 g, 125 mmol, 1 equiv) and 2-(ethoxymethylene)malononitrile (6a) (15.27 g, 125 mmol, 1 equiv) in 160 mL of dry THF was left at room temperature under Ar overnight. After THF was evaporated, the residue was crystallized from 100 mL of cold MeOH to afford the target product (7a) as an orange solid (24.12 g, 75%).

### 2-Chloro-6-methyl-5,6,7,8-tetrahydro-1,6-naphthyridine-3-carbonitrile (8a)

A suspension of 2-((1-methyl-4-morpholino-1,2,5,6-tetrahydropyridin-3-yl)methylene)malononitrile (7a) (24.02 g, 93 mmol) in 300 mL i-PrOH was cooled to 0° C., followed by saturation with gaseous HCl during 1 h and subsequent reflux for 3 h. The solvent was removed in vacuo, the residue was treated with 250 mL of sat. aq. solution of NH₃ and cooled to +4° C. The precipitated crystals were filtered off, washed with water and dried. The resultant product was recrystallized from 120 mL of MeOH to afford the target compound (8a) as a yellow solid (10.82 g, 56%).

### Ethyl 3-amino-6-methyl-5,6,7,8-tetrahydrothieno[2,3-b][1,6]naphthyridine-2-carboxylate (10a)

Sodium metal (126 mg, 5.5 mmol, 1.1 equiv) was dissolved in 26 mL of absolute EtOH. To this mixture were added 2-chloro-6-methyl-5,6,7,8-tetrahydro-1,6-naphthyridine-3-carbonitrile (8a) (1.04 g, 5 mmol, 1 equiv) and 613 μL of ethyl 2-mercaptoacetate (9a). The reaction mixture was refluxed for 5 h. After the solvent was evaporated, the residue was mixed with 70 mL of cold water, the precipitated solid was filtered off and recrystallized from 30 mL of EtOH to afford the target compound (10a) as a yellowish solid (1.16 g, 79%).

### 9-Methyl-7,8,9,10-tetrahydropyrimido[4′,5′:4,5]thieno[2,3-b][1,6]naphthyridin-4(3H)-one (11a)

Into a 50-mL flask were placed ethyl 3-amino-6-methyl-5,6,7,8-tetrahydrothieno[2,3-b][1,6]naphthyridine-2-carboxylate (10a) (1.16 g, 4 mmol) and 20 mL of formamide. The mixture was refluxed under Ar for 4 h followed by concentration in vacuo (heating up to 95° C./8 Torr). MTBE (30 mL) was added to the residue and the reaction mixture was put into a fridge (−18° C.). The precipitate formed was filtered off, washed with cold MTBE and dried in vacuo to afford the target compound (11a) as a grey solid (991 mg, 91%). Compound (11a) is soluble in H₂O.

### 4-Chloro-9-methyl-7,8,9,10-tetrahydropyrimido[4′,5′:4,5]thieno[2,3-b][1,6]-naphthyridine (12a)

Into a 50-mL flask were placed 9-methyl-7,8,9,10-tetrahydropyrimido[4′,5′:4,5]thieno[2,3-b][1,6]naphthyridin-4(3H)-one (11a) (980 mg, 3.6 mmol), 15 mL of POCl₃ and 0.5 mL of pyridine. The mixture was refluxed for 5 h. The solvent was removed in vacuo, the residue was mixed with 100 mL of sat. aq. NaHCO₃ solution and cooled to +4° C. The precipitate formed was filtered off and dried in vacuo to afford the product (12a) as a brown solid (288 mg, 27%).

### N-(3-Fluoro-4-methoxyphenethyl)-9-methyl-7,8,9,10-tetrahydropyrimido[4′,5′:4,5]-thieno[2,3-b][1,6]naphthyridin-4-amine (106)

In a 10-mL flask were mixed 4-chloro-9-methyl-7,8,9,10-tetrahydropyrimido[4′,5′:4,5]-thieno[2,3-b][1,6]naphthyridine (12a) (73 mg, 0.25 mmol, 1 equiv) and 2-(3-fluoro-4-methoxyphenyl)ethanamine (13a) (64 mg, 0.375 mmol, 1.5 equiv) in 5 mL of EtOH. The reaction mixture was refluxed for 8 h. The solvent was removed in vacuo, the residue was subjected to CC (eluent: DCM/MeOH=9/1). Fractions with the product were collected and concentrated, the residue was recrystallized from MeOH to afford the target product as a sandy-brown solid (21 mg, 20%).

¹H NMR (400 MHz, DMSO-d₆) δ 8.56 (s, 1H), 8.35 (s, 1H), 8.01 (m, 1H), 7.17-6.99 (m, 3H), 3.79 (s, 3H), 3.71 (m, 4H), 3.10 (m, 2H), 2.89 (m, 2H), 2.78 (m, 2H), 2.50 (s, 3H)

### Synthetic Details for Compounds 109:

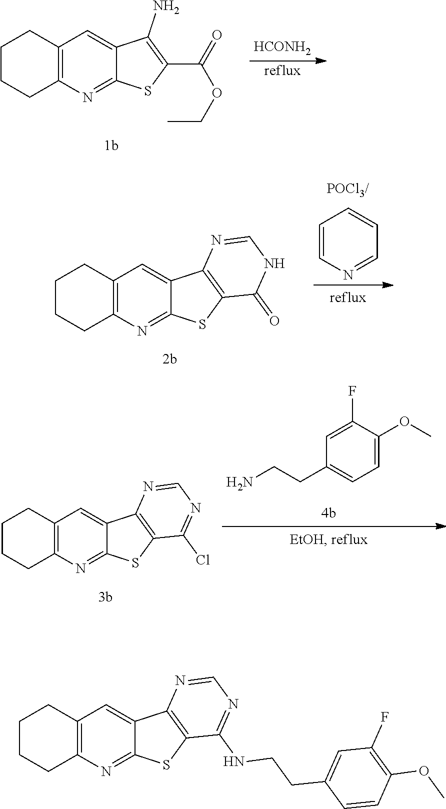

### 7,8,9,10-Tetrahydropyrimido[4′,5′:4,5]thieno[2,3-b]quinolin-4(3H)-one (2b)

Into a 10-mL flask were placed ethyl 3-amino-5,6,7,8-tetrahydrothieno[2,3-b]quinoline-2-carboxylate (1b) (276 mg, 1 mmol) and 5 mL of formamide. The mixture was refluxed under Ar for 4 h. The reaction mixture was allowed to cool to rt followed by addition of 50 mL of H₂O. The resultant mixture was put into a fridge (+4° C.) and the precipitate formed was filtered off and dried in vacuo to afford the target compound (2b) as a yellow solid (182 mg, 71%).

### 4-Chloro-7,8,9,10-tetrahydropyrimido[4′,5′:4,5]thieno[2,3-b]quinoline (3b)

Into a 10-mL flask were placed 7,8,9,10-tetrahydropyrimido[4′,5′:4,5]thieno[2,3-b]quinolin-4(3H)-one (2b) (182 mg, 0.71 mmol), 5 mL of POCl₃ and 0.25 mL of pyridine. The mixture was refluxed for 24 h. The solvent was removed in vacuo, the residue was mixed with 75 mL of sat. aq. NaHCO₃ solution and cooled to +4° C. The precipitate formed was filtered off, washed with water and dried in vacuo to afford the product (3b) as a purple solid (169 mg, 86%).

### N-(3-Fluoro-4-methoxyphenethyl)-7,8,9,10-tetrahydropyrimido[4′,5′:4,5]-thieno[2,3-b]quinolin-4-amine

In a 10-mL flask were mixed 4-chloro-7,8,9,10-tetrahydropyrimido[4′,5′:4,5]thieno[2,3-b]quinoline (3b) (69 mg, 0.25 mmol, 1 equiv) and 2-(3-fluoro-4-methoxyphenyl)ethanamine (4b) (64 mg, 0.375 mmol, 1.5 equiv) in 5 mL of EtOH. The reaction mixture was refluxed for 6 h. The solvent was removed in vacuo, the residue was subjected to CC (eluent: PE/EA=9/1). Fractions with the product were collected and concentrated, the residue was recrystallized twice from EtOH to afford the target product as a beige solid (16 mg, 16%).

¹H NMR (400 MHz, DMSO-d₆) δ 8.56 (s, 1H), 8.35 (s, 1H), 8.01 (m, 1H), 7.17-6.99 (m, 3H), 3.79 (s, 3H), 3.71 (m, 4H), 3.10-2.89 (m, 6H), 1.93-1.78 (m, 4H)

### Synthetic Details for Compound 108:

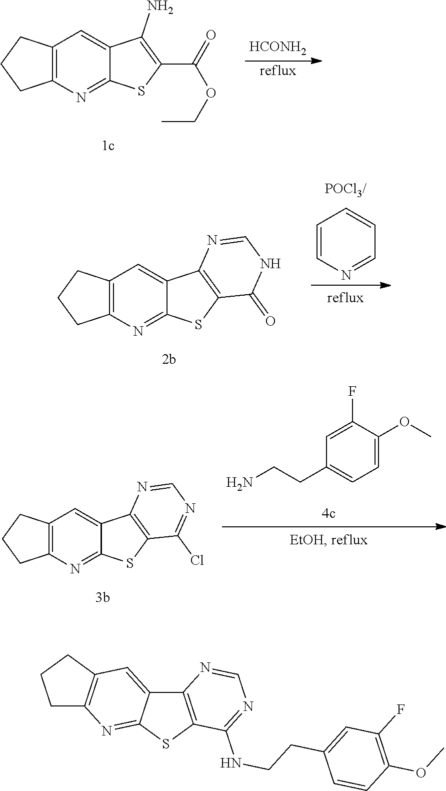

### 8,9-Dihydro-3H-cyclopenta[5′,6′]pyrido[3′,2′:4,5]thieno[3,2-d]pyrimidin-4(7H)-one (2c)

Into a 10-mL flask were placed ethyl 3-amino-6,7-dihydro-5H-cyclopenta[b]thieno[3,2-e]pyridine-2-carboxylate (1c) (262 mg, 1 mmol) and 5 mL of formamide. The mixture was refluxed under Ar for 4 h. The reaction mixture was allowed to cool to rt followed by addition of 50 mL of H₂O. The resultant mixture was put into a fridge (+4° C.) and the precipitate formed was filtered off and dried in vacuo to afford the target compound (2c) as a white solid (187 mg, 77%).

### 4-Chloro-8,9-dihydro-7H-cyclopenta[5′,6′]pyrido[3′,2′:4,5]thieno[3,2-d]pyrimidine (3c)

Into a 10-mL flask were placed 8,9-dihydro-3H-cyclopenta[5′,6′]pyrido[3′,2′:4,5]thieno[3,2-d]pyrimidin-4(7H)-one (2c) (187 mg, 0.77 mmol), 5 mL of POCl₃ and 0.25 mL of pyridine. The mixture was refluxed for 8 h. The solvent was removed in vacuo, the residue was mixed with 75 mL of sat. aq. NaHCO₃ solution and cooled to +4° C. The precipitate formed was filtered off, washed with water and dried in vacuo to afford the product (3c) as a purple solid (176 mg, 87%).

### N-(3-Fluoro-4-methoxyphenethyl)-8,9-dihydro-7H-cyclopenta[5′,6′]pyrido-[3′,2′:4,5]thieno[3,2-d]pyrimidin-4-amine

In a 10-mL flask were mixed 4-chloro-8,9-dihydro-7H-cyclopenta[5′,6′]pyrido-[3′,2′:4,5]thieno[3,2-d]pyrimidine (3c) (65 mg, 0.25 mmol, 1 equiv) and 2-(3-fluoro-4-methoxyphenyl)ethanamine (4c) (64 mg, 0.375 mmol, 1.5 equiv) in 5 mL of EtOH. The reaction mixture was refluxed for 6 h. The solvent was removed in vacuo, the residue was subjected to CC (eluent: PE/EA=9/1). Fractions with the product were collected and concentrated, the residue was recrystallized from MeOH to afford the target product as a pink solid (39 mg, 40%).

¹H NMR (400 MHz, DMSO-d₆) δ 8.56 (s, 1H), 8.35 (s, 1H), 8.01 (m, 1H), 7.17-6.99 (m, 3H), 3.79 (s, 3H), 3.71 (m, 4H), 3.09 (m, 4H), 2.89 (m, 2H), 2.19 (m, 2H)

### Synthetic Details for Compound 107:

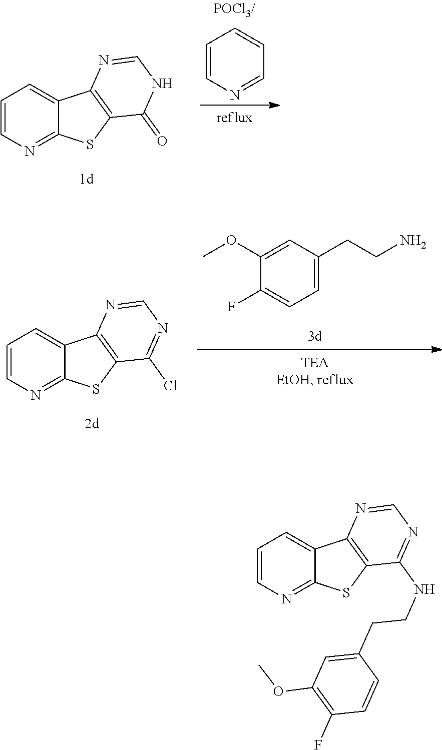 [SMILES: O=c1[nH]cnc2c1sc1ncccc12 ; c1ccncc1 ; Clc1ncnc2c1sc1ncccc12 ; COc1cc(CCN)ccc1F ; COc1cc(CCNc2ncnc3c2sc2ncccc23)ccc1F]

### 4-Chloropyrido[3′,2′:4,5]thieno[3,2-d]pyrimidine

Into a 10-mL flask were placed pyrido[3′,2′:4,5]thieno[3,2-d]pyrimidin-4(3H)-one (1d) (122 mg, 0.6 mmol), 5 mL of POCl₃ and 170 μL of pyridine. The mixture was refluxed for 5 h. The solvent was removed in vacuo, the residue was mixed with 150 mL of sat. aq. NaHCO₃ solution and the precipitate formed was filtered off and dried in vacuo to afford the product (2d) as a white solid (108 mg, 81%).

### N-(3-Fluoro-4-methoxyphenethyl)pyrido[3′,2′:4,5]thieno[3,2-d]pyrimidin-4-amine

In a 10-mL flask were mixed 4-chloropyrido[3′,2′:4,5]thieno[3,2-d]pyrimidine (2d) (41 mg, 0.18 mmol, 1 equiv), 2-(4-fluoro-3-methoxyphenyl)ethanamine (3d) (37 mg, 0.22 mmol, 1.2 equiv) and TEA (80 μL, 0.6 mmol, 4 equiv) in 5 mL of EtOH. The reaction mixture was refluxed for 8 h. The solvent was removed in vacuo, the residue was subjected to CC (eluent: PE/EA=9/1). Fractions with the product were collected, concentrated and triturated with pentane to afford the target product as a white solid (31 mg, 48%).

¹H NMR (400 MHz, DMSO-d₆) δ 8.83 (s, 1H), 8.64 (s, 2H), 8.12 (m, 1H), 7.65 (m, 1H), 7.12 (m, 2H), 6.90 (m, 1H), 3.79 (m, 5H), 2.89 (m, 2H)

### Synthetic Details for Compound 321:

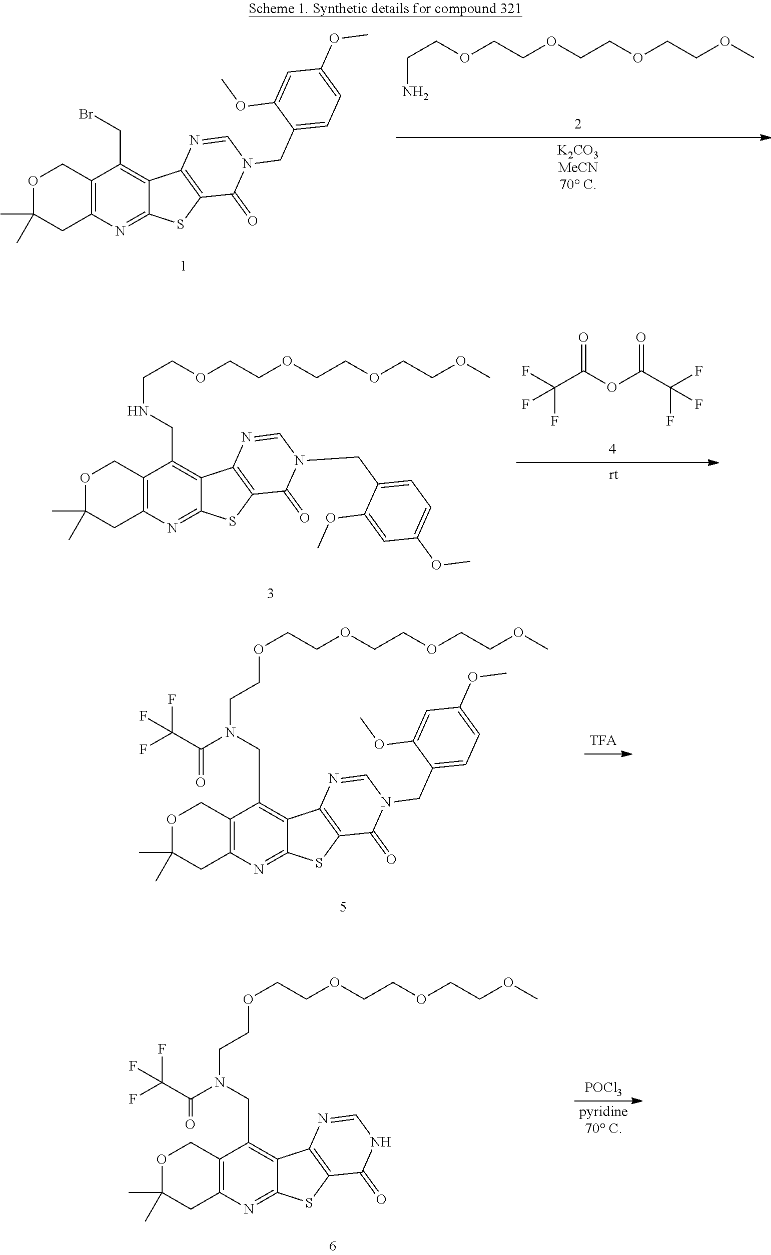

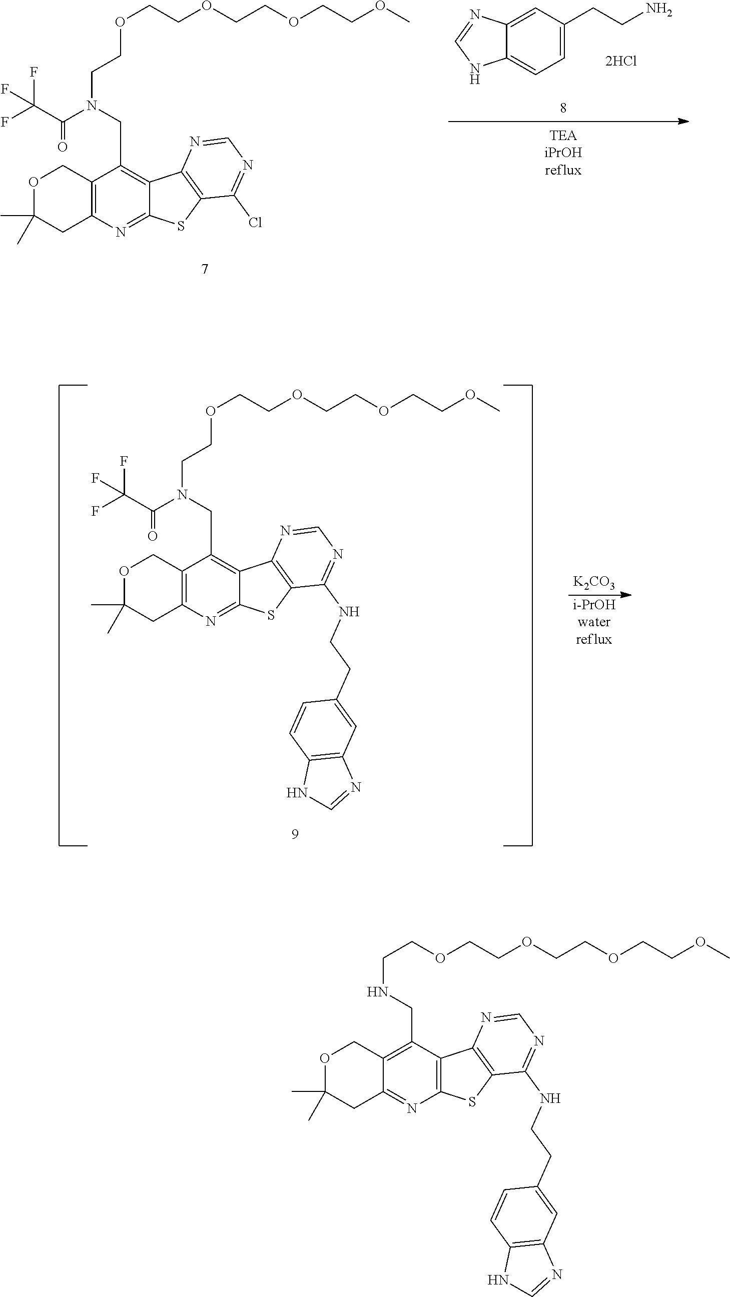

### 3-(2,4-Dimethoxybenzyl)-8,8-dimethyl-11-(5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (3) (Scheme 1)

A mixture of 2,5,8,11-tetraoxatridecan-13-amine (2) (440 mg, 2.12 mmol, 3.2 equiv) and K₂CO₃ (91 mg, 0.66 mmol, 1 equiv) in 10 mL of MeCN was heated to 70° C. and 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (350 mg, 0.66 mmol, 1 equiv) was added in small portions to the hot mixture during 1 h. Upon addition completed, the reaction mixture was stirred for 10 more minutes, coated on SiO₂ and subjected to CC (eluent:MeOH in DCM: 3%→13%). Fractions with the product were collected and concentrated to afford the target product (3) as a yellow oil (390 mg, 90%).

### N-((3-(2,4-Dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (5) (Scheme 1)

3-(2,4-Dimethoxybenzyl)-8,8-dimethyl-11-(5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (3) (390 mg, 0.594 mmol) was mixed with trifluoroacetic anhydride (4) (5 mL) and reaction mixture was stirred at rt for overnight. The mixture was concentrated and re-evaporated twice with toluene to afford crude target product (5) as a brown oil (447 mg) which was used in the next stage without any purification.

### N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (6) (Scheme 1)

Crude N-((3-(2,4-dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (5) (447 mg, 0.59 mmol) was mixed with trifluoroacetic acid (5 mL) and reaction mixture was stirred at rt for overnight. Magenta reaction mixture was concentrated, mixed with 5 mL of MeCN and poured into aq phosphate buffer (pH 7, 100 mL). The precipitate formed was filtered off, washed with water and dried in a vacuum oven to afford crude product (6) as a dark-brown solid (155 mg, 44%).

### N-((4-Chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (7) (Scheme 1)

N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (6) (crude 155 mg, 0.257 mmol) was suspended in 3 mL of POCl₃ and 0.3 mL of pyridine. The resultant mixture was heated at 70° C. for 3 h, then carefully and slowly poured into 200 mL of ice-cold water. Aqueous fraction was extracted with a mixture t-BuOH/CHCl₃ 1/1 (4×30 mL). Organics were combined, dried over Na₂SO₄, filtered and concentrated to afford crude target product (7) as a dark-brown solid (146 mg, 90%).

### N-(2-(1H-Benzo[d]imidazol-5-yl)ethyl)-8,8-dimethyl-11-(5,8,11,14-tetraoxa-2-azapentadecyl)-7,10-dihydro-8H-pyrano[3″,4″:5′,6′]pyrido[3′,2′:4,5]thieno[3,2-d]pyrimidin-4-amine (Compound 321) (Scheme 1)

A suspension of N-((4-chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (7) (crude 146 mg, 0.234 mmol, 1 equiv), 2-(1H-benzo[d]imidazol-5-yl)ethan-1-amine dihydrochloride (8) (66 mg, 0.281 mmol, 1.2 equiv) and TEA (0.2 mL, 1.4 mmol, 6 equiv) in 25 mL of i-PrOH was refluxed for 8 h. TLC showed full consumption of chloride (7) and formation of the reaction product—intermediate (9) which was not isolated in a pure form but subjected to deprotection in situ. K₂CO₃ (48 mg, 0.351 mmol, 1.5 equiv) and water (5 mL) were added and the reaction mixture was refluxed for further 3.5 h, then poured into 200 mL of water. Aqueous layer was extracted with a mixture t-BuOH/CHCl₃ 1/1 (3×30 mL). Organics were combined, dried over Na₂SO₄, filtered, coated on SiO₂ and subjected to CC (eluent:MeOH in DCM: 25%). Fractions with the product were collected, partially concentrated and subjected to PTLC on SiO₂ plates (1500 μm) using a mixture EA/MeOH+4.5 vv % aq NH₃ as an eluent. The solid product obtained was triturated with EA+Et₂O to afford the target product (Compound 321) (13 mg, 3% after 4 stages) as a beige solid.

### Synthetic Details for Compound 320

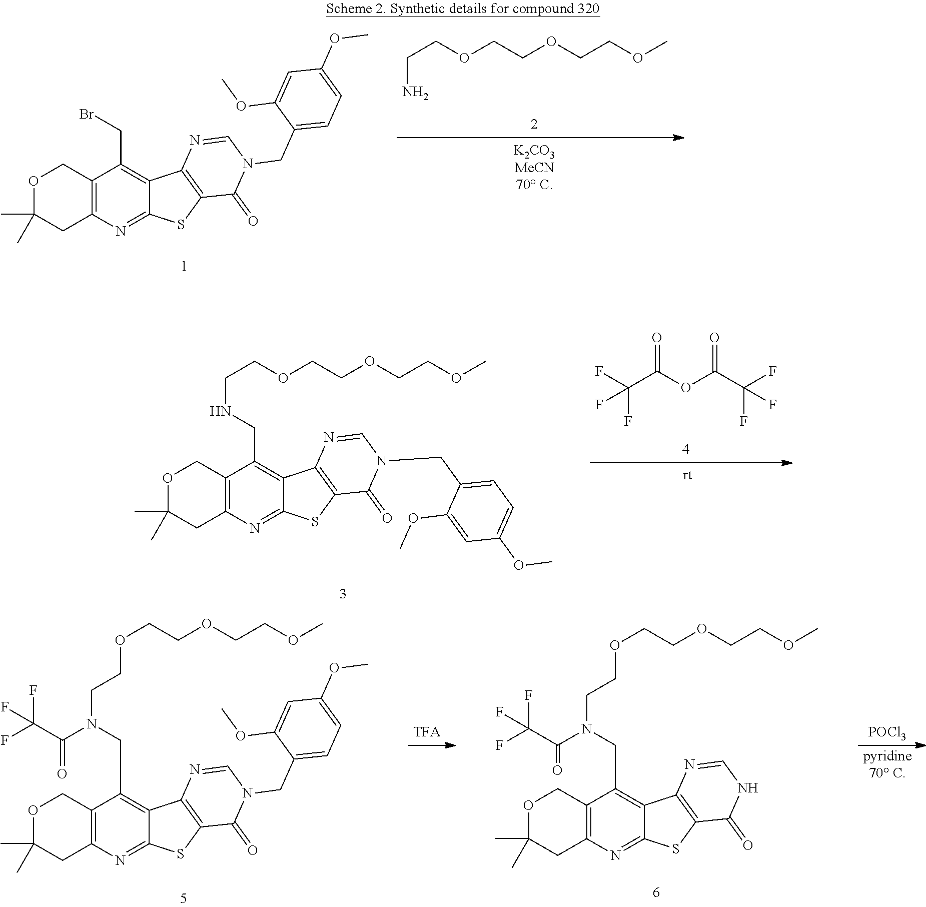

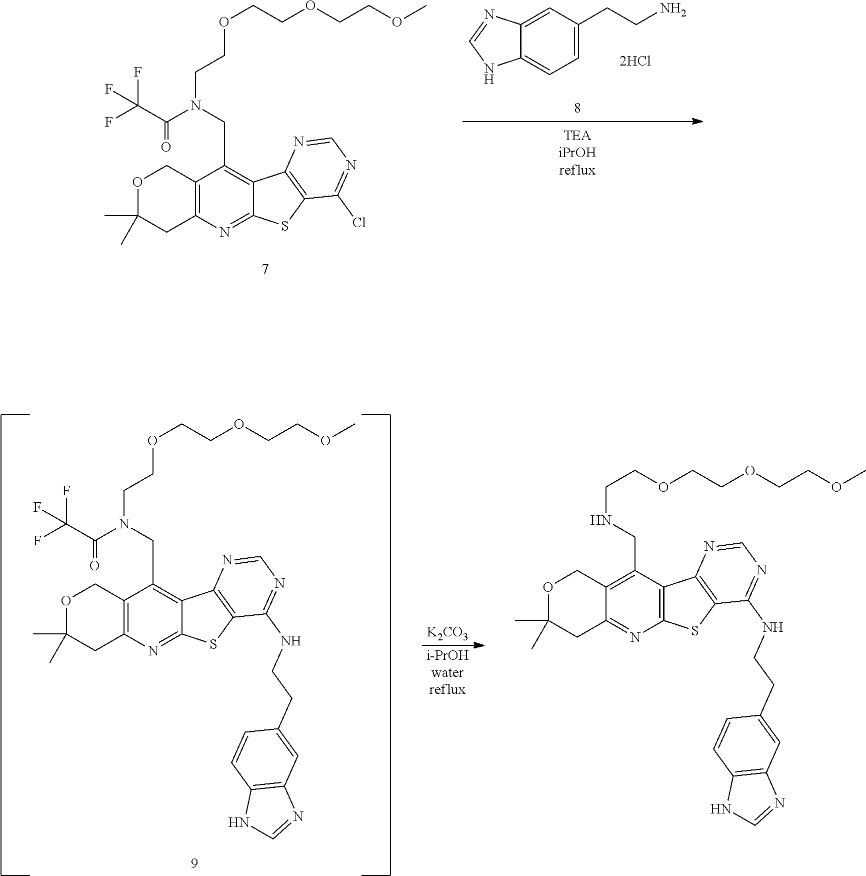

### 3-(2,4-Dimethoxybenzyl)-11-(5,8,11-trioxa-2-azadodecyl)-8,8-dimethyl-7,10-dihydro-8H-pyrano[3″,4″:5′,6′]pyrido[3′,2′:4,5]thieno[3,2-d]pyrimidin-4(3H)-one (3) (Scheme 2)

A mixture of 2-(2-(2-methoxyethoxy)ethoxy)ethan-1-amine (2) (1.08 g, 6.6 mmol, 5 equiv) and K₂CO₃ (90 mg, 0.66 mmol, 1 equiv) in 10 mL of MeCN was heated to 65° C. and 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (350 mg, 0.66 mmol, 1 equiv) was added in small portions to the hot mixture during 1 h. Upon addition completed, the reaction mixture was stirred for 10 more minutes, coated on SiO₂ and subjected to CC (eluent:MeOH in DCM: 3%→6%). Fractions with the product were collected and concentrated to afford the target product (3) as a pail-yellow oil (430 mg, >100%).

### N-((3-(2,4-Dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (5) (Scheme 2)

3-(2,4-Dimethoxybenzyl)-11-(5,8,11-trioxa-2-azadodecyl)-8,8-dimethyl-7,10-dihydro-8H-pyrano[3″,4″:5′,6′]pyrido[3′,2′:4,5]thieno[3,2-d]pyrimidin-4(3H)-one (3) (405 mg, 0.66 mmol) was mixed with trifluoroacetic anhydride (4) (5 mL) and reaction mixture was stirred at rt for 2 days. The mixture was concentrated and re-evaporated twice with toluene to afford crude target product (5) as a brown viscous oil (468 mg) which was used in the next stage without any purification.

### N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (6) (Scheme 2)

Crude N-((3-(2,4-dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (5) (468 mg, 0.66 mmol) was mixed with trifluoroacetic acid (5 mL) and reaction mixture was stirred at rt for overnight. Magenta reaction mixture was concentrated, mixed with 5 mL of MeCN and poured into aq phosphate buffer (pH 7, 100 mL). The precipitate formed was filtered off, washed with water and dried in a vacuum oven (temperature should not exceed 40° C.) to afford crude product (6) as a light-brown solid (275 mg, 75%).

### N-((4-Chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (7) (Scheme 2)

N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (6) (crude 275 mg, 0.492 mmol) was suspended in 3 mL of POCl₃ and 0.3 mL of pyridine. The resultant mixture was heated at 70° C. for 4.5 h, then carefully and slowly poured into 200 mL of ice-cold water. Aqueous fraction was extracted with a mixture t-BuOH/CHCl₃ 1/1 (4×30 mL). Organics were combined, dried over Na₂SO₄, filtered and concentrated to afford crude target product (7) as a dark-brown solid (254 mg, 89%).

### N-(2-(1H-Benzo[d]imidazol-5-yl)ethyl)-11-(5,8,11-trioxa-2-azadodecyl)-8,8-dimethyl-7,10-dihydro-8H-pyrano[3″,4″:5′,6′]pyrido[3′,2′:4,5]thieno[3,2-d]pyrimidin-4-amine (Compound 320) (Scheme 2)

A suspension of N-((4-chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2-(2-(2-methoxyethoxy)ethoxy)ethyl)acetamide (7) (crude 254 mg, 0.439 mmol, 1 equiv), 2-(1H-benzo[d]imidazol-5-yl)ethan-1-amine dihydrochloride (8) (123 mg, 0.527 mmol, 1.2 equiv) and TEA (0.37 mL, 2.634 mmol, 6 equiv) in 25 mL of i-PrOH was refluxed for 8 h. TLC showed full consumption of chloride (7) and formation of the reaction product—intermediate (9) which was not isolated in a pure form but subjected to deprotection in situ. K₂CO₃ (90 mg, 0.659 mmol, 1.5 equiv) and water (5 mL) were added and the reaction mixture was refluxed for further 6 h, then poured into 200 mL of water. Aqueous layer was extracted with a mixture t-BuOH/CHCl₃ 1/1 (4×30 mL). Organics were combined, dried over Na₂SO₄, filtered, coated on SiO₂ and subjected to CC (eluent:MeOH in DCM: 22%→30%). Fractions with the product were collected and concentrated. The residue was recrystallized from a hot mixture EA+a bit MeOH to afford the target product (Compound 320) (53 mg, 13% after 5 stages) as an off-white solid.

### Synthetic Details for Compound 319

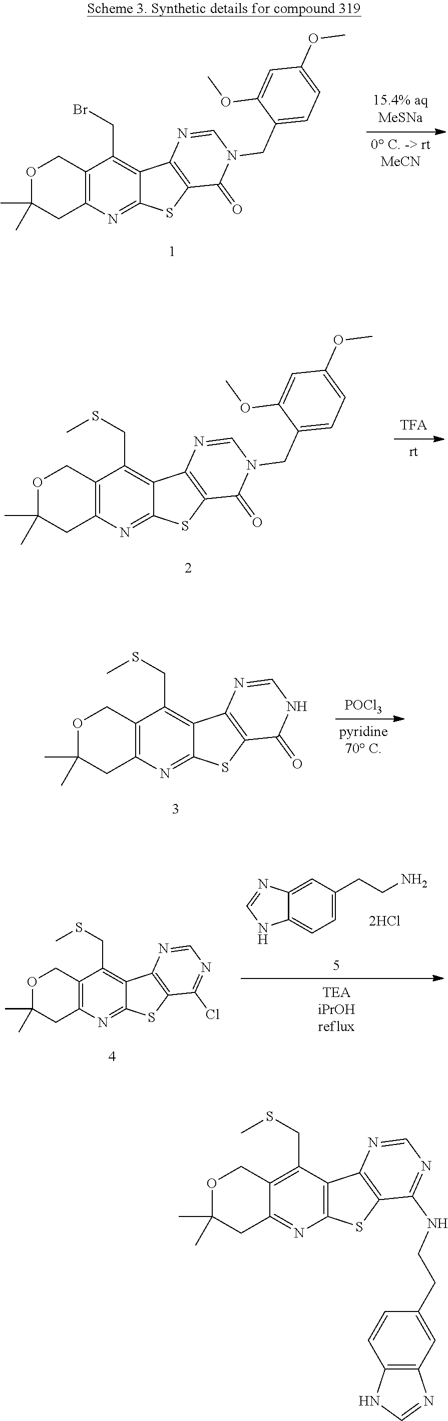

### 3-(2,4-Dimethoxybenzyl)-8,8-dimethyl-11-((methylthio)methyl)-7,10-dihydro-8H-pyrano[3″,4″:5′,6′]pyrido[3′,2′:4,5]thieno[3,2-d]pyrimidin-4(3H)-one (2) (Scheme 3)

11-(Bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (100 mg, 0.21 mmol, 1 equiv) was suspended in 10 mL of MeCN and the mixture was cooled to 0-4° C. Aqueous solution of sodium methanethiolate (15.4%, 194 mg, 0.42 mmol, 2 equiv) was added and the reaction mixture was stirred at 55° C. for 2.5 h. Solvent was evaporated in vacuo and water (25 mL) was added to reaction mixture. The precipitate was filtered off, washed with water and recrystallized from 7 mL of MeCN to afford the target product (2) as a white needle-like solid (58 mg, 62%).

### 8,8-Dimethyl-11-((methylthio)methyl)-7,10-dihydro-8H-pyrano[3″,4″:5′,6′]pyrido[3′,2′:4,5]thieno[3,2-d]pyrimidin-4(3H)-one (3) (Scheme 3)

3-(2,4-Dimethoxybenzyl)-8,8-dimethyl-11-((methylthio)methyl)-7,10-dihydro-8H-pyrano[3″,4″:5′,6′]pyrido[3′,2′:4,5]thieno[3,2-d]pyrimidin-4(3H)-one (2) (160 mg, 0.32 mmol) was stirred in 5 mL of trifluoroacetic acid at rt for 2 h. Magenta reaction mixture was concentrated and mixed with 5 mL of MeCN, then poured into 100 mL of cold sat aq NaHCO₃ solution. The precipitate formed was filtered off, washed with water and dried at 80° C. to afford the target product (3) as a white solid (174 mg, >100%).

### 4-Chloro-8,8-dimethyl-11-((methylthio)methyl)-7,10-dihydro-8H-pyrano[3″,4″:5′,6′]pyrido[3′,2′:4,5]thieno[3,2-d]pyrimidine (4) (Scheme 3)

8,8-Dimethyl-11-((methylthio)methyl)-7,10-dihydro-8H-pyrano[3″,4″:5′,6′]pyrido[3′,2′:4,5]thieno[3,2-d]pyrimidin-4(3H)-one (3) (crude 174 mg, 0.5 mmol) was suspended in 3 mL of POCl₃ and 0.3 mL of pyridine. The resultant mixture was heated at 70° C. for 12 h, then carefully and slowly poured into 200 mL of ice-cold water. Aqueous fraction was extracted with DCM (3×30 mL), and the organics were combined, dried over Na₂SO₄, filtered and concentrated to afford crude target product (4) as an orange solid (155 mg).

### N-(2-(1H-Benzo[d]imidazol-5-yl)ethyl)-8,8-dimethyl-11-((methylthio)methyl)-7,10-dihydro-8H-pyrano[3″,4″:5′,6′]pyrido[3′,2′:4,5]thieno[3,2-d]pyrimidin-4-amine (Compound 319) (Scheme 3)

In a 50-mL flask were mixed 4-chloro-8,8-dimethyl-11-((methylthio)methyl)-7,10-dihydro-8H-pyrano[3″,4″:5′,6′]pyrido[3′,2′:4,5]thieno[3,2-d]pyrimidine (4) (crude 145 mg, 0.4 mmol, 1 equiv), 2-(1H-benzo[d]imidazol-5-yl)ethan-1-amine dihydrochloride (5) (186 mg, 0.8 mmol, 2 equiv) and TEA (0.3 mL, 2.4 mmol, 6 equiv) in 25 mL of i-PrOH. The reaction mixture was refluxed for 16 h and hot-filtered from undissolved solids (not the product, solids were discarded). The alcoholic solution was partially concentrated to V˜5 mL and poured into 100 mL of water. The precipitate formed was collected by filtration, dissolved in a mixture DCM/MeOH 1/1, coated on SiO₂ and subjected to CC (eluent: DCM/MeOH=5%→7%). Fractions with the product were collected and concentrated. The solid residue was dissolved in a hot mixture EA/MeOH 3/1, hot-filtered and again concentrated, then triturated with hot EA to afford the target product (Compound 319) as an off-white solid (84 mg, 53% after 3 stages).

### Synthetic Details for Compound 326

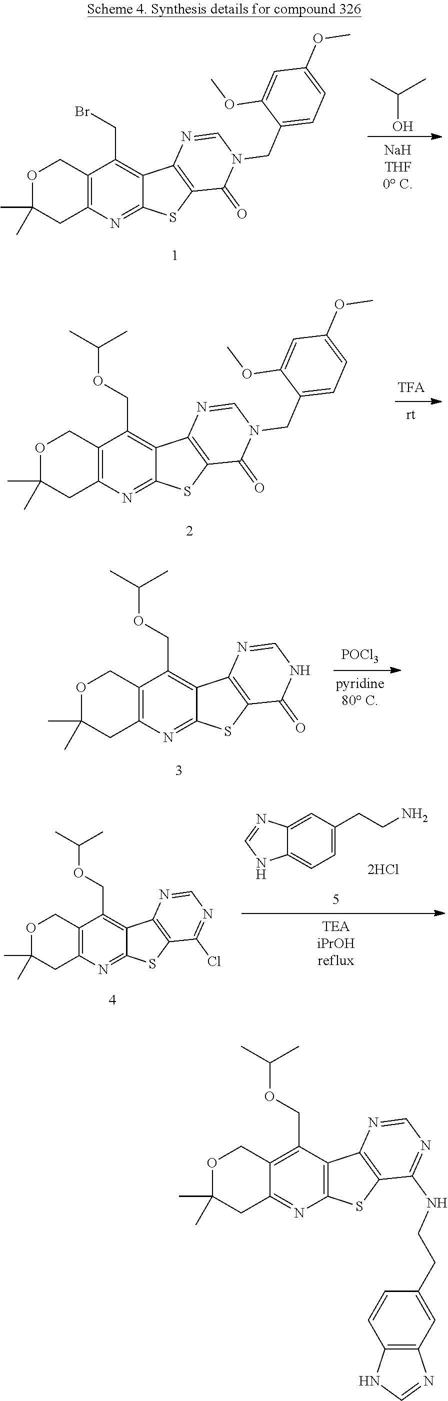

### 3-(2,4-Dimethoxybenzyl)-11-(isopropoxymethyl)-8,8-dimethyl-7,10-dihydro-8H-pyrano[3″,4″:5′,6′]pyrido[3′,2′:4,5]thieno[3,2-d]pyrimidin-4(3H)-one (2) (Scheme 4)

In the 1^{st} flask a mixture of isopropanol (1.8 mL, 23.55 mmol, 50 equiv) in 10 mL of dry THF was cooled to 0° C. under Ar. To this solution was added NaH (60% in mineral oil) (470 mg, 19.6 mmol, 25 equiv) in portions and the mixture was stirred under cooling for 10 min. In the 2^{nd} flask a suspension of 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv) in dry THF (10 mL) was also cooled to 0° C. under Ar. A mixture (1 mL) from the 1^{st} flask was added to the 2^{nd} flask and resultant reaction mixture was stirred under cooling for 1 h. During next 2 h two portions (2×0.5 mL) of the mixture from the 1⁴ flask was added to the 2^{nd} flask and stirring was continued. Reaction mixture was poured into 200 mL of water. Aqueous layer was extracted with a mixture t-BuOH/CHCl₃ 1/1 (2×60 mL). Organics were combined, dried over Na₂SO₄, filtered, coated on SiO₂ and subjected to CC (eluent: DCM/MeOH=0.5%→1%). Fractions with the product were collected and concentrated to afford the target product (2) as a colourless glass-like substance (100 mg, 42%).

### 11-(Isopropoxymethyl)-8,8-dimethyl-7,10-dihydro-8H-pyrano[3″,4″:5′,6′]pyrido[3′,2′:4,5]thieno[3,2-d]pyrimidin-4(3H)-one (3) (Scheme 4)

3-(2,4-Dimethoxybenzyl)-11-(isopropoxymethyl)-8,8-dimethyl-7,10-dihydro-8H-pyrano[3″,4″:5′,6′]pyrido[3′,2′:4,5]thieno[3,2-d]pyrimidin-4(3H)-one (2) (100 mg, 0.2 mmol) was stirred in 5 mL of trifluoroacetic acid at rt for overnight. Magenta reaction mixture was concentrated and mixed with 5 mL of MeCN, then poured into 150 mL of cold water. Aqueous layer was extracted with a mixture t-BuOH/CHCl₃ 1/1 (3×50 mL). Organics were combined, dried over Na₂SO₄, filtered and concentrated to afford crude target product (3) as a pale-brown solid (80 mg) which was used in the next step without further purification.

### 4-Chloro-11-(isopropoxymethyl)-8,8-dimethyl-7,10-dihydro-8H-pyrano[3″,4″:5′,6′]pyrido[3′,2′:4,5]thieno[3,2-d]pyrimidine (4) (Scheme 4)

11-(Isopropoxymethyl)-8,8-dimethyl-7,10-dihydro-8H-pyrano[3″,4″:5′,6′]pyrido[3′,2′:4,5]thieno[3,2-d]pyrimidin-4(3H)-one (3) (crude 80 mg, 0.22 mmol) was suspended in 3 mL of POCl₃ and 0.3 mL of pyridine. The resultant mixture was heated at 80° C. for 10 h, then carefully and slowly poured into 150 mL of ice-cold water. The precipitate formed was filtered off, washed with water and dried to afford crude target product (4) as a brown solid (40 mg).

### N-(2-(1H-Benzo[d]imidazol-5-yl)ethyl)-11-((cyclohexyloxy)methyl)-8,8-dimethyl-7,10-dihydro-8H-pyrano[3″,4″:5′,6′]pyrido[3′,2′:4,5]thieno[3,2-d]pyrimidin-4-amine (compound 326) (Scheme 4)

In a 50-mL flask were mixed 4-chloro-11-(isopropoxymethyl)-8,8-dimethyl-7,10-dihydro-8H-pyrano[3″,4″:5′,6′]pyrido[3′,2′:4,5]thieno[3,2-d]pyrimidine (4) (crude 40 mg, 0.106 mmol, 1 equiv), 2-(1H-benzo[d]imidazol-5-yl)ethan-1-amine dihydrochloride (5) (30 mg, 0.127 mmol, 1.2 equiv) and TEA (0.1 mL, 0.636 mmol, 6 equiv) in 20 mL of i-PrOH. The reaction mixture was refluxed for 24 h. More starting (5) (7 mg, 0.03 mmol, 0.3 equiv) was added and the mixture was refluxed for further 24 h. The mixture was poured into 200 mL of water and extracted with DCM (4×50 mL). Organics were combined, dried over Na₂SO₄, filtered, coated on SiO₂ and subjected to CC (eluent: DCM/MeOH=6%→7%). Fractions with the product were combined and concentrated. The residue was triturated with boiling EA to afford the target product (compound 326) as a yellowish-white solid (9 mg, 17%).

### Synthetic Details for Compound 327

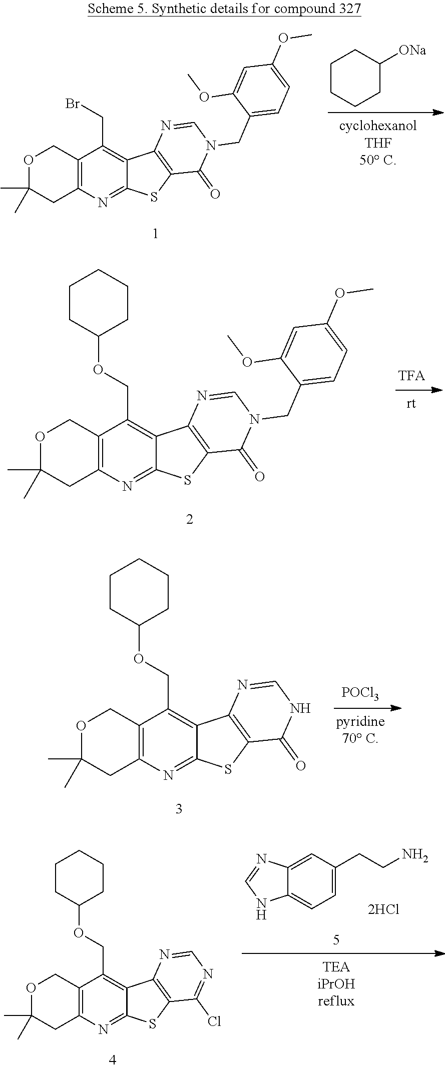

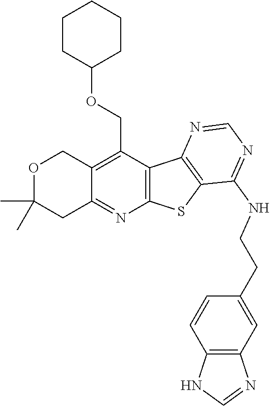 [SMILES: CC1(C)Cc2nc3sc4c(NCCc5ccc6[nH]cnc6c5)ncnc4c3c(COC3CCCCC3)c2CO1]

### 11-((Cyclohexyloxy)methyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (2) (Scheme 5)

Sodium metal (122 mg, 5.31 mmol, 10 equiv) was added to a mixture of cyclohexanol (3 mL) and dry THF (7 mL) under Ar. Prepared this way mixture was brought to reflux for 30 min and 2 mL of it was added to a suspension of 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv) in dry THF (10 mL). The reaction mixture was stirred at 50° C. for 3 h and poured into 200 mL of water. Aqueous layer was extracted with a mixture t-BuOH/CHCl₃ 1/1 (4×60 mL). Organics were combined, dried over Na₂SO₄, filtered, coated on SiO₂ and subjected to CC (eluent: DCM/MeOH=0.5%→1%). Fractions with the product were collected and concentrated to afford the target product (2) as an off-white solid (100 mg, 38%).

### 11-((Cyclohexyloxy)methyl)-8,8-dimethyl-7,10-dihydro-8H-pyrano[3″,4″:5′,6′]pyrido[3′,2′:4,5]thieno[3,2-d]pyrimidin-4(3H)-one (3) (Scheme 5)

11-((Cyclohexyloxy)methyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (2) (100 mg, 0.18 mmol) was stirred in 5 mL of trifluoroacetic acid at rt for overnight. Magenta reaction mixture was concentrated and mixed with 5 mL of MeCN, then poured into 150 mL of cold water. Aqueous layer was extracted with a mixture t-BuOH/CHCl₃ 1/1 (3×50 mL). Organics were combined, dried over Na₂SO₄, filtered, coated on SiO₂ and subjected to CC (eluent: DCM/MeOH=2%→4%). Fractions with the product were collected and concentrated to afford the target product (3) as a white solid (70 mg, 96%).

### 4-Chloro-11-((cyclohexyloxy)methyl)-8,8-dimethyl-7,10-dihydro-8H-pyrano[3″,4″:5′,6′]pyrido[3′,2′:4,5]thieno[3,2-d]pyrimidine (4) (Scheme 5)

11-((Cyclohexyloxy)methyl)-8,8-dimethyl-7,10-dihydro-8H-pyrano[3″,4″:5′,6′]pyrido[3′,2′:4,5]thieno[3,2-d]pyrimidin-4(3H)-one (3) (70 mg, 0.175 mmol) was suspended in 3 mL of POCl₃ and 0.3 mL of pyridine. The resultant mixture was heated at 70° C. for 6 h, then carefully and slowly poured into 150 mL of ice-cold water. Aqueous layer was extracted with a mixture t-BuOH/CHCl₃ 1/1 (3×30 mL). Organics were combined, dried over Na₂SO₄ and filtered to afford crude target product (4) as a brown solid (61 mg).

### N-(2-(1H-Benzo[d]imidazol-5-yl)ethyl)-11-((cyclohexyloxy)methyl)-8,8-dimethyl-7,10-dihydro-8H-pyrano[3″,4″:5′,6′]pyrido[3′,2′:4,5]thieno[3,2-d]pyrimidin-4-amine (compound 327) (Scheme 5)

In a 50-mL flask were mixed 4-chloro-11-((cyclohexyloxy)methyl)-8,8-dimethyl-7,10-dihydro-8H-pyrano[3″,4″:5′,6′]pyrido[3′,2′:4,5]thieno[3,2-d]pyrimidine (4) (crude 61 mg, 0.146 mmol, 1 equiv), 2-(1H-benzo[d]imidazol-5-yl)ethan-1-amine dihydrochloride (5) (51 mg, 0.219 mmol, 1.5 equiv) and TEA (0.1 mL, 0.876 mmol, 6 equiv) in 10 mL of i-PrOH. The reaction mixture was refluxed for 24 h. The mixture was poured into 300 mL of water and extracted with a mixture t-BuOH/CHCl₃ 1/1 (2×50 mL). Organics were combined, dried over Na₂SO₄, filtered, coated on SiO₂ and subjected to CC (eluent: DCM/MeOH=2.5%→4%). Fractions with the product were combined, dried over Na₂SO₄, filtered, partially concentrated and subjected to preparative TLC (eluent: DCM/MeOH=93/7) on SiO₂-plates. The collected product was triturated with EA to afford the target product (compound 327) as a white solid (13 mg, 16%).

### Synthetic Details for Compound 328

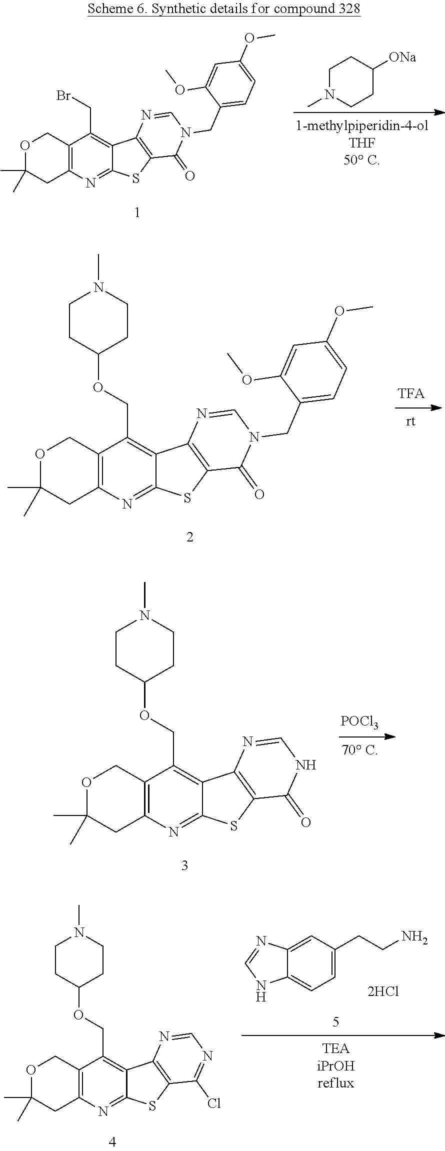

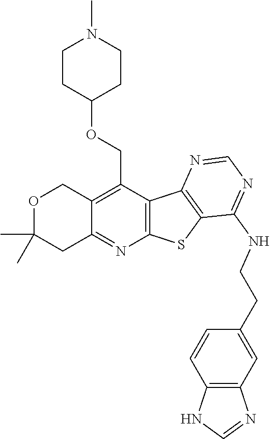 [SMILES: CN1CCC(OCc2c3c(nc4sc5c(NCCc6ccc7[nH]cnc7c6)ncnc5c24)CC(C)(C)OC3)CC1]

### 3-(2,4-Dimethoxybenzyl)-8,8-dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidin-4(3H)-one (2) (Scheme 6)

Sodium metal (244 mg, 10.62 mmol, 20 equiv) was added to a mixture of 1-methylpiperidin-4-ol (3 mL) and dry THF (3 mL) under Ar. Prepared this way mixture was brought to reflux for 1 h and 2 mL of it was added to a suspension of 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv) in dry THF (5 mL). The reaction mixture was stirred at 55° C. for 2 h and poured into 200 mL of brine. Aqueous layer was extracted with DCM (4×60 mL). Organics were combined, dried over Na₂SO₄, filtered, coated on SiO₂ and subjected to CC (eluent: DCM/MeOH=2%→5%). Fractions with the product were collected and concentrated to afford the target product (2) as an off-white solid (120 mg, 45%).

### 8,8-Dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidin-4(3H)-one (3) (Scheme 6)

3-(2,4-Dimethoxybenzyl)-8,8-dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidin-4(3H)-one (2) (120 mg, 0.21 mmol) was stirred at rt for overnight. Magenta reaction mixture was mixed with 10 mL of toluene and concentrated in vacuo. Another 15 mL of toluene was added, followed by SiO₂, and reaction mixture was again concentrated, then subjected to CC (eluent: DCM saturated with 10% v/v NH₃+6%→8% v/v MeOH). Fractions with the product were collected and concentrated to afford the target product (3) as an off-white solid (49 mg, 25% after two stages).

### 4-Chloro-8,8-dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidine (4) (Scheme 6)

8,8-Dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidin-4(3H)-one (3) (48 mg, 0.116 mmol) was suspended in 3 mL of POCl₃ and the resultant mixture was heated at 70° C. for 4 h. Reaction mixture was twice mixed with 10 mL of toluene and twice concentrated in vacuo to afford crude target product (4) (45 mg) which was used in the next step without any purification.

### N-(2-(1H-Benzo[d]imidazol-5-yl)ethyl)-8,8-dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidin-4-amine (compound 328) (Scheme 6)

A suspension of 4-chloro-8,8-dimethyl-11-(((1-methylpiperidin-4-yl)oxy)methyl)-7,10-dihydro-8H-pyrano[3″,4″:5′,6′]pyrido[3′,2′:4,5]thieno[3,2-d]pyrimidine (4) (crude 45 mg, 0.1 mmol, 1 equiv), 2-(1H-benzo[d]imidazol-5-yl)ethan-1-amine dihydrochloride (5) (47 mg, 0.2 mmol, 2 equiv) and TEA (0.1 mL, 0.6 mmol, 6 equiv) in 10 mL of i-PrOH was refluxed for 30 h. The reaction mixture was concentrated to V˜5-7 mL and poured into 250 mL of cold water. Aqueous layer was extracted with DCM (4×50 mL). Organics were combined, dried over Na₂SO₄, filtered, coated on SiO₂ and subjected to CC (eluent: pure MeOH). Fractions with the product were collected, partially concentrated and subjected to PTLC on SiO₂ plates (1500 m) using a mixture DCM saturated with 10% v/v NH₃+8% v/v MeOH as an eluent. The solid product obtained was triturated with EA to afford the target product (compound 328) (7 mg, 12.5%) as a white solid.

### Synthetic Details for Compound 324

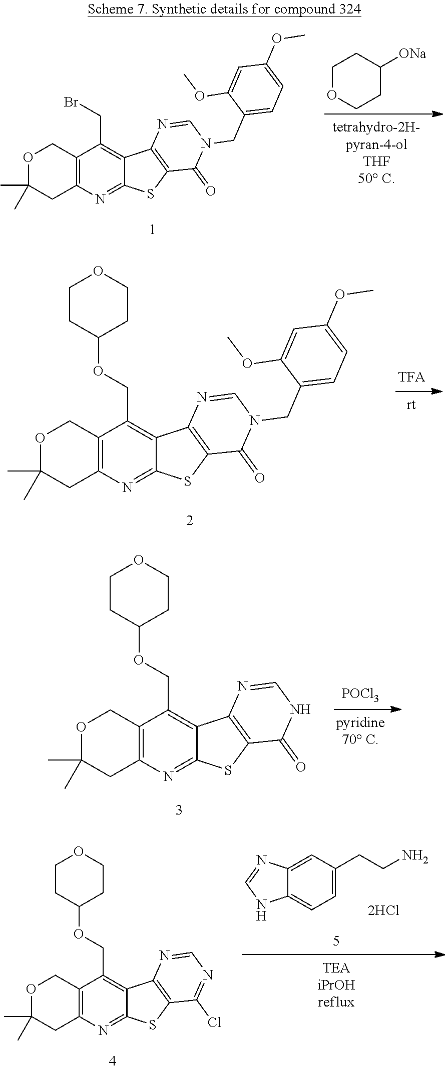

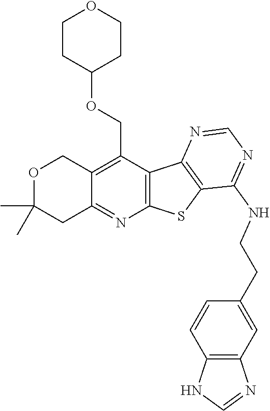 [SMILES: CC1(C)Cc2nc3sc4c(NCCc5ccc6[nH]cnc6c5)ncnc4c3c(COC3CCOCC3)c2CO1]

### 3-(2,4-Dimethoxybenzyl)-8,8-dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidin-4(3H)-one (2) (Scheme 7)

Sodium metal (122 mg, 5.31 mmol, 10 equiv) was added to a mixture of tetrahydro-2H-pyran-4-ol (2 mL) and dry THF (2 mL) under Ar. Prepared this way mixture was brought to reflux for 30 min and 2 mL of it was added to a suspension of 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv) in dry THF (2 mL). The reaction mixture was stirred at 50° C. for 3 h and poured into 150 mL of water. Solid precipitated was filtered off, washed with water and dried to afford the target product (2) as a white solid (193 mg, 74%).

### 8,8-Dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidin-4(3H)-one (3) (Scheme 7)

3-(2,4-Dimethoxybenzyl)-8,8-dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidin-4(3H)-one (2) (193 mg, 0.35 mmol) was stirred in 5 mL of trifluoroacetic acid at rt for overnight. Magenta reaction mixture was concentrated and mixed with 5 mL of MeCN, then poured into 150 mL of cold water. Aqueous layer was extracted with a mixture t-BuOH/CHCl₃ 1/1 (3×60 mL). Organics were combined, dried over Na₂SO₄, filtered, coated on SiO₂ and subjected to CC (eluent:MeOH in DCM=3%). Fractions with the product were collected and concentrated to afford the target product (3) as a white solid (98 mg, 70%).

### 4-Chloro-8,8-dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidine (4) (Scheme 7)

8,8-Dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidin-4(3H)-one (3) (95 mg, 0.237 mmol) was suspended in 3 mL of POCl₃ and 0.3 mL of pyridine. The resultant mixture was heated at 70° C. for 2 h when the precipitate formed on the walls of the flask. MeCN (20 mL) was added to homogenize the reaction mixture and heating was continued for further 4 h. The mixture was concentrated to V˜5-7 mL and poured into 150 mL of ice-cold water. Solid precipitated was filtered off, washed with water and dried to afford crude target product (4) as a brown solid (70 mg).

### N-(2-(1H-Benzo[d]imidazol-5-yl)ethyl)-8,8-dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidin-4-amine (compound 324) (Scheme 7)

In a 50-mL flask were mixed 4-chloro-8,8-dimethyl-11-(((tetrahydro-2H-pyran-4-yl)oxy)methyl)-7,10-dihydro-8H-pyrano[3″,4″:5′,6′]pyrido[3′,2′:4,5]thieno[3,2-d]pyrimidine (4) (crude 70 mg, 0.167 mmol, 1 equiv), 2-(1H-benzo[d]imidazol-5-yl)ethan-1-amine dihydrochloride (5) (47 mg, 0.2 mmol, 1.2 equiv) and TEA (0.15 mL, 1 mmol, 6 equiv) in 10 mL of i-PrOH. The reaction mixture was refluxed for 24 h with extra addition of starting (5) (8 mg, 0.03 mmol, 0.2 equiv). The mixture was poured into 200 mL of water and extracted with a mixture t-BuOH/CHCl₃ 1/1 (2×50 mL). Organics were combined, dried over Na₂SO₄, filtered, coated on SiO₂ and subjected to CC (eluent:MeOH in DCM=8%→10%). Fractions with the product were combined, dried over Na₂SO₄, filtered and concentrated. The residue was triturated with a mixture EA/Et₂O 1/1 to afford the target product (compound 324) as a white solid (23 mg, 25%).

### Synthetic Details for Compound 322

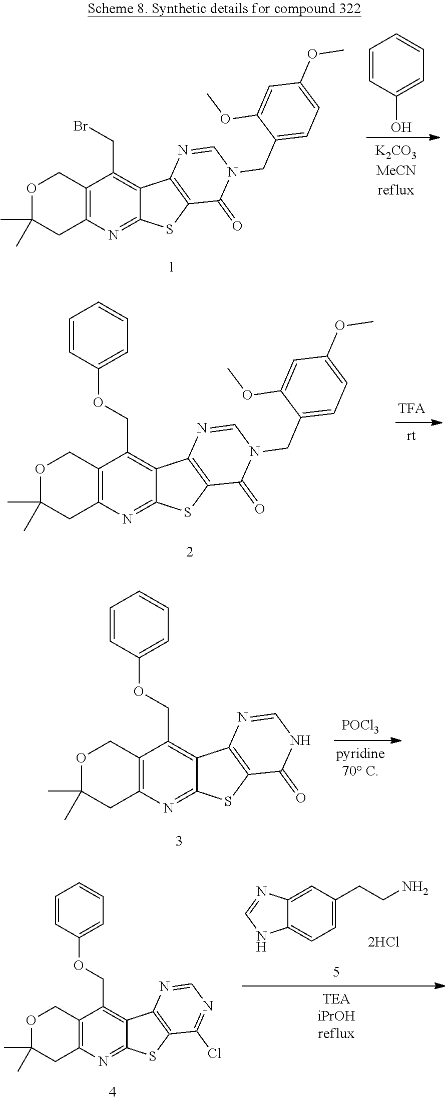

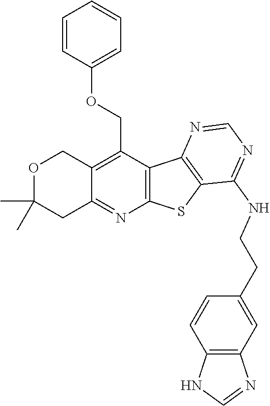 [SMILES: CC1(C)Cc2nc3sc4c(NCCc5ccc6[nH]cnc6c5)ncnc4c3c(COc3ccccc3)c2CO1]

### 3-(2,4-Dimethoxybenzyl)-8,8-dimethyl-11-(phenoxymethyl)-7,10-dihydro-8H-pyrano[3″,4″:5′,6′]pyrido[3′,2′:4,5]thieno[3,2-d]pyrimidin-4(3H)-one (2) (Scheme 8)

11-(Bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv), phenol (67 mg, 0.71 mmol, 1.5 equiv) and K₂CO₃ (98 mg, 0.71 mmol, 1.5 equiv) were suspended in 15 mL of MeCN and reaction mixture was refluxed for 10 h, then poured into 150 mL of water. The precipitate formed was too fine for successful filtration, so suspension had to be transferred to a separatory funnel and extracted with a mixture t-BuOH/CHCl₃ 1/1 (3×70 mL). Organics were combined, dried over Na₂SO₄, filtered and concentrated to afford crude target product (2) as a yellowish oily solid (329 mg, contaminated with phenol).

### 8,8-Dimethyl-11-(phenoxymethyl)-7,10-dihydro-8H-pyrano[3″,4″:5′,6′]pyrido[3′,2′:4,5]thieno[3,2-d]pyrimidin-4(3H)-one (3) (Scheme 8)

3-(2,4-Dimethoxybenzyl)-8,8-dimethyl-11-(phenoxymethyl)-7,10-dihydro-8H-pyrano[3″,4″:5′,6′]pyrido[3′,2′:4,5]thieno[3,2-d]pyrimidin-4(3H)-one (2) (256 mg, 0.47 mmol) was stirred in 5 mL of trifluoroacetic acid at rt for overnight. Magenta reaction mixture was concentrated and mixed with 5 mL of MeCN, then poured into 100 mL of cold water. Aqueous layer was extracted with a mixture t-BuOH/CHCl₃ 1/1 (3×30 mL). Organics were combined, dried over Na₂SO₄, filtered, coated on SiO₂ and subjected to CC (eluent: DCM/MeOH=1.5%→2.5%). Fractions with the product were collected and concentrated to afford the target product (3) as a white solid (159 mg, 86%).

### 4-Chloro-8,8-dimethyl-11-(phenoxymethyl)-7,10-dihydro-8H-pyrano[3″,4″:5′,6′]pyrido[3′,2′:4,5]thieno[3,2-d]pyrimidine (4) (Scheme 8)

8,8-Dimethyl-11-(phenoxymethyl)-7,10-dihydro-8H-pyrano[3″,4″:5′,6′]pyrido[3′,2′:4,5]thieno[3,2-d]pyrimidin-4(3H)-one (3) (147 mg, 0.37 mmol) was suspended in 3 mL of POCl₃ and 0.3 mL of pyridine. The resultant mixture was heated at 70° C. for 10 h, then carefully and slowly poured into 150 mL of ice-cold water. The precipitate formed was filtered off, washed with water and dried to afford target product (4) as a brown-red solid (137 mg. 89%).

### N-(2-(1H-Benzo[d]imidazol-5-yl)ethyl)-8,8-dimethyl-11-(phenoxymethyl)-7,10-dihydro-8H-pyrano[3″,4″:5′,6′]pyrido[3′,2′:4,5]thieno[3,2-d]pyrimidin-4-amine (compound 322) (Scheme 8)

In a 50-mL flask were mixed 4-chloro-8,8-dimethyl-11-(phenoxymethyl)-7,10-dihydro-8H-pyrano[3″,4″:5′,6′]pyrido[3′,2′:4,5]thieno[3,2-d]pyrimidine (4) (132 mg, 0.32 mmol, 1 equiv), 2-(1H-benzo[d]imidazol-5-yl)ethan-1-amine dihydrochloride (5) (97 mg, 0.42 mmol, 1.3 equiv) and TEA (0.3 mL, 1.92 mmol, 6 equiv) in 25 mL of i-PrOH. The reaction mixture was refluxed for 48 h with extra addition of starting (5) (23 mg, 0.096 mmol, 0.3 equiv). The mixture was poured into 300 mL of water and extracted with a mixture t-BuOH/CHCl₃ 1/1 (3×60 mL). Organics were combined, dried over Na₂SO₄, filtered, coated on SiO₂ and subjected to CC (eluent: DCM/MeOH=5%). Fractions with the product were collected, concentrated and triturated with pentane to afford the target product (compound 322) as a beige solid (57 mg, 33%).

### Synthetic Details for Compound 323

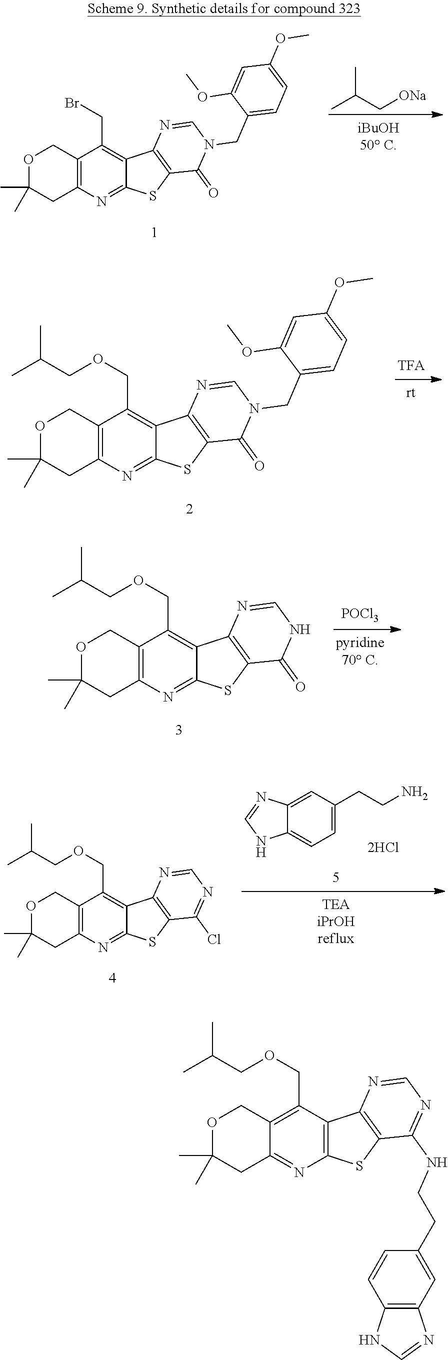

### 3-(2,4-Dimethoxybenzyl)-11-(isobutoxymethyl)-8,8-dimethyl-7,10-dihydro-8H-pyrano[3″,4″:5′,6′]pyrido[3′,2′:4,5]thieno[3,2-d]pyrimidin-4(3H)-one (2) (Scheme 9)

Sodium metal (108 mg, 4.71 mmol, 10 equiv) was dissolved in iso-butyl alcohol (5 mL) under Ar. Prepared this way solution was warmed to 40-50° C. and 2 mL of it was added to a suspension of 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (250 mg, 0.47 mmol, 1 equiv) in iso-butanol (2 mL). The reaction mixture was stirred at 50° C. for 3 h and concentrated in vacuo. The residue was dissolved in 3 mL of MeOH and poured into 100 mL of brine. Aqueous layer was extracted with a mixture t-BuOH/CHCl₃ 1/1 (3×70 mL). Organics were combined, dried over Na₂SO₄, filtered, coated on SiO₂ and subjected to CC (eluent: DCM/MeOH=0.5%→1%). Fractions with the product were collected and concentrated to afford the target product (2) as an off-white solid (201 mg, 81%).

### 11-(Isobutoxymethyl)-8,8-dimethyl-7,10-dihydro-8H-pyrano[3″,4″:5′,6′]pyrido[3′,2′:4,5]thieno[3,2-d]pyrimidin-4(3H)-one (3) (Scheme 9)

3-(2,4-Dimethoxybenzyl)-11-(isobutoxymethyl)-8,8-dimethyl-7,10-dihydro-8H-pyrano[3″,4″:5′,6′]pyrido[3′,2′:4,5]thieno[3,2-d]pyrimidin-4(3H)-one (2) (200 mg, 0.38 mmol) was stirred in 5 mL of trifluoroacetic acid at rt for overnight. Magenta reaction mixture was concentrated and mixed with 5 mL of MeCN, then poured into 100 mL of cold water. Aqueous layer was extracted with a mixture t-BuOH/CHCl₃ 1/1 (3×30 mL). Organics were combined, dried over Na₂SO₄, filtered, coated on SiO₂ and subjected to CC (eluent: DCM/MeOH=1.5%→2.5%). Fractions with the product were collected and concentrated to afford the target product (3) as a white solid (183 mg, >100%).

### 4-Chloro-11-(isobutoxymethyl)-8,8-dimethyl-7,10-dihydro-8H-pyrano[3″,4″:5′,6′]pyrido[3′,2′:4,5]thieno[3,2-d]pyrimidine (4) (Scheme 9)

11-(Isobutoxymethyl)-8,8-dimethyl-7,10-dihydro-8H-pyrano[3″,4″:5′,6′]pyrido[3′,2′:4,5]thieno[3,2-d]pyrimidin-4(3H)-one (3) (160 mg, 0.43 mmol) was suspended in 3 mL of POCl₃ and 0.3 mL of pyridine. The resultant mixture was heated at 70° C. for 5 h, then carefully and slowly poured into 100 mL of ice-cold water. The precipitate formed was filtered off, washed with water and dried to afford target product (4) as a light-brown solid (112 mg, 67%).

### N-(2-(1H-Benzo[d]imidazol-5-yl)ethyl)-11-(isobutoxymethyl)-8,8-dimethyl-7,10-dihydro-8H-pyrano[3″,4″:5′,6′]pyrido[3′,2′:4,5]thieno[3,2-d]pyrimidin-4-amine (compound 323) (Scheme 9)

In a 50-mL flask were mixed 4-chloro-11-(isobutoxymethyl)-8,8-dimethyl-7,10-dihydro-8H-pyrano[3″,4″:5′,6′]pyrido[3′,2′:4,5]thieno[3,2-d]pyrimidine (4) (109 mg, 0.28 mmol, 1 equiv), 2-(1H-benzo[d]imidazol-5-yl)ethan-1-amine dihydrochloride (5) (85 mg, 0.36 mmol, 1.3 equiv) and TEA (0.25 mL, 1.68 mmol, 6 equiv) in 25 mL of i-PrOH. The reaction mixture was refluxed for 18 h. The mixture was poured into 300 mL of water and extracted with a mixture t-BuOH/CHCl₃ 1/1 (3×60 mL). Organics were combined, dried over Na₂SO₄, filtered, coated on SiO₂ and subjected to CC (eluent: DCM/MeOH=7%). Fractions with the product were collected and concentrated to V˜10 mL when the product started to precipitate. The cloudy solution was quickly filtered from mechanical impurities and put to a fridge (+4° C.) for overnight. The precipitate formed was filtered off and dried to afford the target product (compound 323) as a beige solid (58 mg, 40%).

### Synthetic Details for Compound 330

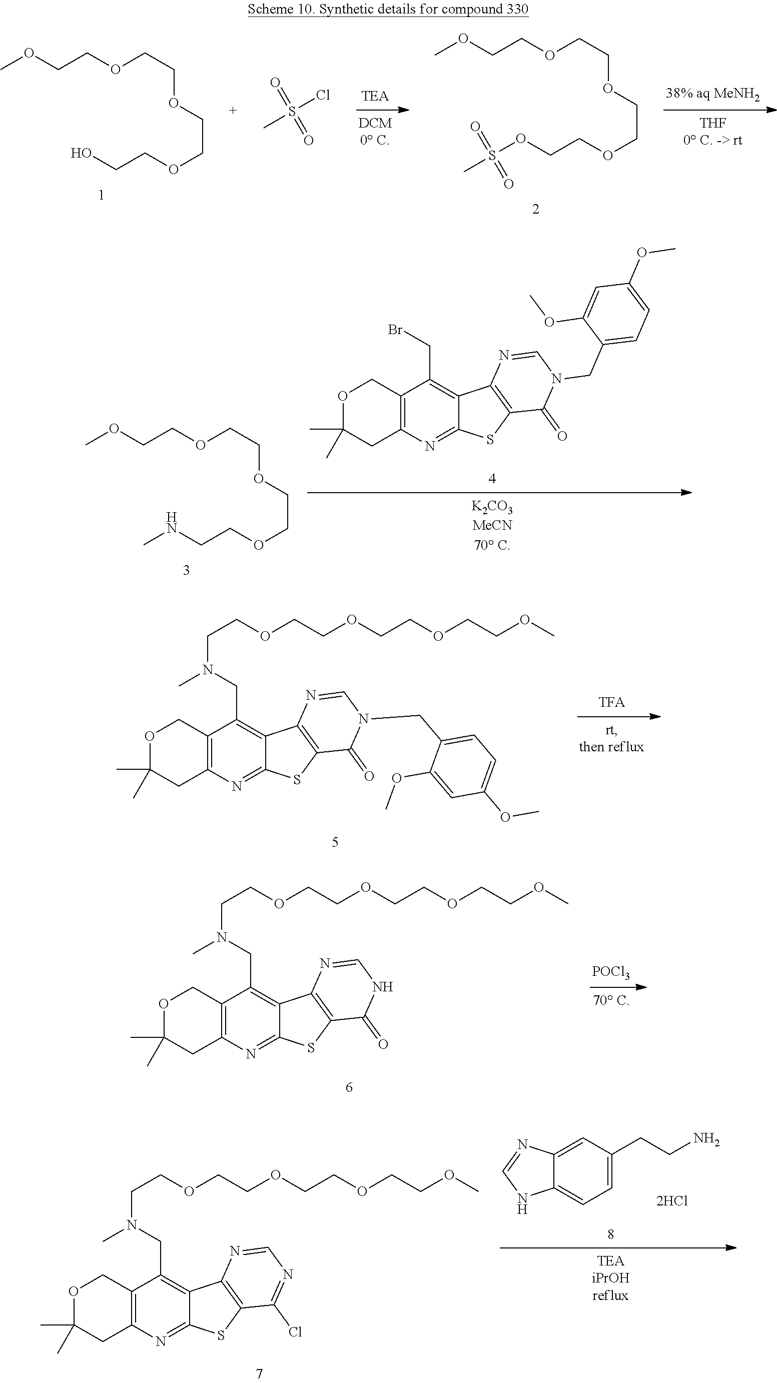

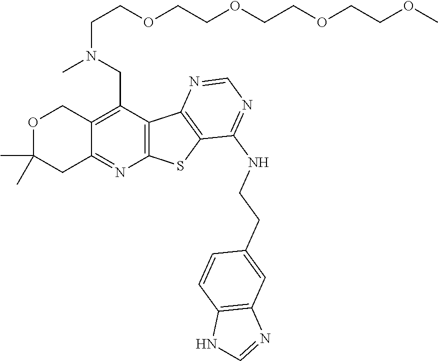

### 2,5,8,11-Tetraoxatridecan-13-yl Methanesulfonate (2) (Scheme 10)

A mixture of 2,5,8,11-tetraoxatridecan-13-ol (1) (1 g, 4.8 mmol, 1 equiv) and TEA (0.9 mL, 6.2 mmol, 1.3 equiv) in 10 mL of dry DCM was cooled to 0° C. under Ar. A solution of methanesulfonyl chloride (0.56 mL, 7.2 mmol, 1.5 equiv) in dry DCM (5 mL) was added dropwise and the reaction mixture was allowed to warm to rt and stirred at rt for 30 min. A solution was poured into 1N HCl (300 mL) and resultant mixture was transferred to a separatory funnel. More DCM (150 mL) was added and organic layer was washed with water (3×15 mL), dried over Na₂SO₄, filtered and concentrated to afford crude target product (2) as a yellow oil (1.94 g, >100%).

### N-Methyl-2,5,8,11-tetraoxatridecan-13-amine (3) (Scheme 10)

2,5,8,11-Tetraoxatridecan-13-yl methanesulfonate (2) (crude 1 g, 3.49 mmol, 1 equiv) was dissolved in 30 mL of THF and cooled to 0° C. Methylamine (2 mL, 38% aq solution) was added under cooling and reaction mixture was stirred for 48 h gradually warming to rt. More methylamine (5 mL, 38% aq solution) was added and the mixture was stirred at rt to further 3 days. A solution was poured into water (200 mL) and extracted with DCM (5×100 mL). Organics were combined, dried over Na₂SO₄, filtered, coated on SiO₂ and subjected to CC (eluent: DCM saturated with 10% v/v NH₃+8% v/v MeOH). Fractions with the product were collected and concentrated to afford the target product (3) as a yellow oil (490 mg, 46% after 2 stages).

### 3-(2,4-Dimethoxybenzyl)-8,8-dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (5) (Scheme 10)

A mixture of N-methyl-2,5,8,11-tetraoxatridecan-13-amine (3) (417 mg, 1.89 mmol, 5 equiv) and K₂CO₃ (52 mg, 0.38 mmol, 1 equiv) in 20 mL of MeCN was heated to 70° C. and 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (200 mg, 0.38 mmol, 1 equiv) was added in small portions to the hot mixture during 1 h. Upon addition completed, the reaction mixture was stirred for 10 more minutes, filtered and concentrated to afford crude target product (5) as a yellow oil (253 mg, 100%).

### 8,8-Dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (6) (Scheme 10)

Crude 3-(2,4-dimethoxybenzyl)-8,8-dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (5) (253 mg, 0.38 mmol) was mixed with trifluoroacetic acid (5 mL) and reaction mixture was stirred first at rt for 3 days, then at 35° C. for 8 h, then refluxed for 12 h. Magenta reaction mixture was concentrated, mixed with 10 mL of toluene and coated on SiO₂, then subjected to CC (eluent: DCM saturated with 10% v/v NH₃+2%→4% v/v MeOH). Fractions with the product were collected and concentrated to afford the target product (6) as a reddish oil (172 mg, 88% after 2 stages).

### N-((4-Chloro-8,8-dimethyl-7,10-dihydro-8H-pyrano[3″,4″:5′,6′]pyrido[3′,2′:4,5]thieno[3,2-d]pyrimidin-11-yl)methyl)-N-methyl-2,5,8,11-tetraoxatridecan-13-amine (7) (Scheme 10)

8,8-Dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (6) (172 mg, 0.33 mmol) was suspended in 5 mL of POCl₃. The resultant mixture was heated at 70° C. for 20 h. Reaction mixture was twice mixed with 10 mL of toluene and twice concentrated in vacuo. The residue was dissolved in 10 mL of MeOH, coated on SiO₂ and subjected to CC (eluent:MeOH in DCM: 1.5%→2.5%). Fractions with the product were collected and concentrated to afford the target product (7) as a light-brown oil (27 mg, 16%) which solidifies upon staying.

### N-(2-(1H-Benzo[d]imidazol-5-yl)ethyl)-8,8-dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4-amine (compound 330) (Scheme 10)

A suspension of N-((4-chloro-8,8-dimethyl-7,10-dihydro-8H-pyrano[3″,4″:5′,6′]pyrido[3′,2′:4,5]thieno[3,2-d]pyrimidin-11-yl)methyl)-N-methyl-2,5,8,11-tetraoxatridecan-13-amine (7) (27 mg, 0.05 mmol, 1 equiv), 2-(1H-benzo[d]imidazol-5-yl)ethan-1-amine dihydrochloride (8) (18 mg, 0.075 mmol, 1.5 equiv) and TEA (0.04 mL, 0.3 mmol, 6 equiv) in 25 mL of i-PrOH was refluxed for 10 h. The reaction mixture was concentrated to V˜5-7 mL and poured into 150 mL of cold water. Aqueous layer was extracted with DCM (6×50 mL). Organics were combined, dried over Na₂SO₄, filtered, coated on SiO₂ and subjected to CC (eluent:MeOH in DCM: 6%→50%). Fractions with the product were collected, partially concentrated and subjected to PTLC on SiO₂ plates (1500 m) using a mixture DCM saturated with 10% v/v NH₃+6% v/v MeOH as an eluent. The solid product obtained was triturated with pentane. No filtration was applied since the product started to blur on the filter. Pentane was removed with a pipette from the vial and the solid was dried in a vial in a vacuum oven at 40° C. to afford the target product (compound 330) (9 mg, 27%) as a yellowish solid.

### Synthetic Details for Compound 331

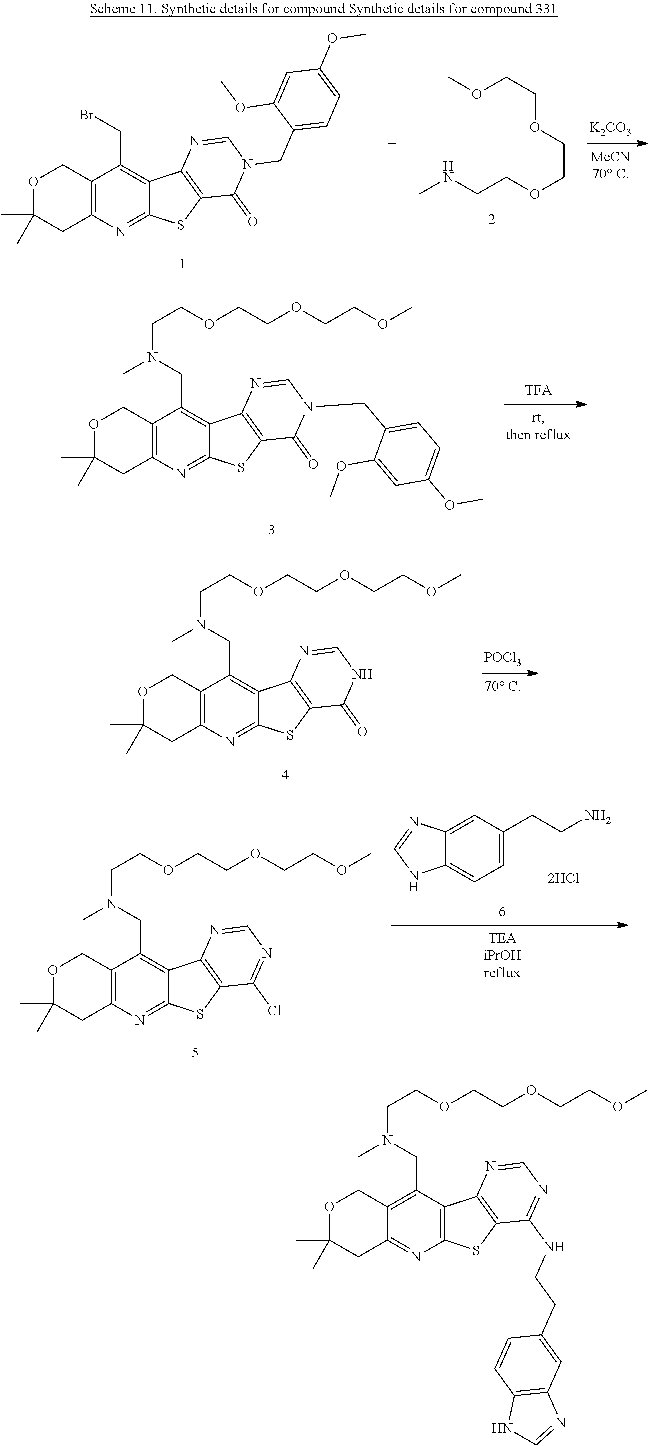

### 3-(2,4-Dimethoxybenzyl)-8,8-dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (3) (Scheme 11)

A mixture of 2-(2-(2-methoxyethoxy)ethoxy)-N-methylethan-1-amine (2) (334 mg, 1.89 mmol, 5 equiv) and K₂CO₃ (52 mg, 0.38 mmol, 1 equiv) in 20 mL of MeCN was heated to 70° C. and 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (1) (200 mg, 0.38 mmol, 1 equiv) was added in small portions to the hot mixture during 1 h. Upon addition completed, the reaction mixture was stirred for 10 more minutes, filtered and concentrated to afford crude target product (3) as a yellow oil (236 mg, 100%).

### 8,8-Dimethyl-11-(2-methyl-5,8,11-trioxa-2-azadodecyl)-7,10-dihydro-8H-pyrano[3″,4″:5′,6′]pyrido[3′,2′:4,5]thieno[3,2-d]pyrimidin-4(3H)-one (4) (Scheme 11)

Crude 3-(2,4-dimethoxybenzyl)-8,8-dimethyl-11-(2-methyl-5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one (3) (236 mg, 0.38 mmol) was mixed with trifluoroacetic acid (5 mL) and reaction mixture was stirred first at rt for 3 days, then at 35° C. for 8 h, then refluxed for 12 h. Magenta reaction mixture was concentrated, mixed with 10 mL of toluene and coated on SiO₂, then subjected to CC (eluent: DCM saturated with 10% v/v NH₃+2%→4% v/v MeOH). Fractions with the product were collected and concentrated to afford the target product (4) as a brown oil (160 mg, 88% after 2 stages).

### N-((4-Chloro-8,8-dimethyl-7,10-dihydro-8H-pyrano[3″,4″:5′,6′]pyrido[3′,2′:4,5]thieno[3,2-d]pyrimidin-11-yl)methyl)-2-(2-(2-methoxyethoxy)ethoxy)-N-methylethan-1-amine (5) (Scheme 11)

8,8-Dimethyl-11-(2-methyl-5,8,11-trioxa-2-azadodecyl)-7,10-dihydro-8H-pyrano[3″,4″:5′,6′]pyrido[3′,2′:4,5]thieno[3,2-d]pyrimidin-4(3H)-one (4) (160 mg, 0.34 mmol) was suspended in 5 mL of POCl₃. The resultant mixture was heated at 70° C. for 20 h. Reaction mixture was twice mixed with 10 mL of toluene and twice concentrated in vacuo. The residue was dissolved in 10 mL of MeOH, coated on SiO₂ and subjected to CC (eluent:MeOH in DCM: 1.5%→2.5%). Fractions with the product were collected and concentrated to afford the target product (5) as a yellow oil (40 mg, 24%) which solidifies upon staying.

### N-(2-(1H-Benzo[d]imidazol-5-yl)ethyl)-8,8-dimethyl-11-(2-methyl-5,8,11-trioxa-2-azadodecyl)-7,10-dihydro-8H-pyrano[3″,4″:5′,6′]pyrido[3′,2′:4,5]thieno[3,2-d]pyrimidin-4-amine (compound 331) (Scheme 11)

A suspension of N-((4-chloro-8,8-dimethyl-7,10-dihydro-8H-pyrano[3″,4″:5′,6′]pyrido[3′,2′:4,5]thieno[3,2-d]pyrimidin-11-yl)methyl)-2-(2-(2-methoxyethoxy)ethoxy)-N-methylethan-1-amine (5) (30 mg, 0.061 mmol, 1 equiv), 2-(1H-benzo[d]imidazol-5-yl)ethan-1-amine dihydrochloride (6) (21 mg, 0.091 mmol, 1.5 equiv) and TEA (0.05 mL, 0.37 mmol, 6 equiv) in 25 mL of i-PrOH was refluxed for 26 h. The reaction mixture was concentrated to V˜5-7 mL and poured into 150 mL of cold water. Aqueous layer was extracted with DCM (6×50 mL). Organics were combined, dried over Na₂SO₄, filtered, coated on SiO₂ and subjected to CC (eluent:MeOH in DCM: 10%→30%). Fractions with the product were collected, partially concentrated and subjected to PTLC on SiO₂ plates (1500 m) using a mixture DCM saturated with 10% v/v NH₃+5% v/v MeOH as an eluent (the general procedure A for PTLC is as described for Compound 334). The solid product obtained was triturated with pentane. No filtration was applied since the product started to blur on the filter. Pentane was removed with a pipette from the vial and the solid was dried in a vial in a vacuum oven at 40° C. to afford the target product (compound 331) (14 mg, 37%) as a yellowish solid.

### Synthetic Details for Compound 309

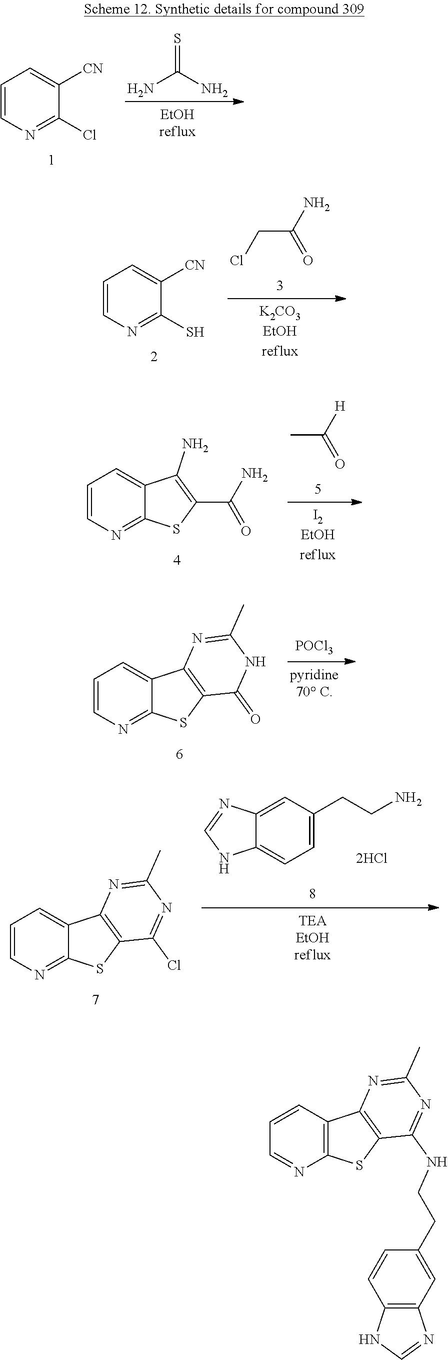

### 2-Mercaptonicotinonitrile (2) (Scheme 12)

A mixture of 2-chloronicotinonitrile (1) (15 g, 108.3 mmol, 1 equiv) and thiourea (9.88 g, 130 mmol, 1.2 equiv) was refluxed in 150 mL of EtOH for 3.5 h. Reaction mixture was cooled to rt and mixed with 1N NaOH (150 mL), water (200 mL) and brine (50 mL). Aqueous layer was washed with EA (2×60 mL) and acidified with 6N HCl to pH 3-4. The precipitate formed was filtered, thoroughly washed with water and dried to afford the target product (2) as a yellow solid (12.1 g, 82%).

### 3-Aminothieno[2,3-b]pyridine-2-carboxamide (4) (Scheme 12)

A mixture of 2-mercaptonicotinonitrile (2) (12 g, 90.2 mmol, 1 equiv), 2-chloroacetamide (3) (8.44 g, 90.2 mmol, 1 equiv) and K₂CO₃ (25.26 g, 180.5 mmol, 2 equiv) was refluxed in 250 mL of EtOH for 12 h. Reaction mixture was hot-filtered from K₂CO₃ and put to a fridge (−18° C.) for overnight. The precipitate formed was filtered, washed with water, cold EtOH, cold MTBE and dried to afford the target product (4) as a yellow solid (12.6 g, 72%).

### 2-Methylpyrido[3′,2′:4,5]thieno[3,2-d]pyrimidin-4(3H)-one (6) (Scheme 12)

A mixture of 3-aminothieno[2,3-b]pyridine-2-carboxamide (4) (483 mg, 2.5 mmol, 1 equiv), acetaldehyde (5) (220 mg, 5 mmol, 2 equiv) and iodine (762 mg, 3 mmol, 1.2 equiv) was refluxed in 10 mL of EtOH for 6 h. Reaction mixture was cooled to rt and carefully poured into a preliminary prepared solution of K₂CO₃ (0.5 g) and Na₂S₂O₃*5H₂O (1 g) in 50 mL of water. The precipitate formed was filtered, washed with water, cold EtOH, cold acetone and dried to afford the target product (6) as a beige solid (490 mg, 90%).

### 4-Chloro-2-methylpyrido[3′,2′:4,5]thieno[3,2-d]pyrimidine (7) (Scheme 12)

2-Methylpyrido[3′,2′:4,5]thieno[3,2-d]pyrimidin-4(3H)-one (6) (217 mg, 1 mmol) was suspended in 5 mL of POCl₃ and 0.2 mL of pyridine. The resultant mixture was heated at 70° C. for 4 h, then carefully and slowly poured into 150 mL of ice-cold water. The precipitate formed was filtered, washed with water and PE, dried at 65° C. to afford crude target product (7) as an orange-brown solid (77 mg, 33%).

### N-(2-(1H-Benzo[d]imidazol-5-yl)ethyl)-2-methylpyrido[3′,2′:4,5]thieno[3,2-d]pyrimidin-4-amine (compound 309) (Scheme 12)

In a 50-mL flask were mixed 4-chloro-2-methylpyrido[3′,2′:4,5]thieno[3,2-d]pyrimidine (7) (crude 74 mg, 0.31 mmol, 1 equiv), 2-(1H-benzo[d]imidazol-5-yl)ethan-1-amine dihydrochloride (8) (147 mg, 0.63 mmol, 2 equiv) and TEA (0.25 mL, 1.86 mmol, 6 equiv) in 10 mL of EtOH. The reaction mixture was refluxed for 8 h, then poured into 150 mL of water and left in a fridge for overnight. The precipitate formed was collected by filtration, dissolved in hot i-PrOH, coated on SiO₂ and subjected to CC (eluent:MeOH in DCM=2%→10%). Fractions with the product were collected and concentrated. The residue was triturated with EA, filtered and dried at 80° C. in a vacuum oven for 10 h to afford the target product (compound 309) as a peach-coloured solid (73 mg, 65%).

### Synthetic Details for Compound 307

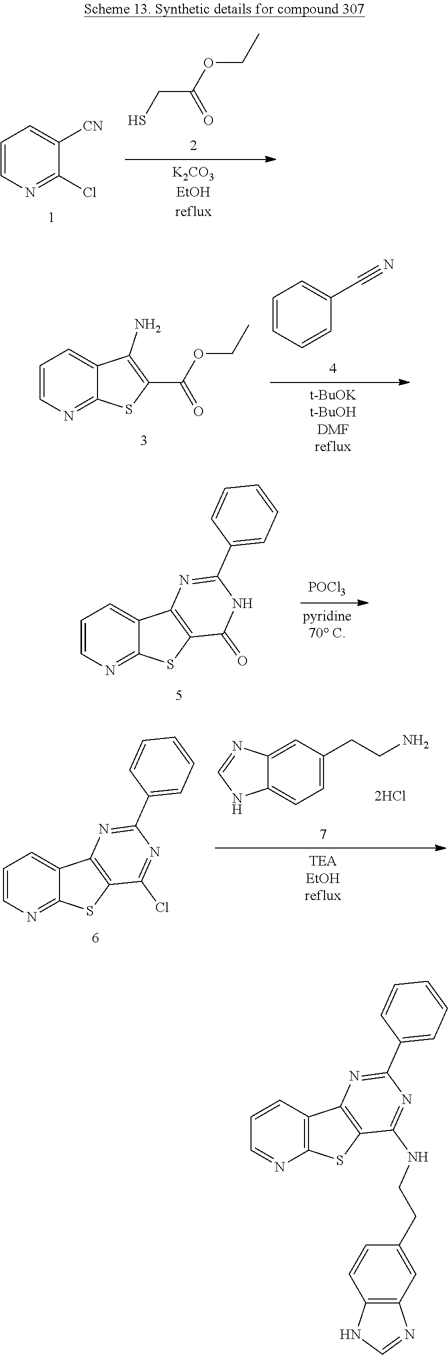

### Ethyl 3-aminothieno[2,3-b]pyridine-2-carboxylate (3) (Scheme 13)

A mixture of 2-chloronicotinonitrile (1) (6.93 g, 50 mmol, 1 equiv), ethyl 2-mercaptoacetate (2) (6 g, 50 mmol, 1 equiv) and K₂CO₃ (11.1 g, 50 mmol, 1 equiv) was refluxed in 50 mL of EtOH under Ar for 48 h. Reaction mixture was hot-filtered from K₂CO₃ and put to a fridge (−18° C.) for overnight. The precipitate formed was filtered, washed with water and recrystallized from EtOH to afford the target product (3) as a yellow solid (4.15 g, 37%).

### 2-Phenylpyrido[3′,2′:4,5]thieno[3,2-d]pyrimidin-4(3H)-one (5) (Scheme 13)

A mixture of ethyl 3-aminothieno[2,3-b]pyridine-2-carboxylate (3) (500 mg, 2.25 mmol, 1 equiv), benzonitrile (4) (257 mg, 2.5 mmol, 1.11 equiv) and t-BuOK (270 mg, 2.41 mmol, 1.07 equiv) was refluxed under Ar in a mixture of abs t-BuOH (5 mL) and dry DMF (4 mL) for 3 h. The mixture was concentrated in vacuo, mixed with 10 mL of MeOH and poured into cold 5% aq NH₄Cl (100 mL). The precipitate formed was filtered, washed with water, hot EtOH and dried to afford the target product (5) as an orange solid (280 mg, 46%).

### 4-Chloro-2-methylpyrido[3′,2′:4,5]thieno[3,2-d]pyrimidine (6) (Scheme 13)

2-Phenylpyrido[3′,2′:4,5]thieno[3,2-d]pyrimidin-4(3H)-one (5) (280 mg, 1 mmol) was suspended in 5 mL of POCl₃ and 0.2 mL of pyridine. The resultant mixture was heated at 70° C. for 4 h, then carefully and slowly poured into 150 mL of ice-cold water. The precipitate formed was filtered, washed with water and dried at 65° C. to afford crude target product (6) as a grey solid (157 mg, 52%).

### N-(2-(1H-Benzo[d]imidazol-5-yl)ethyl)-2-phenylpyrido[3′,2′:4,5]thieno[3,2-d]pyrimidin-4-amine (compound 307) (Scheme 13)

In a 50-mL flask were mixed 4-chloro-2-methylpyrido[3′,2′:4,5]thieno[3,2-d]pyrimidine (6) (crude 71 mg, 0.24 mmol, 1 equiv), 2-(1H-benzo[d]imidazol-5-yl)ethan-1-amine dihydrochloride (7) (112 mg, 0.48 mmol, 2 equiv) and TEA (0.2 mL, 1.46 mmol, 6 equiv) in 50 mL of EtOH. The reaction mixture was refluxed for 24 h, partially concentrated to V˜5-7 mL and poured into 100 mL of water. The solid precipitated was filtered off, washed off the filter with hot i-PrOH and alcoholic mother liquid was coated on SiO₂. Product was isolated via CC (eluent:MeOH in DCM=2%→6%). Fractions with the product were collected and concentrated. The residue was fully dissolved in boiling EtOH (˜50 mL)+DMF (5 mL), hot-filtered from mechanical impurities and again concentrated in vacuo. The residue was triturated with hot EA, filtered and dried at 90° C. in a vacuum oven for 10 h to afford the target product (compound 307) as an off-white solid (18 mg, 18%).

### Synthetic Details for Compound 312

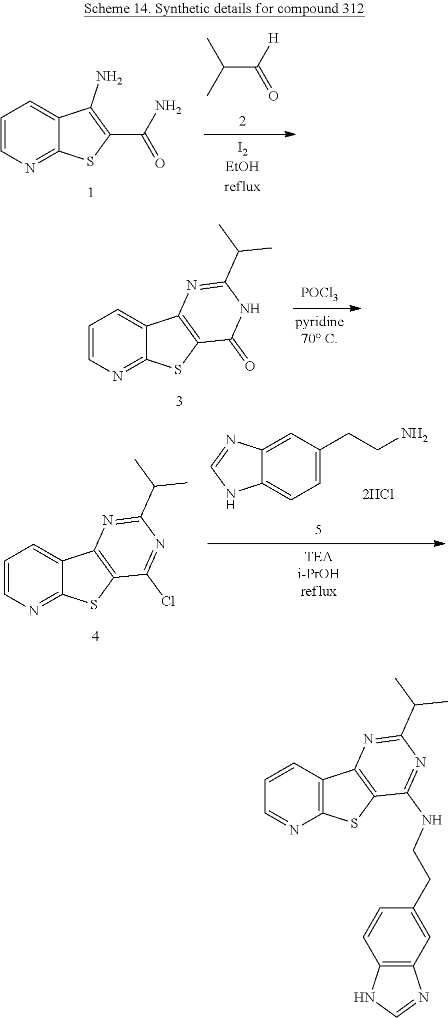

### 2-Isopropylpyrido[3′,2′:4,5]thieno[3,2-d]pyrimidin-4(3H)-one (3) (Scheme 14)

A mixture of 3-aminothieno[2,3-b]pyridine-2-carboxamide (1) (483 mg, 2.5 mmol, 1 equiv), isobutyraldehyde (2) (360 mg, 5 mmol, 2 equiv) and iodine (762 mg, 3 mmol, 1.2 equiv) was refluxed in 10 mL of EtOH for 6 h. Reaction mixture was cooled to rt and carefully poured into a preliminary prepared solution of K₂CO₃ (1 g) and Na₂S₂O₃*5H₂O (1 g) in 50 mL of water. The precipitate formed was filtered, washed with water, cold EtOH, cold MTBE and dried to afford the target product (3) as a colourless solid (300 mg, 49%).

### 4-Chloro-2-isopropylpyrido[3′,2′:4,5]thieno[3,2-d]pyrimidine (4) (Scheme 14)

2-Isopropylpyrido[3′,2′:4,5]thieno[3,2-d]pyrimidin-4(3H)-one (3) (200 mg, 0.82 mmol) was suspended in 5 mL of POCl₃ and 0.2 mL of pyridine. The resultant mixture was heated at 70° C. for 4 h, then carefully and slowly poured into 150 mL of ice-cold water. The precipitate formed was filtered, washed with water and PE, dried at 65° C. to afford crude target product (4) as an off-white solid (139 mg, 64%).

### N-(2-(1H-Benzo[d]imidazol-5-yl)ethyl)-2-isopropylpyrido[3′,2′:4,5]thieno[3,2-d]pyrimidin-4-amine (compound 312) (Scheme 14)

In a 50-mL flask were mixed 4-chloro-2-isopropylpyrido[3′,2′:4,5]thieno[3,2-d]pyrimidine (4) (crude 100 mg, 0.38 mmol, 1 equiv), 2-(1H-benzo[d]imidazol-5-yl)ethan-1-amine dihydrochloride (5) (177 mg, 0.76 mmol, 2 equiv) and TEA (0.3 mL, 2.27 mmol, 6 equiv) in 25 mL of i-PrOH. The reaction mixture was refluxed for 16 h, partially concentrated to V˜5-7 mL and poured into 150 mL of water. The solid precipitated was filtered off, washed off the filter with hot i-PrOH and alcoholic mother liquid was coated on SiO₂. Product was isolated via CC (eluent:MeOH in DCM=3%→9%). Fractions with the product were collected and concentrated. The residue was triturated with hot EA, filtered and dried at 90° C. in a vacuum oven for 10 h to afford the target product (compound 312) as a white solid (73 mg, 50%).

### Synthetic Details for Compound 316

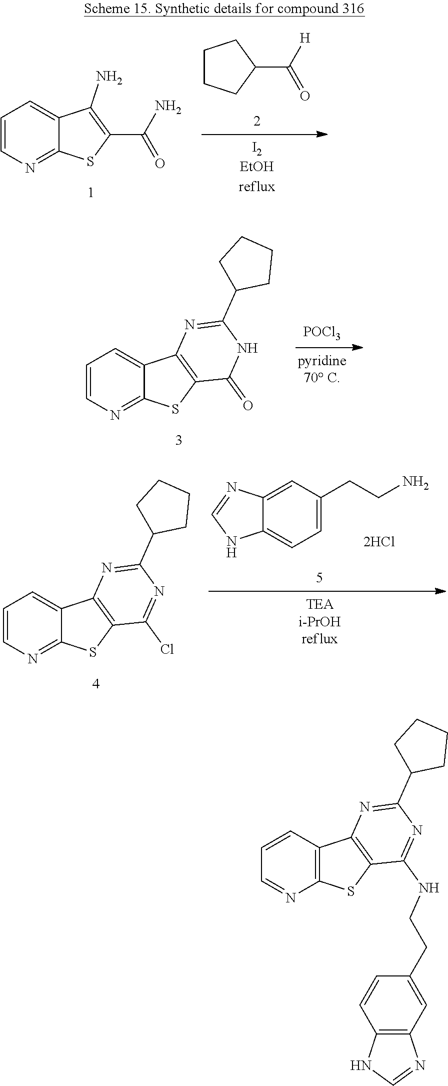

### 2-Cyclopentylpyrido[3′,2′:4,5]thieno[3,2-d]pyrimidin-4(3H)-one (3) (Scheme 15)

A mixture of 3-aminothieno[2,3-b]pyridine-2-carboxamide (1) (483 mg, 2.5 mmol, 1 equiv), cyclopentanecarbaldehyde (2) (490 mg, 5 mmol, 2 equiv) and iodine (762 mg, 3 mmol, 1.2 equiv) was refluxed in 10 mL of EtOH for 6 h. Reaction mixture was cooled to rt and carefully poured into a preliminary prepared solution of K₂CO₃ (1.5 g) and Na₂S₂O₃*5H₂O (3 g) in 50 mL of water. The precipitate formed was filtered, washed with water, cold EtOH, cold MTBE and dried to afford the target product (3) as a grey solid (625 mg, 92%).

### 4-Chloro-2-cyclopentylpyrido[3′,2′:4,5]thieno[3,2-d]pyrimidine (4) (Scheme 15)

2-Cyclopentylpyrido[3′,2′:4,5]thieno[3,2-d]pyrimidin-4(3H)-one (3) (300 mg, 1.11 mmol) was suspended in 5 mL of POCl₃ and 0.2 mL of pyridine. The resultant mixture was heated at 70° C. for 8 h, then carefully and slowly poured into 150 mL of ice-cold water. The precipitate formed was filtered and washed with water. In order to get rid of water, the product on the filter was washed with PE. But it turned out that the product partially dissolved in PE and could be washed off the filter with PE, all dirty impurities being left undissolved on a filter. Mother liquid containing the product was diluted with EA, dried over Na₂SO₄, filtered and concentrated to afford pure target product (4) as an off-white solid (90 mg, 28%).

### N-(2-(1H-Benzo[d]imidazol-5-yl)ethyl)-2-cyclopentylpyrido[3′,2′:4,5]thieno[3,2-d]pyrimidin-4-amine (compound 316) (Scheme 15)

In a 50-mL flask were mixed 4-chloro-2-cyclopentylpyrido[3′,2′:4,5]thieno[3,2-d]pyrimidine (4) (85 mg, 0.29 mmol, 1 equiv), 2-(1H-benzo[d]imidazol-5-yl)ethan-1-amine dihydrochloride (5) (137 mg, 0.59 mmol, 2 equiv) and TEA (0.25 mL, 1.76 mmol, 6 equiv) in 15 mL of i-PrOH. The reaction mixture was refluxed for 16 h, partially concentrated to V˜5-7 mL and poured into 150 mL of water. Aqueous layer was extracted with a mixture t-BuOH/CHCl₃ 1/1 (3×30 mL). Organics were combined, dried over Na₂SO₄, filtered, coated on SiO₂ and subjected to CC (eluent:MeOH in DCM: 4%→7%). Fractions with the product were collected and concentrated. The residue was dissolved in boiling i-PrOH, hot-filtered and again concentrated. The new residue was triturated with hot EA, filtered and dried at 70° C. in a vacuum oven for 10 h to afford the target product (compound 316) as a white solid (83 mg, 69%).

### Synthetic Details for Compound 310

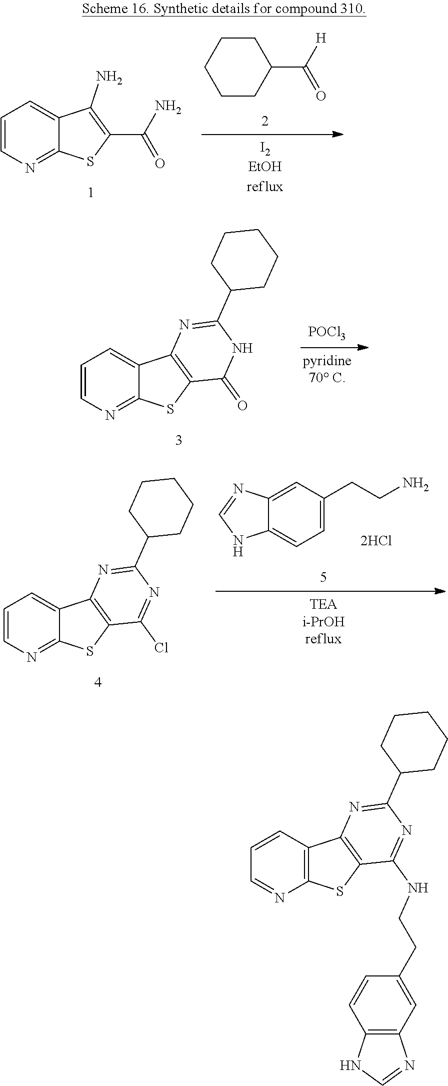

### 2-Cyclohexylpyrido[3′,2′:4,5]thieno[3,2-d]pyrimidin-4(3H)-one (3) (Scheme 16)

A mixture of 3-aminothieno[2,3-b]pyridine-2-carboxamide (1) (483 mg, 2.5 mmol, 1 equiv), cyclohexanecarbaldehyde (2) (560 mg, 5 mmol, 2 equiv) and iodine (762 mg, 3 mmol, 1.2 equiv) was refluxed in 10 mL of EtOH for 5 h. Reaction mixture was cooled to rt and carefully poured into a preliminary prepared solution of K₂CO₃ (0.5 g) and Na₂S₂O₃*5H₂O (1 g) in 50 mL of water. The precipitate formed was filtered, washed with water, cold EtOH and recrystallized from EtOH to afford the target product (3) as a colourless solid (415 mg, 58%).

### 4-Chloro-2-cyclohexylpyrido[3′,2′:4,5]thieno[3,2-d]pyrimidine (4) (Scheme 16)

2-Cyclohexylpyrido[3′,2′:4,5]thieno[3,2-d]pyrimidin-4(3H)-one (3) (150 mg, 0.53 mmol) was suspended in 5 mL of POCl₃ and 0.2 mL of pyridine. The resultant mixture was heated at 70° C. for 2 h, then carefully and slowly poured into 150 mL of ice-cold water. The precipitate formed was filtered, washed with water and dried to afford crude target product (4) as an off-white solid (120 mg, 80%).

### N-(2-(1H-Benzo[d]imidazol-5-yl)ethyl)-2-cyclohexylpyrido[3′,2′:4,5]thieno[3,2-d]pyrimidin-4-amine (compound 310) (Scheme 16)

In a 50-mL flask were mixed 4-chloro-2-cyclohexylpyrido[3′,2′:4,5]thieno[3,2-d]pyrimidine (4) (80 mg, 0.26 mmol, 1 equiv), 2-(1H-benzo[d]imidazol-5-yl)ethan-1-amine dihydrochloride (5) (123 mg, 0.52 mmol, 2 equiv) and TEA (0.25 mL, 1.76 mmol, 6 equiv) in 25 mL of i-PrOH. The reaction mixture was refluxed for 10 h, partially concentrated to V˜5-7 mL and poured into 150 mL of water. The solid precipitated was filtered off, washed off the filter with hot i-PrOH and alcoholic mother liquid was coated on SiO₂. Product was isolated via CC (eluent:MeOH in DCM=3%→9%). Fractions with the product were collected and concentrated. The residue was triturated with EA, filtered and dried at 70° C. in a vacuum oven for 10 h to afford the target product (compound 310) as a white solid (78 mg, 70%).

General Procedure for the Synthesis of Amides According to this Invention (Amide 15 in FIG. 1B) Illustrated on an Exemplary Synthesis of Compound 317:

### Ethyl 3-cyano-7,7-dimethyl-2-thioxo-2,5,7,8-tetrahydro-1H-pyrano[4,3-b]pyridine-4-carboxylate (2, R═CO₂Et, FIG. 1B, 1C)

Sodium (2.53 g, 110 mmol, 1.1 equiv) was dissolved in 200 mL of EtOH, freshly distilled over CaH₂. 2,2-Dimethyldihydro-2H-pyran-4(3H)-one (1) (12.8 g, 100 mmol, 1 equiv) was added to the prepared EtONa/EtOH solution and the mixture was stirred at rt for 10 min under Ar atmosphere. Diethyl oxalate (14.6 g, 100 mmol, 1 equiv) was added and the reaction mixture was stirred at rt overnight. 2-Cyanoethanethioamide (10 g, 100 mmol, 1 equiv) was added and the reaction mixture was stirred at rt overnight, then poured into 1 L of 10% aq AcOH and stirred for further 30 min. The solid formed was filtered-off, washed with water (2×100 mL) and Et₂O (100 mL+50 mL) and dried to afford the target product as a brown solid (18.35 g, 63%).

### Ethyl 3-amino-2-carbamoyl-7,7-dimethyl-7,8-dihydro-5H-pyrano[4,3-b]thieno[3,2-e]pyridine-4-carboxylate (3, R═CO₂Et, R¹═NH₂, FIG. 1B)

Ethyl 3-cyano-7,7-dimethyl-2-thioxo-2,5,7,8-tetrahydro-1H-pyrano[4,3-b]pyridine-4-carboxylate (2, R═CO₂Et) (18.3 g, 62.7 mmol, 1 equiv), 2-chloroacetamide (5.9 g, 62.7 mmol, 1 equiv) and K₂CO₃ (17.3 g, 125.3 mmol, 2 equiv) were suspended in 350 mL of EtOH. The reaction mixture was refluxed under Ar atmosphere for 1 h, and while hot was filtered from K₂CO₃. The filtrate was cooled to 8° C., the solid precipitated was filtered off, washed with EtOH (2*100 mL) and dried at 45° C. to afford the target product as a yellow solid (11.6 g). The mother liquid was concentrated to V˜150 mL and cooled to 8° C. The solid precipitated was filtered off, washed with EtOH (2*50 mL), then Et₂O (10 mL) and dried at 45° C. to afford another portion of the target product (5 g). Total yield of the reaction product as a yellow solid was 16.6 g (76%).

### Ethyl 8,8-dimethyl-4-oxo-4,7,8,10-tetrahydro-3H-pyrano[3″,4″:5′,6′]pyrido-[3′,2′:4,5]thieno[3,2-d]pyrimidine-11-carboxylate (4, R═CO₂Et, FIG. 1B)

Into a 1 L flask were placed ethyl 3-amino-2-carbamoyl-7,7-dimethyl-7,8-dihydro-5H-pyrano[4,3-b]thieno[3,2-e]pyridine-4-carboxylate (3, R═CO₂Et, R¹═NH₂, FIG. 1B) (9.4 g, 26.9 mmol) and 350 mL of triethyl orthoformate. The reaction mixture was refluxed under Ar for 24 h, then cooled to rt. The solid formed was filtered-off, washed with EtOH (30 mL) and MTBE (30 mL) and dried to afford the target product as a yellow solid (6.4 g, 66%).

### Ethyl 4-chloro-8,8-dimethyl-8,10-dihydro-7H-pyrano[3″,4″:5′,6′]pyrido[3′,2′:4,5]-thieno[3,2-d]pyrimidine-11-carboxylate (5, R═CO₂Et, FIG. 1B)

In a 50-mL flask were mixed ethyl 8,8-dimethyl-4-oxo-4,7,8,10-tetrahydro-3H-pyrano[3″,4″:5′,6′]pyrido-[3′,2′:4,5]thieno[3,2-d]pyrimidine-11-carboxylate (4, R═CO₂Et, FIG. 1B) (460 mg, 1.28 mmol, 1 equiv), 12 mL of POCl₃ and 1 mL of pyridine. The reaction mixture was heated at 65-70° C. for 2 h, cooled to rt and carefully poured into 500 mL of cold H₂O. The resultant mixture was put into a fridge (+4° C.) and the precipitate formed was filtered off and dried to afford the target product as a tan solid (280 mg, 75%).

### Ethyl 4-((2-(1H-benzo[d]imidazol-5-yl)ethyl)amino)-8,8-dimethyl-8,10-dihydro-7H-pyrano[3″,4″:5′,6′]pyrido[3′,2′:4,5]thieno[3,2-d]pyrimidine-11-carboxylate (170, FIG. 1B)

In a 10-mL flask were mixed ethyl 4-chloro-8,8-dimethyl-8,10-dihydro-7H-pyrano[3″,4″:5′,6′]pyrido[3′,2′:4,5]-thieno[3,2-d]pyrimidine-11-carboxylate (5, R═CO₂Et, FIG. 1B) (280 mg, 0.74 mmol, 1 equiv), 2-(1H-benzo[d]imidazol-5-yl)ethan-1-amine dihydrochloride (260 mg, 1.11 mmol, 1.5 equiv) and TEA (0.5 mL, 3.7 mmol, 5 equiv) in 10 mL of EtOH. The reaction mixture was refluxed for 24 h, then coated on SiO₂ and subjected to CC (eluent: DCM/MeOH=93/7). Fractions with the product were collected, concentrated and the residue was recrystallized from EtOH to afford the target product as a tan solid (273 mg, 74%).

### 4-((2-(1H-Benzo[d]imidazol-6-yl)ethyl)amino)-8,8-dimethyl-8,10-dihydro-7H-pyrano[3″,4″:5′,6′]pyrido[3′,2′:4,5]thieno[3,2-d]pyrimidine-11-carboxylic Acid (171, FIG. 1B)

Lithium hydroxide monohydrate (29 mg, 0.7 mmol, 10 equiv) was dissolved in 1.5 mL of water and added to the preliminary prepared suspension of ethyl 4-((2-(1H-benzo[d]imidazol-5-yl)ethyl)amino)-8,8-dimethyl-8,10-dihydro-7H-pyrano-[3″,4″:5′,6′]pyrido[3′,2′:4,5]thieno[3,2-d]pyrimidine-11-carboxylate (170) (35 mg, 0.07 mmol, 1 equiv) in 3.5 mL of EtOH. The resultant heterogeneous mixture was refluxed for 1 h while it was gradually becoming homogeneous. After cooling to rt, the reaction mixture was adjusted to pH 6 with 1N HCl and the precipitate formed was filtered off, washed with water and recrystallized from EtOH to afford the target product (Compound 171, FIG. 1B) as a white solid (11 mg, 33%).

### Synthetic Details for Compound 317

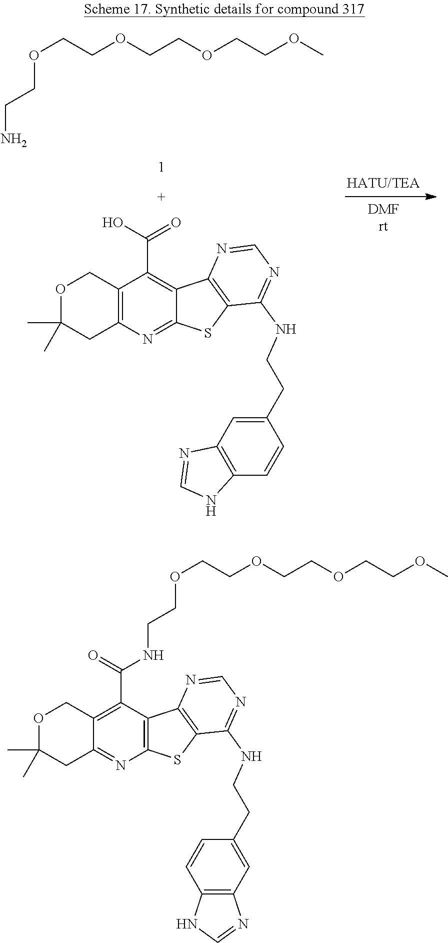

### 4-((2-(1H-Benzo[d]imidazol-5-yl)ethyl)amino)-8,8-dimethyl-N-(2,5,8,11-tetraoxatridecan-13-yl)-7,10-dihydro-8H-pyrano[3″,4″:5′,6′]pyrido[3′,2′:4,5]thieno[3,2-d]pyrimidine-11-carboxamide (compound 317) (Scheme 17)

A mixture of 4-((2-(1H-benzo[d]imidazol-5-yl)ethyl)amino)-8,8-dimethyl-7,10-dihydro-8H-pyrano[3″,4″:5′,6′]pyrido[3′,2′:4,5]thieno[3,2-d]pyrimidine-11-carboxylic acid (Compound 171) (113 mg, 0.24 mmol, 1 equiv), 2,5,8,11-tetraoxatridecan-13-amine (1) (99 mg, 0.48 mmol, 2 equiv), HATU (317 mg, 0.83 mmol, 3.5 equiv) and TEA (0.2 mL, 1.19 mmol, 5 equiv) in 3 mL of dry DMF was stirred at rt for 16 h. The reaction mixture was concentrated in vacuo, mixed with 10 mL of MeOH and poured into 150 mL of water. Aqueous layer was extracted with a mixture t-BuOH/CHCl₃ 1/1 (3×35 mL). Organics were combined, dried over Na₂SO₄, filtered, coated on SiO₂ and subjected to CC (eluent:MeOH in DCM: 3%→7%). Fractions with the product were collected and concentrated. The residue was recrystallized from a mixture EA+MeOH to afford the target product (compound 317) as a white solid (5 mg, 3%).

General Procedure for the Synthesis of Intermediates (10, FIG. 1C) and their Transformations to Target Compounds Illustrated on the Exemplary Synthesis of Compound 308:

### Ethyl 3-amino-2-((2,4-dimethoxybenzyl)carbamoyl)-7,7-dimethyl-7,8-dihydro-5H-pyrano[4,3-b]thieno[3,2-e]pyridine-4-carboxylate (6, FIG. 1C)

Ethyl 3-cyano-7,7-dimethyl-2-thioxo-1,5,7,8-tetrahydro-2H-pyrano[4,3-b]pyridine-4-carboxylate (2, R═CO₂Et, FIG. 1C) (16.8 g, 57.4 mmol, 1.05 equiv), 2-chloro-N-(2,4-dimethoxybenzyl)acetamide (13.3 g, 54.7 mmol, 1 equiv) and K₂CO₃ (15.1 g, 109.4 mmol, 2 equiv) were suspended in 600 mL of absolute EtOH. The reaction mixture was refluxed for 45 min. Inorganic salts were filtered off, washed with EtOH and discarded. Clear reaction mixture was coated on SiO₂ and subjected to CC (eluent: DCM/MeOH=1%→2%). Fractions with the product were collected and concentrated to afford the target product as a red oil (23.2 g, 85%).

### 3-Amino-N-(2,4-dimethoxybenzyl)-4-(hydroxymethyl)-7,7-dimethyl-7,8-dihydro-5H-pyrano[4,3-b]thieno[3,2-e]pyridine-2-carboxamide (7, FIG. 1C)

Ethyl 3-amino-2-((2,4-dimethoxybenzyl)carbamoyl)-7,7-dimethyl-7,8-dihydro-5H-pyrano[4,3-b]thieno[3,2-e]pyridine-4-carboxylate (6, FIG. 1C) (23.2 g, 46.4 mmol, 1 equiv) was dissolved in a mixture THF/MeOH 1/1 (100 mL) and to this solution in small portions under vigorous stirring was added LiBH₄ (3.04 g, 139.3 mmol, 3 equiv). After the reaction mixture was stirred at rt for 2 h, it was poured into 1 L of brine. Aqueous layer was extracted with EA (4×250 mL). Organics were combined, dried over Na₂SO₄, filtered, coated on SiO₂ and subjected to CC (eluent: DCM/MeOH=1%→2%). Fractions with the product were collected and concentrated to afford the target product as a yellow solid (11.75 g, 56%).

### 3-(2,4-Dimethoxybenzyl)-11-(hydroxymethyl)-8,8-dimethyl-7,10-dihydro-8H-pyrano[3″,4″:5′,6′]pyrido[3′,2′:4,5]thieno[3,2-d]pyrimidin-4(3H)-one (8, FIG. 1C)

A mixture of 3-amino-N-(2,4-dimethoxybenzyl)-4-(hydroxymethyl)-7,7-dimethyl-7,8-dihydro-5H-pyrano[4,3-b]thieno[3,2-e]pyridine-2-carboxamide (7, FIG. 1C) (11.5 g, 25.1 mmol, 1 equiv) and 100 mL of formamide (11.5 g, 25.1 mmol, 1 equiv) was refluxed under Ar for 1 h, then cooled to −100° C. and poured into 1 L of H₂O. The precipitate formed was filtered off, washed with water and dissolved in DCM under stirring. Water excess was separated and organic layer was dried over MgSO₄. The reaction mixture was filtered, coated on SiO₂ and subjected to CC (eluent: first, pure DCM, then DCM/MeOH=1%). Fractions with the product were collected, concentrated and mixed with 100 mL of EtOH. After refluxing in ethanol for 10 min and cooling to −18° C., the precipitate formed was filtered-off, washed with cold EtOH (50 mL), PE (100 mL) and dried on air to afford the target product as a colourless solid (9.55 g, 82%).

### 11-(Bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (9, FIG. 1C)

3-(2,4-Dimethoxybenzyl)-11-(hydroxymethyl)-8,8-dimethyl-7,10-dihydro-8H-pyrano[3″,4″:5′,6′]pyrido[3′,2′:4,5]thieno[3,2-d]pyrimidin-4(3H)-one (8, FIG. 1C) (0.95 g, 2.03 mmol, 1 equiv) and tetrabromomethane (1.01 g, 3.05 mmol, 1.5 equiv) were dissolved in 25 mL of dry DCM. The mixture was cooled to 0-2° C. and a solution of triphenylphosphine (1.06 g, 4.06 mmol, 2 equiv) in 10 mL of dry DCM was added dropwise slowly. The reaction mixture was stirred at rt for 3 days, starting alcohol (8) did not react completely. The solution was coated on SiO₂ and subjected to CC (eluent: DCM/MeOH=1%→3%). Fractions with the product were collected, concentrated and recrystallized from EtOH to afford the target product as a white solid (0.85 g, 79%).

### 3-(2,4-Dimethoxybenzyl)-11-(methoxymethyl)-8,8-dimethyl-7,10-dihydro-8H-pyrano[3″,4″:5′,6′]pyrido[3′,2′:4,5]thieno[3,2-d]pyrimidin-4(3H)-one (10, R⁵═OMe, FIG. 1C)

Sodium metal (0.11 g, 4.98 mmol, 2 equiv) was dissolved in absolute MeOH (50 mL) under Ar. Solid 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (9, FIG. 1C) (1.32 g, 2.49 mmol, 1 equiv) was added in portions to the solution of above prepared NaOMe in MeOH, followed by dry THF (30 mL). The reaction mixture was heated at 50° C. for 2 h. Solvents were removed in vacuo, the residue was partioned between water (50 mL) and CHCl₃ (20 mL). Aqueous layer was extracted with CHCl₃ (2×50 mL). Organics were combined, dried over Na₂SO₄, filtered and concentrated to afford the target product as a beige powder (1.18 g, 98%).

### 11-(Methoxymethyl)-8,8-dimethyl-7,10-dihydro-8H-pyrano[3″,4″:5′,6′]pyrido[3′,2′:4,5]thieno[3,2-d]pyrimidin-4(3H)-one (4, R═CH₂OMe, FIG. 1C)

3-(2,4-Dimethoxybenzyl)-11-(methoxymethyl)-8,8-dimethyl-7,10-dihydro-8H-pyrano[3″,4″:5′,6′]pyrido[3′,2′:4,5]thieno[3,2-d]pyrimidin-4(3H)-one (10, R⁵═OMe, FIG. 1C) (1.18 g, 2.45 mmol) was dissolved in 10 mL of trifluoroacetic acid and the reaction mixture was left for overnight, gradually turning magenta. All solids from the walls of the flask were washed with MeCN and the reaction mixture was concentrated in vacuo. The residue was mixed with 5-7 mL of MeCN and poured into 200 mL of water. Water layer was basified with 30% aq NaOH to pH 6-7. Solid formed was filtered off, washed with water and dried in a vacuum oven at 70° C. to afford the target product as a white solid (1.04 g). Mother liquid was extracted with a mixture CHCl₃/t-BuOH 1/1 (13×50 mL). Organics were combined, dried over Na₂SO₄, filtered, coated on SiO₂ and subjected to CC (eluent: DCM/MeOH=2%→5%). Fractions with the product were collected and concentrated to afford additional amount of the target product as a beige powder (95 mg). Total yield was 1.135 g (>100%).

### 4-Chloro-11-(methoxymethyl)-8,8-dimethyl-7,10-dihydro-8H-pyrano[3″,4″:5′,6′]pyrido[3′,2′:4,5]thieno[3,2-d]pyrimidine (5, R═CH₂OMe, FIG. 1C)

To a suspension of crude 11-(methoxymethyl)-8,8-dimethyl-7,10-dihydro-8H-pyrano[3″,4″:5′,6′]pyrido[3′,2′:4,5]thieno[3,2-d]pyrimidin-4(3H)-one (4, R═CH₂OMe, FIG. 1C) (1.135 g, 3.4 mmol) in 150 mL of MeCN were added 20 mL of POCl₃ and 2 mL of pyridine. The resultant suspension was refluxed for 12 h, gradually turning brown. Though solids did not dissolve completely, after 12 h of reflux starting pyrimidin-4(3H)-one (4, R═CH₂OMe) fully disappeared. Reaction mixture was concentrated to V˜10-15 mL and poured into ice-cold water (300 mL). The precipitate formed was filtered-off, rinsed with water and dried in a vacuum oven at 70° C. to afford the target product as a beige solid (1.06 g, >100% relative to starting 10, R⁵═OMe (FIG. 1C).

### N-(2-(1H-Benzo[d]imidazol-5-yl)ethyl)-11-(methoxymethyl)-8,8-dimethyl-7,10-dihydro-8H-pyrano[3″,4″:5′,6′]pyrido[3′,2′:4,5]thieno[3,2-d]pyrimidin-4-amine (Compound 308)

A suspension of crude 4-chloro-11-(methoxymethyl)-8,8-dimethyl-7,10-dihydro-8H-pyrano[3″,4″:5′,6′]pyrido[3′,2′:4,5]thieno[3,2-d]pyrimidine (5, R═CH₂OMe, FIG. 1C) (1.06 g, 3.03 mmol, 1 equiv), 2-(1H-benzo[d]imidazol-5-yl)ethan-1-amine dihydrochloride (0.922 g, 3.94 mmol, 1.3 equiv) and TEA (2.54 mL, 18.18 mmol, 6 equiv) in 150 mL of i-PrOH was refluxed for 20 h. Since starting (5, R═CH₂OMe, FIG. 1C) was present in the reaction mixture, more amine (0.184 g, 0.79 mmol, 0.2 equiv) was added and reflux was continued for the next 8 h. Chloride (5, R═CH₂OMe, FIG. 1C) disappeared completely, the reaction was heterogeneous. The suspension was concentrated to V˜15-20 mL and poured into ice-cold water (400 mL). The precipitate formed was filtered-off and thoroughly washed with water. Precipitate was suspended in 150 mL of EtOH and stirred under reflux for 30 min, then quickly hot-filtered. Precipitate left on a filter was discarded since it was not the target product but some impurity that did not dissolve even in hot DMF or DMSO. Mother liquid, contained the product, was coated on SiO₂ and subjected to CC (eluent: DCM/MeOH=3%→4%). Fractions with the product were filtered from mechanical impurities, collected together and concentrated to V˜20-30 mL when the product started to precipitate in the flask upon evaporation of solvents. The flask with suspension was left in a fridge (+4° C.) for overnight. The precipitate formed was filtered off, rinsed with cold MeOH and dried in a vacuum oven at 100° C. to afford the target product (Compound 308) as a white solid (543 mg, 47% after 3 stages).

General Procedure for the Synthesis of Esters (14, R═CO₂(CH₂CH₂O)ₙ-Me, Scheme 16a) Illustrated on Exemplary Synthesis of Compound 165 (FIG. 1B)

### 8,8-Dimethyl-4-oxo-4,7,8,10-tetrahydro-3H-pyrano[3″,4″:5′,6′]pyrido[3′,2′:4,5]thieno[3,2-d]pyrimidine-11-carboxylic Acid (13, FIG. 1B)

Sodium hydroxide (1.4 g, 35 mmol, 5 equiv) was dissolved in 30 mL of water and added to the preliminary prepared suspension of ethyl 8,8-dimethyl-4-oxo-4,7,8,10-tetrahydro-3H-pyrano[3″,4″:5′,6′]pyrido-[3′,2′:4,5]thieno[3,2-d]pyrimidine-11-carboxylate (4, R═CO₂Et, FIG. 1B) (2.51 g, 7 mmol, 1 equiv) in 30 mL of MeOH. The resultant heterogeneous mixture was stirred at 35° C. for 48 h while it was gradually becoming homogeneous. After cooling to rt, the reaction mixture was adjusted to pH 6 with 1N HCl and the precipitate formed was filtered off, washed with water and dried to afford compound (13, FIG. 1B) as a yellowish solid (1.57 g, 68%).

### 2-(2-(2-Methoxyethoxy)ethoxy)ethyl 8,8-dimethyl-4-oxo-4,7,8,10-tetrahydro-3H-pyrano[3″,4″:5′,6′]pyrido[3′,2′:4,5]thieno[3,2-d]pyrimidine-11-carboxylate (4, R═CO₂(CH₂CH₂O)₃-Me, FIG. 1B)

8,8-Dimethyl-4-oxo-4,7,8,10-tetrahydro-3H-pyrano[3″,4″:5′,6′]pyrido[3′,2′:4,5]thieno[3,2-d]pyrimidine-11-carboxylic acid (13, FIG. 1B) (331 mg, 1 mmol, 1 equiv), Ph₃P (315 mg, 1.2 mmol, 1.2 equiv), DIAD (243 mg, 1.2 mmol, 1.2 equiv) and 2-(2-(2-methoxyethoxy)ethoxy)ethanol (164 mg, 1 mmol, 1 equiv) were suspended in 15 mL of THF. The reaction mixture was stirred at rt for 2 h, then refluxed for 30 min. The mixture was concentrated and subjected to CC (eluent: DCM/MeOH=97/3). Fractions with the product were collected, concentrated and the residue was washed with Et₂O to afford the target product as a colorless solid (285 mg, 40%).

### 2-(2-(2-Methoxyethoxy)ethoxy)ethyl 4-chloro-8,8-dimethyl-8,10-dihydro-7H-pyrano[3″,4″:5′,6′]pyrido[3′,2′:4,5]thieno[3,2-d]pyrimidine-11-carboxylate (5, R═CO₂(CH₂CH₂O)₃-Me, FIG. 1B)

In a 10-mL flask were mixed 2-(2-(2-methoxyethoxy)ethoxy)ethyl 8,8-dimethyl-4-oxo-4,7,8,10-tetrahydro-3H-pyrano[3″,4″:5′,6′]pyrido[3′,2′:4,5]thieno[3,2-d]pyrimidine-11-carboxylate (4, R═CO₂(CH₂CH₂O)₃-Me, FIG. 1B) (275 mg, 0.58 mmol, 1 equiv), 3 mL of POCl₃ and 0.3 mL of pyridine. The reaction mixture was heated at 70° C. for 2 h, then at 85° C. for 1 h, cooled to rt and carefully poured into 100 mL of cold H₂O. The resultant mixture was put into a fridge (+4° C.) and the precipitate formed was filtered off and dried to afford the target product (5, FIG. 1B) as a pinkish solid (53 mg, 19%).

### 2-(2-(2-Methoxyethoxy)ethoxy)ethyl 4-((2-(1H-benzo[d]imidazol-6-yl)ethyl)amino)-8,8-dimethyl-8,10-dihydro-7H-pyrano[3″,4″:5′,6′]pyrido[3′,2′:4,5]thieno[3,2-d]pyrimidine-11-carboxylate (Compound 165, FIG. 1B)

In a 10-mL flask were mixed 2-(2-(2-methoxyethoxy)ethoxy)ethyl 4-chloro-8,8-dimethyl-8,10-dihydro-7H-pyrano[3″,4″:5′,6′]pyrido[3′,2′:4,5]thieno[3,2-d]pyrimidine-11-carboxylate (5, R═CO₂(CH₂CH₂O)₃-Me, FIG. 1B) (53 mg, 0.107 mmol, 1 equiv), 2-(1H-benzo[d]imidazol-5-yl)ethan-1-amine dihydrochloride (37 mg, 0.16 mmol, 1.5 equiv) and TEA (0.07 mL, 0.535 mmol, 5 equiv) in 5 mL of EtOH. The reaction mixture was refluxed for 4 h, then partioned between water (50 mL) and EtOAc (25 mL). The aqueous phase was extracted with EtOAc (3*20 mL), and the organics were combined, dried over Na₂SO₄, filtered, partially concentrated and subjected to preparative TLC (eluent: DCM/MeOH=9/1) on SiO₂-plates. The collected product was triturated with pentane to afford the target product (Compound 165, (14), FIG. 1B) as an off-white solid (23 mg, 35%).

### General Procedure for the Synthesis of Amides (16, FIG. 1C) Illustrated on Exemplary Synthesis of Compound 321

### N-((3-(2,4-Dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (10, R⁵═CH₂—N(COCF₃)((CH₂CH₂O)₄-Me, FIG. 1C)

A mixture of 2,5,8,11-tetraoxatridecan-13-amine (440 mg, 2.12 mmol, 3.2 equiv) and K₂CO₃ (91 mg, 0.66 mmol, 1 equiv) in 10 mL of MeCN was heated to 70° C. and 11-(bromomethyl)-3-(2,4-dimethoxybenzyl)-8,8-dimethyl-7,10-dihydro-8H-pyrano[3″,4″:5′,6′]pyrido[3′,2′:4,5]thieno[3,2-d]pyrimidin-4(3H)-one (9) (350 mg, 0.66 mmol, 1 equiv) was added in small portions to the hot mixture during 1 h. Upon addition completed, the reaction mixture was stirred for 10 more minutes, coated on SiO₂ and subjected to CC (eluent:MeOH in DCM: 3%→13%). Fractions with the product were collected and concentrated to afford intermediate 3-(2,4-dimethoxybenzyl)-8,8-dimethyl-11-(5,8,11,14-tetraoxa-2-azapentadecyl)-7,10-dihydro-8H-pyrano[3″,4″:5′,6′]pyrido[3′,2′:4,5]thieno[3,2-d]pyrimidin-4(3H)-one as a yellow oil (390 mg, 90%). Intermediate obtained (390 mg, 0.594 mmol) was mixed with trifluoroacetic anhydride (TFAA) (5 mL) and reaction mixture was stirred at rt for overnight. The mixture was concentrated and re-evaporated twice with toluene to afford crude target product as a brown oil (447 mg) which was used in the next stage without any purification.

### N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (4, R⁵═CH₂—N(COCF₃)((CH₂CH₂O)₄-Me, FIG. 1C)

Crude N-((3-(2,4-dimethoxybenzyl)-8,8-dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (10, R⁵═CH₂—N(COCF₃)((CH₂CH₂O)₄-Me), FIG. 1C) (447 mg, 0.59 mmol) was mixed with trifluoroacetic acid (5 mL) and reaction mixture was stirred at rt for overnight. Magenta reaction mixture was concentrated, mixed with 5 mL of MeCN and poured into aq phosphate buffer (pH 7, 100 mL). The precipitate formed was filtered off, washed with water and dried in a vacuum oven to afford crude product as a dark-brown solid (155 mg, 44%).

### N-((4-Chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (5, R⁵═CH₂—N(COCF₃)((CH₂CH₂O)₄-Me, FIG. 1C)

N-((8,8-Dimethyl-4-oxo-3,4,7,10-tetrahydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (4, R⁵═CH₂—N(COCF₃)((CH₂CH₂O)₄-Me), FIG. 1C) (crude 155 mg, 0.257 mmol) was suspended in 3 mL of POCl₃ and 0.3 mL of pyridine. The resultant mixture was heated at 70° C. for 3 h, then carefully and slowly poured into 200 mL of ice-cold water. Aqueous fraction was extracted with a mixture t-BuOH/CHCl₃ 1/1 (4×30 mL). Organics were combined, dried over Na₂SO₄, filtered and concentrated to afford crude target product as a dark-brown solid (146 mg, 90%).

### N-(2-(1H-Benzo[d]imidazol-5-yl)ethyl)-8,8-dimethyl-11-(5,8,11,14-tetraoxa-2-azapentadecyl)-7,10-dihydro-8H-pyrano[3″,4″:5′,6′]pyrido[3′,2′:4,5]thieno[3,2-d]pyrimidin-4-amine (Compound 321, FIG. 1C)

A suspension of N-((4-chloro-8,8-dimethyl-7,10-dihydro-8H-pyrano[3″,4″:5′,6′]pyrido[3′,2′:4,5]thieno[3,2-d]pyrimidin-11-yl)methyl)-2,2,2-trifluoro-N-(2,5,8,11-tetraoxatridecan-13-yl)acetamide (5, R⁵═CH₂—N(COCF₃)(CH₂CH₂O)₄-Me, FIG. 1C) (crude 146 mg, 0.234 mmol, 1 equiv), 2-(1H-benzo[d]imidazol-5-yl)ethan-1-amine dihydrochloride (66 mg, 0.281 mmol, 1.2 equiv) and TEA (0.2 mL, 1.4 mmol, 6 equiv) in 25 mL of i-PrOH was refluxed for 8 h. TLC showed full consumption of chloride (5, R⁵═CH₂—N(COCF₃)(CH₂CH₂O)₄-Me, FIG. 1C) and formation of the reaction product—intermediate (14, R⁵═CH₂—N(COCF₃)((CH₂CH₂O)₄-Me), FIG. 1C) which was not isolated in a pure form but subjected to deprotection in situ. K₂CO₃ (48 mg, 0.351 mmol, 1.5 equiv) and water (5 mL) were added and the reaction mixture was refluxed for further 3.5 h, then poured into 200 mL of water. Aqueous layer was extracted with a mixture t-BuOH/CHCl₃ 1/1 (3×30 mL). Organics were combined, dried over Na₂SO₄, filtered, coated on SiO₂ and subjected to CC (eluent:MeOH in DCM: 25%). Fractions with the product were collected, partially concentrated and subjected to PTLC on SiO₂ plates (1500 m) using a mixture EA/MeOH+4.5 vv % aq NH₃ as an eluent. The solid product obtained was triturated with EA+Et₂O to afford the target product (Compound 321, 16, FIG. 1C) (13 mg, 3% after 4 stages) as a beige solid.

### Synthetic Details for Compound 325

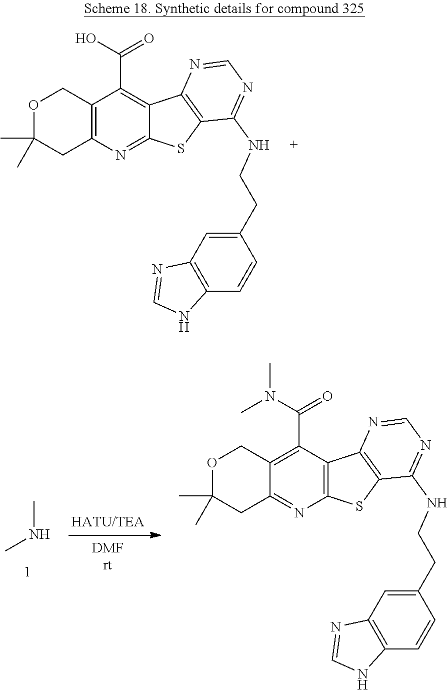

### 4-((2-(1H-Benzo[d]imidazol-5-yl)ethyl)amino)-8,8-dimethyl-N-(2,5,8,11-tetraoxatridecan-13-yl)-7,10-dihydro-8H-pyrano[3″,4″:5′,6′]pyrido[3′,2′:4,5]thieno[3,2-d]pyrimidine-11-carboxamide (Compound 325)

A mixture of 4-((2-(1H-benzo[d]imidazol-5-yl)ethyl)amino)-8,8-dimethyl-7,10-dihydro-8H-pyrano[3″,4″:5′,6′]pyrido[3′,2′:4,5]thieno[3,2-d]pyrimidine-11-carboxylic acid (Compound 171) (150 mg, 0.32 mmol, 1 equiv), dimethylamine (1) (12 wt % in benzene, 590 mg, 1.58 mmol, 5 equiv), HATU (300 mg, 0.79 mmol, 2.5 equiv) and TEA (0.25 mL, 1.9 mmol, 6 equiv) in 4 mL of dry DMF was stirred under Ar at rt for 16 h. The reaction mixture was concentrated in vacuo, mixed with 10 mL of MeOH and poured into 150 mL of water. Aqueous layer was extracted with a DCM (4×50 mL). Organics were combined, coated on SiO₂ and subjected to CC (eluent:MeOH in DCM: 5%→7%). Fractions with the product were collected and concentrated. The residue was recrystallized from MeOH to afford the target product (Compound 325) as a white solid (23 mg, 14.5%).

### Example 2

### Biological Activity of Compounds of the Invention

Identification of compounds that target specifically SDH-deficient cells was done by screening a library of small-molecule by using a differential toxicity screen with SDHB-proficient vs. SDHB-deficient cells (the Loss of SDH causes succinate accumulation cells were described in Cardaci 2015). Compounds that have showed specific activity against the SDHB-deficient cells were selected for further development validation and development.

### Cellular Toxicity Assay

Cells Used for the Differential Assays: SDH-WT; SDH-KO-HRAS

Cell Culture Conditions:
Medium: DMEM-F₁₂ medium+1 mM Glutamine+3% FBS
Plates: 96 well plate—Black, clear flat bottom
Incubation time: 3 days
Compound concentration: 10 point dilution starting from 10 uM to 0.0065 uM or a lower range when needed.
Viability assay at the end of incubation: Celltiter-Glo (CTG) from Promega.

Protocol:
Day 1: Plate cells in 96 well plate together with the appropriate concentration of compound.

Incubate for 3 days at 37° C. in 5% CO₂ tissue culture incubator.
Day 4: Check viability of cells by adding CTG reagent and read luminescence in plate reader

Calculate EC₅₀ using cells treated with solvent (DMSO) as 100% growth

TABLE 2
Toxicity results in two cell types.

| | EC₅₀ (uM) | |
|---|---|---|
| Com- | SDH- | SDH-KO- |
| pound No. | WT | HTAS-C17 |
| 100 | + | ++ |
| 101 | + | + |
| 102 | + | ++ |
| 103 | + | + |
| 104 | ++ | +++ |
| 105 | ++ | ++ |
| 106 | ++ | ++ |
| 107 | + | + |
| 108 | +++ | + |
| 109 | + | + |
| 110 | + | +++ |
| 111 | + | ++++ |
| 112 | + | ++ |
| 113 | ++++ | ++ |
| 114 | + | + |
| 115 | +++ | ++ |
| 116 | ++ | +++ |
| 117 | + | ++ |
| 120 | + | + |
| 121 | + | ++ |
| 122 | + | ++++ |
| 123 | + | + |
| 124 | + | + |
| 125 | + | +++ |
| 126 | ++ | +++ |
| 127 | + | + |
| 128 | ++ | ++ |
| 129 | + | ++++ |
| 130 | + | + |
| 131 | + | + |
| 132 | + | + |
| 133 | + | ++ |
| 134 | + | + |
| 135 | + | + |
| 136 | + | + |
| 137 | + | + |
| 138 | + | + |
| 140 | ++ | +++ |
| 141 | + | ++++ |
| 142 | ++ | ++++ |
| 143 | + | ++++ |
| 144 | + | ++++ |
| 145 | ++++ | ++++ |
| 146 | + | ++ |
| 147 | ++ | ++++ |
| 148 | + | ++++ |
| 149 | + | + |
| 150 | + | +++ |
| 151 | ++ | ++++ |
| 152 | ++ | ++ |
| 153 | + | + |
| 154 | ++ | ++++ |
| 156 | + | +++ |
| 157 | + | + |
| 158 | + | ++++ |
| 159 | + | + |
| 160 | + | + |
| 161 | ++ | ++ |
| 162 | ++++ | ++++ |
| 163 | + | +++ |
| 164 | ++ | ++++ |
| 165 | + | +++ |
| 166 | ++ | ++ |
| 166 | ++ | ++ |
| 167 | ++++ | ++++ |
| 168 | +++ | ++++ |
| 169 | +++ | ++++ |
| 170 | ++++ | ++++ |
| 171 | ++ | ++ |
| 172 | ++ | ++ |
| 173 | ++ | ++++ |
| 174 | ++++ | ++ |
| 175 | ++ | +++ |
| 176 | ++ | ++++ |
| 177 | ++ | +++ |
| 178 | ++ | ++++ |
| 179 | ++ | ++++ |
| 180 | ++ | ++++ |
| 181 | ++ | ++ |
| 182 | ++ | +++ |
| 183 | ++ | ++++ |
| 184 | ++ | ++ |
| 185 | ++ | +++ |
| 186 | ++ | +++ |
| 187 | +++ | ++ |
| 188 | +++ | ++ |
| 189 | +++ | ++++ |
| 190 | ++++ | +++ |
| 191 | ++ | +++ |
| 192 | ++++ | ++++ |
| 193 | +++ | ++++ |
| 194 | ++++ | ++++ |
| 195 | ++++ | ++++ |
| 196 | ++ | ++++ |
| 197 | ++ | +++ |
| 198 | ++ | ++++ |
| 199 | +++ | +++ |
| 200 | ++ | +++ |
| 201 | +++ | ++++ |
| 202 | ++ | ++++ |
| 203 | + | ++++ |
| 204 | + | +++ |
| 205 | ++ | ++++ |
| 206 | ++++ | ++++ |
| 207 | +++ | ++ |
| 208 | + | + |
| 209 | + | + |
| 210 | ++++ | ++ |
| 211 | ++ | ++ |
| 212 | ++ | ++ |
| 213 | + | +++ |
| 214 | + | +++ |
| 215 | + | + |
| 216 | + | + |
| 217 | ++ | +++ |
| 218 | + | + |
| 219 | + | ++ |
| 220 | + | + |
| 221 | + | + |
| 222 | + | + |
| 223 | + | + |
| 224 | + | + |
| 225 | + | + |
| 226 | + | + |
| 227 | + | + |
| 228 | + | + |
| 229 | + | ++ |
| 230 | + | + |
| 231 | + | + |
| 232 | + | + |
| 233 | + | + |
| 234 | + | + |
| 235 | + | + |
| 236 | + | + |
| 237 | + | ++ |
| 238 | + | + |
| 239 | + | + |
| 240 | + | ++ |
| 241 | + | + |
| 242 | + | + |
| 243 | ++ | ++ |
| 244 | + | + |
| 245 | + | + |
| 246 | + | + |
| 247 | + | ++ |
| 248 | + | + |
| 249 | + | + |
| 250 | + | + |
| 251 | + | + |
| 252 | + | + |
| 253 | + | + |
| 254 | + | + |
| 255 | + | + |
| 256 | ++ | ++ |
| 257 | + | + |
| 258 | + | + |
| 259 | + | + |
| 260 | + | + |
| 261 | + | + |
| 262 | + | + |
| 263 | + | + |
| 264 | + | ++ |
| 265 | ++ | + |
| 266 | ++ | + |
| 267 | + | + |
| 268 | + | + |
| 269 | + | + |
| 270 | + | + |
| 271 | ++ | + |
| 272 | + | + |
| 273 | + | ++ |
| 274 | + | + |
| 275 | + | + |
| 276 | ++ | + |
| 277 | + | + |
| 278 | + | + |
| 279 | + | + |
| 280 | + | + |
| 281 | + | ++ |
| 282 | + | + |
| 283 | + | + |
| 284 | + | + |
| 285 | + | + |
| 286 | + | + |
| 287 | + | + |
| 288 | + | + |
| 289 | + | ++ |
| 290 | + | + |
| 291 | + | + |
| 292 | + | + |
| 293 | + | + |
| 294 | + | + |
| 295 | + | + |
| 296 | + | ++ |
| 297 | + | ++ |
| 298 | + | + |
| 299 | + | + |
| 300 | ++ | ++ |
| 301 | + | + |
| 302 | + | + |
| 303 | + | ++++ |
| 304 | ++ | ++++ |
| 305 | + | ++++ |
| 306 | + | + |
| 307 | ++ | + |
| 308 | +++ | ++++ |
| 309 | + | ++ |
| 310 | + | + |
| 311 | + | +++ |
| 312 | ++ | ++ |
| 313 | ++ | ++ |
| 314 | ++ | ++ |
| 315 | ++ | +++ |
| 316 | ++ | ++ |
| 317 | ++ | ++ |
| 318 | ++++ | ++++ |
| 319 | +++ | ++++ |
| 320 | ++ | ++ |
| 321 | ++ | +++ |
| 322 | ++++ | ++++ |
| 323 | ++++ | ++++ |
| 324 | ++++ | ++++ |
| 325 | ++ | ++ |
| 326 | ++ | ++++ |
| 327 | ++++ | ++++ |
| 328 | ++ | ++ |
| 329 | ++++ | ++++ |
| 330 | ++ | ++++ |
| 331 | ++ | ++++ |
| 332 | +++ | ++++ |
| 333 | +++ | ++++ |
| 334 | ++ | ++++ |
| 335 | ++ | ++++ |

+ >10 uM
++ >3uM < 10 uM
+++ >1 uM < 3 uM
++++ <1 uM

### Example 3

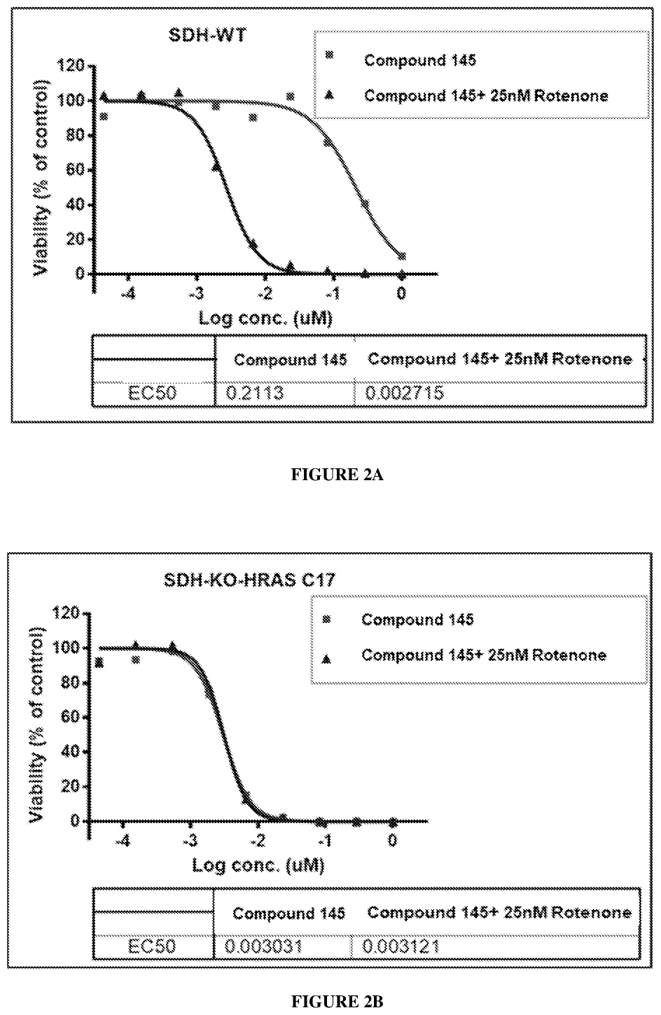

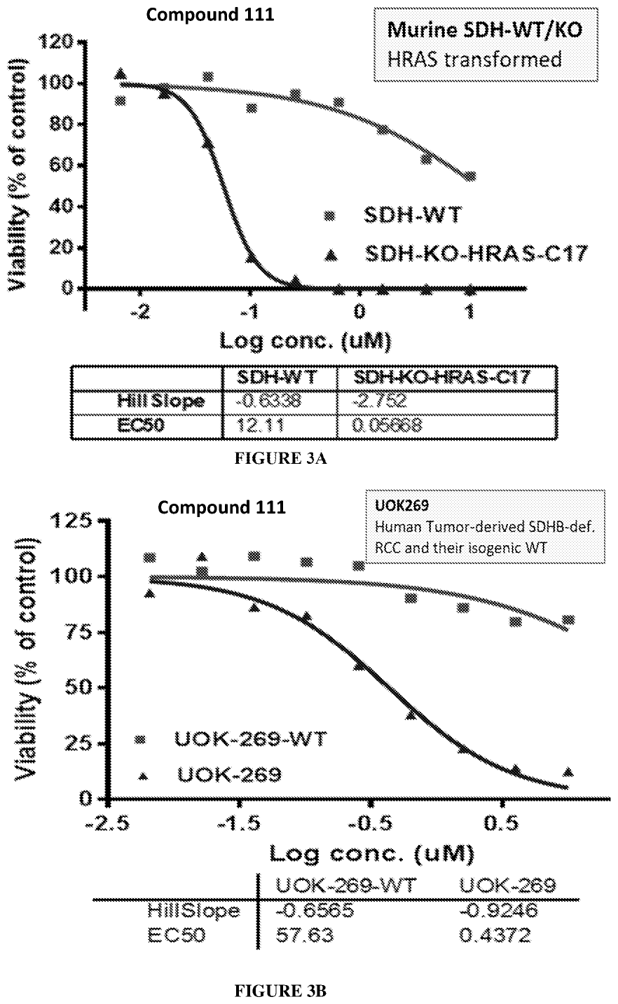

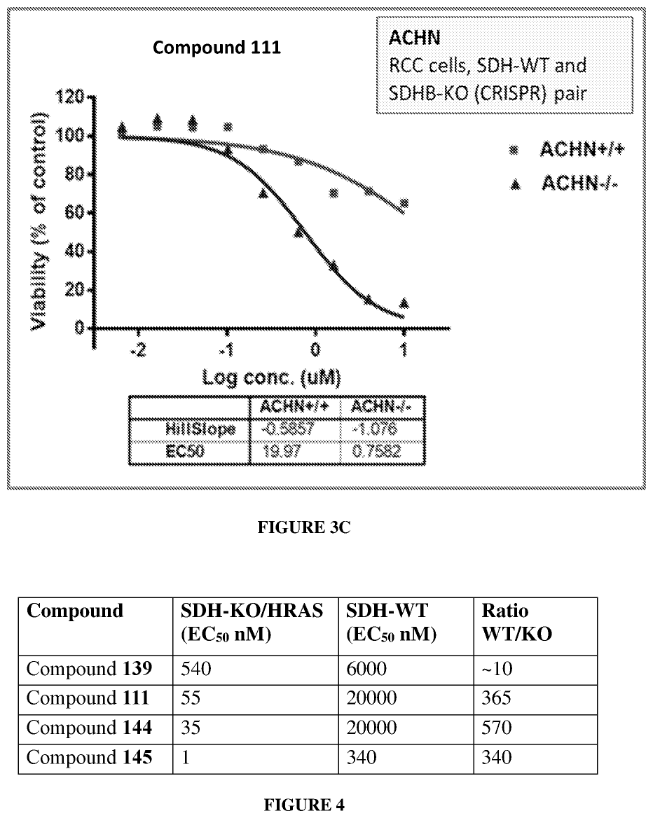

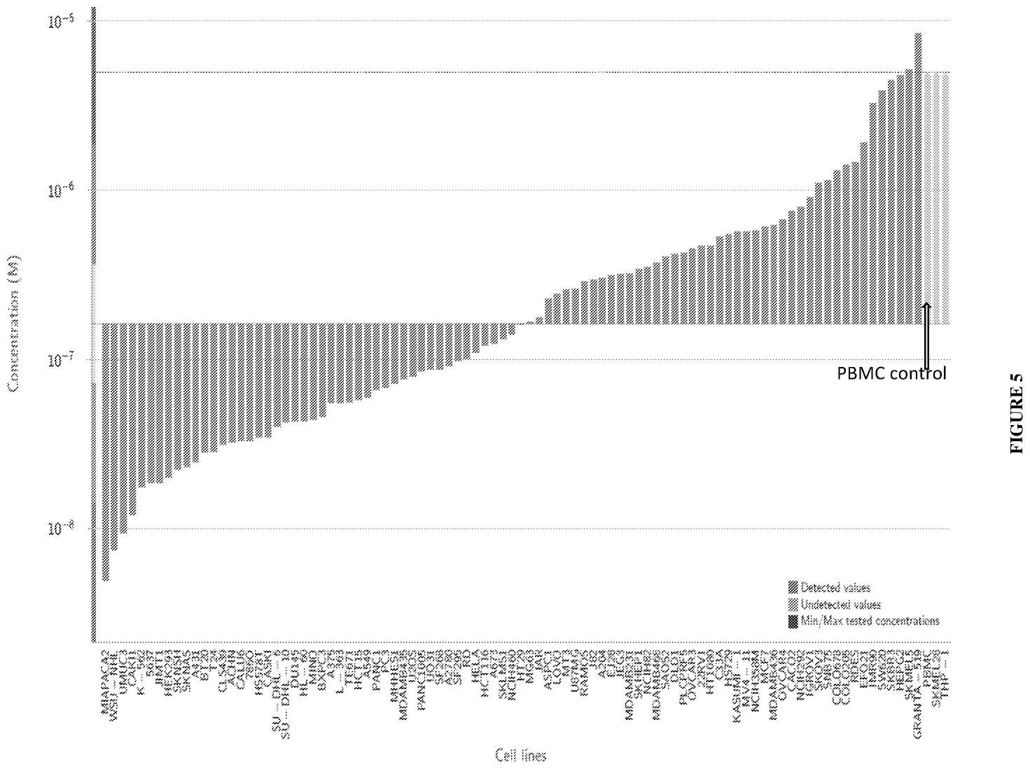

### Biological Activity for Compound 145 and Compound 111 (FIG. 2A-2B)

### A Synergistic Effect with OxPhos Inhibitor Rotenone in SDH-Proficient Cancer Cells

In order to demonstrate that compounds according to this invention have differential effect against SDH deficient cells, cells with a “broken TCA cycle”, they were combined with Rotenone, an OxPhos inhibitor. Rotenone is a chemical inhibitor of the complex I of the mitochondrial respiratory chain and as so, it leads to a “broken TCA cycle” phenotype in a similar way as SDH-deficiency.

Table 3 and Table 4 below, depict the synergistic effect for compound 145 and compound 111 respectively with rotenone in several SDH-proficient cancer cells.

Toxicity assay: Cells were growing in 96 well plates in DMEM F₁₂ with 17 mM glucose, 0.5 mM Sodium Pyruvate, 1 mM Glutamine and 3% FBS. The cells were treated with the indicated compounds (10 different concentrations) with or without 25 nM rotenone. Viability was determined after 72 hours of treatment using CellTiter Glo reagent (Promega). EC₅₀ was calculated using Prism software.

TABLE 3
Synergistic effect for compound 145 and Rotenone.

| | | Com- | Compound 145 |
|---|---|---|---|
| | | pound 145 | with Rotenone |
| Cell line | Type | EC₅₀ (nM) | EC₅₀ (nM) |
| SDH-WT | Murine HRAS-transformed | 210 | 3 |
| | cells | | |
| SDH-KO | Murine HRAS-transformed, | 3 | 3 |
| | SDHB-deficient cells | | |
| KG1 | Acute Myelogenous Leukemia | 3200 | 16 |
| THP1 | Acute Monocytic Leukemia | 1100 | 16 |
| OV-90 | Ovarian cancer | 825 | 15 |

TABLE 4
Synergistic effect for Compound 111 and Rotenone.

| | | Com- | Compound 111 |
|---|---|---|---|
| | | pound 111 | with Rotenone |
| Cell line | Type | EC₅₀ (nM) | EC₅₀ (nM) |
| MiaPaca2 | Pancreatic cancer | 37000 | 61 |
| Panc1 | Pancreatic cancer | 136000 | 91 |
| MDA-MB-468 | Breast cancer | >>30000 | 1400 |
| MM.1S | Multiple myeloma | 79000 | 52 |
| HCT116 | Colon cancer | 9700 | 510 |

A synergistic effect of compound 145 and compound 111 with OxPhos inhibitor Rotenone is demonstrated in SDH-proficient cells. No effect for Rotenone is demonstrated in SDH-deficient cells. The effect of the compounds depends on the status of the TCA cycle. Cells with a “broken TCA cycle”, either genetically or chemically, rely on glycolysis and thus are sensitive to the compound whereas cells with intact TCA cycle are much less sensitive (FIG. 2A, 21B).

### Example 4

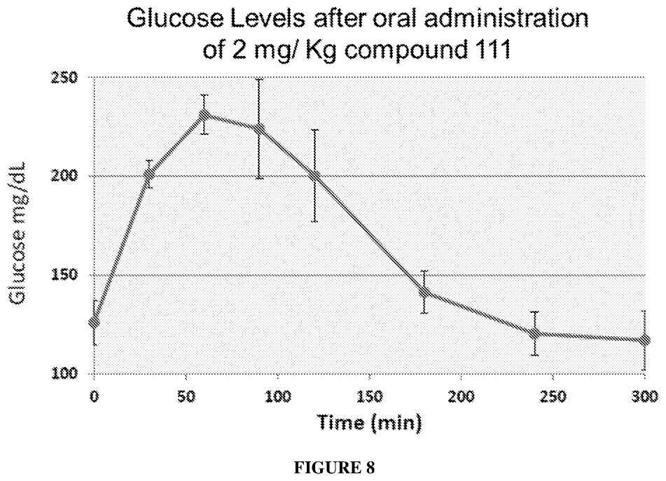

### In Vivo Data for Compound 111 (FIGS. 8-10)

### Compound 111 Elevates Plasma Glucose Levels in Mice

FIG. 8 depicts the glucose levels in mice plasma after oral administration of 2 mg/Kg of compound 111. Compound 111 at 2 mg/kg was orally administrated to mice. Blood samples were taken at different time points thereafter and Glucose level was determined.

Oral administration in mice of compound 111 at 2 mg/Kg lead to an increase in level of glucose in the plasma as is expected for compound that inhibits glucose uptake by the cells (FIG. 8). The glucose pick level was at ˜1 h after the administration of the compound.

Glucose Levels as Measured in Mice after Different Dosages of Compound 111 at Different Diets.

Ketogenic diet doubled the Maximum Tolerated Dose (MTD) and delayed elevation in plasma glucose levels after compound 111 treatment (FIG. 9).

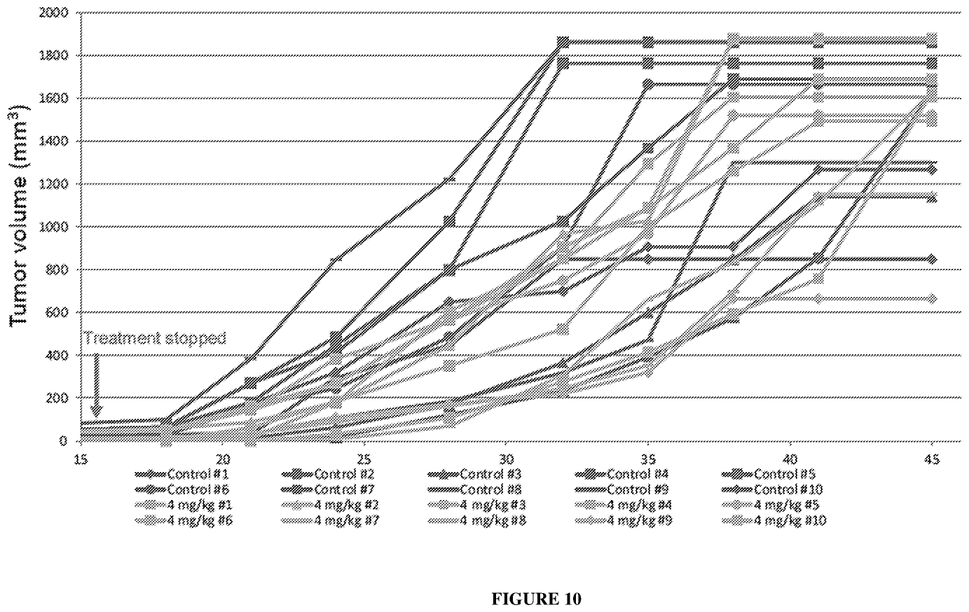

FIG. 10 depicts the tumor volume of SDH-KO cells vs. time in mice on ketogenic diet, treated for two weeks post-injection, but prior to tumor formation. Pre-treatment with compound 111 delays tumor onset and growth.

The results demonstrate that the compound is active in-vivo. Treatment of mice with compound 111 leads to an elevation of blood glucose level as expected from an inhibitors of glucose transporters. In addition, compound 111 inhibits in-vivo the growth of SDH-deficient tumors as expected from it's glucose uptake inhibition as the mode of action and the sensitivity of these tumors for glucose uptake inhibition.

In summary, compounds according to this invention demonstrated an effective inhibition of glucose transporters causing energy stress and cell death in SDH-deficient cells specifically. The compounds appear to cross the BBB extensively (possibly through an active transport). Treatment of mice with Glut inhibitors caused acute elevation in plasma glucose levels (FIG. 8). Ketogenic diet delayed plasma glucose levels elevation and enabled tolerability of up to 4 mg/Kg for two weeks of treatment (FIG. 9). Compound 111 demonstrated initial in-vivo efficacy in delaying tumor growth of SDH-deficient transformed cells (FIG. 10).

### Example 5

### Mechanistic Study of Compound Targeting Specifically SDH-Deficient Tumors

The aim of this study was to identify potential mechanisms and molecules that target specifically SDH-deficient tumors, and potentially also other tumor types possessing the broken TCA cycle phenotype.

An isogenic SDH-deficient/proficient cell pair was used in a differential viability phenotypic screen of a diverse library of small molecules which resulted in the discovery of compounds that selectively inhibited SDH-deficient cells while sparing their WT counterparts.

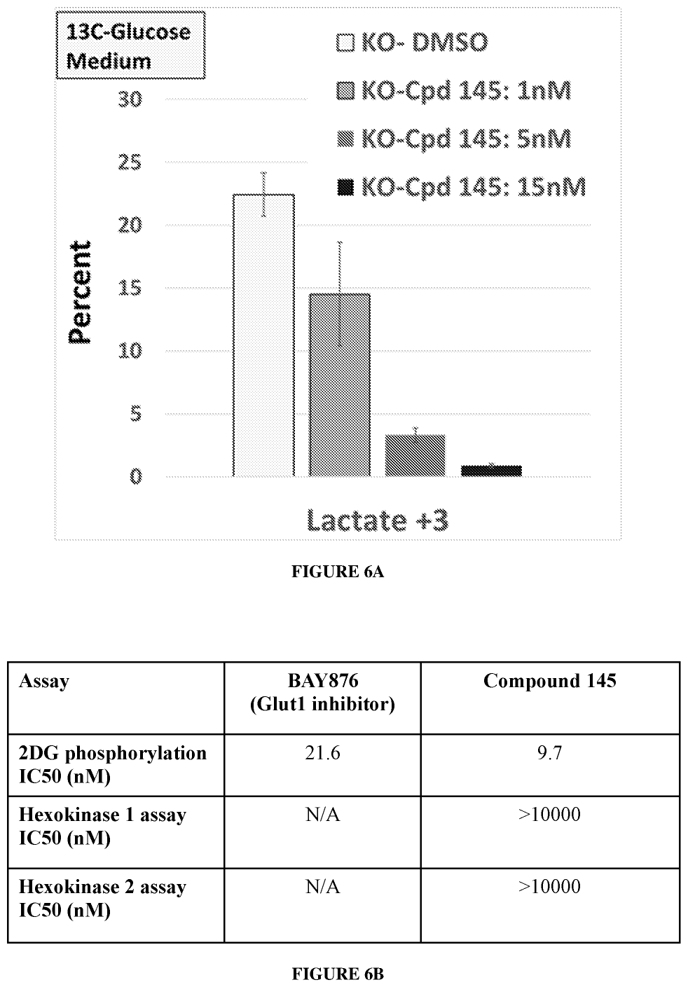

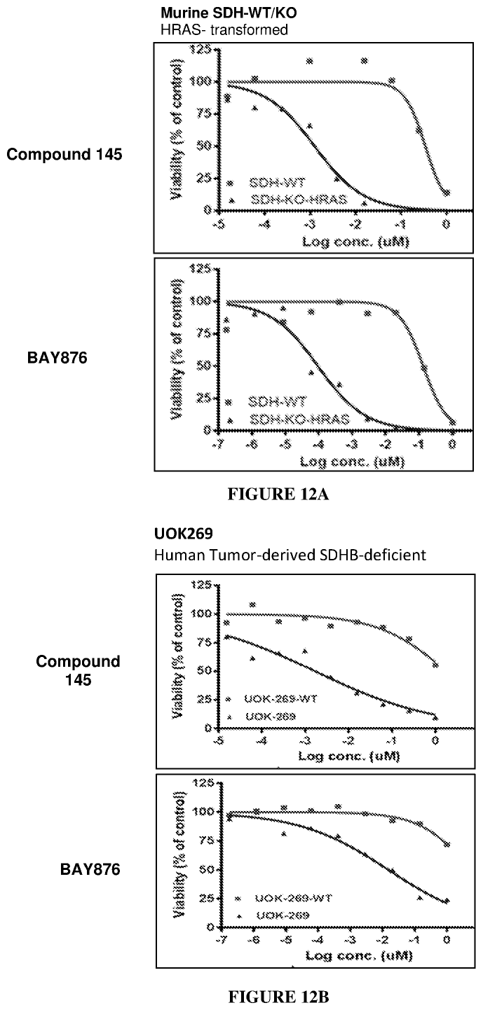

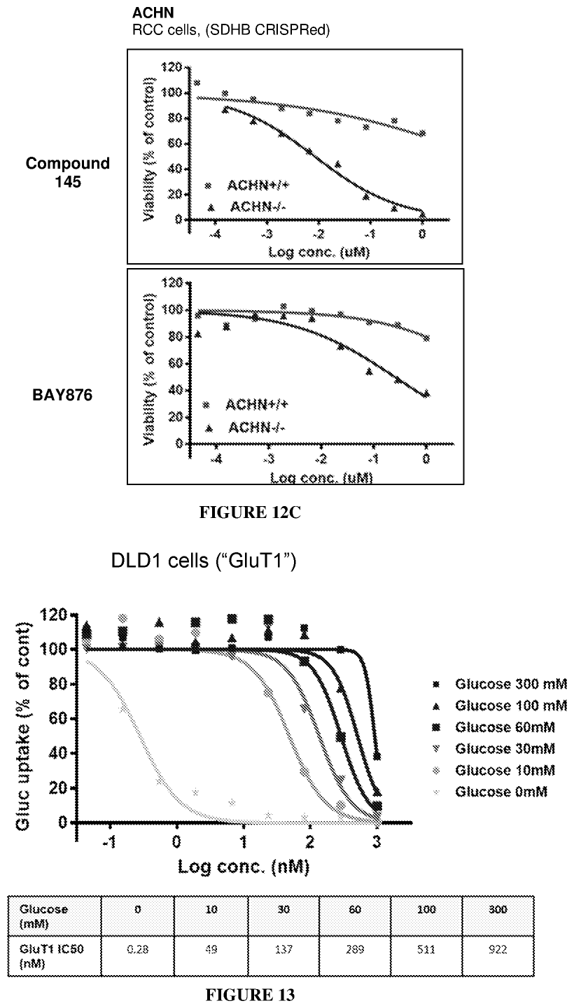

Compound 145, a prototype of one of the chemical series identified, showed differential toxicity in 3 distinct SDH-deficient/proficient cells pairs (FIG. 12) and strong synergistic effect with the OXPHOS inhibitor Rotenone in a variety of cells with intact TCA cycle (FIG. 2). A series of metabolomic experiments, utilizing primarily isotopically labelled glucose, were undertaken to identify the molecular target(s) of this compound (FIGS. 6A-B).

SDH-KO cells were treated with different concentrations of compound 145 for 30 min together with (U-13C)-Glucose. Lactate labeling from (U-13C)-Glucose (Lactate m+3) was determined by LC-MS analysis and was presented as percent of the total lactate in the medium.

It was found that compound 145 inhibits secretion of lactate to the medium which is one of the end points of glycolysis (FIG. 6A). The results suggest that compound 145 inhibits glycolysis upstream of lactate secretion.

Compound 145 also inhibits upstream glycolysis step as 2-DeoxyGlucose (2 DG) phosphorylation without inhibiting Hexokinase 1 (HK1) and Hexokinase 2 (HK2) (FIG. 6B).

IC₅₀ of compound 145 and GluT1 inhibitor BAY-786 in a 2 DG phosphorylation assay was done using murine SDH-WT cells. The cells were treated for 15 min with the indicated compound at different concentrations in the presence of 2 DG. 2 DG phosphorylation was determined by LC-MS analysis and the inhibition IC₅₀ was determined by Prism software. Both compounds inhibit 2 DG phosphorylation at low nM range (9.7 nM for compound 145 and 21.6 nM for the Glut1 inhibitor BAY876 (Siebeneicher et al, “Identification and Optimization of the First Highly Selective GLUT1 Inhibitor BAY-876”, ChemMedChem. 2016)).

HK1/2 assays were done using recombinant proteins. The assay readout was ADP level by ADP-Glo™ Kinase Assay (Promega).

The inhibition of glucose phosphorylation without affecting Hexokinase activity suggests that compound 145 inhibits glucose uptake. GluT1i (BAY-876) was served as GluT1 inhibitor control. Inhibition of 2 DG phosphorylation can be due to inhibition of glucose uptake or inhibition of HK1/2 activities. In order to test the second option, we tested the effect of compound 145 on the activity of HK1 and HK2 in biochemical assays using recombinant HK1 and HK2 proteins. The assay is using glucose as a substrate and measured the level of ADP. The results presented in FIG. 6B show that compound 145 didn't inhibit HK1/2 activities also at the highest concentration of 100 uM. These results demonstrate that compound 145 inhibits glycolysis upstream of HK activity, most likely by inhibiting glucose uptake.

### Example 6

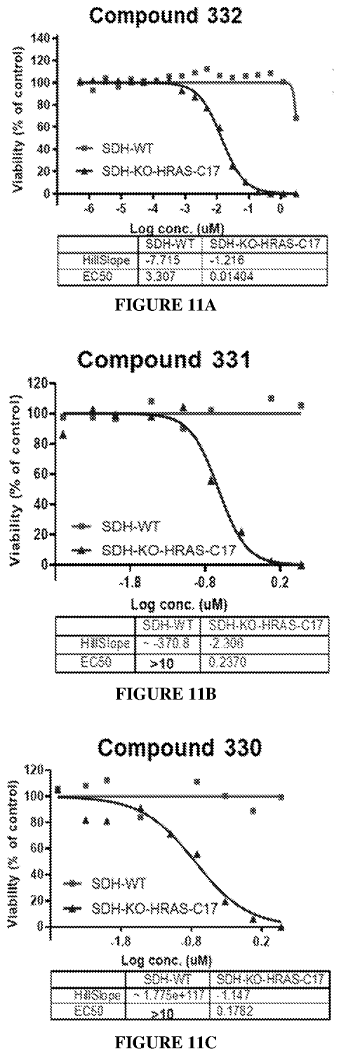

### Differential Toxicity for Compound 332, Compound 331 and Compound 330 in SDH-WT and SDH-KO Cells (FIG. 11)

The differential toxicity of compounds 332, 332 and 330 was measured in SDH-WT and SDH-KO cells.

SDH-WT and SDH-KO cells were grown in DMEM F₁₂ supplemented with 1 mM Glutamine+3% FBS were treated with different concentrations of the indicated compounds in FIG. 11. Cell viability was determined 72 hours later by CellTiter Glue reagent (Promega), the luminescence was measured with ClarioStar reader (BMG) and the EC₅₀s were calculated by Prism.

### Results:

All compounds were found to be highly potent and showed differential effect against SDH-KO cells compared to SDH-WT cells in the viability assays. The differential toxicity was 235, >42 and >56 for Compound 332, Compound 331 and Compound 330 respectively.

### Example 7

### Differential Toxicity Assay and Glycolysis Inhibition by Compound 145 and Compound 308 in SDH-WT and SDH-KO Cells

The Differential toxicity and glycolysis inhibition by Compound 145 and Compound 308 were measured in SDH-WT and SDH-KO cells.

### A Differential Toxicity Assay (EC₅₀ Tox).

SDH-WT and SDH-KO cells were grown in DMEM F₁₂ supplemented with 1 mM Glutamine+3% FBS were treated with different concentrations of the indicated compounds. Cell viability was determined 72 hours later by CellTiter Glue reagent (Promega), the luminescence was measured with ClarioStar reader (BMG) and the EC₅₀s were calculated by Prism.

Glycolysis Inhibition (IC₅₀ Lactate m+3)

Cell were plated in DMEM, 25 mM Glucose, 1 mM Pyruvate, 1 mM Glutamine and 10% Dialyzed serum in 96 well plates in triplicates. The day after the medium was replaced to fresh medium with 25 mM ¹³C-glucose and treated with different concentrations of the compounds as indicated. One hour later the medium was collected and the level of m+3 lactate was determined by LC-MS. The IC₅₀ of inhibition of m+3 lactate secretion into the medium was calculated with Prism.

### Results:

The differential toxicity assay and glycolysis inhibition by Compound 145 and Compound 308 in SDH-WT and SDH-KO cells are depicted in Table 6.

TABLE 6
IC₅₀ and EC₅₀ results for compounds 145 and 308

| | SDH-WT cells | | SDH-KO cells | |
|---|---|---|---|---|
| | Compound | Compound | Compound | Compound |
| | 145 | 308 | 145 | 308 |
| IC₅₀ Lactate | 1.7 | 0.15 | 6.1 | 0.03 |
| m + 3 (nM) | | | | |
| EC₅₀ Tox (nM) | 139 | 2174 | 1.8 | 0.004 |

Both compounds were highly potent against SDH-KO cells and showed significant differential effect between SDH-WT and KO cells in the cell viability assays (EC₅₀ Tox). The differential effects were around 80 folds for Compound 145 and more then 500,000 for Compound 308. Both compounds also inhibited glycolysis as measured by the secretion of m+3 Lactate into the medium (IC₅₀ Lactate m+3). The glycolysis inhibition potencies were comparable between the two cell-lines, 1.7 and 6.1 nM for Compound 145 and 0.15 and 0.03 nM for Compound 308 in SDH-WT and SDH-KO respectively. These results demonstrate the differential sensitivity of SDH-KO cells to inhibition of glycolysis.

### Example 8

### Biological Activity for Compound 145

### Comparison of Compound 145 to the Glucose Uptake Inhibitor, BAY876 (FIG. 12)

The EC₅₀ of Compound 145 and the selective glucose transporter 1 inhibitor (GluT1i) BAY-876 was measured in the following SDH-deficient/proficient isogenic pairs; murine kidney immortalized cells, HRAS transformed and SDHB-deficient (SDH-KO-HRAS) or its WT isogenic cells (SDH-WT).

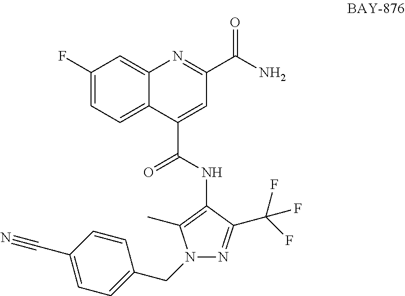 [SMILES: Cc1c(NC(=O)c2cc(C(N)=O)nc3cc(F)ccc23)c(C(F)(F)F)nn1Cc1ccc(C#N)cc1]

The cells were grown in DMEM F₁₂ supplemented with 1 mM Glutamine+3% FBS were treated with different concentrations of the indicated compounds. Cell viability was determined 72 hours later by CellTiter Glue reagent (Promega), the luminescence was measured with ClarioStar reader (BMG) and the EC₅₀s were calculated by Prism. Murine SDHB-WT/KO, HRAS transformed isogenic pair (FIG. 12A); UOK269, Human Tumor-derived SDHB-deficient RCC in their WT counterpart with re-expression of SDHB (FIG. 12B); and ACHN, RCC cells, and their SDHB-KO (SDHB CRISPRed out) isogenic cells (FIG. 12C).

### Results:

The EC₅₀ results are summarized in Table 7.

TABLE 7
EC₅₀ results of compound 145 with respect to the GLUT1
inhibitor BAY-876 in SDH-deficient/proficient isogenic pairs.

| | | EC₅₀ (nM) | | Ratio |
|---|---|---|---|---|
| | | Compound | BAY- | BAY-876/ |
| Type | Cells | 145 | 876 | COMP 145 |
| Trans- | Murine SDH-WT | 340 | 134 | 0.4 |
| formed | Murine SDH-KO | 1.2 | 0.95 | 0.8 |
| Tumor- | UOK269-WT | 1826 | 5296 | 2.9 |
| derived | UOK269 (SDHB-def.) | 1.7 | 15 | 8.8 |
| Cancer | ACHN WT | 13800 | 24190 | 1.7 |
| cells | ACHN SDHB −/− | 8.4 | 256 | 30 |

Compound 145 and BAY876 show a similar activity against SDH-KO cells but compound 145 show a larger differential effect compared to SDH-WT cells.

The results demonstrate that although both compounds show similar potencies against the transformed murine SDH-KO cells, Compound 145 is significantly more potent against the tumor-derived SDH KO cells (UK269 and ACHN SDHB−/−). The superior activity of compound 145 against the tumor derived cells is most likely due to the different selectivity profile of the compound with its better potency against several different GluTs.

### Example 9

### Compound 145 is a Glucose Competitive Inhibitor of Glucose Uptake (FIG. 13)

Glucose uptake assay with Compound 145, in DLD1 cells with different concentrations of glucose.

The assay was performed by measuring ATP production by Cell-Titer Glo reagent (Promega) in glucose-starved DLD1 cells (“GluT1”). DLD1-WT (Glut1 cells were plated overnight in 96-well plate at 80% confluency in starvation medium (DMEM without glucose, 0.5 mM glutamine, 1 mM pyruvate and 1% FBS). The cells were then pretreated for 30 min with 1 uM Rotenone and the inhibitors with different concentrations. The assay was initiated by adding different glucose levels as indicated and the plates were incubated for 15 min at 37° C. At the end of the incubation Cell-titer Glo was added to the wells and luminescence was measured. The inhibition IC₅₀ was calculated using Prism software.

### Results:

Compound 145 IC₅₀ was dependent of the level of glucose in the medium. The IC₅₀ was lower in the low glucose conditions and increased with the higher level of glucose. As the glucose concentration in the assay is rising, the inhibition decreases, which suggests that Compound 145 is a Glucose-competitive inhibitor of Glucose uptake.

### Example 10

### Compound 145 Selectivity Towards Glucose Transporters

TABLE 8
Glucose transporters selectivity assays. Different
selectivity profile of Compound 145 against GluT1/2/3.

| | | | | DLD- |
|---|---|---|---|---|
| | | DLD1- | DLD- | Glut1−/− |
| | Cells | WT | Glut1−/− | (Glut2) |
| | Glut | Glust1 | Glut3 | Glut2 |
| IC₅₀ | BAY-876 | 16 | 5920 | >10,000 |
| (nM) | Glut1i Bayer | | | |
| | Compound 145 | 19 | 2 | 14 |

Glucose transporter selectivity assays were done as described by Siebeneicher et al (ChemMedChem 2016, 11, 1-12) with the following adjustments; DLD1 cells for GLUT1 transporter, DLD1-GLUT1−/− cells (Horizon discovery) for GLUT3 transporter and DLD1-GLUT1−/− overexpressing hGLUT2 cells for GLUT2 transporter. The cells were plated overnight in 96-well plate at 80% confluency in starvation medium (DMEM without glucose, 0.5 mM glutamine, 1 mM pyruvate and 1% FBS). The cells were then pretreated for 30 min with 1 uM Rotenone and the inhibitors (compound 145) at different concentrations. The assay was initiated by adding glucose (5 mM for DLD1 cells, 10 mM for DLD1-Glut1−/−) or fructose (10 mM for DLD1-Glut1−/−(hGLUT2) cells) and the plates were incubated for 15 min at 37° C. At the end of the incubation Cell-titer Glo was added to the wells and luminescence was measured. The inhibition IC₅₀ was calculated using Prism software. BAY-876 was used as Glu1 inhibitor reference.

### Results:

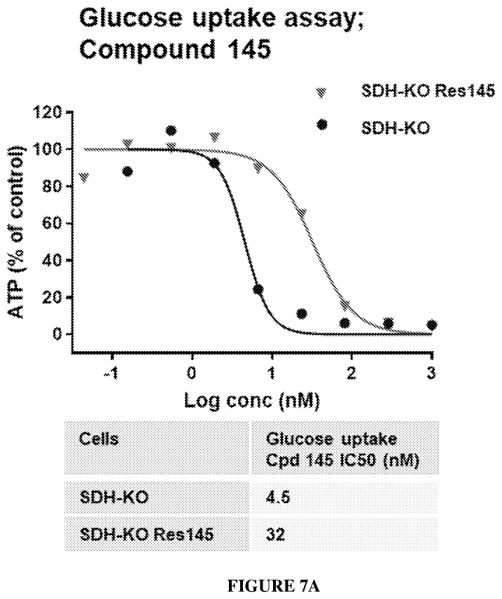

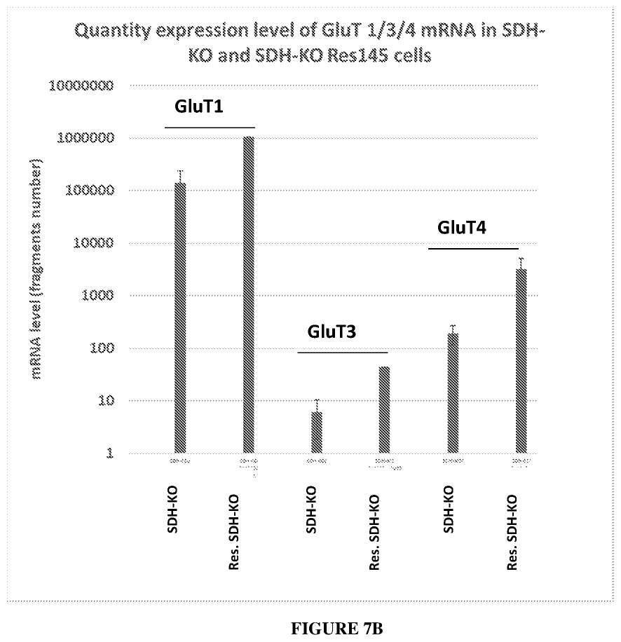

BAY-876 was selective towards Glut1 as expected with IC₅₀ of 16 nM for Glut1 and 5920 nM and >10,000 nM for Glut3 and Glut2 respectively. Compound 145 showed similar IC₅₀ for Glut1 (19 nM) but showed much better potencies against the other GluTs with IC₅₀ for Glut3 and Glut2 of 2 nM and 14 nM respectively. These results suggest that these compounds are Glucose transporter inhibitors with a different selectivity profile than BAY-876. The less selective profile may explain the better activity of Compound 145 against the tumor-derived SDH-deficient cells (UK269 and ACHN SDHB−/−). It was found that SDH-KO cells that are selected to be more resistant to the inhibitors have a higher expression of glucose transporters than the parental and more sensitive SDH-deficient cells (FIG. 7B), strengthening further the identified mode of action of the compounds.

Inhibition of GluTs with these compounds in SDH-deficient cells, but not in SDH-proficient cells, caused a rapid energy crisis followed by rapid cell death.

In summary, a novel series of GluT inhibitors that target the energetic vulnerabilities of tumors characterized by broken TCA cycle phenotype were identified.

### Example 11

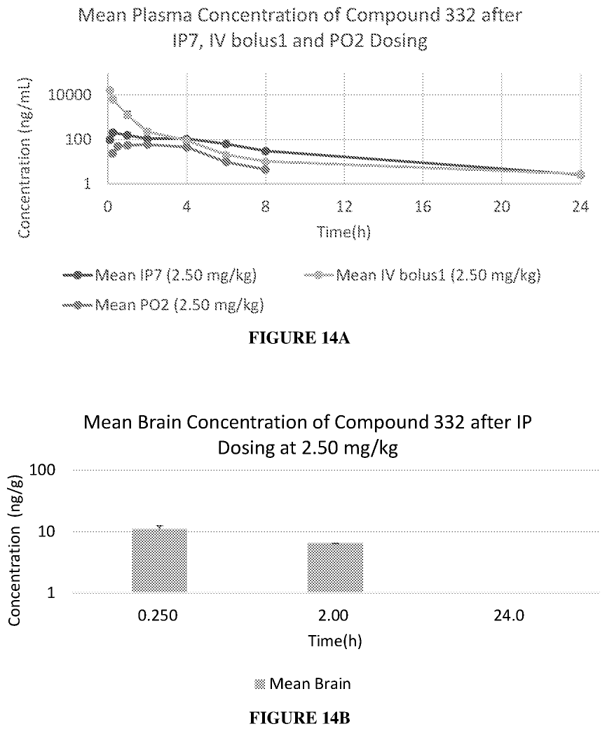

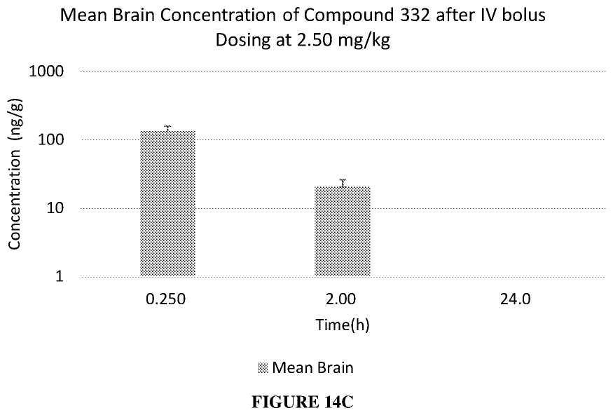

### PK Profile of Compound 332 (FIG. 14)

FIG. 14 describes the pharmacokinetics of compound 332. The drug's exposure is demonstrated by characterizing its absorption, distribution, bioavailability, metabolism, and excretion as a function of time (PK profile).

FIG. 14A depicts the Mean Plasma Concentration of Compound 332 after IP7, IV bolus1 and PO2 Dosing. FIG. 14B depicts the Mean Brain Concentration of Compound 332 after IP Dosing at 2.50 mg/kg and FIG. 14C depicts the Mean Brain Concentration of Compound 332 after IV bolus Dosing at 2.50 mg/kg.

### Results.

Compound 332 presented low oral bioavailability (FIG. 14A). FIG. 14B and FIG. 14C demonstrate that the compound is brain penetrant.

1-52. (canceled)

53. A glucose uptake inhibitor compound, represented by the structure of formula (II): 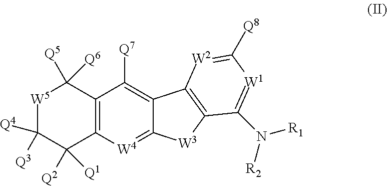
wherein
Q¹, Q², Q³, Q⁴, Q⁵, Q⁶, and Q⁸ are each independently H, F, Cl, Br, I, OH, SH, R₈—OH, R₈—SH, —R₈—O—R₁₀, CF₃, CN, NO₂, —CH₂CN, —R₈CN, NH₂, NHR, N(R)₂, R₈—N(R₁₀)(R₁₁), —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched alkyl (e.g., methyl, isopropyl), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring, substituted or unsubstituted aryl (e.g., phenyl);
Q⁷ is H, C(O)O—R₁₃ (e.g., n=3, 4, 5), R₈—O—R₁₃ (e.g., CH₂—O—(CH₂)₂O—CH₃, CH₂—[O—(CH₂)₂O]₃—CH₃, CH₂—[O—(CH₂)₂O]₄—CH₃, CH₂—[O—(CH₂)₂O]₅—CH₃), C(O)O—(CH₂)ₖ—COOH (e.g., k=7, 11, 15), F, Cl, Br, I, OH, SH, R₈—OH (e.g., (CH₂)—OH), R₈—SH, —R₈—O—R₁₀ (e.g., (CH₂)—OH, (CH₂)—O—CH₃, (CH₂)₄—OCH₃, (CH₂)—O-cyclopentyl, (CH₂)—O-cyclohexyl, (CH₂)—O-(1-methyl-piperidine), (CH₂)—O-Ph, (CH₂)—O-isobutyl, (CH₂)—O-tetrahydro-2H-pyran or (CH₂)—O-iPr), —R₈—S—R₁₀ (e.g., CH₂—S—CH₃), CF₃, CN, NO₂, —CH₂CN, —R₈CN, NH₂, NHR, N(R)₂, R₈—N(R₁₀)(R₁₁) (e.g., (CH₂)—N(CH₃)₂, (CH₂)—N(CH₃)([(CH₂)₂O]ₙ—CH₃) wherein n is 1, 3, 4), —OC(O)CF₃, —OCH₂Ph, NHC(O)—R₁₀, NHCO—N(R₁₀)(R₁₁), COOH, —C(O)Ph, C(O)O—R₁₀ (e.g. C(═O)O—C₂H₅), R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁) (e.g., C(O)N(CH₃)₂, C(O)N(H)(R₁₃) wherein n is 4), SO₂R (e.g., SO₂—CH₃), SO₂N(R₁₀)(R₁₁), CH(CF₃)(NH—R₁₀), C₁-C₅ linear or branched alkyl (e.g., methyl, propyl, isopropyl), C₁-C₅ linear or branched haloalkyl, C₁-C₅ linear or branched or C₃-C₈ cyclic alkoxy, C₁-C₅ linear or branched thioalkyl (e.g. S—CH₃), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopentyl, cyclohexyl), substituted or unsubstituted C₃-C₈ heterocyclic ring (e.g., thiazole), substituted or unsubstituted aryl (e.g., phenyl);
n is an integer number between 1 and 20 (e.g., 1, 3, 4, and 5);
k is an integer number between 1 and 20 (e.g., 7, 11, and 15);
R₁ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholine, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁);
R₂ is H, substituted or unsubstituted C₁-C₅ linear or branched alkyl (e.g., methyl, ethyl, propyl, butyl, pentyl, neopentyl, isobutyl, tBu, propanol), C₁-C₅ linear or branched alkenyl (e.g., propylene), C₁-C₅ linear or branched haloalkyl, C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclohexyl), substituted or unsubstituted C₃-C₈ heterocyclic ring, substituted or unsubstituted aryl, F, Cl, Br, I, OH, SH, R₈-R₁₂, R₁₂, R₈—OH, R₈—SH, —R₈—O—R₁₀ (e.g., (CH₂)₂—O—(CH₂)₂—OH, (CH₂)₂—O—(CH₃), (CH₂)₃—O—(CH₃)), CF₃, CN, NO₂, —CH₂CN, —R₈CN, NH₂, NHR, N(R)₂, R₈—N(R₁₀)(R₁₁) (e.g., (CH₂)₃-morpholine, (CH₂)₃—N(Et)₂), —OC(O)CF₃, —OCH₂Ph, —NHCO—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)—CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁); or
R₂ is represented by the structure of formula A: 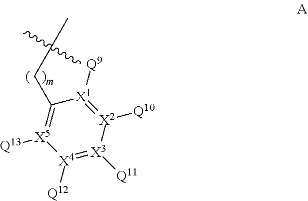
wherein
Q⁹, Q¹⁰, Q¹¹, Q¹² and Q¹³ are each independently H, F, Cl, Br, I, OH, SH, R₈—OH (e.g., CH₂—OH), R₈—SH, —R₈—O—R₁₀, (e.g., —CH₂—O—CH₃), CF₃, CN, NO₂, —CH₂CN, —R₈CN, NH₂, NHR, NH—R₈—R₁₀ (e.g., NH—CH₂—C(O)—CH₃), N(R)₂, N(R₁₀)(R₁₁) (e.g., N[C(O)CF₃][CH₂C(O)CH₃]), R₈—N(R₁₀)(R₁₁) (e.g., CH₂—N(CH₃)₂), R₈—C(O)—CH₂—O—R₁₀ (e.g., CH₂—C(O)—CH₂—OC(O)—CH₃), OH (e.g., CH₂—OH), —OC(O)CF₃, —OC(O)NH₂, —NHC(O)NH₂, —OCH₂Ph, NHC(O)—R₁₀ (e.g., NHC(O)CF₃, NHC(O)CH₃), NHCO—N(R₁₀)(R₁₁) (e.g., NHC(O)N(CH₃)₂), N(R₁₀)C(O)R (e.g., N(CH₃)C(O)(CH₃), NHC(O)—Ph, NHC(O)-iPr), —NHC(O)—C(H)(CH₃)—O—R₁₀, (e.g., —NHC(O)C(H)(CH₃)—OC(O)—CH₃), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)NH(CH₃), C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), C₁-C₅ linear or branched alkyl (e.g., methyl, ethyl, propyl, iso-propyl, t-Bu, iso-butyl, pentyl), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl) (wherein substitutions include: F, Cl, Br, I, C₁-C₅ linear or branched alkyl, OH, alkoxy, N(R)₂, CF₃, CN or NO₂), CH(CF₃)(NH—R₁₀);
or Q⁹ and Q¹⁰ are joined to form a 5 or 6 membered substituted or unsubstituted, aliphatic or aromatic, carbocyclic or heterocyclic ring (e.g., 1,4- or 1,3-dioxane, pyrrole);
or Q¹⁰ and Q¹¹ are joined to form a 5 or 6 membered substituted or unsubstituted, aliphatic or aromatic, carbocyclic or heterocyclic ring (e.g., 1,4- or 1,3-dioxane, pyrrole, imidazole, 1-methylimidazole, oxazole, triazole, furane, 1H-imidazol-2(3H)-one);
X¹, X², X³, X⁴ and X⁵ are each independently C or N,
wherein
if X¹, X², X³, X⁴ and/or X⁵ is N then Q⁹, Q¹⁰, Q¹¹, Q¹² and/or Q¹³ is absent respectively;
or R₂ is represented by the structure of formula B: 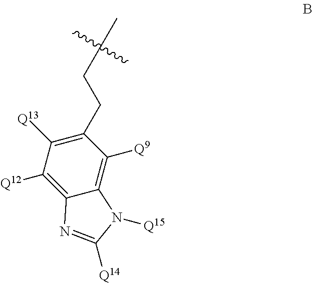
wherein
Q¹⁴ is H, F, Cl, Br, I, OH, SH, R₈—OH (e.g., CH₂—OH), R₈—SH, —R₈—O—R₁₀, (e.g., —CH₂—O—(C═O)—CH₃, CH₂—O—CH₃), C₁-C₅ linear or branched alkylester (e.g., —CH₂—O—(C═O)—CH₃, CH(CH₃)—O(C═O)—CH₃), CF₃, CN, NO₂, —CH₂CN, —R₈CN, NH₂, NHR, N(R)₂, R₈—N(R₁₀)(R₁₁) (e.g., CH₂—N(CH₃)₂), —OC(O)CF₃, —OC(O)NH₂, —NHC(O)NH₂, —OCH₂Ph, NHC(O)—R₁₀ (e.g., NHC(O)CH₃), NHCO—N(R₁₀)(R₁₁) (e.g., NHC(O)N(CH₃)₂), N(R₁₀)C(O)R (e.g., N(CH₃)C(O)(CH₃)), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), substituted or unsubstituted C₁-C₅ linear or branched alkyl (e.g., methyl, ethyl, propyl, iso-propyl, t-Bu, iso-butyl, pentyl, CH(OH)CH₃), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl), CH(CF₃)(NH—R₁₀); and
Q¹⁵ is H, C₁-C₅ linear or branched alkyl (e.g., methyl, ethyl, propyl, iso-propyl, t-Bu, iso-butyl, pentyl), substituted or unsubstituted C₃-C₈ heterocyclic ring (e.g., 1-methyl-pyrrolidine-2-one);
or R₁ and R₂ are joined to form a 5 or 6 membered substituted or unsubstituted, heterocyclic ring (e.g., pyrrolidine, morpoline, piperazine, piperidine, 4-(3-fluoro-4-methoxyphenyl)-1-piperidine, 2-(piperazin-1-yl)ethanol);
R₈ is [CH₂]ₚ
wherein p is between 1 and 10 (e.g., 2);
R₁₀ and R₁₁ are each independently H, C₁-C₅ linear or branched alkyl (e.g., methyl, ethyl, iso-propyl, iso-butyl), substituted or unsubstituted C₃-C₈ cycloalkyl (e.g., cyclopentyl, cyclohexyl), substituted or unsubstituted C₃-C₈ heterocyclic ring (e.g., 1-methyl piperidine, tetrahydro-2H-pirane), aryl (e.g., phenyl), C(O)R (e.g., C(O)CH₃), O(C═O)R (e.g., O(C═O)—CH₃), R₈—C(O)R (e.g., CH₂—C(O)CH₃), R₁₃ (e.g., [(CH₂)₂O]₃—CH₃, [(CH₂)₂O]₄—CH₃) or S(O)₂R;
or R₁₀ and R₁₁ are joined to form a 5 or 6 membered substituted or unsubstituted, carbocyclic or heterocyclic ring (e.g., morpholine); R₁₂ is H, C₁-C₅ linear or branched alkyl (e.g., CH(CH₃)₂), C₁-C₅ linear or branched haloalkyl, substituted or unsubstituted C₃-C₁₂ single or fused cycloalkyl, substituted or unsubstituted single or fused C₃-C₁₂ heterocyclic ring (e.g. benzimidazole, 2-, 3- or 4-tetrahydropyranyl, 1H-benzo[d]imidazol-2(3H)-one), substituted or unsubstituted single or fused aryl (e.g., phenyl, 2-nitrophenolyl, N-methyl-2-nitroaniline, 2,2,2-trifluoro-N-(2-nitrophenyl)acetamide, 3-(2-aminophenyl)-2-oxopropyl acetate, 3-(2-nitrophenyl)-2-oxopropyl acetate, 1-((2-aminophenyl)amino)-1-oxopropan-2-yl acetate, 1-((2-fluorophenyl)amino)propan-2-one, N-(2-aminophenyl)benzamide, benzene-1,2-diamine, 2-, 3-, or 4-methoxyphenyl, 4-fluoro-3-methoxyphenyl, 3-fluoro-4-methoxyphenyl, 3,4-difluorophenyl, 2,6-3,5-2,3-2,4- or 3,4-dimethoxyphenyl, 4-acetamide-phenyl, 4-benzoicacid, 3-methoxy-4-propoxyphenyl, methyl-4-benzoate, 2,3-dihydrobenzo[b][1,4]dioxinyl, 3-fluoro-4-hydroxyphenyl, 4-phenylurea, 4-methylbenzamide, 4-phenylcarbamate, 4-benzamide, 4-N,N-dimethylbenzamide, aniline, N-phenylmethanesulfonamide, 4-N-methyl-N-phenylmethanesulfonamide, 4-phenol, methyl benzenesulfonate, N-methyl-N-phenylacetamide, 4-methoxy-3-(trifluoromethyl)benzene), substituted or unsubstituted single or fused heteroaryl (e.g., indolyl, 2-, 3- or 4-pyridinyl, pyrimidinyl, benzimidazolyl, 1-methyl-benzimidazolyl, benzooxazolyl, 2-methyl-1H-benzo[d]imidazole, 2-methoxy-1H-benzo[d]imidazole), C₁-C₅ linear or branched or C₃-C₈ cyclic alkoxy, C₁-C₅ linear or branched thioalkoxy, C₁-C₅ linear or branched haloalkoxy, C₁-C₅ linear or branched alkoxyalkyl;
R₁₃ is [(CH₂)₂O]ₙ—CH₃
wherein n is between 1 and 20 (e.g., 1, 3, 4, 5);
R is H, C₁-C₅ linear or branched alkyl, C₁-C₅ linear or branched alkoxy, C₁-C₅ linear or branched haloalkyl (e.g. CF₃), C₁-C₅ linear or branched alkyl ester (e.g., CH₂—O—(CO)—CH₃), (C═O)—CH(CH₃)—O—(C═O)—CH₃, (CH₂—(C═O)—CH₃), phenyl, aryl or heteroaryl, —(C═O)—CH₃, —(C═O)—CF₃, —(C═O)-Ph, or two gem R substituents are joined to form a 5 or 6 membered heterocyclic ring;
wherein substitutions include: F, Cl, Br, I, substituted or unsubstituted C₁-C₅ linear or branched alkyl, (e.g. methyl, isopropyl, CH₂—OH, CH(OH)—CH₃), substituted or unsubstituted C₃-C₈ carbocyclic or heterocyclic ring (e.g., furan, tetrahydrofuran, morpholine), aryl, benzyl, OH, alkoxy (e.g., ethoxy), O(C═O)—R₁₀, COOH, COO—R₁₀, R₈—R₁₀ (e.g., CH₂—(C═O)—CH₂—O(C═O)—CH₃, CH₂—O(C═O)—CH₃), substituted or unsubstituted C₁-C₅ linear or branched alkylester (e.g., CH(CH₃)—O—(C═O)—CH₃), N(R)₂, (e.g., NH₂), NHR (e.g., NH(C═O)(CH₃), NH(CH₃), NH(CH₂—(C═O)—CH₃), NH(C═O)Ph), NH(C═O)(CF₃), NH(C═O)—CH(CH₃)—O—(C═O)—CH₃)), aryl (e.g., phenyl), CF₃, CN or NO₂;
W¹, W² and W⁴ are each independently CH, C(R) or N;
W³ is S, SO, SO₂, O, N—OH, CH₂, C(R)₂ or N—OMe;
W⁵ is a bond, S, O, NH, N(R) (e.g., N—CH₃), N(R)₂, CH₂, CH(R), C(R)₂, N—OH, or
N—OR;
or its pharmaceutically acceptable salt, stereoisomer, tautomer, hydrate, N-oxide, isotopic variant, pharmaceutical product or any combination thereof

54. The compound of claim 53, represented by the structure of formula (IV): 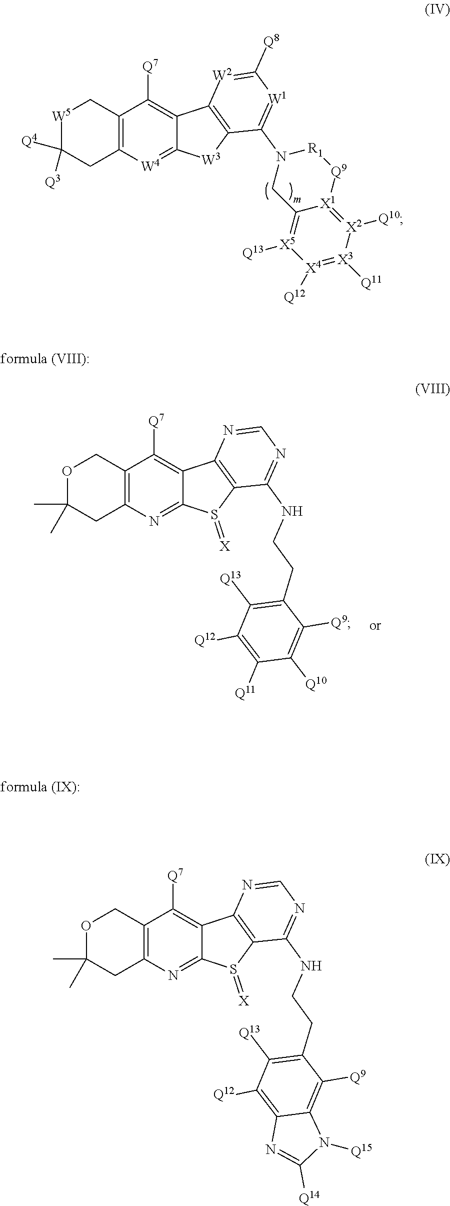
wherein
S═X is S, S═O or SO₂;
Q¹⁴ is H, F, Cl, Br, I, OH, SH, R₈—OH (e.g., CH₂—OH), R₈—SH, —R₈—O—R₁₀, (e.g., —CH₂—O—(C═O)—CH₃, CH₂—O—CH₃), C₁-C₅ linear or branched alkylester (e.g., —CH₂—O—(C═O)—CH₃, CH(CH₃)—O(C═O)—CH₃), CF₃, CN, NO₂, —CH₂CN, —R₈CN, NH₂, NHR, N(R)₂, R₈—N(R₁₀)(R₁₁) (e.g., CH₂—N(CH₃)₂), —OC(O)CF₃, —OC(O)NH₂, —NHC(O)NH₂, —OCH₂Ph, NHC(O)—R₁₀ (e.g., NHC(O)CH₃), NHCO—N(R₁₀)(R₁₁) (e.g., NHC(O)N(CH₃)₂), N(R₁₀)C(O)R (e.g., N(CH₃)C(O)(CH₃)), COOH, —C(O)Ph, C(O)O—R₁₀ (e.g. C(O)O—CH₃, C(O)O—CH₂CH₃), R₈—C(O)—R₁₀ (e.g., CH₂C(O)CH₃), C(O)H, C(O)—R₁₀ (e.g., C(O)—CH₃, C(O)—CH₂CH₃, C(O)—CH₂CH₂CH₃), C₁-C₅ linear or branched C(O)-haloalkyl (e.g., C(O)—CF₃), —C(O)NH₂, C(O)NHR (e.g. C(O)NH—CH₃), C(O)N(R₁₀)(R₁₁) (e.g., C(O)N(CH₃)₂), OSO₂R (e.g., OSO₂(CH₃)), NHSO₂R (e.g., NHSO₂(CH₃)), N(R₁₀)SO₂R (e.g., N(CH₃)SO₂(CH₃)), SO₂R, SO₂N(R₁₀)(R₁₁) (e.g., SO₂N(CH₃)₂), substituted or unsubstituted C₁-C₅ linear or branched alkyl (e.g., methyl, ethyl, propyl, iso-propyl, t-Bu, iso-butyl, pentyl, CH(OH)CH₃), C₁-C₅ linear or branched haloalkyl (e.g., CF₂CH₃, CH₂CF₃), C₁-C₅ linear or branched or C₃-C₈ cyclic alkoxy (e.g. methoxy, ethoxy, propoxy, isopropoxy, O—CH₂-cyclopropyl), C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopentyl), substituted or unsubstituted C₃-C₈ heterocyclic ring (e.g., thiophene, oxazole, thiazole, imidazole, furane, triazole, pyridine (2, 3, or 4-pyridine), pyrimidine, pyrazine, pyrrolidone), substituted or unsubstituted aryl (e.g., phenyl), CH(CF₃)(NH—R₁₀); and
Q¹⁵ is H, C₁-C₅ linear or branched alkyl (e.g., methyl, ethyl, propyl, iso-propyl, t-Bu, iso-butyl, pentyl), substituted or unsubstituted C₃-C₈ heterocyclic ring (e.g., 1-methyl-pyrrolidine-2-one).

55. The compound of claim 54, wherein
said substituted comprises at least one substitution selected from the list of: F, Cl, Br, I, substituted or unsubstituted C₁-C₅ linear or branched alkyl, methyl, isopropyl, CH₂—OH, CH(OH)—CH₃, substituted or unsubstituted C₃-C₈ carbocyclic or heterocyclic ring, furan, tetrahydrofuran, morpholine, aryl, phenyl, benzyl, OH, alkoxy, ethoxy, O(C═O)—R₁₀, COOH, COO—R₁₀, R₈—R₁₀, CH₂—(C═O)—CH₂—O(C═O)—CH₃, CH₂—O(C═O)—CH₃, substituted or unsubstituted C₁-C₅ linear or branched alkylester, CH(CH₃)—O—(C═O)—CH₃, N(R)₂, NH₂, NHR, NH(C═O)(CH₃), NH(CH₃), NH(CH₂—(C═O)—CH₃), NH(C═O)Ph, NH(C═O)(CF₃), NH(C═O)—CH(CH₃)—O—(C═O)—CH₃), aryl, phenyl, CF₃, CN and NO₂;
Q¹, Q², Q⁵ and Q⁶ are each independently H,
Q³, and Q⁴ are both substituted or unsubstituted C₁-C₅ linear or branched alkyl (preferably methyl);
Q⁷ is H, C(O)O—R₁₃ (preferably wherein n=3, 4 or 5), R₈—O—R₁₃ (preferably CH₂—O—(CH₂)₂O—CH₃, CH₂—[O—(CH₂)₂O]₃—CH₃, CH₂—[O—(CH₂)₂O]₄—CH₃, or CH₂—[O—(CH₂)₂O]₅—CH₃), C(O)O—(CH₂)ₖ—COOH (preferably wherein k=7, 11 or 15), R₈—OH (preferably (CH₂)—OH), —R₈—O—R₁₀ (preferably (CH₂)—O—CH₃, (CH₂)₄—OCH₃, (CH₂)—O-cyclopentyl, (CH₂)—O— cyclohexyl, (CH₂)—O-(1-methyl-piperidine), (CH₂)—O-Ph, (CH₂)—O-isobutyl, (CH₂)—O-tetrahydro-2H-pyran or (CH₂)—O-iPr)), —R₈—S—R₁₀ (preferably CH₂—S—CH₃), R₈—N(R₁₀)(R₁₁) (preferably (CH₂)—N(CH₃)₂ or (CH₂)—N(CH₃)([(CH₂)₂O]ₙ—CH₃) wherein n is 1, 3 or 4), C(O)N(R₁₀)(R₁₁) (preferably C(O)N(CH₃)₂ or C(O)N(H)(R₁₃)), C₁-C₅ linear or branched thioalkyl (preferably S—CH₃), C(O)O—R₁₀ (preferably C(═O)O—C₂H₅), C₁-C₅ linear or branched alkyl (preferably methyl, propyl or isopropyl), C₁-C₅ linear or branched alkoxyalkyl, substituted or unsubstituted C₃-C₈ cycloalkyl (preferably cyclopentyl, cyclohexyl), substituted or unsubstituted C₃-C₈ heterocyclic ring (preferably thiazole) or substituted or unsubstituted aryl (preferably phenyl);
Q⁸ is H, C₁-C₅ linear or branched alkyl (preferably methyl or isopropyl), substituted or unsubstituted C₃-C₈ cycloalkyl (preferably cyclopentyl or cyclohexyl) or substituted or unsubstituted aryl (preferably phenyl)
Q⁹, Q¹⁰, Q¹¹, Q¹² and Q¹³ are each independently H, F, OH, NH₂, NO₂, —OC(O)NH₂, —NHC(O)NH₂, —N(R₁₀)C(O)R (preferably N(CH₃)C(O)(CH₃), NHC(O)-Ph or NHC(O)-iPr), COOH, —C(O)NH₂, C(O)N(R₁₀)(R₁₁) (preferably C(O)NH(CH₃) or C(O)N(CH₃)₂), C₁-C₅ linear or branched or C₃-C₈ cyclic alkoxy (preferably methoxy, ethoxy, propoxy, isopropoxy or O—CH₂-cyclopropyl), or C₃-C₈ heterocyclic ring (preferably thiophene, oxazole or thiazole);
Q¹⁴ is H, substituted or unsubstituted C₁-C₅ linear or branched alkyl (preferably methyl), C₁-C₅ linear or branched or C₃-C₈ cyclic alkoxy (preferably methoxy), NH₂ or substituted or unsubstituted aryl (preferably phenyl);
Q¹⁵ is H, or substituted or unsubstituted C₃-C₈ heterocyclic ring (preferably 1-methyl-pyrrolidine-2-one);
R₁ is H or C₁-C₅ linear or branched alkyl (preferably methyl);
R₂ is C₁-C₅ linear or branched alkyl (preferably butyl), R₈-R₁₂, represented by the structure of formula A, or represented by the structure of formula B;
R₁₀ and R₁₁ are each independently H, C₁-C₅ linear or branched alkyl (preferably methyl, ethyl, iso-propyl or iso-butyl), substituted or unsubstituted C₃-C₈ cycloalkyl (preferably cyclopentyl or cyclohexyl), substituted or unsubstituted C₃-C₈ heterocyclic ring (preferably 1-methyl piperidine or tetrahydro-2H-pirane), aryl (preferably phenyl), R₁₃ (preferably [(CH₂)₂O]₃—CH₃ or [(CH₂)₂O]₄—CH₃);
or R₁₀ and Ru are joined to form a 5 or 6 membered unsubstituted, carbocyclic or heterocyclic ring (preferably morpholine);
R₁₂ is substituted aryl or substituted or unsubstituted single or fused heteroaryl (preferably indolyl, benzoxazolyl, 1H-benzo[d][1,2,3]triazolyl or benzimidazolyl);
R₁₃ is [(CH₂)₂O]ₙ—CH₃ wherein n is preferably 1, 3, 4 or 5;
R is —(C═O)—CH₃;
W¹, W² and W⁴ are N;
W³ is S or O;
W⁵ is O;
or any combination thereof.

56. The compound of claim 54, wherein R₁ is H, R₂ is represented by formula B, Q⁷ is H, R₈—O—R₁₃ (preferably CH₂—O—(CH₂)₂O—CH₃) or —R₈—O—R₁₀ (preferably (CH₂)—O—CH₃), Q¹⁴ and Q¹⁵ are H, W¹, W² and W⁴ are N; W³ is S; and W⁵ is O.

57. The compound of claim 53, selected from the following:

| Compound | |
|---|---|
| No. | Structure |
| 100 | 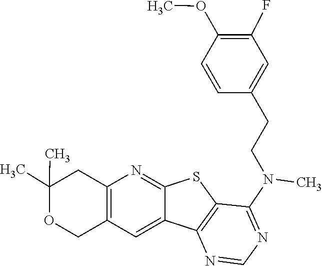 |
| 101 | 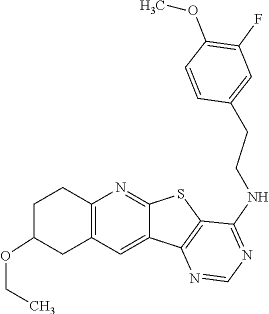 [SMILES: CCOC1CCc2nc3sc4c(NCCc5ccc(OC)c(F)c5)ncnc4c3cc2C1] |
| 102 | 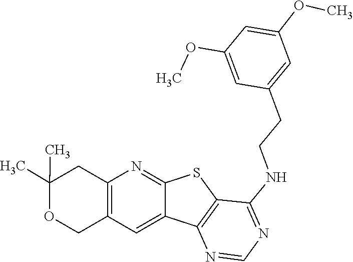 |
| 103 | 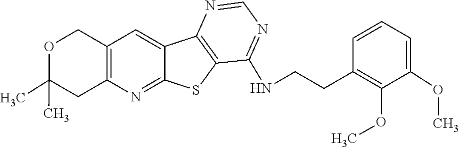 [SMILES: COc1cccc(CCNc2ncnc3c2sc2nc4c(cc23)COC(C)(C)C4)c1OC] |
| 105 | 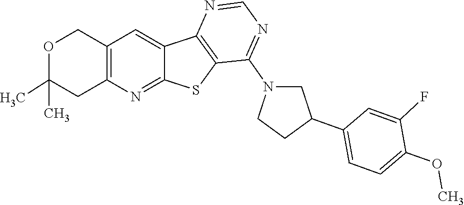 |
| 106 | 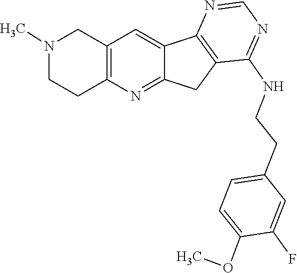 [SMILES: COc1ccc(CCNc2ncnc3c2Cc2nc4c(cc2-3)CN(C)CC4)cc1F] |
| 109 | 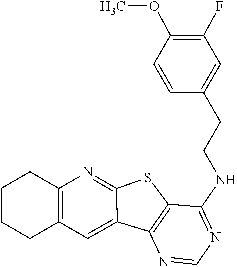 [SMILES: COc1ccc(CCNc2ncnc3c2sc2nc4c(cc23)CCCC4)cc1F] |
| 111 | 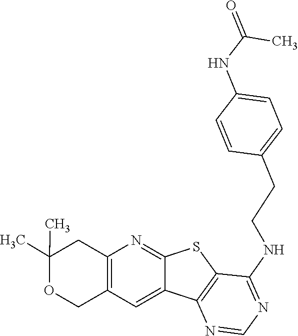 [SMILES: CC(=O)Nc1ccc(CCNc2ncnc3c2sc2nc4c(cc23)COC(C)(C)C4)cc1] |
| 112 | 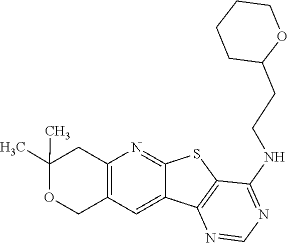 [SMILES: CC1(C)Cc2nc3sc4c(NCCC5CCCCO5)ncnc4c3cc2CO1] |
| 113 | 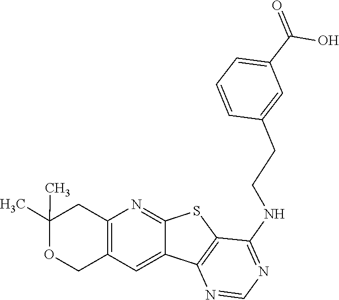 [SMILES: CC1(C)Cc2nc3sc4c(NCCc5cccc(C(=O)O)c5)ncnc4c3cc2CO1] |
| 114 | 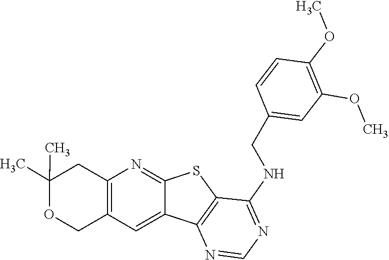 |
| 115 | 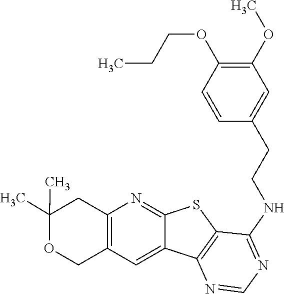 [SMILES: CCCOc1ccc(CCNc2ncnc3c2sc2nc4c(cc23)COC(C)(C)C4)cc1OC] |
| 116 | 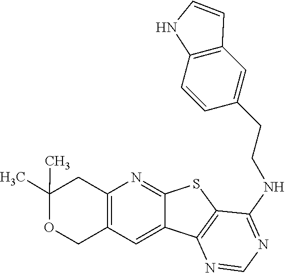 [SMILES: CC1(C)Cc2nc3sc4c(NCCc5ccc6[nH]ccc6c5)ncnc4c3cc2CO1] |
| 117 | 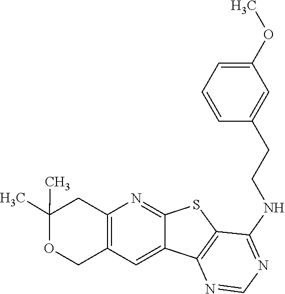 [SMILES: COc1cccc(CCNc2ncnc3c2sc2nc4c(cc23)COC(C)(C)C4)c1] |
| 120 | 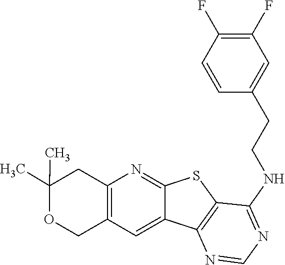 [SMILES: CC1(C)Cc2nc3sc4c(NCCc5ccc(F)c(F)c5)ncnc4c3cc2CO1] |
| 121 | 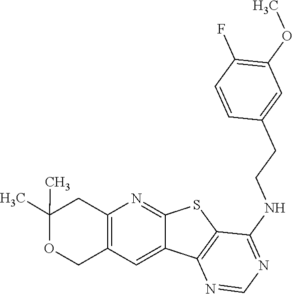 [SMILES: COc1cc(CCNc2ncnc3c2sc2nc4c(cc23)COC(C)(C)C4)ccc1F] |
| 122 | 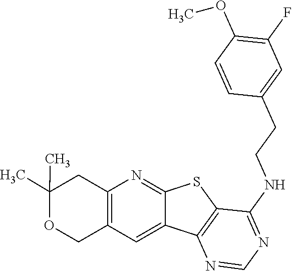 [SMILES: COc1ccc(CCNc2ncnc3c2sc2nc4c(cc23)COC(C)(C)C4)cc1F] |
| 123 | 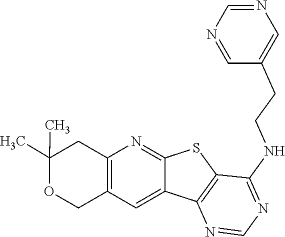 [SMILES: CC1(C)Cc2nc3sc4c(NCCc5cncnc5)ncnc4c3cc2CO1] |
| 124 | 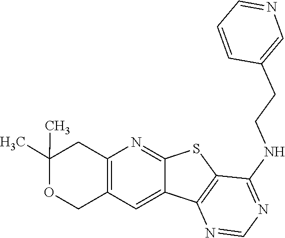 [SMILES: CC1(C)Cc2nc3sc4c(NCCc5cccnc5)ncnc4c3cc2CO1] |
| 125 | 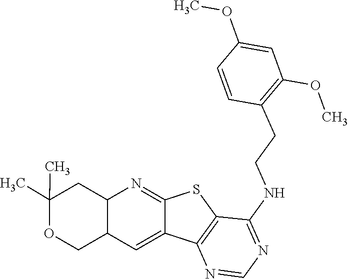 |
| 127 | 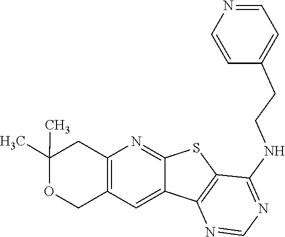 [SMILES: CC1(C)Cc2nc3sc4c(NCCc5ccncc5)ncnc4c3cc2CO1] |
| 128 | 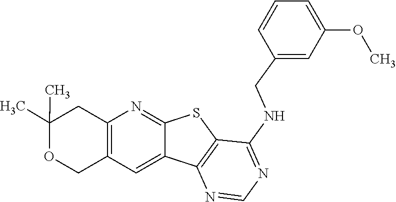 |
| 129 | 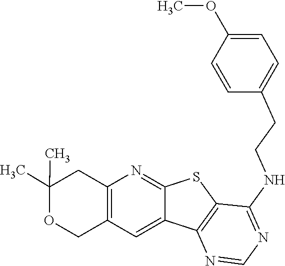 [SMILES: COc1ccc(CCNc2ncnc3c2sc2nc4c(cc23)COC(C)(C)C4)cc1] |
| 130 | 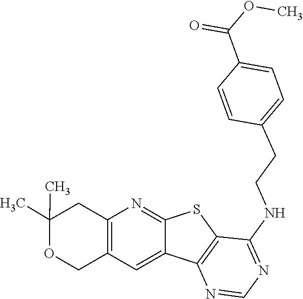 [SMILES: COC(=O)c1ccc(CCNc2ncnc3c2sc2nc4c(cc23)COC(C)(C)C4)cc1] |
| 131 | 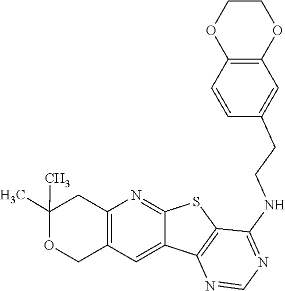 [SMILES: CC1(C)Cc2nc3sc4c(NCCc5ccc6c(c5)OCCO6)ncnc4c3cc2CO1] |
| 132 | 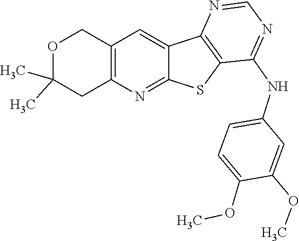 [SMILES: COc1ccc(Nc2ncnc3c2sc2nc4c(cc23)COC(C)(C)C4)cc1OC] |
| 133 | 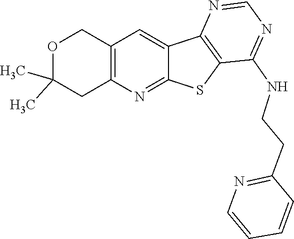 [SMILES: CC1(C)Cc2nc3sc4c(NCCc5ccccn5)ncnc4c3cc2CO1] |
| 134 | 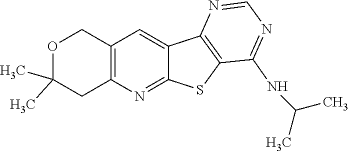 [SMILES: CC(C)Nc1ncnc2c1sc1nc3c(cc12)COC(C)(C)C3] |
| 135 | 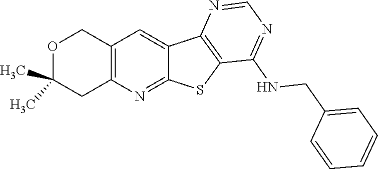 |
| 136 | 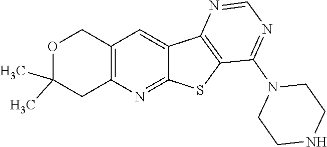 [SMILES: CC1(C)Cc2nc3sc4c(N5CCNCC5)ncnc4c3cc2CO1] |
| 137 | 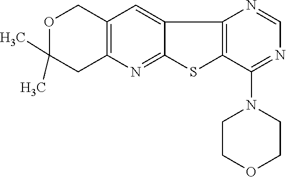 [SMILES: CC1(C)Cc2nc3sc4c(N5CCOCC5)ncnc4c3cc2CO1] |
| 138 | 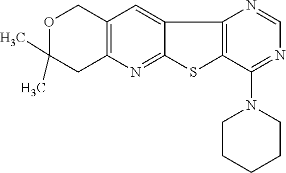 [SMILES: CC1(C)Cc2nc3sc4c(N5CCCCC5)ncnc4c3cc2CO1] |
| 139 | 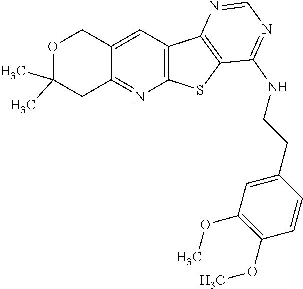 [SMILES: COc1ccc(CCNc2ncnc3c2sc2nc4c(cc23)COC(C)(C)C4)cc1OC] |
| 140 | 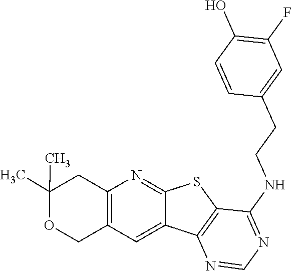 [SMILES: CC1(C)Cc2nc3sc4c(NCCc5ccc(O)c(F)c5)ncnc4c3cc2CO1] |
| 141 | 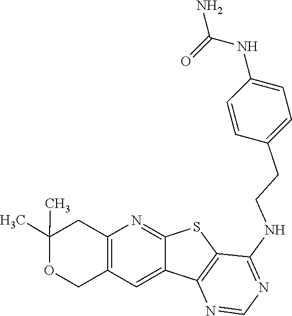 [SMILES: CC1(C)Cc2nc3sc4c(NCCc5ccc(NC(N)=O)cc5)ncnc4c3cc2CO1] |
| 142 | 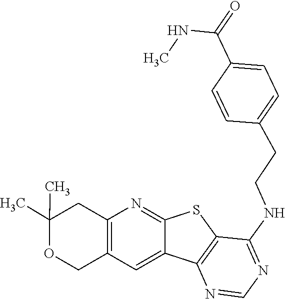 [SMILES: CNC(=O)c1ccc(CCNc2ncnc3c2sc2nc4c(cc23)COC(C)(C)C4)cc1] |
| 143 | 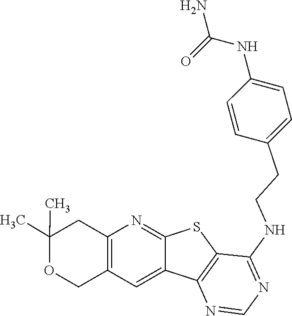 [SMILES: CC1(C)Cc2nc3sc4c(NCCc5ccc(NC(N)=O)cc5)ncnc4c3cc2CO1] |
| 144 | 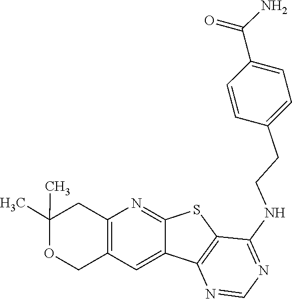 [SMILES: CC1(C)Cc2nc3sc4c(NCCc5ccc(C(N)=O)cc5)ncnc4c3cc2CO1] |
| 145 | 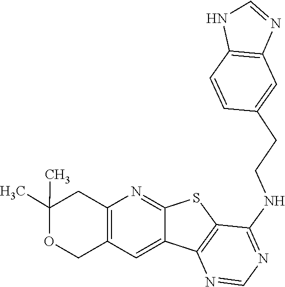 [SMILES: CC1(C)Cc2nc3sc4c(NCCc5ccc6[nH]cnc6c5)ncnc4c3cc2CO1] |
| 146 | 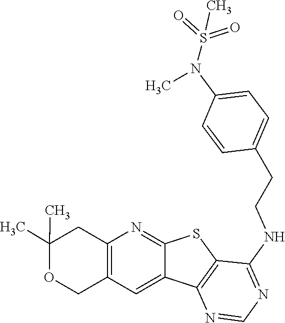 [SMILES: CN(c1ccc(CCNc2ncnc3c2sc2nc4c(cc23)COC(C)(C)C4)cc1)S(C)(=O)=O] |
| 147 | 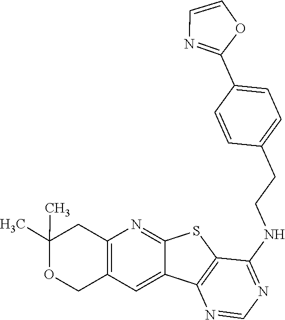 [SMILES: CC1(C)Cc2nc3sc4c(NCCc5ccc(-c6ncco6)cc5)ncnc4c3cc2CO1] |
| 148 | 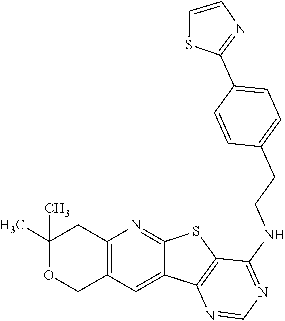 [SMILES: CC1(C)Cc2nc3sc4c(NCCc5ccc(-c6nccs6)cc5)ncnc4c3cc2CO1] |
| 149 | 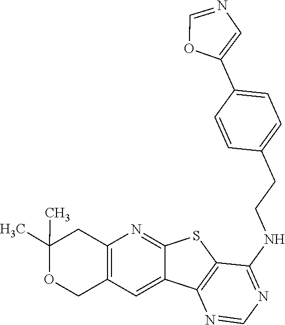 [SMILES: CC1(C)Cc2nc3sc4c(NCCc5ccc(-c6cnco6)cc5)ncnc4c3cc2CO1] |
| 150 | 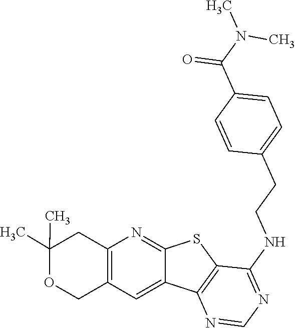 [SMILES: CN(C)C(=O)c1ccc(CCNc2ncnc3c2sc2nc4c(cc23)COC(C)(C)C4)cc1] |
| 151 | 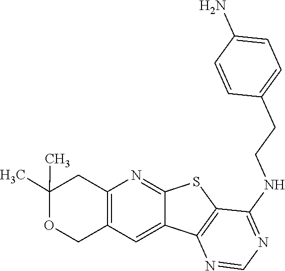 [SMILES: CC1(C)Cc2nc3sc4c(NCCc5ccc(N)cc5)ncnc4c3cc2CO1] |
| 152 | 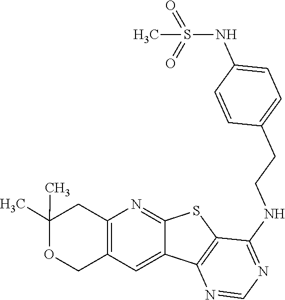 [SMILES: CC1(C)Cc2nc3sc4c(NCCc5ccc(NS(C)(=O)=O)cc5)ncnc4c3cc2CO1] |
| 153 | 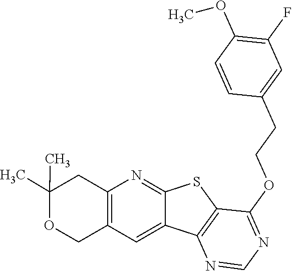 [SMILES: COc1ccc(CCOc2ncnc3c2sc2nc4c(cc23)COC(C)(C)C4)cc1F] |
| 154 | 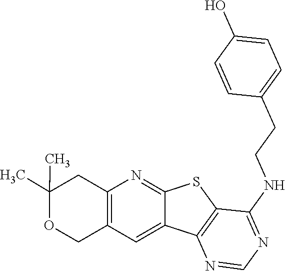 [SMILES: CC1(C)Cc2nc3sc4c(NCCc5ccc(O)cc5)ncnc4c3cc2CO1] |
| 156 | 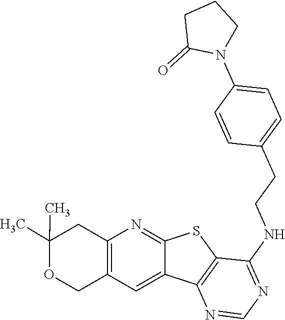 [SMILES: CC1(C)Cc2nc3sc4c(NCCc5ccc(N6CCCC6=O)cc5)ncnc4c3cc2CO1] |
| 157 | 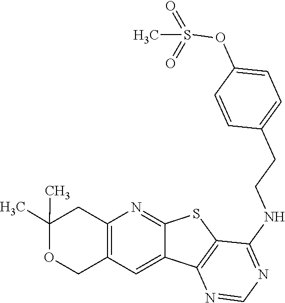 [SMILES: CC1(C)Cc2nc3sc4c(NCCc5ccc(OS(C)(=O)=O)cc5)ncnc4c3cc2CO1] |
| 158 | 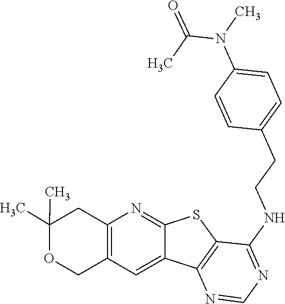 [SMILES: CC(=O)N(C)c1ccc(CCNc2ncnc3c2sc2nc4c(cc23)COC(C)(C)C4)cc1] |
| 159 | 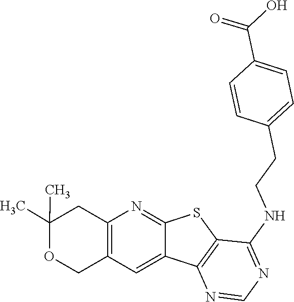 [SMILES: CC1(C)Cc2nc3sc4c(NCCc5ccc(C(=O)O)cc5)ncnc4c3cc2CO1] |
| 160 | 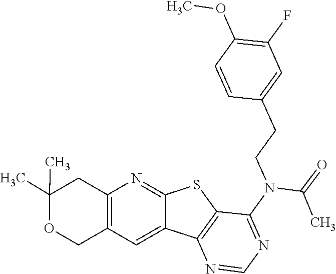 [SMILES: COc1ccc(CCN(C(C)=O)c2ncnc3c2sc2nc4c(cc23)COC(C)(C)C4)cc1F] |
| 161 | 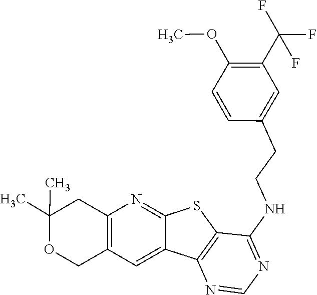 [SMILES: COc1ccc(CCNc2ncnc3c2sc2nc4c(cc23)COC(C)(C)C4)cc1C(F)(F)F] |
| 162 | 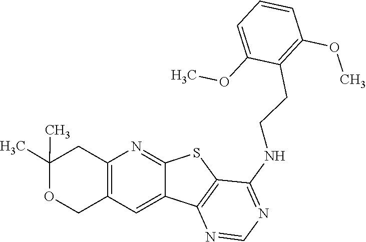 |
| 163 | 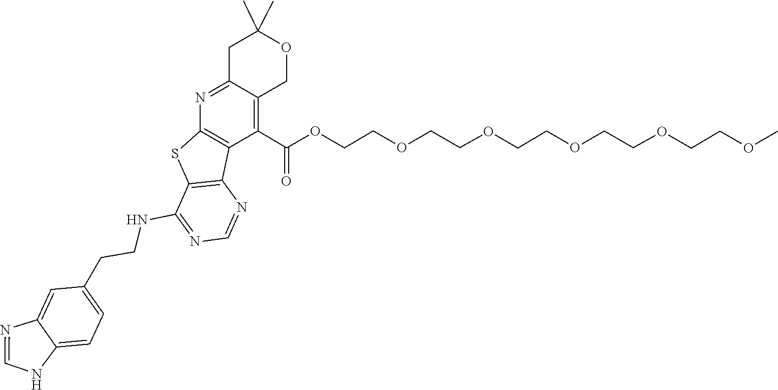 [SMILES: COCCOCCOCCOCCOCCOC(=O)c1c2c(nc3sc4c(NCCc5ccc6[nH]cnc6c5)ncnc4c13)CC(C)(C)OC2] |
| 164 | 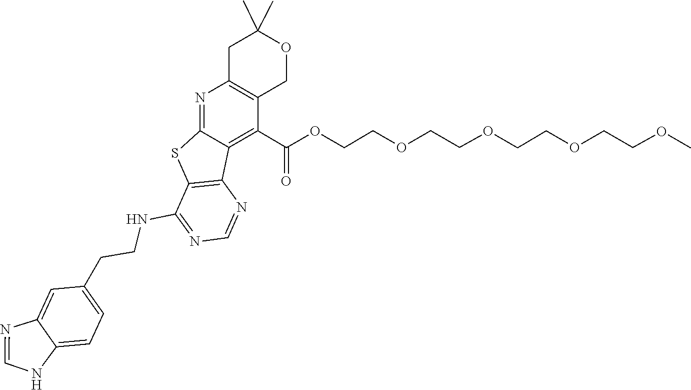 [SMILES: COCCOCCOCCOCCOC(=O)c1c2c(nc3sc4c(NCCc5ccc6[nH]cnc6c5)ncnc4c13)CC(C)(C)OC2] |
| 165 | 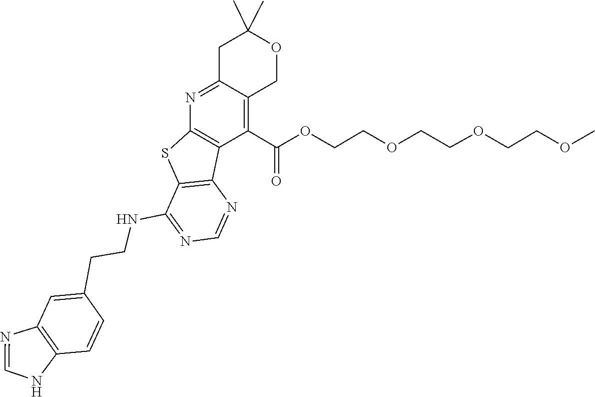 [SMILES: COCCOCCOCCOC(=O)c1c2c(nc3sc4c(NCCc5ccc6[nH]cnc6c5)ncnc4c13)CC(C)(C)OC2] |
| 166 | 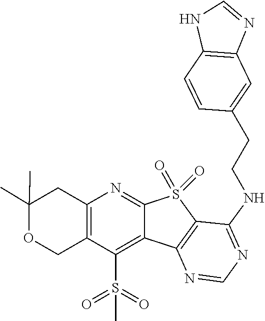 [SMILES: CC1(C)Cc2nc3c(c(S(C)(=O)=O)c2CO1)-c1ncnc(NCCc2ccc4[nH]cnc4c2)c1S3(=O)=O] |
| 167 | 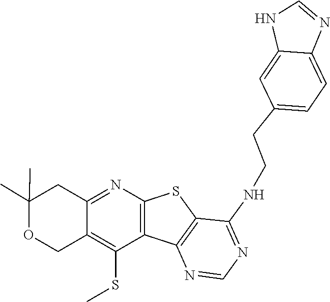 [SMILES: CSc1c2c(nc3sc4c(NCCc5ccc6nc[nH]c6c5)ncnc4c13)CC(C)(C)OC2] |
| 168 | 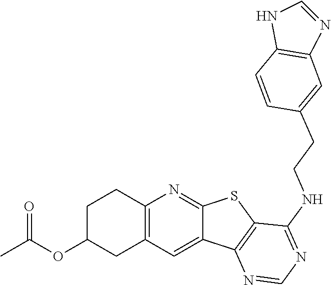 [SMILES: CC(=O)OC1CCc2nc3sc4c(NCCc5ccc6[nH]cnc6c5)ncnc4c3cc2C1] |
| 169 | 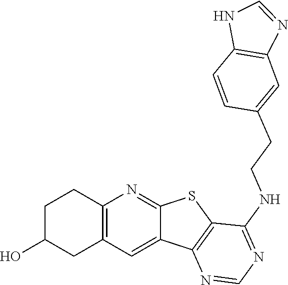 [SMILES: OC1CCc2nc3sc4c(NCCc5ccc6[nH]cnc6c5)ncnc4c3cc2C1] |
| 170 | 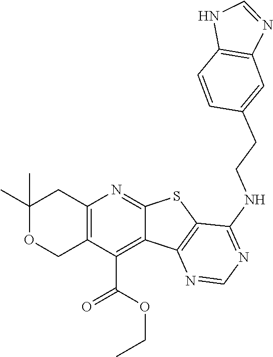 [SMILES: CCOC(=O)c1c2c(nc3sc4c(NCCc5ccc6[nH]cnc6c5)ncnc4c13)CC(C)(C)OC2] |
| 171 | 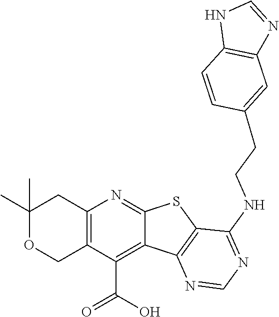 [SMILES: CC1(C)Cc2nc3sc4c(NCCc5ccc6[nH]cnc6c5)ncnc4c3c(C(=O)O)c2CO1] |
| 172 | 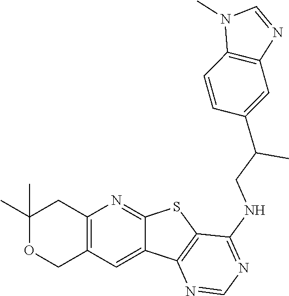 [SMILES: CC(CNc1ncnc2c1sc1nc3c(cc12)COC(C)(C)C3)c1ccc2c(c1)ncn2C] |
| 173 | 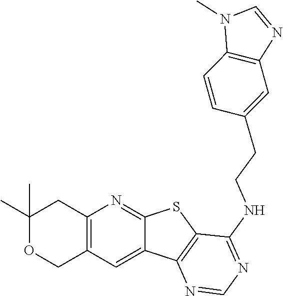 [SMILES: Cn1cnc2cc(CCNc3ncnc4c3sc3nc5c(cc34)COC(C)(C)C5)ccc21] |
| 174 | 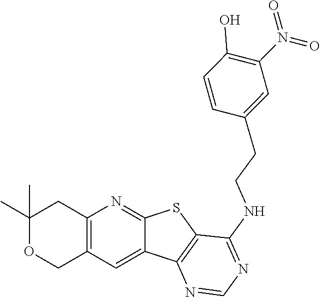 [SMILES: CC1(C)Cc2nc3sc4c(NCCc5ccc(O)c([N+](=O)[O-])c5)ncnc4c3cc2CO1] |
| 175 | 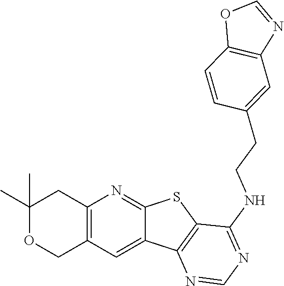 [SMILES: CC1(C)Cc2nc3sc4c(NCCc5ccc6ocnc6c5)ncnc4c3cc2CO1] |
| 176 | 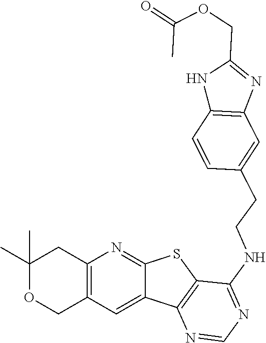 [SMILES: CC(=O)OCc1nc2cc(CCNc3ncnc4c3sc3nc5c(cc34)COC(C)(C)C5)ccc2[nH]1] |
| 177 | 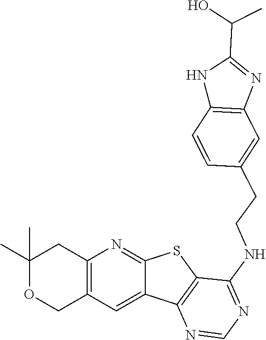 [SMILES: CC(O)c1nc2cc(CCNc3ncnc4c3sc3nc5c(cc34)COC(C)(C)C5)ccc2[nH]1] |
| 178 | 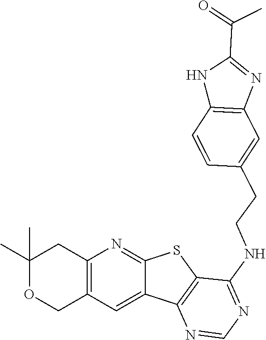 [SMILES: CC(=O)c1nc2cc(CCNc3ncnc4c3sc3nc5c(cc34)COC(C)(C)C5)ccc2[nH]1] |
| 179 | 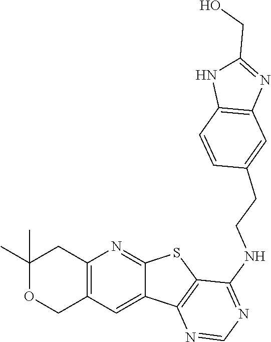 [SMILES: CC1(C)Cc2nc3sc4c(NCCc5ccc6[nH]c(CO)nc6c5)ncnc4c3cc2CO1] |
| 180 | 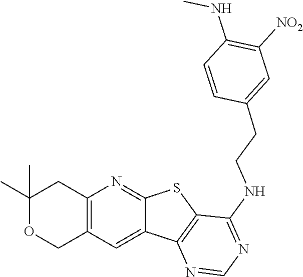 [SMILES: CNc1ccc(CCNc2ncnc3c2sc2nc4c(cc23)COC(C)(C)C4)cc1[N+](=O)[O-]] |
| 181 | 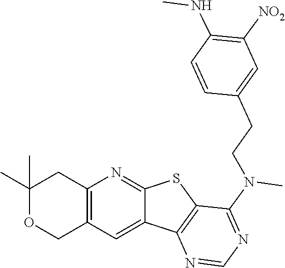 |
| 182 | 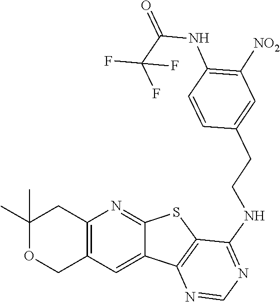 [SMILES: CC1(C)Cc2nc3sc4c(NCCc5ccc(NC(=O)C(F)(F)F)c([N+](=O)[O-])c5)ncnc4c3cc2CO1] |
| 183 | 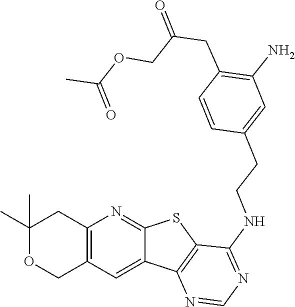 [SMILES: CC(=O)OCC(=O)Cc1ccc(CCNc2ncnc3c2sc2nc4c(cc23)COC(C)(C)C4)cc1N] |
| 184 | 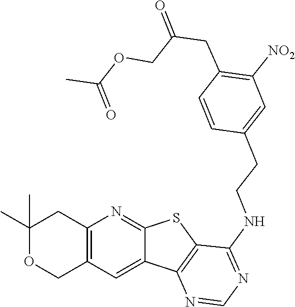 [SMILES: CC(=O)OCC(=O)Cc1ccc(CCNc2ncnc3c2sc2nc4c(cc23)COC(C)(C)C4)cc1[N+](=O)[O-]] |
| 185 | 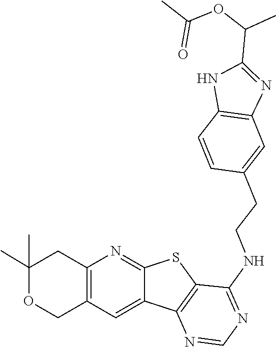 [SMILES: CC(=O)OC(C)c1nc2cc(CCNc3ncnc4c3sc3nc5c(cc34)COC(C)(C)C5)ccc2[nH]1] |
| 186 | 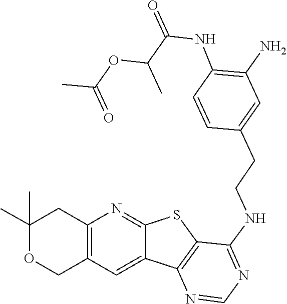 [SMILES: CC(=O)OC(C)C(=O)Nc1ccc(CCNc2ncnc3c2sc2nc4c(cc23)COC(C)(C)C4)cc1N] |
| 187 | 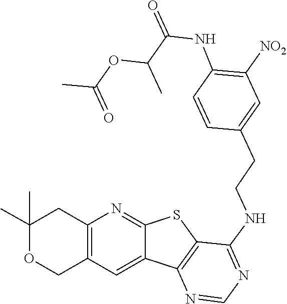 [SMILES: CC(=O)OC(C)C(=O)Nc1ccc(CCNc2ncnc3c2sc2nc4c(cc23)COC(C)(C)C4)cc1[N+](=O)[O-]] |
| 188 | 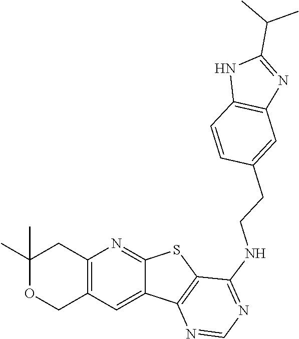 [SMILES: CC(C)c1nc2cc(CCNc3ncnc4c3sc3nc5c(cc34)COC(C)(C)C5)ccc2[nH]1] |
| 189 | 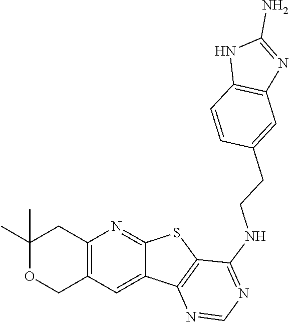 [SMILES: CC1(C)Cc2nc3sc4c(NCCc5ccc6[nH]c(N)nc6c5)ncnc4c3cc2CO1] |
| 190 | 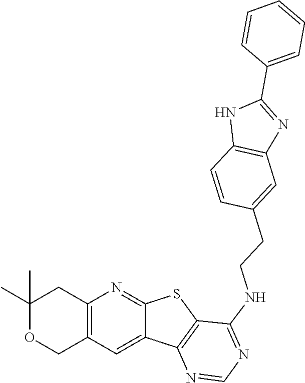 [SMILES: CC1(C)Cc2nc3sc4c(NCCc5ccc6[nH]c(-c7ccccc7)nc6c5)ncnc4c3cc2CO1] |
| 191 | 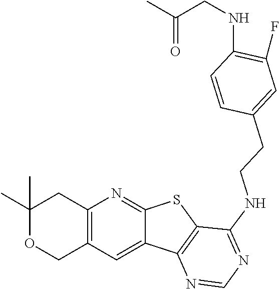 [SMILES: CC(=O)CNc1ccc(CCNc2ncnc3c2sc2nc4c(cc23)COC(C)(C)C4)cc1F] |
| 192 | 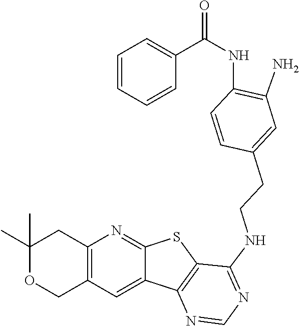 [SMILES: CC1(C)Cc2nc3sc4c(NCCc5ccc(NC(=O)c6ccccc6)c(N)c5)ncnc4c3cc2CO1] |
| 193 | 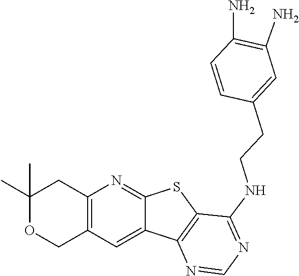 [SMILES: CC1(C)Cc2nc3sc4c(NCCc5ccc(N)c(N)c5)ncnc4c3cc2CO1] |
| 194 | 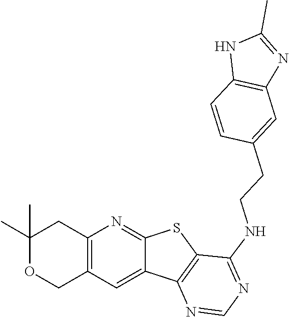 [SMILES: Cc1nc2cc(CCNc3ncnc4c3sc3nc5c(cc34)COC(C)(C)C5)ccc2[nH]1] |
| 195 | 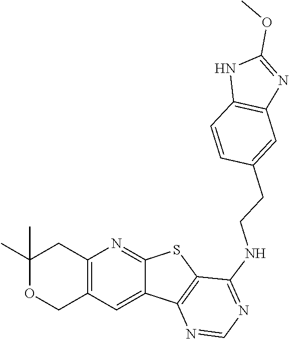 [SMILES: COc1nc2cc(CCNc3ncnc4c3sc3nc5c(cc34)COC(C)(C)C5)ccc2[nH]1] |
| 196 | 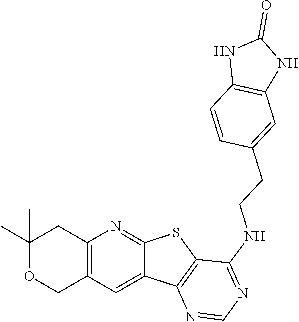 [SMILES: CC1(C)Cc2nc3sc4c(NCCc5ccc6[nH]c(=O)[nH]c6c5)ncnc4c3cc2CO1] |
| 197 | 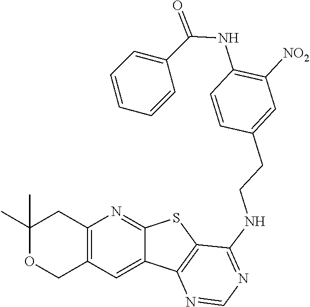 [SMILES: CC1(C)Cc2nc3sc4c(NCCc5ccc(NC(=O)c6ccccc6)c([N+](=O)[O-])c5)ncnc4c3cc2CO1] |
| 198 | 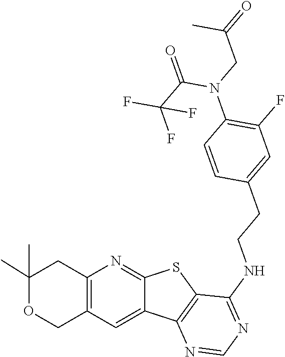 [SMILES: CC(=O)CN(C(=O)C(F)(F)F)c1ccc(CCNc2ncnc3c2sc2nc4c(cc23)COC(C)(C)C4)cc1F] |
| 199 | 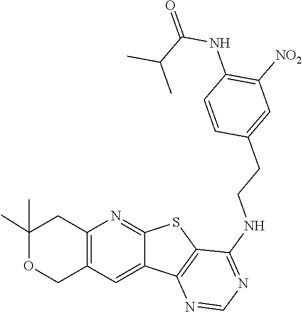 [SMILES: CC(C)C(=O)Nc1ccc(CCNc2ncnc3c2sc2nc4c(cc23)COC(C)(C)C4)cc1[N+](=O)[O-]] |
| 200 | 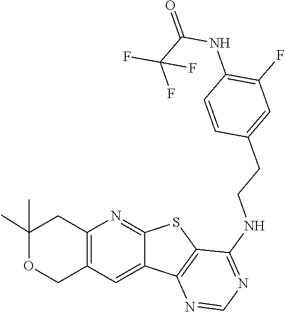 [SMILES: CC1(C)Cc2nc3sc4c(NCCc5ccc(NC(=O)C(F)(F)F)c(F)c5)ncnc4c3cc2CO1] |
| 201 | 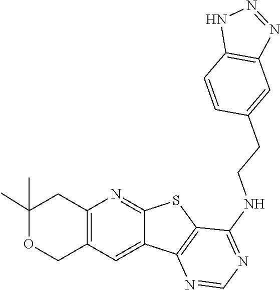 [SMILES: CC1(C)Cc2nc3sc4c(NCCc5ccc6[nH]nnc6c5)ncnc4c3cc2CO1] |
| 202 | 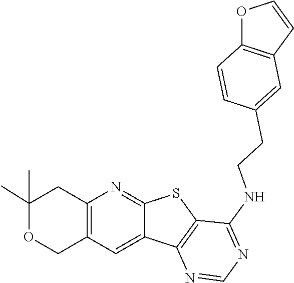 [SMILES: CC1(C)Cc2nc3sc4c(NCCc5ccc6occc6c5)ncnc4c3cc2CO1] |
| 203 | 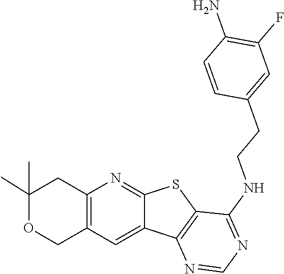 [SMILES: CC1(C)Cc2nc3sc4c(NCCc5ccc(N)c(F)c5)ncnc4c3cc2CO1] |
| 204 | 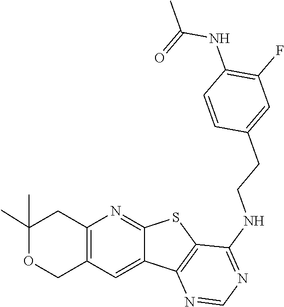 [SMILES: CC(=O)Nc1ccc(CCNc2ncnc3c2sc2nc4c(cc23)COC(C)(C)C4)cc1F] |
| 205 | 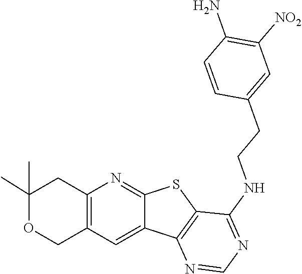 [SMILES: CC1(C)Cc2nc3sc4c(NCCc5ccc(N)c([N+](=O)[O-])c5)ncnc4c3cc2CO1] |
| 206 | 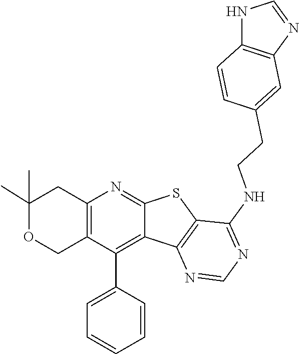 [SMILES: CC1(C)Cc2nc3sc4c(NCCc5ccc6[nH]cnc6c5)ncnc4c3c(-c3ccccc3)c2CO1] |
| 207 | 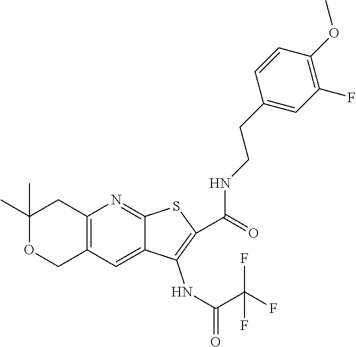 [SMILES: COc1ccc(CCNC(=O)c2sc3nc4c(cc3c2NC(=O)C(F)(F)F)COC(C)(C)C4)cc1F] |
| 208 | 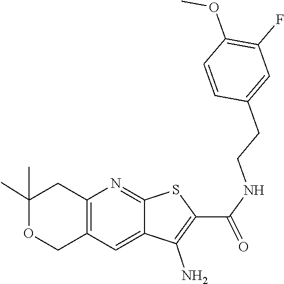 [SMILES: COc1ccc(CCNC(=O)c2sc3nc4c(cc3c2N)COC(C)(C)C4)cc1F] |
| 209 | 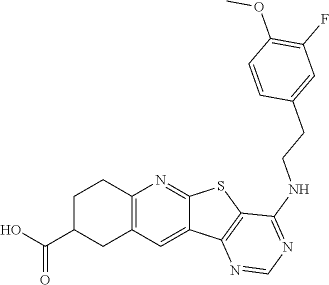 [SMILES: COc1ccc(CCNc2ncnc3c2sc2nc4c(cc23)CC(C(=O)O)CC4)cc1F] |
| 210 | 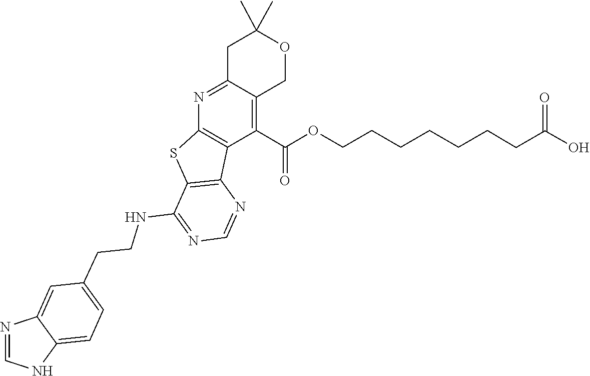 [SMILES: CC1(C)Cc2nc3sc4c(NCCc5ccc6[nH]cnc6c5)ncnc4c3c(C(=O)OCCCCCCCC(=O)O)c2CO1] |
| 211 | 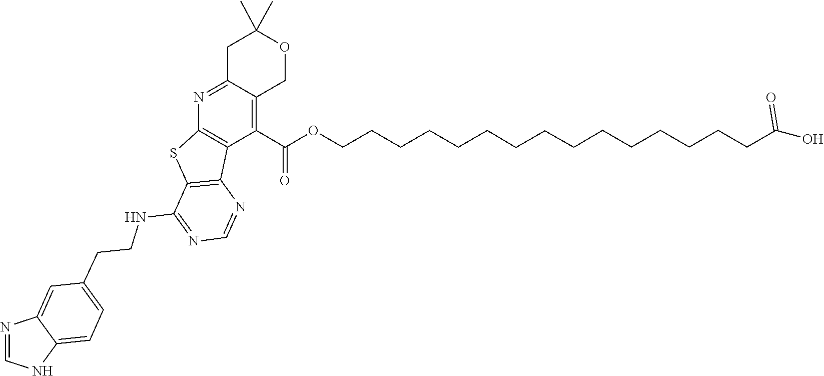 [SMILES: CC1(C)Cc2nc3sc4c(NCCc5ccc6[nH]cnc6c5)ncnc4c3c(C(=O)OCCCCCCCCCCCCCCCC(=O)O)c2CO1] |
| 212 | 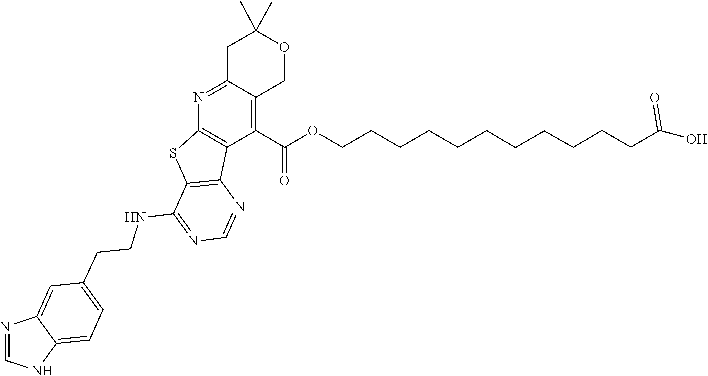 [SMILES: CC1(C)Cc2nc3sc4c(NCCc5ccc6[nH]cnc6c5)ncnc4c3c(C(=O)OCCCCCCCCCCCC(=O)O)c2CO1] |
| 213 | 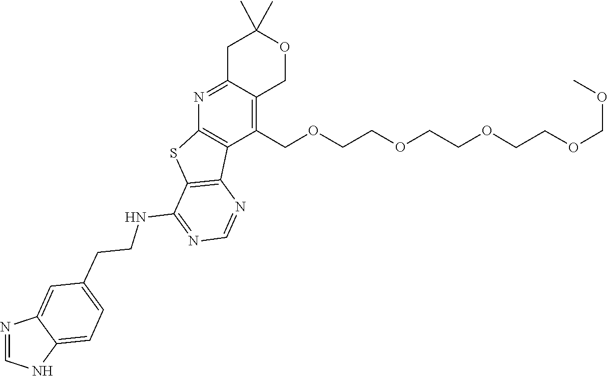 [SMILES: COCOCCOCCOCCOCc1c2c(nc3sc4c(NCCc5ccc6[nH]cnc6c5)ncnc4c13)CC(C)(C)OC2] |
| 214 | 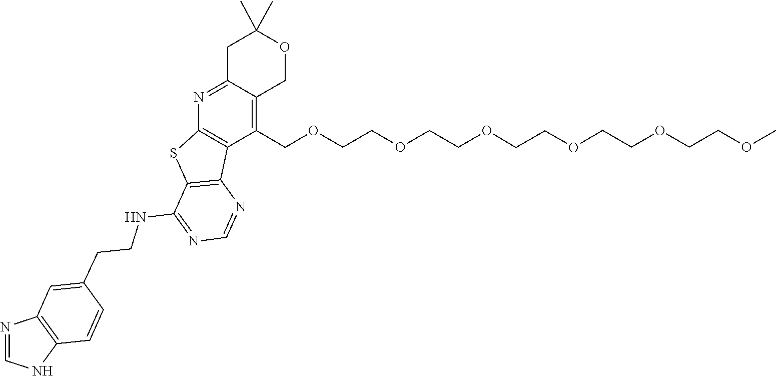 [SMILES: COCCOCCOCCOCCOCCOCc1c2c(nc3sc4c(NCCc5ccc6[nH]cnc6c5)ncnc4c13)CC(C)(C)OC2] |
| 215 | 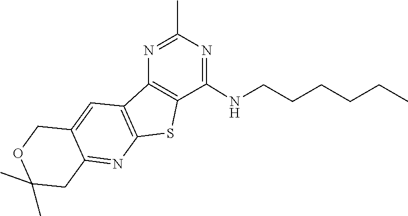 [SMILES: CCCCCCNc1nc(C)nc2c1sc1nc3c(cc12)COC(C)(C)C3] |
| 216 | 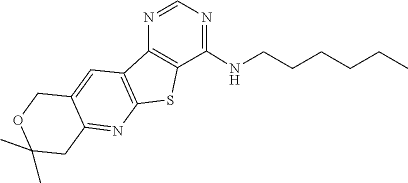 [SMILES: CCCCCCNc1ncnc2c1sc1nc3c(cc12)COC(C)(C)C3] |
| 217 | 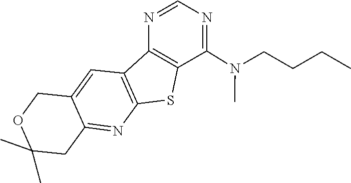 [SMILES: CCCCN(C)c1ncnc2c1sc1nc3c(cc12)COC(C)(C)C3] |
| 218 | 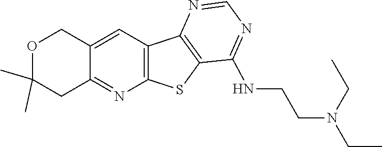 [SMILES: CCN(CC)CCNc1ncnc2c1sc1nc3c(cc12)COC(C)(C)C3] |
| 219 | 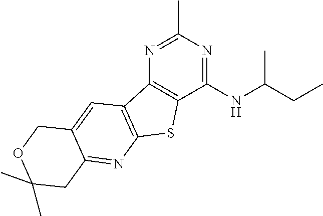 [SMILES: CCC(C)Nc1nc(C)nc2c1sc1nc3c(cc12)COC(C)(C)C3] |
| 220 | 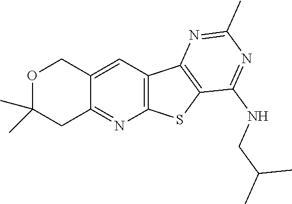 [SMILES: Cc1nc(NCC(C)C)c2sc3nc4c(cc3c2n1)COC(C)(C)C4] |
| 221 | 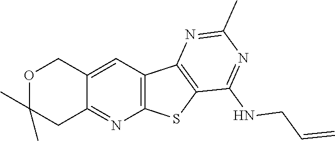 [SMILES: C=CCNc1nc(C)nc2c1sc1nc3c(cc12)COC(C)(C)C3] |
| 222 | 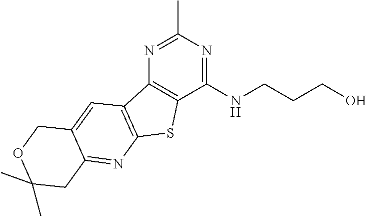 [SMILES: Cc1nc(NCCCO)c2sc3nc4c(cc3c2n1)COC(C)(C)C4] |
| 223 | 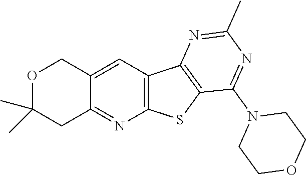 [SMILES: Cc1nc(N2CCOCC2)c2sc3nc4c(cc3c2n1)COC(C)(C)C4] |
| 224 | 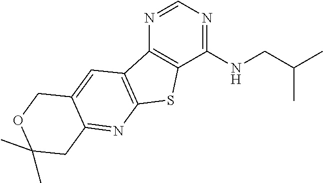 [SMILES: CC(C)CNc1ncnc2c1sc1nc3c(cc12)COC(C)(C)C3] |
| 225 | 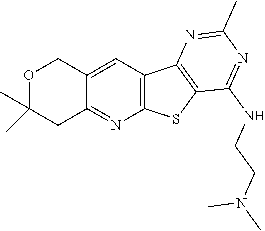 |
| 226 | 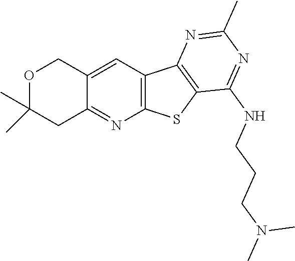 |
| 227 | 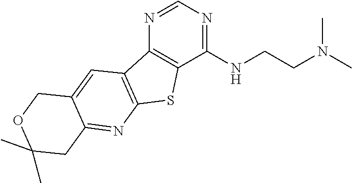 |
| 228 | 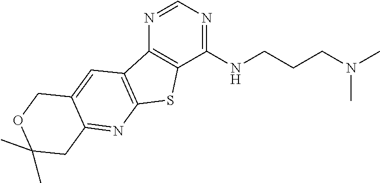 |
| 229 | 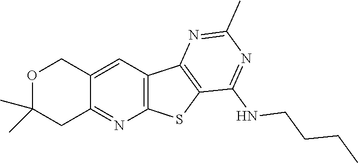 [SMILES: CCCCNc1nc(C)nc2c1sc1nc3c(cc12)COC(C)(C)C3] |
| 230 | 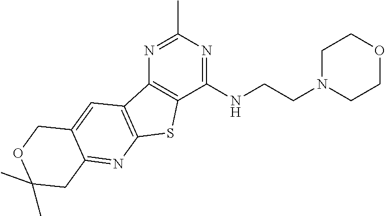 [SMILES: Cc1nc(NCCN2CCOCC2)c2sc3nc4c(cc3c2n1)COC(C)(C)C4] |
| 231 | 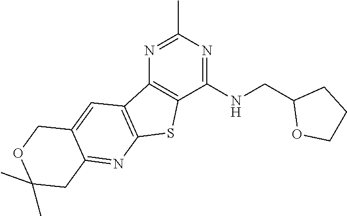 [SMILES: Cc1nc(NCC2CCCO2)c2sc3nc4c(cc3c2n1)COC(C)(C)C4] |
| 232 | 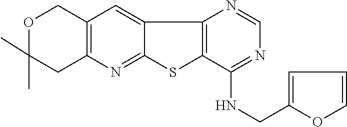 [SMILES: CC1(C)Cc2nc3sc4c(NCc5ccco5)ncnc4c3cc2CO1] |
| 233 | 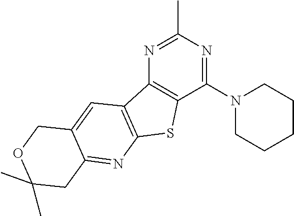 [SMILES: Cc1nc(N2CCCCC2)c2sc3nc4c(cc3c2n1)COC(C)(C)C4] |
| 234 | 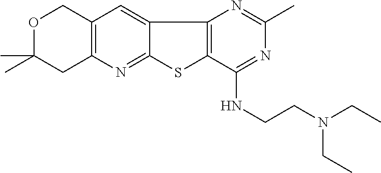 [SMILES: CCN(CC)CCNc1nc(C)nc2c1sc1nc3c(cc12)COC(C)(C)C3] |
| 235 | 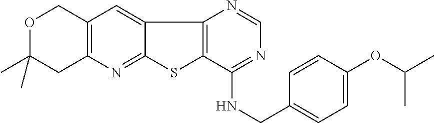 [SMILES: CC(C)Oc1ccc(CNc2ncnc3c2sc2nc4c(cc23)COC(C)(C)C4)cc1] |
| 236 | 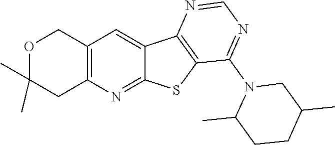 |
| 237 | 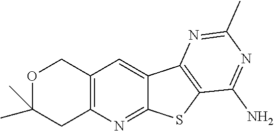 [SMILES: Cc1nc(N)c2sc3nc4c(cc3c2n1)COC(C)(C)C4] |
| 238 | 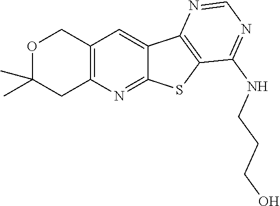 [SMILES: CC1(C)Cc2nc3sc4c(NCCCO)ncnc4c3cc2CO1] |
| 239 | 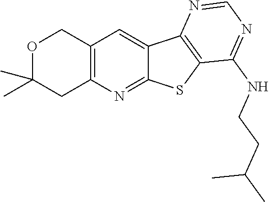 [SMILES: CC(C)CCNc1ncnc2c1sc1nc3c(cc12)COC(C)(C)C3] |
| 240 | 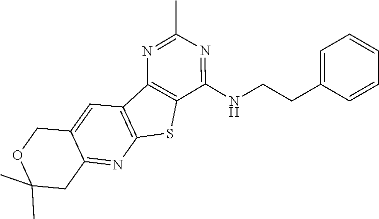 [SMILES: Cc1nc(NCCc2ccccc2)c2sc3nc4c(cc3c2n1)COC(C)(C)C4] |
| 241 | 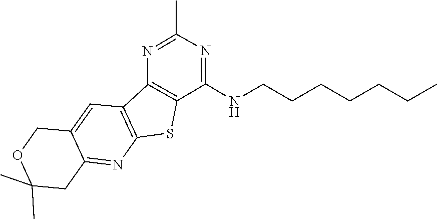 [SMILES: CCCCCCCNc1nc(C)nc2c1sc1nc3c(cc12)COC(C)(C)C3] |
| 242 | 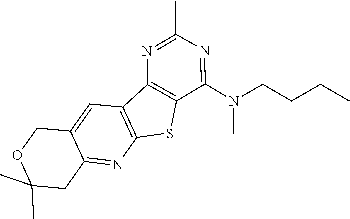 [SMILES: CCCCN(C)c1nc(C)nc2c1sc1nc3c(cc12)COC(C)(C)C3] |
| 243 | 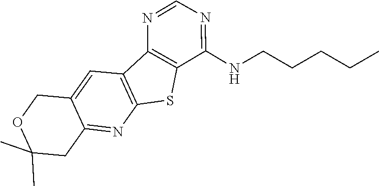 [SMILES: CCCCCNc1ncnc2c1sc1nc3c(cc12)COC(C)(C)C3] |
| 244 | 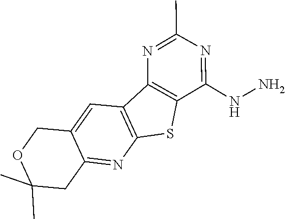 [SMILES: Cc1nc(NN)c2sc3nc4c(cc3c2n1)COC(C)(C)C4] |
| 245 | 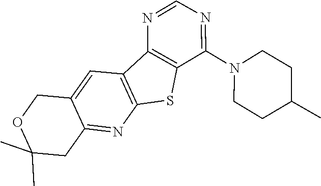 |
| 246 | 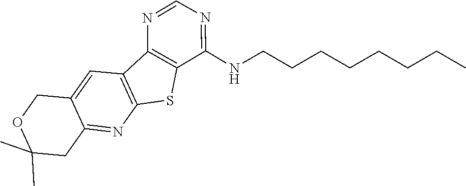 [SMILES: CCCCCCCCNc1ncnc2c1sc1nc3c(cc12)COC(C)(C)C3] |
| 247 | 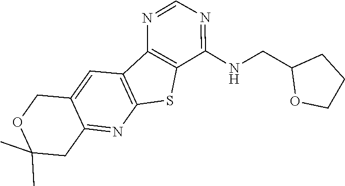 [SMILES: CC1(C)Cc2nc3sc4c(NCC5CCCO5)ncnc4c3cc2CO1] |
| 248 | 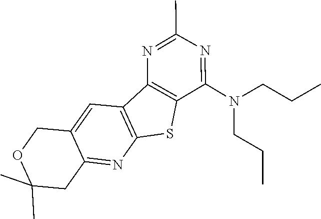 [SMILES: CCCN(CCC)c1nc(C)nc2c1sc1nc3c(cc12)COC(C)(C)C3] |
| 249 | 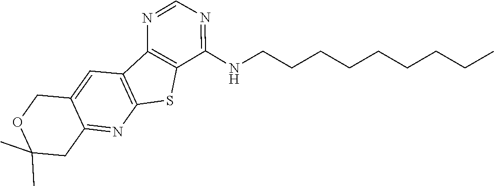 [SMILES: CCCCCCCCCNc1ncnc2c1sc1nc3c(cc12)COC(C)(C)C3] |
| 250 | 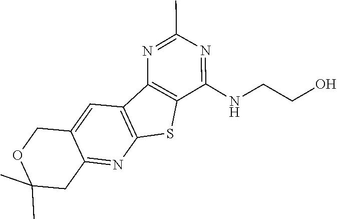 [SMILES: Cc1nc(NCCO)c2sc3nc4c(cc3c2n1)COC(C)(C)C4] |
| 251 | 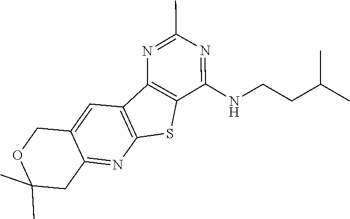 [SMILES: Cc1nc(NCCC(C)C)c2sc3nc4c(cc3c2n1)COC(C)(C)C4] |
| 252 | 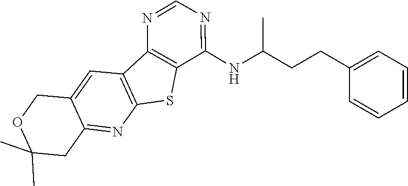 [SMILES: CC(CCc1ccccc1)Nc1ncnc2c1sc1nc3c(cc12)COC(C)(C)C3] |
| 253 | 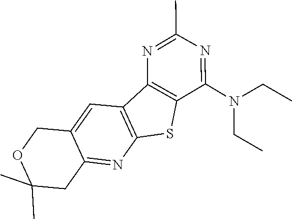 [SMILES: CCN(CC)c1nc(C)nc2c1sc1nc3c(cc12)COC(C)(C)C3] |
| 254 | 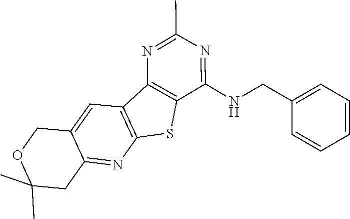 [SMILES: Cc1nc(NCc2ccccc2)c2sc3nc4c(cc3c2n1)COC(C)(C)C4] |
| 255 | 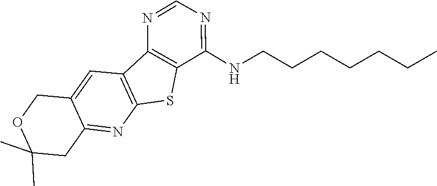 [SMILES: CCCCCCCNc1ncnc2c1sc1nc3c(cc12)COC(C)(C)C3] |
| 256 | 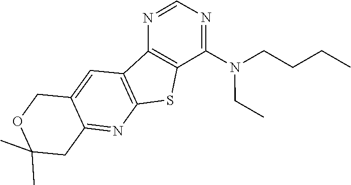 [SMILES: CCCCN(CC)c1ncnc2c1sc1nc3c(cc12)COC(C)(C)C3] |
| 257 | 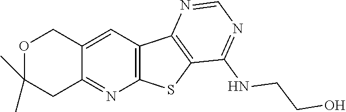 [SMILES: CC1(C)Cc2nc3sc4c(NCCO)ncnc4c3cc2CO1] |
| 258 | 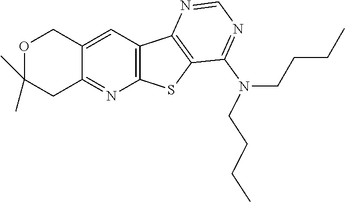 [SMILES: CCCCN(CCCC)c1ncnc2c1sc1nc3c(cc12)COC(C)(C)C3] |
| 259 | 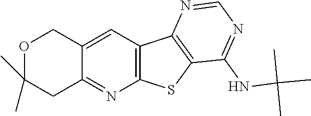 [SMILES: CC(C)(C)Nc1ncnc2c1sc1nc3c(cc12)COC(C)(C)C3] |
| 260 | 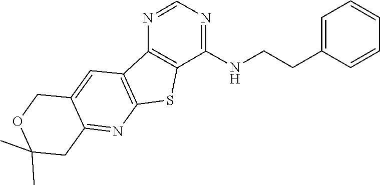 [SMILES: CC1(C)Cc2nc3sc4c(NCCc5ccccc5)ncnc4c3cc2CO1] |
| 261 | 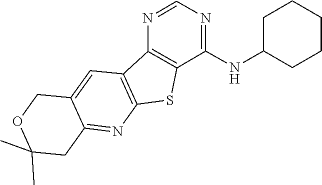 [SMILES: CC1(C)Cc2nc3sc4c(NC5CCCCC5)ncnc4c3cc2CO1] |
| 262 | 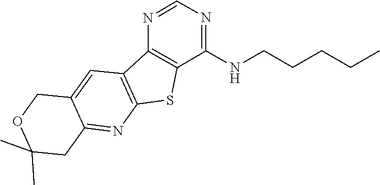 [SMILES: CCCCCNc1ncnc2c1sc1nc3c(cc12)COC(C)(C)C3] |
| 263 | 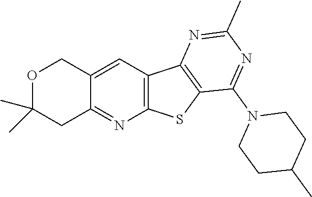 |
| 264 | 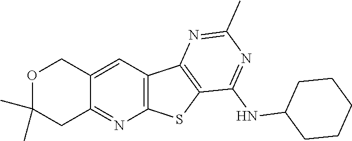 [SMILES: Cc1nc(NC2CCCCC2)c2sc3nc4c(cc3c2n1)COC(C)(C)C4] |
| 265 | 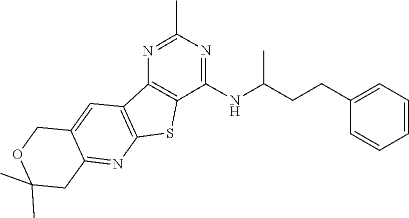 [SMILES: Cc1nc(NC(C)CCc2ccccc2)c2sc3nc4c(cc3c2n1)COC(C)(C)C4] |
| 266 | 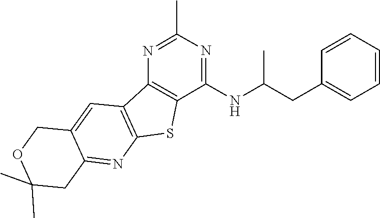 [SMILES: Cc1nc(NC(C)Cc2ccccc2)c2sc3nc4c(cc3c2n1)COC(C)(C)C4] |
| 267 | 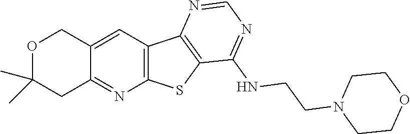 [SMILES: CC1(C)Cc2nc3sc4c(NCCN5CCOCC5)ncnc4c3cc2CO1] |
| 268 | 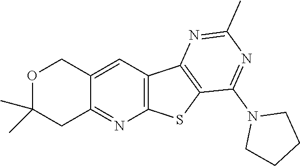 [SMILES: Cc1nc(N2CCCC2)c2sc3nc4c(cc3c2n1)COC(C)(C)C4] |
| 269 | 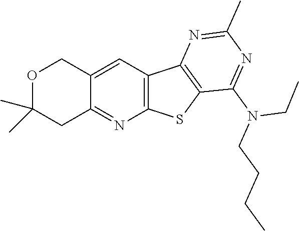 [SMILES: CCCCN(CC)c1nc(C)nc2c1sc1nc3c(cc12)COC(C)(C)C3] |
| 270 | 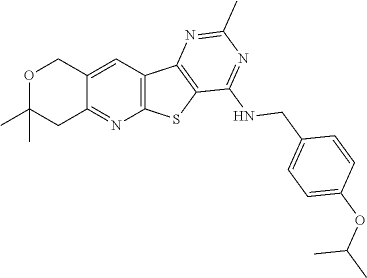 [SMILES: Cc1nc(NCc2ccc(OC(C)C)cc2)c2sc3nc4c(cc3c2n1)COC(C)(C)C4] |
| 271 | 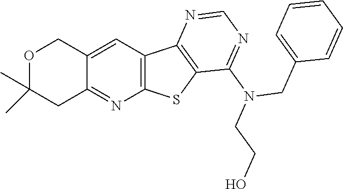 [SMILES: CC1(C)Cc2nc3sc4c(N(CCO)Cc5ccccc5)ncnc4c3cc2CO1] |
| 272 | 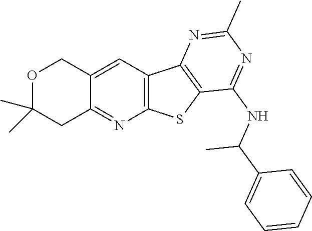 [SMILES: Cc1nc(NC(C)c2ccccc2)c2sc3nc4c(cc3c2n1)COC(C)(C)C4] |
| 273 | 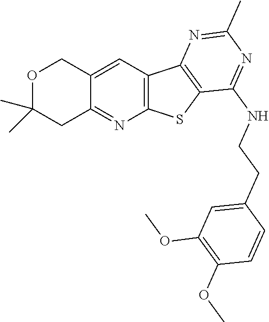 [SMILES: COc1ccc(CCNc2nc(C)nc3c2sc2nc4c(cc23)COC(C)(C)C4)cc1OC] |
| 274 | 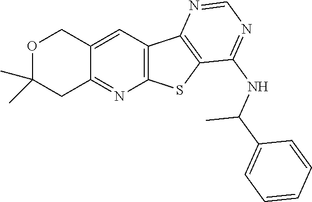 [SMILES: CC(Nc1ncnc2c1sc1nc3c(cc12)COC(C)(C)C3)c1ccccc1] |
| 275 | 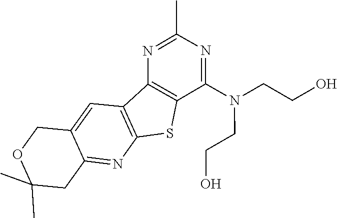 [SMILES: Cc1nc(N(CCO)CCO)c2sc3nc4c(cc3c2n1)COC(C)(C)C4] |
| 276 | 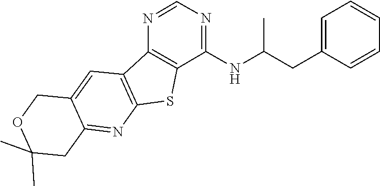 [SMILES: CC(Cc1ccccc1)Nc1ncnc2c1sc1nc3c(cc12)COC(C)(C)C3] |
| 277 | 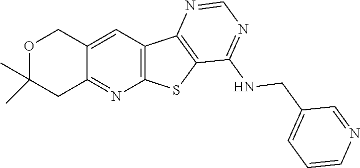 [SMILES: CC1(C)Cc2nc3sc4c(NCc5cccnc5)ncnc4c3cc2CO1] |
| 278 | 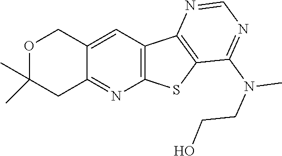 |
| 279 | 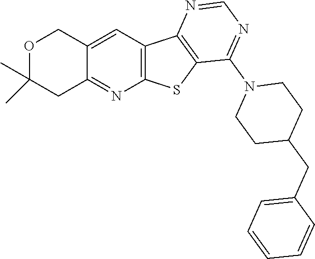 [SMILES: CC1(C)Cc2nc3sc4c(N5CCC(Cc6ccccc6)CC5)ncnc4c3cc2CO1] |
| 280 | 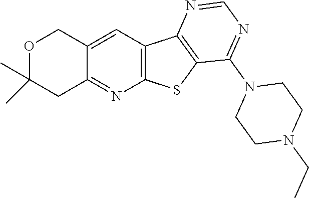 [SMILES: CCN1CCN(c2ncnc3c2sc2nc4c(cc23)COC(C)(C)C4)CC1] |
| 281 | 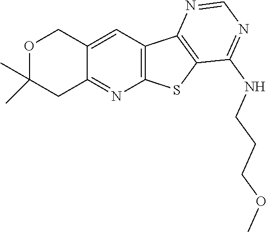 [SMILES: COCCCNc1ncnc2c1sc1nc3c(cc12)COC(C)(C)C3] |
| 282 | 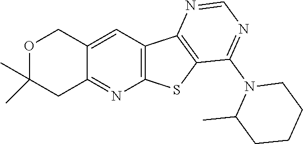 [SMILES: CC1CCCCN1c1ncnc2c1sc1nc3c(cc12)COC(C)(C)C3] |
| 283 | 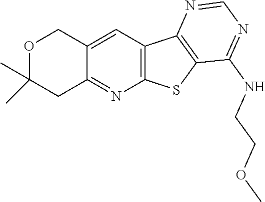 |
| 284 | 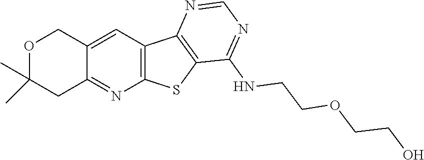 [SMILES: CC1(C)Cc2nc3sc4c(NCCOCCO)ncnc4c3cc2CO1] |
| 285 | 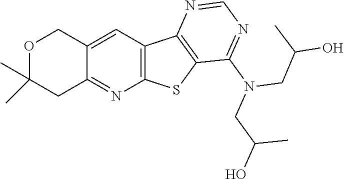 [SMILES: CC(O)CN(CC(C)O)c1ncnc2c1sc1nc3c(cc12)COC(C)(C)C3] |
| 286 | 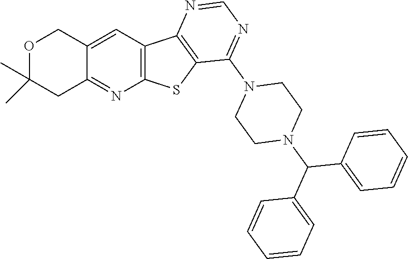 [SMILES: CC1(C)Cc2nc3sc4c(N5CCN(C(c6ccccc6)c6ccccc6)CC5)ncnc4c3cc2CO1] |
| 287 | 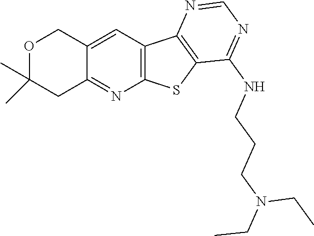 [SMILES: CCN(CC)CCCNc1ncnc2c1sc1nc3c(cc12)COC(C)(C)C3] |
| 288 | 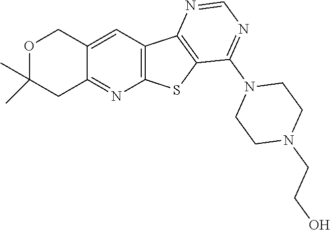 [SMILES: CC1(C)Cc2nc3sc4c(N5CCN(CCO)CC5)ncnc4c3cc2CO1] |
| 289 | 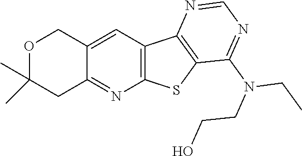 [SMILES: CCN(CCO)c1ncnc2c1sc1nc3c(cc12)COC(C)(C)C3] |
| 290 | 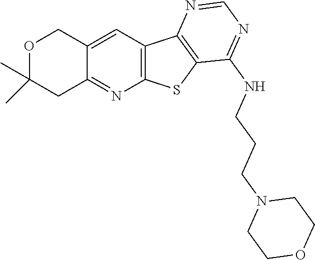 [SMILES: CC1(C)Cc2nc3sc4c(NCCCN5CCOCC5)ncnc4c3cc2CO1] |
| 291 | 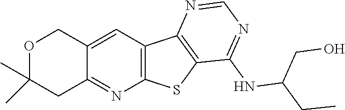 [SMILES: CCC(CO)Nc1ncnc2c1sc1nc3c(cc12)COC(C)(C)C3] |
| 292 | 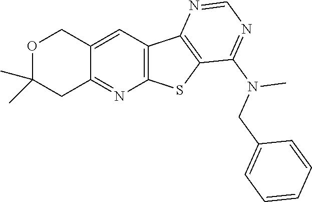 [SMILES: CN(Cc1ccccc1)c1ncnc2c1sc1nc3c(cc12)COC(C)(C)C3] |
| 293 | 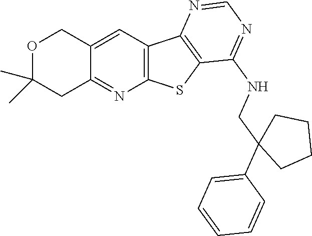 [SMILES: CC1(C)Cc2nc3sc4c(NCC5(c6ccccc6)CCCC5)ncnc4c3cc2CO1] |
| 294 | 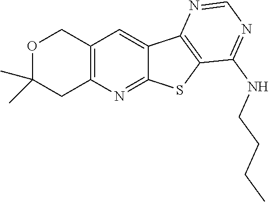 [SMILES: CCCCNc1ncnc2c1sc1nc3c(cc12)COC(C)(C)C3] |
| 295 | 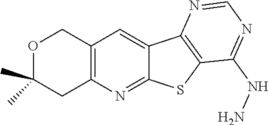 |
| 296 | 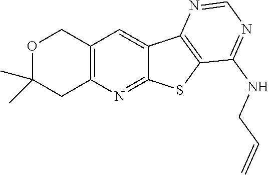 [SMILES: C=CCNc1ncnc2c1sc1nc3c(cc12)COC(C)(C)C3] |
| 297 | 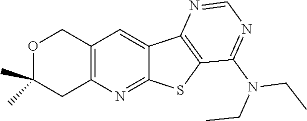 |
| 298 | 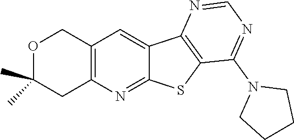 |
| 299 | 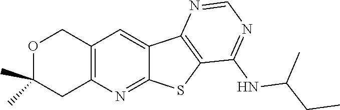 |
| 300 | 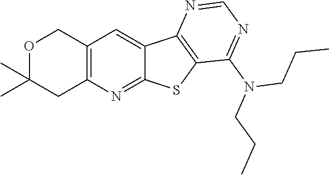 [SMILES: CCCN(CCC)c1ncnc2c1sc1nc3c(cc12)COC(C)(C)C3] |
| 301 | 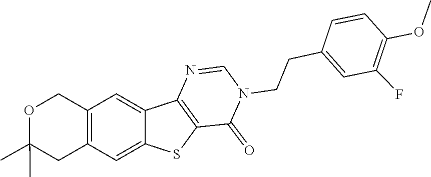 |
| 302 | 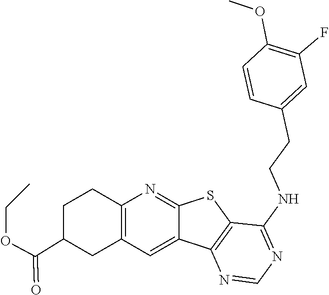 [SMILES: CCOC(=O)C1CCc2nc3sc4c(NCCc5ccc(OC)c(F)c5)ncnc4c3cc2C1] |
| 303 | 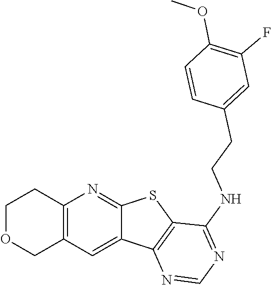 [SMILES: COc1ccc(CCNc2ncnc3c2sc2nc4c(cc23)COCC4)cc1F] |
| 304 | 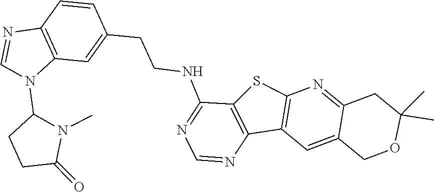 [SMILES: CN1C(=O)CCC1n1cnc2ccc(CCNc3ncnc4c3sc3nc5c(cc34)COC(C)(C)C5)cc21] |
| 305 | 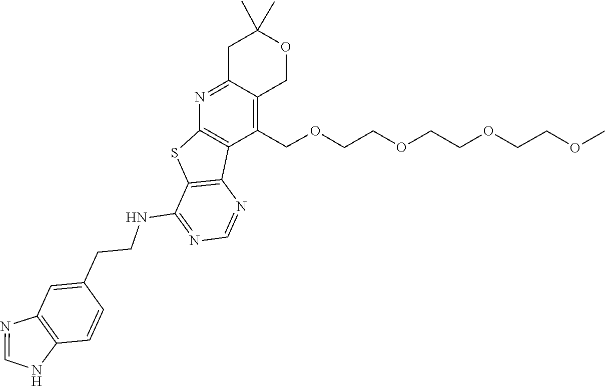 [SMILES: COCCOCCOCCOCc1c2c(nc3sc4c(NCCc5ccc6[nH]cnc6c5)ncnc4c13)CC(C)(C)OC2] |
| 306 | 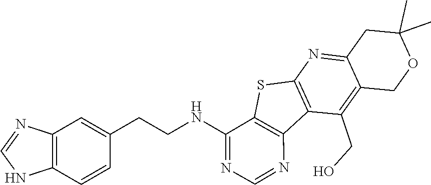 [SMILES: CC1(C)Cc2nc3sc4c(NCCc5ccc6[nH]cnc6c5)ncnc4c3c(CO)c2CO1] |
| 308 | 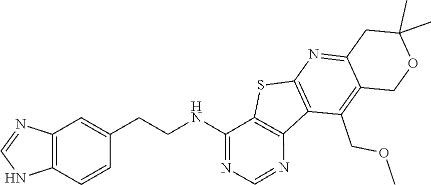 [SMILES: COCc1c2c(nc3sc4c(NCCc5ccc6[nH]cnc6c5)ncnc4c13)CC(C)(C)OC2] |
| 311 | 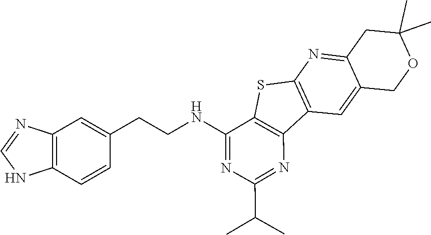 [SMILES: CC(C)c1nc(NCCc2ccc3[nH]cnc3c2)c2sc3nc4c(cc3c2n1)COC(C)(C)C4] |
| 313 | 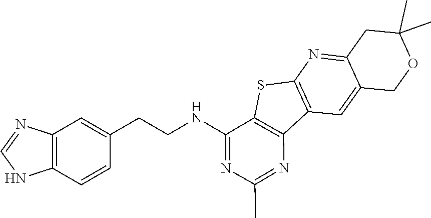 [SMILES: Cc1nc(NCCc2ccc3[nH]cnc3c2)c2sc3nc4c(cc3c2n1)COC(C)(C)C4] |
| 314 | 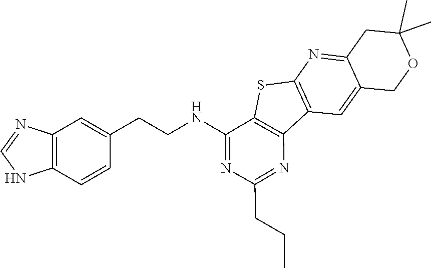 [SMILES: CCCc1nc(NCCc2ccc3[nH]cnc3c2)c2sc3nc4c(cc3c2n1)COC(C)(C)C4] |
| 315 | 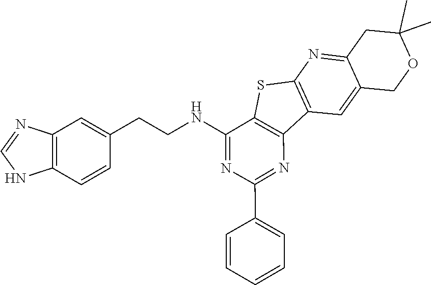 [SMILES: CC1(C)Cc2nc3sc4c(NCCc5ccc6[nH]cnc6c5)nc(-c5ccccc5)nc4c3cc2CO1] |
| 317 | 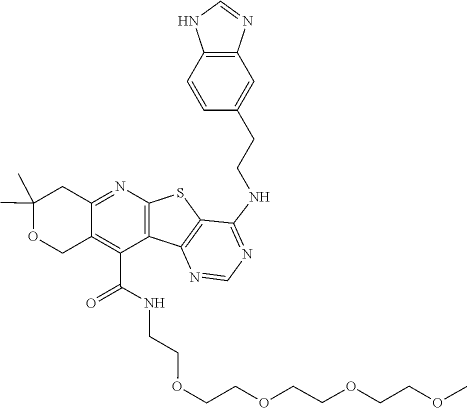 |
| 318 | 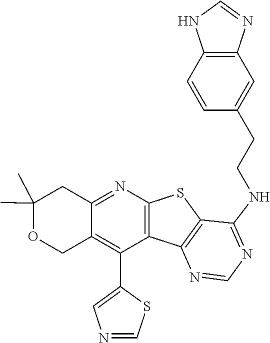 [SMILES: CC1(C)Cc2nc3sc4c(NCCc5ccc6[nH]cnc6c5)ncnc4c3c(-c3cncs3)c2CO1] |
| 319 | 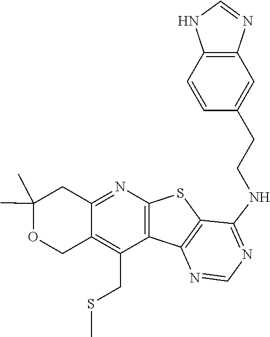 [SMILES: CSCc1c2c(nc3sc4c(NCCc5ccc6[nH]cnc6c5)ncnc4c13)CC(C)(C)OC2] |
| 320 | 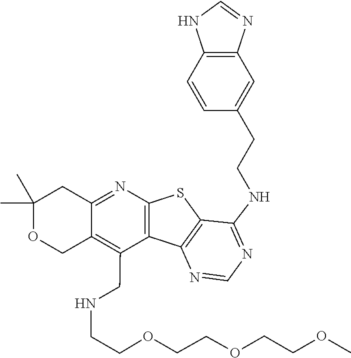 |
| 321 | 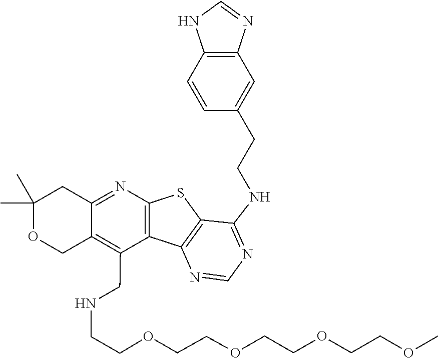 |
| 322 | 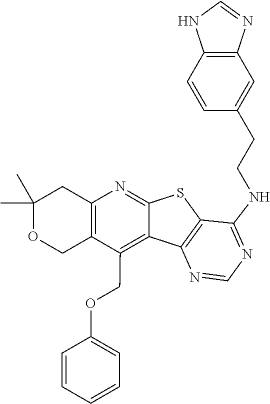 [SMILES: CC1(C)Cc2nc3sc4c(NCCc5ccc6[nH]cnc6c5)ncnc4c3c(COc3ccccc3)c2CO1] |
| 323 | 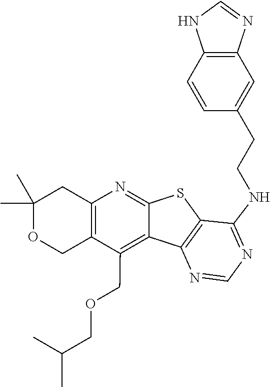 [SMILES: CC(C)COCc1c2c(nc3sc4c(NCCc5ccc6[nH]cnc6c5)ncnc4c13)CC(C)(C)OC2] |
| 324 | 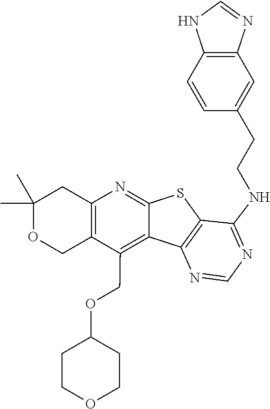 [SMILES: CC1(C)Cc2nc3sc4c(NCCc5ccc6[nH]cnc6c5)ncnc4c3c(COC3CCOCC3)c2CO1] |
| 325 | 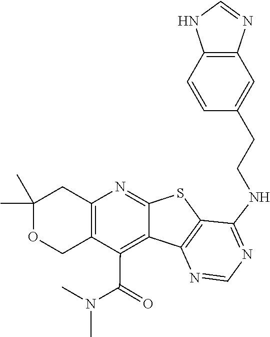 [SMILES: CN(C)C(=O)c1c2c(nc3sc4c(NCCc5ccc6[nH]cnc6c5)ncnc4c13)CC(C)(C)OC2] |
| 326 | 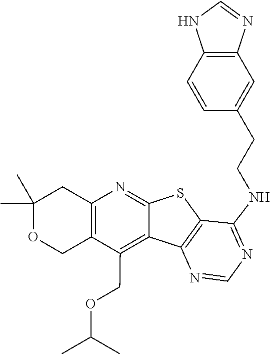 [SMILES: CC(C)OCc1c2c(nc3sc4c(NCCc5ccc6[nH]cnc6c5)ncnc4c13)CC(C)(C)OC2] |
| 327 | 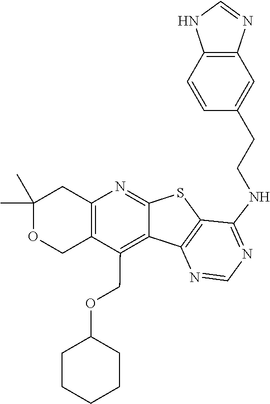 [SMILES: CC1(C)Cc2nc3sc4c(NCCc5ccc6[nH]cnc6c5)ncnc4c3c(COC3CCCCC3)c2CO1] |
| 328 | 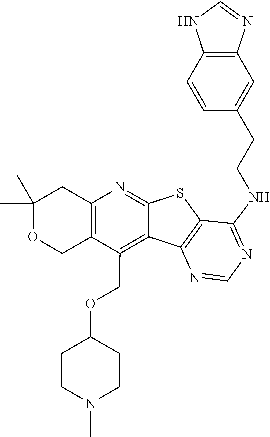 [SMILES: CN1CCC(OCc2c3c(nc4sc5c(NCCc6ccc7[nH]cnc7c6)ncnc5c24)CC(C)(C)OC3)CC1] |
| 329 | 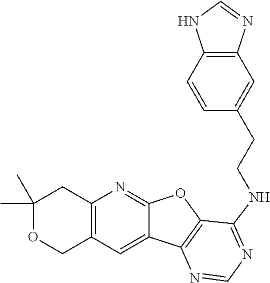 [SMILES: CC1(C)Cc2nc3oc4c(NCCc5ccc6[nH]cnc6c5)ncnc4c3cc2CO1] |
| 330 | 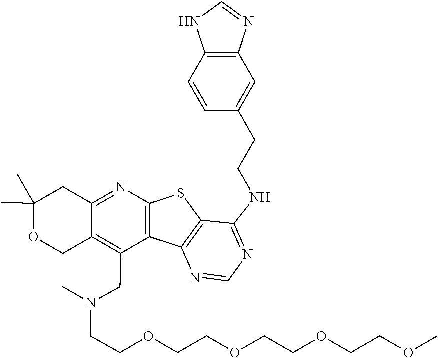 |
| 331 | 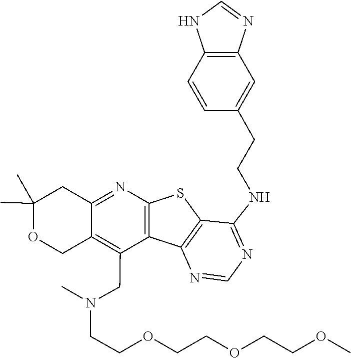 |
| 332 | 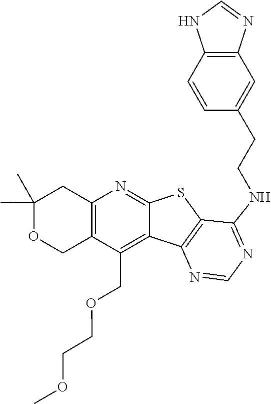 [SMILES: COCCOCc1c2c(nc3sc4c(NCCc5ccc6[nH]cnc6c5)ncnc4c13)CC(C)(C)OC2] |
| 333 | 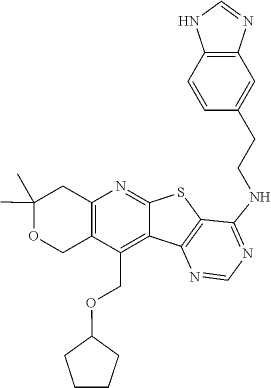 [SMILES: CC1(C)Cc2nc3sc4c(NCCc5ccc6[nH]cnc6c5)ncnc4c3c(COC3CCCC3)c2CO1] |
| 334 | 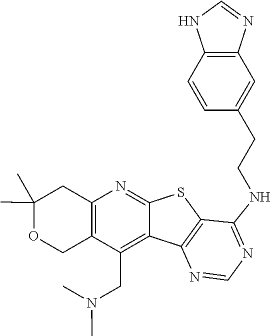 [SMILES: CN(C)Cc1c2c(nc3sc4c(NCCc5ccc6[nH]cnc6c5)ncnc4c13)CC(C)(C)OC2] |
| 335 | 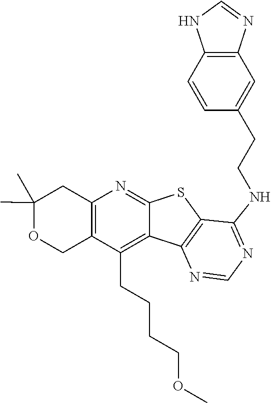 [SMILES: COCCCCc1c2c(nc3sc4c(NCCc5ccc6[nH]cnc6c5)ncnc4c13)CC(C)(C)OC2] |

or its pharmaceutically acceptable salt, stereoisomer, tautomer, hydrate, N-oxide, pharmaceutical product, isotopic variant (e.g. deuterated analogs) or any combination thereof.

58. The compound of claim 53,
wherein the compound destroys SDH-deficient cells, the compound inhibits a GluT receptor, the compound is selective to cells with broken TCA cycle or any combination thereof;
preferably wherein the broken TCA cycle is genetic or chemically induced;
wherein the compound is selective to GluT1, GluT2, GluT3, GluT4, or any combination thereof;
wherein the compound inhibits all classes of GluT receptors;
or any combination thereof.

59. A pharmaceutical composition comprising a compound according to claim 53 and a pharmaceutically acceptable carrier.

60. A method for treating, suppressing, reducing the severity, reducing the risk of developing or inhibiting cancer in a subject, comprising administering a compound according to claim 53 to a subject suffering from cancer under conditions effective to treat, suppress, reduce the severity, reduce the risk of developing, or inhibit said cancer.

61. The method of claim 60,
wherein the cancer is selected from the list of: renal cell carcinoma, leiomyosarcoma, gastrointestinal stromal cancer, paraganglioma (e.g., bladder paraganglioma), pituitary adenoma, pheochromocytoma, colorectal cancer, gastric cancer, ovarian cancer, and/or other cancer types which are characterized by high glycolytic rate;
wherein the cancer is familial, sporadic, early cancer, advanced cancer, invasive cancer, metastatic cancer, drug resistant cancer or any combination thereof;
wherein the subject has been previously treated with chemotherapy, immunotherapy, radiotherapy, biological therapy, surgical intervention, or any combination thereof;
wherein the compound is administered in combination with an anti-cancer therapy, preferably wherein the anti-cancer therapy is chemotherapy, immunotherapy, radiotherapy, biological therapy, surgical intervention, or any combination thereof;
wherein the subject has advanced cancer, metastatic cancer, drug resistant cancer or any combination thereof;
or any combination thereof.

62. A method for suppressing, reducing or inhibiting tumor growth in a subject, comprising administering a compound according to claim 53, to a subject suffering from tumor growth under conditions effective to suppress, reduce or inhibit said tumor growth in said subject.

63. The method of claim 62,
wherein the tumor is selected from the list of: gastrointestinal stromal tumor, pheochromocytoma, pituitary adenoma, pheochromocytoma, leiomyoma (e.g., uterine fibroids), pancreatic neuroendocrine tumor and paraganglioma;
wherein the tumor is benign, invasive, malignant, cancerous, carcinoma, familial, sporadic, or any combination thereof;
wherein the tumor growth is stimulated by a broken TCA cycle, by SDH-deficient cells, by SDH deactivating mutation or any combination thereof;
wherein the tumor growth is suppressed due to destruction of SDH-deficient cells;
or any combination thereof.

64. A method of treating, suppressing, reducing the severity, reducing the risk of developing or inhibiting a disease or condition in a subject,
wherein the disease or condition is selected from:
gastrointestinal stromal tumors (GIST); renal cell carcinoma; paraganglioma; pheochromocytoma (PHEO); pituitary adenoma; colorectal cancer; gastric cancer; ovarian cancer; Leiomyosarcoma; Inflammation; an autoimmune disease; a parasitic or viral infection; diabetes mellitus; diabetic retinopathy; diabetic nephropathy; and metabolic disease;
said method comprises administering a compound according to claim 53, to a subject suffering from said disease or condition under conditions effective to treat, suppress, reduce the severity, reduce the risk of developing, or inhibit said disease or condition.

65. The method of claim 64,
wherein the paraganglioma is familial, sporadic, benign, malignant, and/or bladder paraganglioma;
wherein the pheochromocytoma is familial, sporadic, benign and/or malignant;
wherein the autoimmune disease or disorder is Guillain-Barré syndrome (GBS), Systemic lupus erythematosus (SLE), Rheumatoid arthritis (RA), or any combination thereof;
wherein the parasitic infection is caused by malaria parasite, by HIV or by human cytomegalovirus (CMV);
or any combination thereof.

66. A method of treating, suppressing, reducing the severity, reducing the risk of developing or inhibiting SDH-associated neoplasms in a subject, comprising administering the compound according to claim 53 to a subject suffering from SDH-associated neoplasms under conditions effective to treat, suppress, reduce the severity, reduce the risk of developing, or inhibit the SDH-associated neoplasms in said subject.

67. The method of claim 66, wherein the neoplasms are neuroendocrine neoplasms.

68. A method for inhibiting an SDH-deficient tumor growth in a subject, comprising administering a compound according to claim 53, to a subject under conditions effective to inhibit the growth of said SDH-deficient tumor in said subject.

69. The method of claim 68, wherein the tumor cells have a broken TCA cycle and/or wherein said compound destroys SDH-deficient cells.
